(19) United States
(12) Patent Application Publication
(10) Pub. No.: US 2010/0152053 A1
(43) Pub. Date: Jun. 17, 2010

(54) METHOD FOR IN VITRO MONITORING OF POSTOPERATIVE CHANGES FOLLOWING LIVER TRANSPLANTATION

(75) Inventor: Stefan Russwurm, Jena (DE)

Correspondence Address: PATENT CENTRAL LLC;Stephan A. Pendorf, 1401 Hollywood Boulevard, Hollywood, FL, 33020, US

(73) Assignee: SIRS-LAB GMBH, Jena (DE)

(21) Appl. No.: 12/298,535

(22) PCT Filed: Apr. 11, 2007
(86) PCT No.: PCT/EP2007/002705

§ 371 Date: Nov. 23, 2009

(30) Foreign Application Priority Data
Apr. 26, 2006 (DE) 10 2006 019 480.2

(51) Int. Cl. C12Q 1/68; C40B 30/00; C07H 21/04

(52) U.S. Cl. 506/7; 435/6; 536/23.1

(57) ABSTRACT

The present invention relates to the use of gene expression profiles, obtained in vitro from a patient sample, for detecting any postoperative tissue incompatibility reactions following a liver transplantation, wherein in patients the gene activity of a plurality of specific genes relevant to a liver transplantation is determined in a patient sample, and the specific genes and/or gene fragments for monitoring of postoperative states are selected from the group consisting of: SEQ-ID No. 1 to SEQ-ID No. 532, these preferably being subdivided into several clusters.

The present invention concerns the use of gene expression profiles of samples obtained in vitro from patients for the detection of possible postoperative tissue rejection reactions following a successful liver transplantation according to claim 1, a process for in vitro measurement of a gene expression profile of this type according to claim 13. The invention further concerns a use of the gene expression profile and/or of the therefore employed probe for switching off and/or for changing the activation of target genes and/or for determination of the gene activity for the determination of the risk of rejection in a patient who has received a transplant, according to claim 27, a kit according to claim 30 as well as clusters of polynucleotides according to claims 31 through 34.

The present invention further concerns a new possibility for monitoring postoperative developments following liver transplantations, for the early recognition of a possible risk of rejection.

The liver is one of the most important and largest internal organs in the human body, it is vitally important to almost all metabolic processes (Jecklin, 2004). The liver is not only the most essential organ for metabolism, but rather is also the detoxification central in the human body (Jecklin, 2004; Trebsdorf et al., 1998). However, the liver is liable to a number of diseases. The main causes, besides excessive alcohol consumption, involve bacterial or as the case may be viral infections (e.g. hepatitis) or hereditary factors. Frequently symptoms of a liver illness are diagnosed as everyday aches and pains and are treated too late (www.leber-info.de). Certain liver diseases (Table 1) lead to irreversible damage of the liver. Almost all forms of irreversible liver insufficiency with threat of organ failure will present potential indicators for liver transplantation (Penko and Tirbaso., 1999).

TABLE 1
Typical diseases which determine the necessity
of the liver transplantation.

| | |
|---|---|
| Chronic hepatocellular diseases | Hepatitis B |
| | Hepatitis C |
| | Hepatitis D |
| | Alcoholic cirrhosis of the liver |
| | Autoimmune hepatitis |
| Chronic cholestatic diseases | Primary biliary cirrhosis |
| | Primary sklerotic cholangitis |
| | Biliary Atresia |
| Disfunction of liver metabolism | Haemochromatosis |
| | Wilson Sydrome |
| | Alpha-1-antitrypsin deficiency |
| Congenital metabolic dysfunction | Oxalose Syndrome |
| | Urea cycle defect |

The bolded terms represent the most frequent indicators for liver transplantation (Penko and Tirbaso., 1999).

Damage due to liver disease as well as a late diagnosis could lead to liver failure. Liver failures have a high mortality and influence a variety of other organs (Bauer et al., 2005). Despite a high mortality due to liver failure, liver transplantation remains the preferred method of treatment, in the case that specific indications, such as, for example, King's College or Clichy criteria, prognosticate an unfavorable course of the disease or condition (Bauer et al., 2005). In the year 2003 in Germany alone there were 11,797 patients who were waiting for a donor organ, among these were 1266 persons with an urgent need for liver transplantation (www.dso.de). The problem of the long waiting lists due to the lack of sufficiently large pool of suitable donor organs has in no way been solved despite the efforts from many sides and the development of a European-wide donor organ notification center. The allogenic transplantation must be accompanied by a lifelong immunosuppression therapy, which suppresses the hyperacute, acute or chronic rejection reactions. Besides this, the relevant patients are often burdened with a reinfection, in particular with a viral infection such as Epstein-Barr or cytomegalovirus (CVM) infection (Gao and Zheng, 2004). Added to this are severe complications primarily in the early postoperative course, in particular failure of the transplanted liver and acute rejection crises. The postoperative rejection rate in liver transplantations is approximately 36% (Shimizu et al., 2003). Initial intensive medical monitoring and closely meshed postoperative care are thus of particular importance for the concerned patients (Müller and Neuhaus, 2004).

It is thus the task of the present invention to provide possibilities for in vitro diagnoses, which allow postoperative assessment as to whether a patient's transplanted donor liver is accepted or not and, in particular, in certain cases, prognosis for the occurrence of rejection reactions and for the early implementation of therapeutic measures.

The above task is solved by the uses according to claim 1 and claim 27.

Procedurally the task is solved by claim 13.

A kit according to claim 30 as well as clusters of polynucleotides according to claims 31 through 34 likewise solve the task.

The task is in particular solved by a use of gene expression profiles obtained in vitro from a patient sample for the determination of possible postoperative occurring tissue compatibility reactions following a successful liver transplantation.

The invention further concerns the evaluation of the accompanying therapeutic treatment of patients who have had a liver transplantation. Further the invention makes possible the use of gene expression profiles for the determination of the risk of rejection in a patient who has had a liver transplant.

The present invention can also be used for production of “in silico” expert systems and/or for “in silico”—modeling of cellular signal transmission pathways.

For producing the gene expression profile according to the present invention a plurality of specific genes and/or gene fragments are employed, which are selected from the group in Tables 6-9 comprised of SEQ-ID No. 1 through SEQ-ID No. 532 as well as gene fragments thereof with at least 5-2000, preferably 20-200, more preferably 20-80 nucleotides.

These sequences with the Sequence ID: 1 through Sequence ID: 532 are included within the scope of the present invention and are individually disclosed in the attached sequence protocol comprising 532 sequences, which is thus a component of the description of the present invention, and which is therewith likewise a component of the disclosure of the invention.

Beyond this, the present invention concerns the use of gene expression profiles obtained in vitro from patient samples and/or the therefore employed probes, which are selected from the group comprised of SEQ-ID No. 1 through SEQ-ID No. 532 as well as gene fragments thereof with at least 5-2000, preferably 20-200, more preferably 20-80 nucleotides, for switching off and/or for changing the activation of target genes and/or for determining the gene activity for monitoring postoperative changes following liver transplantation and/or for determining the gene activity for determining the rejection risk of a liver transplant patient.

A further embodiment of the invention is comprised in the generation of gene activity clusters, wherein, in the respective clusters, at least two genes and/or gene fragments from SEQ-ID No. 1 through SEQ-ID No. 532 are grouped, of which the expression profile remain similar over the most diverse patient samples or probes. In a further embodiment these gene activity clusters are compared with each other and make possible therewith a prediction regarding the postoperative changes of patients with liver transplants.

A further embodiment of the invention is characterized thereby, that a specific gene and/or gene fragment is selected from the group comprised of SEQ-ID No. 1 through SEQ-ID No. 532 as well as gene fragments thereof with at least 5-2000, preferably 20-200, more preferably 20-80 nucleotides.

A further embodiment of the invention is thereby characterized, that at least 2 to 100 different cDNAs are employed.

A further embodiment of the invention is thereby characterized, that at least 200 different sequences are employed.

A further embodiment of the invention is thereby characterized, that least 200 to 500 different sequences are employed.

A further embodiment of the invention is thereby characterized, that at least 500 different sequences are employed.

A further embodiment of the invention is thereby characterized, that the genes or gene fragments listed in claim 3 and/or of their RNA derived sequences are replaced by synthetic analogs, aptamers, as well as peptidonucleic acids.

A further embodiment of the invention is thereby characterized, that the synthetic analogs of the genes comprise 5-100, in particular approximately 70 base pairs.

A further embodiment of the invention is thereby characterized, that the gene activities are determined by a hybridization process.

A further embodiment of the invention is thereby characterized, that the gene activity is determined using microarrays.

A further embodiment of the invention is thereby characterized, that the gene activity is determined by hybridization dependent processes, in particular enzymatic and/or chemical hydrolysis and/or amplification processes, preferably PCR, subsequent quantification of the nucleic acids and/or of derivatives and/or fragments thereof.

A further embodiment of the invention is thereby characterized, that the sample is selected from: body fluids, in particular blood, serum, urine, ascites fluid, seminal fluid, saliva, aspirate; cellular contents or a mixture thereof.

A further embodiment of the invention is thereby characterized, that cell samples are in certain cases subjected to a lytic treatment, in order to release their cellular contents.

It is apparent to the person of ordinary skill in the art that the individual characteristics of the invention set forth in the claims can be combined freely with each other without reducing the scope of the invention.

As marker gene in the sense of the invention, all derived DNA-sequences, partial sequences and synthetic analogs (for example peptid nucleic acids, PNA) are understood. The description of the invention with reference to the determination of the gene expression on the RNA-level is not to be construed as a limitation but rather only one exemplary embodiment.

The description of the invention using blood provides only one example for the application of the invention. As biologic fluids in the sense of the invention, all body fluids of the human are to be understood.

For the purposes of a complete disclosure of the present teaching it has been determined that the originally filed claims individually and/or in combination are part of the disclosure for the person of ordinary skill. In particular the use according to one of claims 1 through 12 and a process according to one of claims 13 through 26 and/or for carrying out a process according to one of claims 13 through 26 are disclosed.

Further advantages and characteristics of the present invention can be seen on the basis of the description of an illustrated embodiment as well as on the basis of the figure.

There is shown:

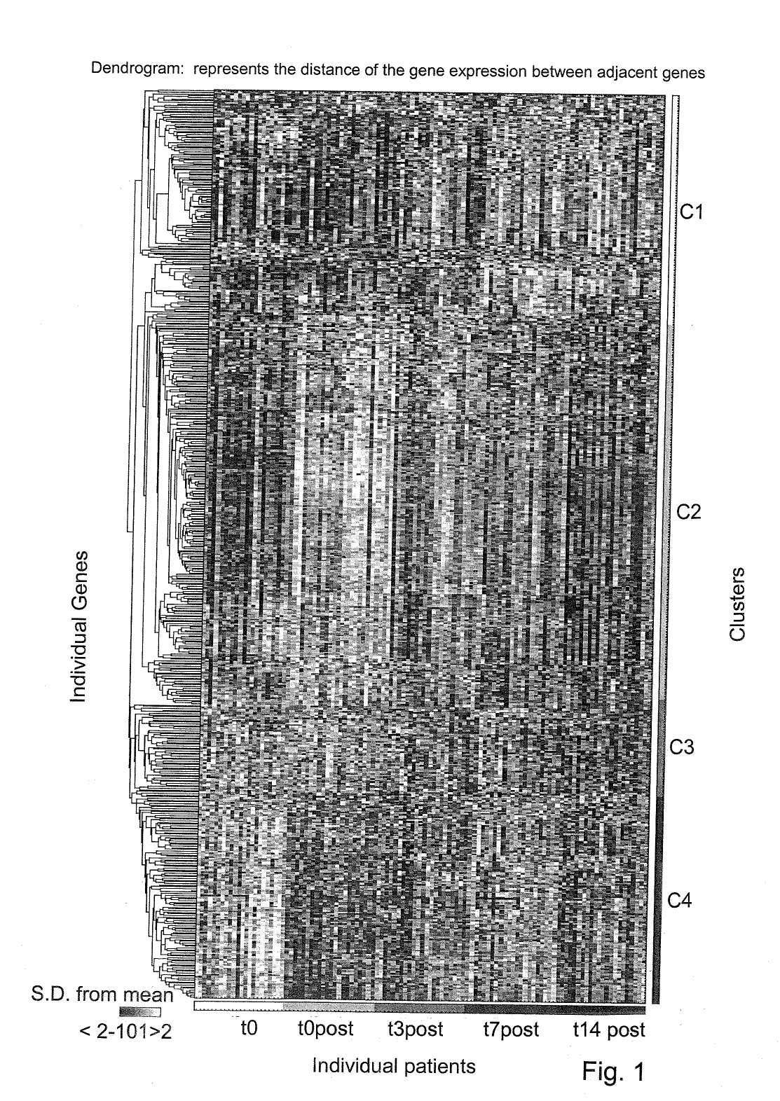

FIG. 1: a color representation of similarities of expression behavior of certain gene associated gene clusters; and

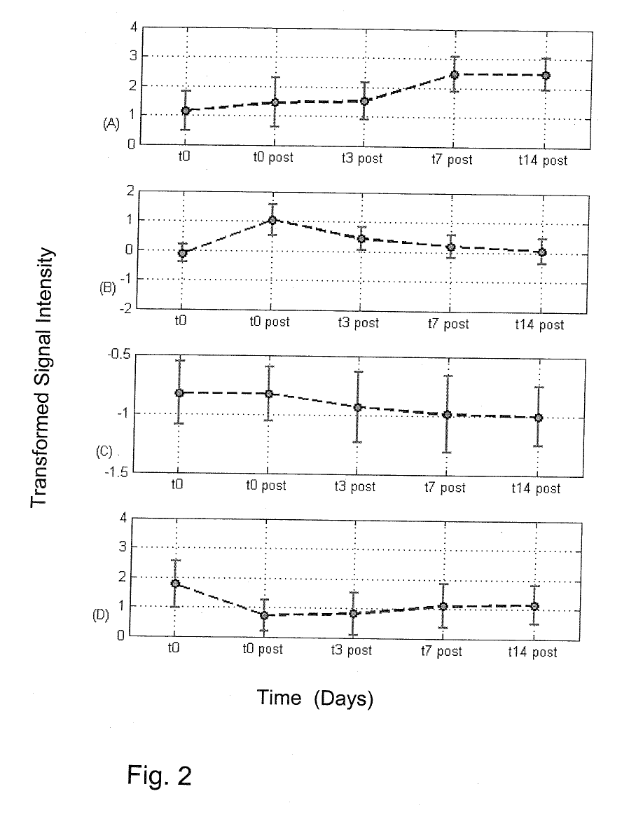

FIG. 2: typical representations of 4 gene activity clusters with specific expression profiles.

### ILLUSTRATIVE EMBODIMENT

For measuring the differential gene expression for monitoring postoperative changes following liver transplantation tests were carried out using full blood samples from a total of 22 patients, who were being treated on operative intensive care stations.

The illustrative embodiment is explained on the basis of the following illustration:
FIG. 1 shows the associated gene cluster for similarities of the expression behavior of certain genes between individual samples.
   Each point of the color matrix represents a relative expression of a gene in a blood sample in relationship to a control, in the case of the example Sig-M5, (vertical: patient sample with increasing identification number; horizontal: 532 examined genes). The respective colors represent the expression of the individual genes, wherein red indicates relative expression greater than the average and green relative expression indicates smaller than the average values; gray fields indicate missing values.
FIG. 2 is concerned with typical representations of the 4 gene activity clusters with specific expression profiles.
   The first gene activity cluster is represented by the gene PPBP (Sequence No. 112) (A); the second gene activity cluster by gene MKNK1 (Sequence No. 220) (B), the third gene activity cluster by the gene IL22RA2 (Sequence No. 3791 (C) and the fourth gene activity duster by the gene IL2RB (Sequence No. 477) (D). The points connected by the dashed line represent the group average value. The gray perpendicular lines mark the average scatter region within the patient group.

The first blood sample was taken immediately prior to the surgical intervention (t0). Directly following the interoperative phase (t0 post) or as the case may be following three (t3 post), seven (t7 post) and fourteen days (t14 post) further postoperative blood samples were obtained from all patients. Following collection of whole blood the total RNA of the sample was isolated using the PAXGene Blood RNA Kit according to the instructions of the manufacturer (Qiagen).

As reference sample there was employed the total RNA from the commercial obtainable monoblast leukamine cell line Sig-M5.

Depending upon the employed method for determination of the gene expression profile other control/reference sample/calibration controls, for example housekeeping or spike sequences, could be used.

### Cell Cultivation

For cell cultivation (control samples) 19 cryocell cultures (SIG-M5) (frozen in liquid nitrogen) were used. The cells were respectively inoculated with 2 ml Isocove's Medium (Biochrom AG) and supplemented with 20% fetal caliber serum (FCS). The cell cultures were subsequently incubated for 24 hours at 37° C. under 5% CO₂ in 12-well plates. Thereafter the content of 18 wells were separated into 2 parts with respectively the same volume, so that in the end 3 plates of the same format (total 36 wells) were available. The cultivation was subsequently carried out for 24 hours using the same conditions. Subsequent thereto the resulting cultures of 11 wells of each plate were combined and centrifuged (1000×g, 5 min, room temperature). The supernatant was discarded and the cell pellet dissolved in 40 ml of the abovementioned medium. These cells dissolved in 40 ml were divided into two 250 ml flasks of equal parts and after 48 hours incubation and addition of 5 ml of above medium again incubated. From the remaining 2 ml of the two remaining plates 80 μl were introduced into empty wells of the same plates, which were already prepared with 1 ml of the above medium. After 48 hours incubation only one of the 12 well-plates was processed as follows: 500 μl were extracted from each well and combined. The therefrom resulting 6 ml were introduced into a 250 ml flask, which contained approximately 10 ml fresh medium. This mixture was centrifuged at 1000×g for 5 minutes at room temperature and dissolved in 10 ml of the abovementioned medium. The subsequent cell count produced the following results: 1.5×10⁷ cells per ml, 10 ml total volume, total number of cells: 1.5×10⁸. Since the cell count was not yet sufficient, 2.5 ml of the above cell suspension in 30 ml of the abovementioned medium were introduced into a 250 ml (75 cm²) flask (total 4 flasks). After 72 hours incubation time respectively 20 ml of fresh medium were introduced into the flasks. After the following 24 hour incubation there occurred a cell count as described above, which resulted in a total of 3.8×10⁸ cells. In order to reach the desired cell count of 2×10⁶ cells the cells were resuspended in 47.5 ml of the abovementioned medium in 4 flasks. After an incubation time of 24 hours the cells were centrifuged and twice washed with phosphate buffer without Ca²⁺ and Mg²⁺ (Biochrom AG).

The isolation of the total-RNA was carried out using the NucleoSpin RNA L Kit (Machery&Nagel) according to the instructions of the manufacturer. The above described procedure was repeated until the required cell count was achieved. This was necessary in order to reach the required amount of 6 mg total-RNA, which corresponded to a yield of 600 μg RNA per 10⁸ cells.

The general data for the patients could be seen from Table 2 and the preoperative data from Table 3.

TABLE 2
General clinical data
General Data of the Patients

| Age [Years] | 54.5 (7.75)¹ |
|---|---|
| Sex [m/f] | 16/6 |
| Body Mass Index (BMI) [kg/m] | 26 (4)¹ |
| Number of the combined transplants (Kidney/Liver) | 2 |
| Number of transplants: Complete organ/Split LT: | 22/0 |

¹Values as medians and the values in parentesis represent the difference of the 75% - and 25%-percentile.

TABLE 3
Pre-operative Patient data

| Test results | Units | Data |
|---|---|---|
| Prothrombin time | [%] | 67.5 (29.5) |
| Leukocyte | [Number/μl] | 5350 (3925) |
| Base deviation | [mmol/l] | −1 (5.1) |
| Fibrinogen | [μmol/l] | 2.6 (2) |
| C-reaktive protein | [mg/l] | 14 (13) |
| Creatinine | [μmol/l] | 81 (39.25) |
| Thrombocytes | [Number/μl] | 100 (142.75) |
| | [×10³] | |
| Red bloodcells | [Number/μl] | 4 (1) |
| Aspartate amino transferase | [U/l] | 1 (1) |
| Alanine amino transferase | [U/l] | 1 (0) |
| Direct bilirubin | [μmol/l)] | 12 (18.5) |
| Total bilirubin | [μmol/l] | 32.5 (45.75) |
| γ-Glutamyltransferase | [U/l] | 1 (1) |
| Prothrombin ratio | Ratio | 1 (0) |
| Partial prothrombin time | [s] | 33 (10) |
| Albumin | [g/l] | 31 (13) |
| Cholinesterase | [U/l] | 46 (23) |
| Urea | [mmol/l] | 6 (3.5) |
| Glucose | [mmol/l] | 6 (3) |
| Lactate | (max) [mmol/l] | 1.9 (1) |

The intra-operative and post-operative data of the transplants are demonstrated in the following tables (Table 4; Table 5).

TABLE 4
Intraoperative Data of the patient cohort.
Intraoperative Data

| | |
|---|---|
| Duration of liver ischaemia after the explanation [min] | 289.5 (208.75) |
| Duration of the surgery [min] | 286.5 (128.75) |
| Duration of the anhepatic phase [min] | 40 (21.75) |
| Blood products: Blood transfusion (EK/24 h [n]): | 9.5 (6.75) |
| Plasma transfusion (FFP/24 h [n]): | 26 (27.25) |
| Thrombocyte concentrate (TK/24 h [n]): | 2 (0) |

All values are given as medians and the values in parentesis represent the difference of the 75%- and 25%-percentile.

TABLE 5
Postoperative characterization of the patient cohort.
Postoperative Data

| | |
|---|---|
| Stay in the Intensive Care Unit [Days] | 6 (3.5) |
| Stay in the hospital [Days] | 26.5 (15) |

All values are given as medians and the values in parentesis represent the difference of the 75%- and 25%-percentile (quantile).

All patient samples were cohybridized on a microarray respectively with the reference sample.

### Reverse Transcription/Marking/Hybridization

After collecting whole blood the total-RNA of the samples was isolated using the PAXGene Blood RNA Kit (PreAnalytiX) according to the instructions of the manufacturer and tested for their quality. From each sample 8 μg total-RNA were aliquoted and transcribed together with 10 μg total-RNA from SIGM5-cells as reference-RNA to complimentary DNA (cDNA) with reverse transcriptase Superscript II (Invitrogen) and the RNA subsequently removed from the preparation by alkaline hydrolysis. In the reaction preparation a part of the dTTP was replaced by aminoallyl-dUTP (AA-dUTP), in order later to make possible the coupling of the fluorescent dye to the cDNA.

After the purification of the reaction preparation with the cDNA of the samples and controls were covalently marked (dye swap) with the fluorescent dye DY-547 and DY-647 of the company Dyomics and hybridized on the microarray of the company SIRS-Lab. On the employed microarray there were 5308 immobilized polynucleotides with a length of 55-70 base pairs, with respectively represented one human gene and control spots for quality assurance. The microarray is subdivided into 28 subarrays with a grid of 15×15 spots.

The hybridization and the subsequent washing or, as the case may be, drying, was carried out in the hybridization station HS 400 (Tecan) according to the instructions of the manufacturer for 10.5 hours at 42° C. The employed hybridization solution was comprised of the respective labeled cDNA-samples, 3.5×SSC (1×SSC containing 150 mM sodium chloride and 15 mM sodium citrate), 0.3% sodium dodecyl sulfate (V/V) 25% formamide (V/V) and respectively 0.8 μg μl-1 cot-1 DNA, yeast t-RNA and poly-A RNA. The subsequent washing of the microarrays was carried out according to the following procedure at room temperature: respectively 90 second rinsing with wash buffer 1 (2×SSC, 0.03% sodium dodecyl sulfate), with wash buffer 2 (1×SSC) and subsequently with wash buffer 3 (0.2×SSC). Thereafter the microarrays were dried under a stream of nitrogen at a pressure of 2.5 bar at 30° C. over 150 seconds.

### Evaluation

Following hybridization the hybridization signals of the microarrays were read out using a GenePix 4000B scanner (Axon) and the expression relationships of the differentially expressed genes determined with the software GenePix Pro 4.0 (Axon). For evaluation, the average intensity of the spot was determined as the median value for the associated spot pixel.

### Correction of Systematic Errors:

The correction of systematic errors occurred according to the approach of Huber and others (Huber et al., 2003). Wherein the additive and the multiplicative bias within a microarray was estimated from 70% of the available gene samples. For all further computations the signals were transformed using arcus sinus hyberbolicus.

### Biostatic Evaluation of Microarrays:

The goal of the data analysis was the extraction of gene expression patterns, which are characteristic for an operational intervention in a liver transplant. Building on the work of Tomic among others (Tomic V., et al., 2005) the analysis was carried out in three steps:

1. Step: In the data analysis those gene samples were included of which the signals were measured with sufficient intensity. Therein the average spot quality was evaluated within the total experiment. Thereafter 4176 samples were considered in the analysis. The resulting data matrix contained approximately 2.3% missing values, which were replaced by estimated values (compare Toyanskaya among others (Troyanskaya, et al., 2001)).
2. Step: Utilizing a suitable statistical test, genes were identified, which changed in similar manner in all patients due to the transplantation. For this, gene for gene, the multivariant single entry sample PC-test according to Laeuter (Laeuter, 1996) was employed, which forms a generalization of the simple t-test. As a result each gene was assigned a p-value, which reflected or represented the statistical difference in the time variation of the expression of the respective gene. In order to determine a threshold of significance, which makes possible a selection of differentially expressed genes, the concept of Storey and Tibshirani was selected (Storey and Tibshirani, 2003). Therein the proportion of the false positive decisions (in English false discovery rate) was estimated with the aid of the so-called q-value. 532 samples with a q-value smaller than 0.05 were inclusion criteria for the final gene list. The threshold was so defined, that an optimal relationship of the discovered significant genes to the associated proportion of the false positives was achieved. According to this evaluation the final list contained approximately 5% (˜26 of 532) false positive samples.
3. Step: In the statistical comparison genes were identified, which as a result of the transplantation were significantly changed in the same manner in all patients. In order to obtain characteristic patterns of the gene expression, the results of the selected 532 expressed genes were arranged with the aid of a hierarchical cluster algorithm and presented in a corresponding pattern. In the cluster algorithm the correlation was selected as the distance dimension. The “average linkage”—methodology served as the basis for the establishment of the associated dendrogram. In the representation of the associated gene cluster the similarities of the expression behavior of particular genes between individual samples was displayed FIG. 1. The tabulated list of the genes with the corresponding average expression values were compiled in Tables 6-9.

In the gene cluster a distribution of the genes in four similar gene groups with differentiated expressed gene expressions were discovered, which exhibited the following biologic interpretable expression changes:

The first group of 132 gene samples, which did not have or had only slightly relationship to the operation, which were expressed following the third or as the case may be the seventh postoperative day (Table 6).

The second group of the 221 gene samples, which reacted with early postoperative elevated gene expressions, reverted after 14 days almost completely to the original gene expression level (Table 7).

The third group of 57 gene samples, which reacted to the transplantation with a small expression magnitude beginning at the third postoperative day (Table 8).

The fourth cluster included approximately 122 genes, which were distributed in counterflow in the second cluster and exhibited a reduced expression after the first postoperative day. Thereafter however an increasing gene activity was to be recognized, which however did not return to the preoperative value after 14 days (Table 9).

Typical representations with specific expression plots are shown in FIG. 2.

TABLE 6
Cluster 1

| Sequence | PC-Test | | Median (±Standard deviation) | | | | | | | | | |
|---|---|---|---|---|---|---|---|---|---|---|---|---|
| No. | p-value | q-value | t0 | | t0 post | | t3 post | | t7 post | | t14 post | |
| 1 | 0.01 | 0.05 | 0.14 | (±0.37) | 0.48 | (±0.59) | 0.14 | (±0.54) | 0.27 | (±0.32) | 0.46 | (±0.45) |
| 2 | 0.00 | 0.02 | −0.11 | (±0.24) | 0.15 | (±0.21) | −0.09 | (±0.22) | −0.05 | (±0.24) | −0.05 | (±0.23) |
| 3 | 0.01 | 0.03 | 0 | (±0.35) | 0.02 | (±0.31) | 0.3 | (±0.47) | 0.24 | (±0.35) | 0.27 | (±0.42) |
| 4 | 0.01 | 0.05 | 2.01 | (±0.56) | 1.97 | (±0.48) | 2.14 | (±0.38) | 1.69 | (±0.7) | 2.16 | (±0.43) |
| 5 | 0.01 | 0.03 | 1.72 | (±0.49) | 1.78 | (±0.5) | 1.83 | (±0.48) | 1.45 | (±0.65) | 1.94 | (±0.43) |
| 6 | 0.01 | 0.03 | 0.54 | (±0.27) | 0.57 | (±0.3) | 0.62 | (±0.26) | 0.53 | (±0.31) | 0.74 | (±0.28) |
| 7 | 0.00 | 0.00 | 0.47 | (±0.24) | 0.93 | (±0.39) | 0.67 | (±0.36) | 0.82 | (±0.36) | 0.94 | (±0.39) |
| 8 | 0.01 | 0.05 | −0.22 | (±0.35) | −0.28 | (±0.36) | −0.29 | (±0.4) | 0 | (±0.65) | 0.11 | (±0.57) |
| 9 | 0.00 | 0.02 | −0.94 | (±0.43) | −0.6 | (±0.34) | −0.63 | (±0.39) | −0.56 | (±0.47) | −0.7 | (±0.46) |
| 10 | 0.00 | 0.00 | −0.75 | (±0.62) | −0.41 | (±0.57) | −0.4 | (±0.43) | −0.35 | (±0.41) | −0.83 | (±0.52) |
| 11 | 0.00 | 0.01 | −0.55 | (±0.2) | −0.36 | (±0.21) | −0.52 | (±0.23) | −0.41 | (±0.27) | −0.56 | (±0.27) |
| 12 | 0.00 | 0.00 | −0.1 | (±0.24) | −0.03 | (±0.29) | 0.28 | (±0.32) | 0.01 | (±0.36) | 0.15 | (±0.27) |
| 13 | 0.00 | 0.01 | −0.05 | (±0.28) | 0.05 | (±0.32) | 0.24 | (±0.35) | −0.04 | (±0.25) | 0.22 | (±0.35) |
| 14 | 0.00 | 0.01 | 0.18 | (±0.32) | −0.13 | (±0.43) | 0.13 | (±0.38) | 0.5 | (±0.41) | 0.22 | (±0.44) |
| 15 | 0.00 | 0.00 | −1.29 | (±0.46) | −1.82 | (±0.4) | −1.49 | (±0.39) | −1.2 | (±0.31) | −1.61 | (±0.33) |
| 16 | 0.00 | 0.03 | −0.04 | (±0.41) | −0.33 | (±0.56) | 0.13 | (±0.52) | 0.14 | (±0.39) | 0.06 | (±0.46) |
| 17 | 0.00 | 0.00 | 0.17 | (±0.33) | −0.21 | (±0.34) | −0.26 | (±0.28) | 0.04 | (±0.35) | −0.04 | (±0.29) |
| 18 | 0.00 | 0.01 | −0.35 | (±0.33) | −0.5 | (±0.2) | −0.25 | (±0.22) | −0.17 | (±0.24) | −0.25 | (±0.29) |
| 19 | 0.00 | 0.01 | −0.14 | (±0.45) | −0.41 | (±0.42) | −0.11 | (±0.26) | −0.07 | (±0.25) | −0.02 | (±0.25) |
| 20 | 0.00 | 0.02 | −0.85 | (±0.35) | −0.85 | (±0.24) | −0.75 | (±0.18) | −0.64 | (±0.21) | −0.65 | (±0.3) |
| 21 | 0.01 | 0.04 | −1.15 | (±0.53) | −0.94 | (±0.33) | −0.9 | (±0.3) | −0.66 | (±0.32) | −0.86 | (±0.34) |
| 22 | 0.01 | 0.04 | −0.65 | (±0.3) | −0.76 | (±0.27) | −0.64 | (±0.24) | −0.48 | (±0.28) | −0.57 | (±0.27) |
| 23 | 0.01 | 0.04 | −0.38 | (±0.3) | −0.66 | (±0.19) | −0.5 | (±0.18) | −0.41 | (±0.21) | −0.49 | (±0.4) |
| 24 | 0.01 | 0.04 | −0.64 | (±0.57) | −0.72 | (±0.44) | −0.55 | (±0.45) | −0.27 | (±0.41) | −0.39 | (±0.49) |
| 25 | 0.01 | 0.04 | −0.75 | (±0.3) | −0.97 | (±0.27) | −0.84 | (±0.31) | −0.69 | (±0.2) | −0.76 | (±0.24) |
| 26 | 0.01 | 0.04 | −1.08 | (±0.34) | −1.39 | (±0.21) | −1.35 | (±0.34) | −1.22 | (±0.35) | −1.29 | (±0.42) |
| 27 | 0.00 | 0.00 | −0.29 | (±0.27) | −0.56 | (±0.19) | −0.46 | (±0.26) | −0.27 | (±0.18) | −0.31 | (±0.3) |
| 28 | 0.00 | 0.00 | −1.88 | (±0.32) | −2.16 | (±0.24) | −2.11 | (±0.25) | −1.91 | (±0.3) | −1.94 | (±0.27) |
| 29 | 0.00 | 0.02 | −0.68 | (±0.31) | −0.87 | (±0.27) | −0.87 | (±0.32) | −0.64 | (±0.17) | −0.71 | (±0.29) |
| 30 | 0.00 | 0.00 | −1.07 | (±0.41) | −1.48 | (±0.44) | −1.21 | (±0.4) | −0.89 | (±0.28) | −1.1 | (±0.48) |
| 31 | 0.00 | 0.00 | −0.76 | (±0.42) | −1.01 | (±0.3) | −0.93 | (±0.36) | −0.53 | (±0.26) | −0.6 | (±0.57) |
| 32 | 0.00 | 0.02 | −0.28 | (±0.26) | −0.38 | (±0.37) | −0.16 | (±0.31) | −0.07 | (±0.24) | −0.09 | (±0.34) |
| 33 | 0.01 | 0.04 | −0.05 | (±0.38) | −0.14 | (±0.2) | −0.07 | (±0.25) | 0.04 | (±0.16) | 0.02 | (±0.33) |
| 34 | 0.00 | 0.03 | −0.14 | (±0.35) | −0.34 | (±0.31) | −0.13 | (±0.34) | −0.04 | (±0.26) | 0.1 | (±0.3) |
| 35 | 0.00 | 0.00 | 0.23 | (±0.3) | 0.33 | (±0.41) | 0.43 | (±0.42) | 0.63 | (±0.4) | 0.7 | (±0.41) |
| 36 | 0.00 | 0.02 | −0.84 | (±0.34) | −0.9 | (±0.24) | −0.77 | (±0.26) | −0.72 | (±0.2) | −0.66 | (±0.28) |
| 37 | 0.00 | 0.02 | 0.9 | (±0.57) | 1.17 | (±0.44) | 1.38 | (±0.39) | 1.28 | (±0.53) | 1.49 | (±0.56) |
| 38 | 0.00 | 0.03 | 0.25 | (±0.36) | 0.27 | (±0.24) | 0.4 | (±0.24) | 0.46 | (±0.19) | 0.43 | (±0.31) |
| 39 | 0.00 | 0.02 | −0.33 | (±0.43) | −0.51 | (±0.44) | −0.24 | (±0.44) | −0.06 | (±0.41) | −0.1 | (±0.3) |
| 40 | 0.00 | 0.01 | −0.61 | (±0.32) | −0.73 | (±0.3) | −0.61 | (±0.32) | −0.29 | (±0.37) | −0.46 | (±0.36) |
| 41 | 0.01 | 0.03 | −0.63 | (±0.25) | −0.62 | (±0.18) | −0.55 | (±0.29) | −0.42 | (±0.29) | −0.44 | (±0.33) |
| 42 | 0.01 | 0.04 | −2.27 | (±0.18) | −2.35 | (±0.21) | −2.37 | (±0.22) | −2.14 | (±0.23) | −2.16 | (±0.29) |
| 43 | 0.00 | 0.01 | −0.45 | (±0.38) | −0.49 | (±0.33) | −0.26 | (±0.55) | −0.18 | (±0.38) | −0.01 | (±0.56) |
| 44 | 0.00 | 0.01 | −0.36 | (±0.21) | −0.28 | (±0.16) | −0.18 | (±0.29) | −0.14 | (±0.25) | −0.05 | (±0.28) |
| 45 | 0.01 | 0.04 | −0.07 | (±0.35) | −0.02 | (±0.33) | 0.05 | (±0.37) | 0.13 | (±0.38) | 0.24 | (±0.39) |
| 46 | 0.00 | 0.01 | 0.18 | (±0.29) | 0.24 | (±0.23) | 0.36 | (±0.39) | 0.39 | (±0.34) | 0.49 | (±0.28) |
| 47 | 0.00 | 0.02 | −0.07 | (±0.32) | −0.25 | (±0.35) | 0.01 | (±0.4) | 0.04 | (±0.37) | 0.16 | (±0.31) |
| 48 | 0.00 | 0.03 | −0.34 | (±0.45) | −0.27 | (±0.37) | −0.1 | (±0.27) | −0.02 | (±0.27) | −0.03 | (±0.24) |
| 49 | 0.00 | 0.00 | −1.23 | (±0.38) | −1.27 | (±0.33) | −1.2 | (±0.41) | −0.89 | (±0.39) | −0.82 | (±0.56) |
| 50 | 0.00 | 0.02 | −1.02 | (±0.27) | −1.28 | (±0.32) | −1.09 | (±0.28) | −0.92 | (±0.27) | −0.93 | (±0.38) |
| 51 | 0.00 | 0.00 | −0.36 | (±0.31) | −0.69 | (±0.27) | −0.59 | (±0.35) | −0.44 | (±0.31) | −0.3 | (±0.39) |
| 52 | 0.00 | 0.01 | −0.1 | (±0.32) | −0.19 | (±0.39) | −0.05 | (±0.33) | 0.07 | (±0.27) | 0.14 | (±0.3) |
| 53 | 0.01 | 0.04 | 0.5 | (±0.4) | 0.4 | (±0.53) | 0.68 | (±0.43) | 0.65 | (±0.29) | 0.78 | (±0.47) |
| 54 | 0.01 | 0.05 | −0.44 | (±0.3) | −0.57 | (±0.31) | −0.42 | (±0.36) | −0.32 | (±0.28) | −0.26 | (±0.31) |
| 55 | 0.00 | 0.00 | −0.02 | (±0.36) | 0.09 | (±0.29) | 0.18 | (±0.26) | 0.26 | (±0.25) | 0.34 | (±0.23) |
| 56 | 0.00 | 0.02 | −0.68 | (±0.55) | −0.65 | (±0.41) | −0.52 | (±0.44) | −0.32 | (±0.35) | −0.39 | (±0.38) |
| 57 | 0.00 | 0.02 | −0.17 | (±0.25) | −0.42 | (±0.34) | −0.16 | (±0.28) | −0.07 | (±0.29) | −0.07 | (±0.21) |
| 58 | 0.01 | 0.03 | 0.26 | (±0.38) | 0.2 | (±0.31) | 0.38 | (±0.34) | 0.47 | (±0.35) | 0.52 | (±0.37) |
| 59 | 0.00 | 0.00 | −0.45 | (±0.51) | −0.47 | (±0.55) | −0.16 | (±0.39) | 0.15 | (±0.44) | 0.27 | (±0.45) |
| 60 | 0.00 | 0.02 | −0.16 | (±0.48) | −0.28 | (±0.47) | 0.05 | (±0.34) | 0.28 | (±0.48) | 0.28 | (±0.52) |
| 61 | 0.00 | 0.00 | −0.23 | (±0.45) | −0.26 | (±0.35) | −0.05 | (±0.35) | 0.15 | (±0.32) | 0.11 | (±0.4) |
| 62 | 0.00 | 0.00 | −0.94 | (±0.44) | −1.05 | (±0.27) | −0.82 | (±0.36) | −0.57 | (±0.35) | −0.66 | (±0.35) |
| 63 | 0.01 | 0.05 | −1.01 | (±0.41) | −1.06 | (±0.37) | −0.98 | (±0.35) | −0.73 | (±0.34) | −0.85 | (±0.39) |
| 64 | 0.00 | 0.00 | −0.84 | (±0.46) | −1.02 | (±0.49) | −0.82 | (±0.49) | −0.46 | (±0.44) | −0.53 | (±0.45) |
| 65 | 0.00 | 0.00 | −0.81 | (±0.66) | −0.86 | (±0.7) | −0.58 | (±0.62) | −0.27 | (±0.59) | −0.33 | (±0.63) |
| 66 | 0.00 | 0.01 | −0.88 | (±0.63) | −0.92 | (±0.62) | −0.68 | (±0.6) | −0.45 | (±0.55) | −0.51 | (±0.6) |
| 67 | 0.00 | 0.00 | −1.06 | (±0.51) | −1.18 | (±0.49) | −0.93 | (±0.52) | −0.66 | (±0.53) | −0.61 | (±0.62) |
| 68 | 0.00 | 0.01 | −1.11 | (±0.34) | −1.18 | (±0.3) | −1.05 | (±0.38) | −0.83 | (±0.42) | −0.87 | (±0.39) |
| 69 | 0.00 | 0.02 | −0.18 | (±0.52) | −0.33 | (±0.41) | −0.12 | (±0.5) | 0.03 | (±0.38) | 0.18 | (±0.59) |
| 70 | 0.00 | 0.01 | 0.08 | (±0.6) | 0 | (±0.3) | 0.23 | (±0.44) | 0.4 | (±0.42) | 0.5 | (±0.46) |
| 71 | 0.00 | 0.01 | −0.67 | (±0.38) | −0.68 | (±0.39) | −0.56 | (±0.41) | −0.35 | (±0.36) | −0.36 | (±0.41) |
| 72 | 0.00 | 0.00 | 0.57 | (±0.79) | 0.54 | (±0.79) | 1.16 | (±1.04) | 1.42 | (±0.84) | 1.81 | (±0.86) |
| 73 | 0.00 | 0.00 | −0.32 | (±0.84) | −0.5 | (±0.75) | 0.07 | (±0.95) | 0.41 | (±0.77) | 0.62 | (±0.81) |
| 74 | 0.00 | 0.01 | −0.49 | (±0.5) | −0.4 | (±0.49) | −0.2 | (±0.33) | 0.04 | (±0.43) | 0.02 | (±0.55) |
| 75 | 0.00 | 0.01 | 0.23 | (±0.56) | 0.09 | (±0.59) | 0.33 | (±0.48) | 0.66 | (±0.49) | 0.61 | (±0.59) |
| 76 | 0.00 | 0.00 | −0.05 | (±0.66) | −0.16 | (±0.63) | 0.1 | (±0.56) | 0.34 | (±0.43) | 0.56 | (±0.64) |
| 77 | 0.00 | 0.03 | −0.33 | (±0.48) | −0.51 | (±0.44) | −0.28 | (±0.54) | −0.11 | (±0.39) | −0.05 | (±0.39) |
| 78 | 0.00 | 0.01 | −0.71 | (±0.52) | −0.77 | (±0.57) | −0.59 | (±0.45) | −0.3 | (±0.36) | −0.38 | (±0.43) |
| 79 | 0.00 | 0.00 | −1.81 | (±0.46) | −2.02 | (±0.44) | −1.83 | (±0.35) | −1.54 | (±0.48) | −1.56 | (±0.54) |
| 80 | 0.00 | 0.00 | −2.04 | (±0.54) | −2.39 | (±0.51) | −2.1 | (±0.43) | −1.74 | (±0.59) | −1.69 | (±0.6) |
| 81 | 0.00 | 0.01 | −0.02 | (±0.21) | 0.02 | (±0.29) | 0.13 | (±0.3) | 0.15 | (±0.24) | 0.26 | (±0.21) |
| 82 | 0.00 | 0.00 | −0.3 | (±0.33) | −0.44 | (±0.35) | −0.44 | (±0.34) | 0.05 | (±0.43) | 0.04 | (±0.48) |
| 83 | 0.00 | 0.00 | 0.78 | (±0.42) | 0.74 | (±0.31) | 0.9 | (±0.43) | 1.12 | (±0.53) | 1.2 | (±0.49) |
| 84 | 0.01 | 0.03 | 0.36 | (±0.38) | 0.36 | (±0.37) | 0.41 | (±0.51) | 0.53 | (±0.56) | 0.72 | (±0.43) |
| 85 | 0.00 | 0.01 | 0.55 | (±0.48) | 0.72 | (±0.46) | 0.89 | (±0.45) | 0.93 | (±0.51) | 1.1 | (±0.6) |
| 86 | 0.00 | 0.01 | 1.24 | (±0.53) | 1.21 | (±0.47) | 1.43 | (±0.47) | 1.47 | (±0.39) | 1.57 | (±0.47) |
| 87 | 0.00 | 0.00 | 1.17 | (±0.45) | 1.28 | (±0.49) | 1.51 | (±0.56) | 1.4 | (±0.57) | 1.7 | (±0.55) |
| 88 | 0.00 | 0.02 | 1.45 | (±0.74) | 1.45 | (±0.62) | 1.9 | (±0.61) | 1.69 | (±0.72) | 1.96 | (±0.76) |
| 89 | 0.00 | 0.00 | −0.08 | (±0.34) | 0.01 | (±0.23) | 0.14 | (±0.26) | 0.18 | (±0.33) | 0.27 | (±0.31) |
| 90 | 0.01 | 0.03 | −1.8 | (±0.57) | −2.21 | (±0.39) | −1.99 | (±0.31) | −1.9 | (±0.41) | −1.9 | (±0.55) |
| 91 | 0.00 | 0.01 | −1.04 | (±0.41) | −1.39 | (±0.19) | −1.22 | (±0.24) | −1.1 | (±0.38) | −1.01 | (±0.44) |
| 92 | 0.00 | 0.02 | 0.03 | (±0.45) | 0.08 | (±0.49) | 0.06 | (±0.44) | 0.39 | (±0.51) | 0.42 | (±0.39) |
| 93 | 0.01 | 0.05 | 0.11 | (±0.33) | −0.02 | (±0.56) | −0.21 | (±0.71) | 0 | (±0.43) | 0.21 | (±0.41) |
| 94 | 0.00 | 0.02 | 0.09 | (±0.55) | −0.21 | (±0.47) | 0.2 | (±0.54) | −0.24 | (±0.56) | −0.18 | (±0.44) |
| 95 | 0.01 | 0.04 | 0.15 | (±0.81) | −0.29 | (±0.57) | −0.45 | (±0.56) | −0.22 | (±0.44) | 0.15 | (±0.73) |
| 96 | 0.01 | 0.05 | −1.13 | (±0.35) | −1.44 | (±0.34) | −1.44 | (±0.35) | −1.2 | (±0.41) | −1.1 | (±0.69) |
| 97 | 0.00 | 0.02 | −0.24 | (±0.48) | −0.63 | (±0.41) | −0.57 | (±0.48) | −0.35 | (±0.34) | −0.29 | (±0.26) |
| 98 | 0.00 | 0.01 | −0.12 | (±0.43) | 0.16 | (±0.35) | 0.09 | (±0.46) | −0.16 | (±0.39) | 0.2 | (±0.44) |
| 99 | 0.00 | 0.02 | 0.06 | (±0.29) | 0.02 | (±0.39) | 0.16 | (±0.36) | 0.26 | (±0.26) | 0.3 | (±0.28) |
| 100 | 0.00 | 0.01 | −0.33 | (±0.26) | −0.27 | (±0.24) | −0.26 | (±0.25) | −0.02 | (±0.19) | −0.21 | (±0.2) |
| 101 | 0.00 | 0.00 | 0.99 | (±0.61) | 0.98 | (±0.81) | 1.37 | (±0.65) | 1.95 | (±0.79) | 1.8 | (±0.56) |
| 102 | 0.00 | 0.00 | 0.84 | (±0.35) | 0.92 | (±0.33) | 1.2 | (±0.3) | 1.15 | (±0.3) | 1.1 | (±0.23) |
| 103 | 0.00 | 0.00 | −1.94 | (±0.31) | −1.92 | (±0.31) | −2.12 | (±0.27) | −1.49 | (±0.43) | −1.66 | (±0.46) |
| 104 | 0.00 | 0.00 | −2.21 | (±0.66) | −2.4 | (±0.52) | −1.79 | (±0.8) | −0.24 | (±0.93) | −1.38 | (±0.66) |
| 105 | 0.00 | 0.00 | −2.85 | (±0.49) | −2.88 | (±0.42) | −2.44 | (±0.68) | −1.38 | (±0.84) | −2.34 | (±0.7) |
| 106 | 0.00 | 0.00 | 0.53 | (±1.01) | 0.83 | (±0.93) | 1.48 | (±0.96) | 2.59 | (±1.1) | 1.44 | (±0.91) |
| 107 | 0.00 | 0.00 | −0.15 | (±0.28) | −0.11 | (±0.31) | −0.04 | (±0.25) | 0.22 | (±0.22) | 0.11 | (±0.25) |
| 108 | 0.00 | 0.00 | −0.2 | (±0.58) | 0.25 | (±0.71) | 0.41 | (±0.46) | 1.45 | (±0.54) | 1.29 | (±0.51) |
| 109 | 0.00 | 0.00 | 3.42 | (±0.62) | 3.9 | (±0.66) | 3.92 | (±0.46) | 4.6 | (±0.77) | 4.61 | (±0.73) |
| 110 | 0.00 | 0.00 | 3.82 | (±0.7) | 4.3 | (±0.77) | 4.33 | (±0.59) | 4.89 | (±0.69) | 5.08 | (±0.73) |
| 111 | 0.00 | 0.00 | 2.61 | (±0.79) | 2.89 | (±0.9) | 2.94 | (±0.99) | 3.79 | (±1.04) | 4.05 | (±0.86) |
| 112 | 0.00 | 0.00 | 1.14 | (±0.69) | 1.45 | (±0.85) | 1.51 | (±0.64) | 2.48 | (±0.6) | 2.49 | (±0.55) |
| 113 | 0.00 | 0.00 | −0.11 | (±0.45) | −0.28 | (±0.51) | −0.06 | (±0.46) | 0.56 | (±0.69) | 0.25 | (±0.5) |
| 114 | 0.00 | 0.00 | 1.27 | (±0.61) | 1.59 | (±0.59) | 1.57 | (±0.44) | 2.17 | (±0.61) | 2.2 | (±0.57) |
| 115 | 0.00 | 0.00 | 0.28 | (±0.62) | 0.53 | (±0.74) | 0.66 | (±0.53) | 1.16 | (±0.6) | 1.12 | (±0.82) |
| 116 | 0.00 | 0.00 | 2.3 | (±0.68) | 2.73 | (±0.72) | 2.77 | (±0.64) | 3.22 | (±0.84) | 3.19 | (±0.63) |
| 117 | 0.00 | 0.00 | 1.09 | (±0.59) | 1.31 | (±0.63) | 1.34 | (±0.54) | 1.84 | (±0.7) | 1.84 | (±0.61) |
| 118 | 0.01 | 0.04 | 0.18 | (±0.25) | −0.02 | (±0.4) | −0.21 | (±0.34) | 0.13 | (±0.3) | 0.12 | (±0.42) |
| 119 | 0.00 | 0.01 | −0.47 | (±0.35) | −0.33 | (±0.37) | −0.69 | (±0.43) | −0.33 | (±0.32) | −0.55 | (±0.3) |
| 120 | 0.00 | 0.01 | −0.13 | (±0.36) | 0.09 | (±0.31) | −0.04 | (±0.26) | 0.2 | (±0.22) | 0.07 | (±0.24) |
| 121 | 0.00 | 0.00 | 0.36 | (±0.52) | 0.63 | (±0.49) | 0.38 | (±0.45) | 0.84 | (±0.43) | 0.44 | (±0.33) |
| 122 | 0.00 | 0.01 | 0.4 | (±0.48) | 0.61 | (±0.47) | 0.41 | (±0.41) | 0.77 | (±0.62) | 0.35 | (±0.33) |
| 123 | 0.00 | 0.00 | 0.5 | (±0.41) | 0.87 | (±0.48) | 0.53 | (±0.51) | 0.95 | (±0.5) | 0.63 | (±0.38) |
| 124 | 0.01 | 0.04 | 0.95 | (±0.53) | 1.3 | (±0.62) | 0.98 | (±0.58) | 1.5 | (±0.68) | 1.24 | (±0.45) |
| 125 | 0.00 | 0.00 | 0.24 | (±0.37) | 0.52 | (±0.3) | 0.58 | (±0.25) | 0.66 | (±0.38) | 0.5 | (±0.3) |
| 126 | 0.00 | 0.00 | −0.26 | (±0.42) | −0.18 | (±0.36) | 0.28 | (±0.48) | 0.05 | (±0.38) | −0.1 | (±0.59) |
| 127 | 0.00 | 0.01 | −1.87 | (±0.37) | −2.08 | (±0.37) | −1.82 | (±0.33) | −1.6 | (±0.44) | −1.65 | (±0.32) |
| 128 | 0.00 | 0.01 | 0.34 | (±0.37) | 0.33 | (±0.3) | 0.51 | (±0.36) | 0.83 | (±0.45) | 0.6 | (±0.31) |
| 129 | 0.00 | 0.02 | 0.05 | (±0.16) | 0.21 | (±0.31) | 0.06 | (±0.24) | 0.21 | (±0.21) | −0.05 | (±0.22) |
| 130 | 0.01 | 0.04 | −0.64 | (±0.51) | −0.36 | (±0.39) | −0.36 | (±0.45) | −0.23 | (±0.34) | −0.46 | (±0.46) |
| 131 | 0.01 | 0.05 | 0.19 | (±0.4) | 0.17 | (±0.33) | 0.54 | (±0.71) | 0.47 | (±0.59) | 0.28 | (±0.45) |
| 132 | 0.00 | 0.02 | −0.03 | (±0.18) | 0.14 | (±0.17) | 0.18 | (±0.22) | 0.06 | (±0.19) | 0.05 | (±0.17) |

TABLE 7
Cluster 2

| Sequence | PC-Test | | Median (±Standard deviation) | | | | | | | | | |
|---|---|---|---|---|---|---|---|---|---|---|---|---|
| No. | p-value | q-value | t0 | | t0 post | | t3 post | | t7 post | | t14 post | |
| 133 | 0.00 | 0.00 | −0.42 | (±0.32) | 0.41 | (±0.36) | 0.02 | (±0.39) | 0.01 | (±0.42) | −0.07 | (±0.38) |
| 134 | 0.00 | 0.00 | 0.02 | (±0.31) | 0.46 | (±0.34) | 0.26 | (±0.23) | 0.12 | (±0.34) | 0.24 | (±0.22) |
| 135 | 0.00 | 0.01 | 0.05 | (±0.42) | 0.36 | (±0.22) | 0.24 | (±0.41) | 0 | (±0.38) | 0.13 | (±0.35) |
| 136 | 0.01 | 0.04 | −0.1 | (±0.23) | −0.04 | (±0.21) | −0.2 | (±0.26) | −0.17 | (±0.21) | −0.3 | (±0.36) |
| 137 | 0.00 | 0.01 | −0.23 | (±0.35) | 0.02 | (±0.41) | 0.11 | (±0.33) | −0.05 | (±0.3) | −0.2 | (±0.49) |
| 138 | 0.01 | 0.04 | 0.02 | (±0.43) | −0.24 | (±0.38) | −0.07 | (±0.29) | 0.12 | (±0.34) | −0.19 | (±0.44) |
| 139 | 0.00 | 0.00 | 0.01 | (±0.48) | 1.4 | (±0.92) | 0.58 | (±0.56) | 0.61 | (±0.67) | 0.5 | (±0.77) |
| 140 | 0.01 | 0.03 | −0.06 | (±0.29) | 0.13 | (±0.28) | 0.13 | (±0.29) | −0.1 | (±0.22) | −0.06 | (±0.25) |
| 141 | 0.01 | 0.04 | 0.34 | (±0.53) | 0.91 | (±0.79) | 0.83 | (±0.69) | 0.13 | (±0.66) | 0.26 | (±0.85) |
| 142 | 0.01 | 0.03 | −0.28 | (±0.31) | −0.1 | (±0.3) | −0.16 | (±0.27) | −0.3 | (±0.27) | −0.38 | (±0.36) |
| 143 | 0.00 | 0.02 | −0.71 | (±0.27) | −0.49 | (±0.27) | −0.63 | (±0.34) | −0.7 | (±0.3) | −0.74 | (±0.24) |
| 144 | 0.00 | 0.00 | −1.01 | (±0.31) | −0.55 | (±0.3) | −0.87 | (±0.37) | −0.92 | (±0.22) | −0.98 | (±0.3) |
| 145 | 0.00 | 0.00 | −0.16 | (±0.23) | −0.01 | (±0.2) | −0.06 | (±0.32) | −0.29 | (±0.24) | −0.25 | (±0.28) |
| 146 | 0.00 | 0.00 | −0.13 | (±0.42) | 0.48 | (±0.51) | 0.18 | (±0.7) | −0.01 | (±0.4) | −0.18 | (±0.48) |
| 147 | 0.00 | 0.03 | −0.24 | (±0.53) | 0.03 | (±0.53) | −0.02 | (±0.43) | −0.06 | (±0.42) | −0.29 | (±0.56) |
| 148 | 0.00 | 0.02 | −0.11 | (±0.47) | −0.1 | (±0.35) | 0.25 | (±0.29) | 0.16 | (±0.4) | 0.11 | (±0.38) |
| 149 | 0 01 | 0.04 | −0.69 | (±0.4) | −0.46 | (±0.25) | −0.54 | (±0.3) | −0.64 | (±0.27) | −0.72 | (±0.31) |
| 150 | 0.00 | 0.00 | −0.23 | (±0.27) | −0.14 | (±0.28) | −0.09 | (±0.22) | −0.03 | (±0.18) | −0.36 | (±0.35) |
| 151 | 0.00 | 0.00 | −0.59 | (±0.36) | −0.06 | (±0.3) | −0.36 | (±0.27) | −0.32 | (±0.28) | −0.55 | (±0.27) |
| 152 | 0.01 | 0.04 | −0.12 | (±0.27) | 0.16 | (±0.37) | 0.09 | (±0.26) | 0.17 | (±0.32) | 0.04 | (±0.23) |
| 153 | 0.00 | 0.01 | 0.97 | (±0.6) | 1.41 | (±0.8) | 1.3 | (±0.63) | 0.85 | (±0.63) | 1.43 | (±0.55) |
| 154 | 0.00 | 0.00 | 0.98 | (±0.39) | 1.4 | (±0.55) | 1.22 | (±0.51) | 0.91 | (±0.5) | 1.28 | (±0.51) |
| 155 | 0.00 | 0.00 | 0.45 | (±0.42) | 0.89 | (±0.52) | 0.6 | (±0.29) | 0.53 | (±0.27) | 0.85 | (±0.51) |
| 156 | 0.00 | 0.00 | 0.27 | (±0.44) | 0.85 | (±0.47) | 0.6 | (±0.3) | 0.29 | (±0.42) | 0.46 | (±0.42) |
| 157 | 0.01 | 0.03 | 0.27 | (±0.49) | 0.52 | (±0.53) | 0.75 | (±0.53) | 0.53 | (±0.63) | 0.45 | (±0.47) |
| 158 | 0.00 | 0.00 | −0.04 | (±0.19) | 0.26 | (±0.26) | 0.16 | (±0.36) | 0.07 | (±0.25) | 0.07 | (±0.28) |
| 159 | 0.00 | 0.00 | −0.04 | (±0.32) | 0.55 | (±0.28) | 0.34 | (±0.32) | 0.09 | (±0.4) | 0.02 | (±0.29) |
| 160 | 0.00 | 0.01 | −0.07 | (±0.2) | 0.2 | (±0.28) | 0.07 | (±0.31) | −0.01 | (±0.33) | −0.24 | (±0.3) |
| 161 | 0.00 | 0.00 | 0.95 | (±0.48) | 1.15 | (±0.66) | 1.71 | (±0.59) | 1.15 | (±0.62) | 0.9 | (±0.44) |
| 162 | 0.01 | 0.05 | 1.75 | (±0.44) | 2.03 | (±0.73) | 1.95 | (±0.64) | 1.58 | (±0.62) | 1.33 | (±0.81) |
| 163 | 0.01 | 0.03 | 0.69 | (±0.36) | 0.71 | (±0.5) | 1.03 | (±0.51) | 0.62 | (±0.36) | 0.47 | (±0.32) |
| 164 | 0.00 | 0.02 | 0.69 | (±0.48) | 0.79 | (±0.51) | 1.05 | (±0.59) | 0.61 | (±0.39) | 0.46 | (±0.45) |
| 165 | 0.01 | 0.03 | 0.82 | (±0.48) | 1.36 | (±0.54) | 1.21 | (±0.5) | 1.19 | (±0.53) | 1.09 | (±0.37) |
| 166 | 0.00 | 0.00 | 0.29 | (±0.58) | 1.27 | (±0.64) | 0.74 | (±0.71) | 0.85 | (±0.54) | 0.5 | (±0.6) |
| 167 | 0.00 | 0.00 | 1.67 | (±1.13) | 3.13 | (±1.04) | 2.75 | (±1.08) | 2.21 | (±0.92) | 2.29 | (±1) |
| 168 | 0.00 | 0.00 | 0.31 | (±0.61) | 2.11 | (±1) | 1.25 | (±0.92) | 1.16 | (±0.67) | 1.15 | (±0.96) |
| 169 | 0.00 | 0.03 | 0.7 | (±0.63) | 2.64 | (±0.97) | 1.63 | (±1) | 1.66 | (±1) | 1.72 | (±1.04) |
| 170 | 0.00 | 0.01 | 0.61 | (±0.66) | 1.34 | (±0.49) | 0.89 | (±0.44) | 0.74 | (±0.54) | 0.66 | (±0.38) |
| 171 | 0.00 | 0.00 | 0.95 | (±0.76) | 1.95 | (±0.64) | 1.68 | (±1.28) | 1.34 | (±0.73) | 1.22 | (±0.7) |
| 172 | 0.01 | 0.03 | 0.43 | (±0.75) | 1.31 | (±0.79) | 0.87 | (±0.72) | 0.63 | (±0.58) | 0.79 | (±0.6) |
| 173 | 0.00 | 0.00 | 1 | (±0.66) | 2.08 | (±0.8) | 2.15 | (±0.64) | 1.54 | (±1) | 1.35 | (±0.54) |
| 174 | 0.00 | 0.01 | −0.55 | (±0.41) | −0.22 | (±0.4) | −0.44 | (±0.48) | −0.52 | (±0.27) | −0.69 | (±0.27) |
| 175 | 0.01 | 0.04 | 0.04 | (±0.29) | 0.2 | (±0.23) | 0.22 | (±0.27) | 0.21 | (±0.26) | 0.05 | (±0.23) |
| 176 | 0.00 | 0.03 | −0.22 | (±0.39) | 0.07 | (±0.34) | 0.06 | (±0.36) | −0.05 | (±0.38) | −0.17 | (±0.37) |
| 177 | 0.01 | 0.03 | −0.24 | (±0.42) | −0.14 | (±0.31) | −0.14 | (±0.42) | −0.26 | (±0.41) | −0.42 | (±0.37) |
| 178 | 0.00 | 0.00 | −0.13 | (±0.45) | −0.14 | (±0.32) | 0.17 | (±0.43) | 0.19 | (±0.36) | −0.17 | (±0.39) |
| 179 | 0.00 | 0.02 | −0.17 | (±0.5) | −0.18 | (±0.35) | 0.13 | (±0.36) | 0.07 | (±0.38) | ±0.15 | (±0.37) |
| 180 | 0.00 | 0.02 | −0.49 | (±0.33) | −0.29 | (±0.38) | −0.15 | (±0.37) | −0.2 | (±0.35) | −0.48 | (±0.31) |
| 181 | 0.01 | 0.04 | −0.23 | (±0.42) | 0.09 | (±0.42) | 0.09 | (±0.43) | 0.07 | (±0.34) | −0.02 | (±0.38) |
| 182 | 0.00 | 0.01 | −0.06 | (±0.56) | 0.67 | (±0.42) | 0.5 | (±0.47) | 0.39 | (±0.4) | 0.47 | (±0.51) |
| 183 | 0.00 | 0.00 | 0.3 | (±0.59) | 0.7 | (±0.63) | 1.08 | (±0.64) | 1.13 | (±0.34) | 0.94 | (±0.59) |
| 184 | 0.01 | 0.04 | −0.09 | (±0.32) | 0.18 | (±0.3) | 0.21 | (±0.29) | 0.2 | (±0.37) | 0.12 | (±0.22) |
| 185 | 0.01 | 0.05 | −0.07 | (±0.44) | −0.11 | (±0.25) | 0.06 | (±0.23) | 0.1 | (±0.17) | 0.66 | (±0.24) |
| 186 | 0.00 | 0.00 | 0.1 | (±0.46) | 0.33 | (±0.3) | 0.51 | (±0.32) | 0.55 | (±0.23) | 0.5 | (±0.36) |
| 187 | 0.00 | 0.02 | 0.33 | (±0.39) | 1.28 | (±0.66) | 0.76 | (±0.84) | 0.62 | (±0.56) | 0.67 | (±0.46) |
| 188 | 0.00 | 0.03 | −0.27 | (±0.52) | 0.42 | (±0.47) | 0.23 | (±0.39) | 0.25 | (±0.38) | 0.06 | (±0.49) |
| 189 | 0.00 | 0.00 | 0.74 | (±0.46) | 1.71 | (±0.38) | 1.36 | (±0.49) | 0.94 | (±0.55) | 0.8 | (±0.54) |
| 190 | 0.00 | 0.00 | −0.15 | (±0.35) | 0.42 | (±0.46) | 0.14 | (±0.39) | 0.15 | (±0.28) | −0.01 | (±0.33) |
| 191 | 0.00 | 0.01 | −0.82 | (±0.27) | −0.32 | (±0.33) | −0.65 | (±0.27) | −0.5 | (±0.17) | −0.57 | (±0.31) |
| 192 | 0.00 | 0.00 | 0.05 | (±0.31) | 0.49 | (±0.34) | 0.2 | (±0.24) | 0.23 | (±0.3) | 0.04 | (±0.31) |
| 193 | 0.00 | 0.00 | −0.19 | (±0.48) | 1.01 | (±0.64) | −0.16 | (±0.49) | 0.45 | (±0.47) | −0.08 | (±0.42) |
| 194 | 0.00 | 0.00 | −0.59 | (±0.38) | 0.01 | (±0.41) | −0.35 | (±0.36) | −0.44 | (±0.35) | −0.54 | (±0.44) |
| 195 | 0.00 | 0.01 | ±0.43 | (±0.43) | 0.13 | (±0.57) | −0.13 | (±0.37) | −0.17 | (±0.34) | −0.27 | (±0.51) |
| 196 | 0.00 | 0.00 | 0.76 | (±0.55) | 1.48 | (±0.47) | 1.48 | (±0.49) | 1.26 | (±0.49) | 1.15 | (±0.46) |
| 197 | 0.00 | 0.00 | 0.68 | (±0.43) | 1.22 | (±0.49) | 1.15 | (±0.32) | 0.94 | (±0.36) | 0.99 | (±0.39) |
| 198 | 0.00 | 0.00 | −0.54 | (±0.36) | −0.07 | (±0.52) | −0.24 | (±0.51) | −0.59 | (±0.51) | −0.85 | (±0.46) |
| 199 | 0.00 | 0.00 | 0.31 | (±0.43) | 0.92 | (±0.48) | 0.63 | (±0.49) | 0.78 | (±0.48) | 0.21 | (±0.5) |
| 200 | 0.00 | 0.01 | −0.49 | (±0.41) | 0.12 | (±0.47) | −0.07 | (±0.35) | −0.05 | (±0.26) | −0.18 | (±0.2) |
| 201 | 0.00 | 0.00 | 0.09 | (±0.53) | 1.05 | (±0.81) | 0.79 | (±0.56) | 0.76 | (±0.6) | 0.54 | (±0.58) |
| 202 | 0.00 | 0.00 | −0.46 | (±0.31) | 0.2 | (±0.44) | −0.03 | (±0.47) | −0.26 | (±0.37) | −0.32 | (±0.4) |
| 203 | 0.00 | 0.01 | −1.76 | (±0.32) | −1.48 | (±0.31) | −1.57 | (±0.28) | −1.44 | (±0.3) | −1.76 | (±0.26) |
| 204 | 0.00 | 0.00 | 0.99 | (±0.55) | 1.43 | (±0.43) | 1.13 | (±0.52) | 1.4 | (±0.52) | 0.99 | (±0.51) |
| 205 | 0.00 | 0.02 | −0.64 | (±0.37) | −0.34 | (±0.38) | −0.53 | (±0.31) | −0.39 | (±0.31) | −0.6 | (±0.34) |
| 206 | 0.00 | 0.02 | 0.13 | (±0.38) | 0.58 | (±0.42) | 0.28 | (±0.37) | 0.36 | (±0.37) | 0.12 | (±0.44) |
| 207 | 0.00 | 0.00 | −0.28 | (±0.36) | 0.17 | (±0.35) | −0.02 | (±0.33) | 0.07 | (±0.32) | −0.08 | (±0.43) |
| 208 | 0.00 | 0.00 | −0.45 | (±0.33) | 0.54 | (±0.52) | 0.01 | (±0.24) | −0.01 | (±0.3) | −0.15 | (±0.6) |
| 209 | 0.00 | 0.00 | 0.16 | (±0.46) | 2.08 | (±0.83) | 0.89 | (±0.79) | 0.76 | (±0.68) | 0.37 | (±0.63) |
| 210 | 0.00 | 0.00 | −0.83 | (±0.43) | −0.18 | (±0.35) | −0.59 | (±0.37) | −0.51 | (±0.29) | −0.73 | (±0.34) |
| 211 | 0.00 | 0.00 | 0.18 | (±0.41) | 1.27 | (±0.55) | 0.79 | (±0.43) | 0.74 | (±0.47) | 0.64 | (±0.52) |
| 212 | 0.00 | 0.00 | 0.94 | (±0.41) | 2.1 | (±0.42) | 1.55 | (±0.43) | 1.39 | (±0.36) | 1.47 | (±0.49) |
| 213 | 0.00 | 0.00 | −0.57 | (±0.44) | 0.47 | (±0.36) | −0.03 | (±0.34) | 0.05 | (±0.35) | −0.12 | (±0.43) |
| 214 | 0.00 | 0.00 | 0.75 | (±0.44) | 1.67 | (±0.45) | 1.4 | (±0.55) | 1.34 | (±0.53) | 1.2 | (±0.62) |
| 215 | 0.00 | 0.00 | 2.08 | (±0.7) | 4.79 | (±0.58) | 4.01 | (±0.68) | 3.9 | (±1.1) | 3.54 | (±0.97) |
| 216 | 0.00 | 0.00 | 0.49 | (±0.53) | 2.08 | (±0.63) | 1.28 | (±0.58) | 1.27 | (±0.58) | 1.01 | (±0.7) |
| 217 | 0.00 | 0.00 | 0.81 | (±0.77) | 2.53 | (±0.92) | 1.4 | (±0.74) | 1.73 | (±0.64) | 1.23 | (±0.57) |
| 218 | 0.00 | 0.00 | 0.24 | (±0.33) | 1.06 | (±0.4) | 0.61 | (±0.49) | 0.63 | (±0.42) | 0.27 | (±0.38) |
| 219 | 0.00 | 0.00 | −0.89 | (±0.43) | 0.57 | (±0.49) | −0.03 | (±0.46) | −0.33 | (±0.32) | −0.71 | (±0.28) |
| 220 | 0.00 | 0.00 | −0.11 | (±0.31) | 1.04 | (±0.52) | 0.43 | (±0.39) | 0.19 | (±0.38) | 0.05 | (±0.42) |
| 221 | 0.00 | 0.00 | 0.78 | (±0.65) | 1.76 | (±0.52) | 1 | (±0.47) | 0.83 | (±0.41) | 0.47 | (±0.44) |
| 222 | 0.00 | 0.00 | −0.08 | (±0.6) | 0.72 | (±0.36) | 0.34 | (±0.34) | 0.03 | (±0.43) | −0.21 | (±0.47) |
| 223 | 0.00 | 0.00 | −0.13 | (±0.48) | 0.7 | (±0.42) | 0.43 | (±0.35) | 0.21 | (±0.37) | 0.04 | (±0.47) |
| 224 | 0.00 | 0.00 | 0.84 | (±0.42) | 1.99 | (±0.5) | 1.3 | (±0.36) | 1.12 | (±0.54) | 1 | (±0.41) |
| 225 | 0.00 | 0.00 | −1.17 | (±0.42) | 0.48 | (±0.56) | −0.59 | (±0.47) | −0.51 | (±0.48) | −0.67 | (±0.46) |
| 226 | 0.00 | 0.00 | 0.32 | (±0.36) | 1.3 | (±0.44) | 0.8 | (±0.47) | 0.72 | (±0.36) | 0.48 | (±0.36) |
| 227 | 0.00 | 0.00 | −0.06 | (±0.37) | 0.89 | (±0.47) | 0.41 | (±0.26) | 0.22 | (±0.38) | 0.18 | (±0.38) |
| 228 | 0.00 | 0.00 | −0.97 | (±0.47) | 0.3 | (±0.36) | −0.29 | (±0.34) | −0.56 | (±0.44) | −0.79 | (±0.41) |
| 229 | 0.00 | 0.00 | −1.04 | (±0.41) | 0.18 | (±0.42) | −0.4 | (±0.29) | −0.83 | (±0.4) | −0.98 | (±0.44) |
| 230 | 0.00 | 0.00 | −1.25 | (±0.44) | −0.03 | (±0.44) | −0.64 | (±0.34) | −0.78 | (±0.33) | −1.06 | (±0.38) |
| 231 | 0.00 | 0.00 | −0.89 | (±0.39) | 0.38 | (±0.45) | −0.27 | (±0.38) | −0.53 | (±0.34) | −0.77 | (±0.42) |
| 232 | 0.00 | 0.00 | 0.07 | (±0.43) | 0.89 | (±0.41) | 0.57 | (±0.35) | 0.39 | (±0.27) | 0.18 | (±0.36) |
| 233 | 0.00 | 0.00 | −0.12 | (±0.42) | 0.62 | (±0.34) | 0.35 | (±0.38) | 0.05 | (±0.33) | 0.01 | (±0.43) |
| 234 | 0.00 | 0.00 | 0.07 | (±0.5) | 0.91 | (±0.35) | 0.55 | (±0.38) | 0.32 | (±0.31) | 0.07 | (±0.38) |
| 235 | 0.00 | 0.00 | 0.05 | (±0.52) | 0.65 | (±0.51) | 0.42 | (±0.36) | 0.18 | (±0.33) | 0.01 | (±0.41) |
| 236 | 0.00 | 0.01 | 0.12 | (±0.59) | 0.69 | (±0.48) | 0.23 | (±0.47) | 0.29 | (±0.31) | −0.05 | (±0.38) |
| 237 | 0.00 | 0.00 | 0.34 | (±0.6) | 1.35 | (±0.59) | 0.62 | (±0.44) | 0.53 | (±0.43) | 0.18 | (±0.52) |
| 238 | 0.00 | 0.00 | −0.52 | (±0.39) | −0.02 | (±0.36) | −0.3 | (±0.38) | −0.25 | (±0.28) | −0.54 | (±0.35) |
| 239 | 0.00 | 0.00 | −0.26 | (±0.44) | 0.49 | (±0.5) | 0.15 | (±0.49) | 0.01 | (±0.31) | −0.23 | (±0.27) |
| 240 | 0.00 | 0.00 | 0.1 | (±0.48) | 1.16 | (±0.4) | 0.82 | (±0.48) | 0.87 | (±0.44) | 0.19 | (±0.47) |
| 241 | 0.00 | 0.00 | 0.24 | (±0.45) | 1.23 | (±0.38) | 0.92 | (±0.45) | 0.91 | (±0.45) | 0.24 | (±0.49) |
| 242 | 0.00 | 0.00 | −0.73 | (±0.35) | −0.24 | (±0.37) | −0.46 | (±0.38) | −0.45 | (±0.37) | −0.69 | (±0.37) |
| 243 | 0.00 | 0.00 | 0.37 | (±0.42) | 0.79 | (±0.31) | 0.58 | (±0.31) | 0.61 | (±0.35) | 0.33 | (±0.39) |
| 244 | 0.00 | 0.00 | 0.21 | (±0.46) | 0.6 | (±0.34) | 0.44 | (±0.45) | 0.47 | (±0.52) | 0.11 | (±0.5) |
| 245 | 0.01 | 0.04 | 0.7 | (±0.52) | 1.13 | (±0.34) | 0.96 | (±0.4) | 0.83 | (±0.48) | 0.76 | (±0.43) |
| 246 | 0.00 | 0.01 | 0.67 | (±0.5) | 1.18 | (±0.38) | 1.08 | (±0.39) | 0.92 | (±0.33) | 0.8 | (±0.4) |
| 247 | 0.00 | 0.01 | −0.62 | (±0.43) | −0.41 | (±0.44) | −0.3 | (±0.41) | −0.24 | (±0.33) | −0.45 | (±0.36) |
| 248 | 0.00 | 0.00 | −0.81 | (±0.46) | −0.41 | (±0.45) | −0.47 | (±0.44) | −0.37 | (±0.31) | −0.75 | (±0.48) |
| 249 | 0.00 | 0.00 | 0.62 | (±0.5) | 1.44 | (±0.42) | 1.29 | (±0.36) | 1.21 | (±0.46) | 0.99 | (±0.42) |
| 250 | 0.00 | 0.01 | −0.11 | (±0.44) | 0.43 | (±0.41) | 0.2 | (±0.42) | 0.3 | (±0.37) | 0.09 | (±0.37) |
| 251 | 0.00 | 0.01 | −0.68 | (±0.47) | −0.07 | (±0.39) | −0.28 | (±0.39) | −0.31 | (±0.34) | −0.44 | (±0.33) |
| 252 | 0.00 | 0.00 | 1.11 | (±0.46) | 1.71 | (±0.51) | 1.61 | (±0.46) | 1.43 | (±0.47) | 1.3 | (±0.43) |
| 253 | 0.00 | 0.00 | 0.43 | (±0.45) | 0.92 | (±0.44) | 0.84 | (±0.43) | 0.67 | (±0.36) | 0.49 | (±0.34) |
| 254 | 0.00 | 0.00 | 0.39 | (±0.57) | 1.02 | (±0.43) | 0.93 | (±0.47) | 0.69 | (±0.42) | 0.56 | (±0.46) |
| 255 | 0.00 | 0.00 | 0.6 | (±0.48) | 1.02 | (±0.37) | 0.99 | (±0.34) | 0.6 | (±0.4) | 0.64 | (±0.32) |
| 256 | 0.00 | 0.00 | 1.88 | (±0.52) | 2.71 | (±0.55) | 2.21 | (±0.49) | 2.08 | (±0.61) | 2.09 | (±0.53) |
| 257 | 0.00 | 0.02 | 1.83 | (±0.55) | 2.48 | (±0.6) | 2.19 | (±0.45) | 2.13 | (±0.55) | 2.08 | (±0.52) |
| 258 | 0.00 | 0.00 | 0.33 | (±0.46) | 0.98 | (±0.44) | 0.7 | (±0.45) | 0.5 | (±0.42) | 0.37 | (±0.46) |
| 259 | 0.00 | 0.00 | 1.72 | (±0.46) | 2.6 | (±0.53) | 2.06 | (±0.41) | 1.94 | (±0.48) | 1.77 | (±0.61) |
| 260 | 0.00 | 0.00 | 0.32 | (±0.36) | 1 | (±0.43) | 0.68 | (±0.37) | 0.57 | (±0.42) | 0.53 | (±0.5) |
| 261 | 0.00 | 0.00 | 0.57 | (±0.37) | 1.24 | (±0.38) | 0.9 | (±0.41) | 0.61 | (±0.44) | 0.6 | (±0.43) |
| 262 | 0.00 | 0.00 | 0.34 | (±0.43) | 1.08 | (±0.4) | 0.86 | (±0.32) | 0.71 | (±0.29) | 0.6 | (±0.4) |
| 263 | 0.00 | 0.00 | 0.28 | (±0.58) | 0.91 | (±0.46) | 0.81 | (±0.42) | 0.67 | (±0.33) | 0.56 | (±0.46) |
| 264 | 0.00 | 0.00 | −0.52 | (±0.39) | 0.03 | (±0.41) | −0.18 | (±0.31) | −0.21 | (±0.28) | −0.37 | (±0.34) |
| 265 | 0.00 | 0.00 | −0.34 | (±0.36) | 0.11 | (±0.45) | −0.14 | (±0.3) | −0.18 | (±0.36) | −0.36 | (±0.38) |
| 266 | 0.00 | 0.00 | 0.66 | (±0.48) | 1.25 | (±0.57) | 1.03 | (±0.52) | 1 | (±0.51) | 0.76 | (±0.55) |
| 267 | 0.00 | 0.01 | 0.75 | (±0.54) | 1.03 | (±0.48) | 1.06 | (±0.6) | 0.99 | (±0.61) | 0.77 | (±0.4) |
| 268 | 0.00 | 0.00 | 0.07 | (±0.46) | 0.44 | (±0.47) | 0.38 | (±0.49) | 0.29 | (±0.35) | −0.08 | (±0.48) |
| 269 | 0.00 | 0.00 | 1.36 | (±0.35) | 1.76 | (±0.39) | 1.82 | (±0.53) | 1.6 | (±0.51) | 1.53 | (±0.39) |
| 270 | 0.01 | 0.04 | 3.53 | (±0.67) | 4.22 | (±0.79) | 3.93 | (±0.81) | 3.73 | (±0.9) | 3.69 | (±0.96) |
| 271 | 0.00 | 0.00 | 3.99 | (±0.55) | 4.5 | (±0.51) | 4.36 | (±0.45) | 4.18 | (±0.64) | 4.13 | (±0.52) |
| 272 | 0.00 | 0.03 | 0.69 | (±0.64) | 1.22 | (±0.47) | 1.15 | (±0.6) | 0.94 | (±0.52) | 0.92 | (±0.56) |
| 273 | 0.00 | 0.00 | 1.42 | (±0.51) | 2.23 | (±0.6) | 1.86 | (±0.58) | 1.75 | (±0.65) | 1.73 | (±0.61) |
| 274 | 0.00 | 0.00 | 0.47 | (±0.28) | 0.85 | (±0.33) | 0.82 | (±0.42) | 0.69 | (±0.39) | 0.65 | (±0.34) |
| 275 | 0.00 | 0.00 | 2.53 | (±0.78) | 3.81 | (±0.8) | 3.45 | (±0.74) | 2.86 | (±0.9) | 2.4 | (±0.68) |
| 276 | 0.00 | 0.00 | 1.17 | (±0.74) | 2.51 | (±0.56) | 2.14 | (±0.62) | 1.76 | (±0.53) | 1.55 | (±0.61) |
| 277 | 0.00 | 0.00 | 1.61 | (±0.71) | 2.31 | (±0.66) | 2.1 | (±0.71) | 2.05 | (±0.63) | 1.68 | (±0.69) |
| 278 | 0.00 | 0.02 | 3.22 | (±0.49) | 3.94 | (±0.57) | 3.64 | (±0.6) | 3.53 | (±0.62) | 3.54 | (±0.5) |
| 279 | 0.00 | 0.00 | 1.76 | (±0.49) | 2.34 | (±0.66) | 1.99 | (±0.53) | 1.72 | (±0.49) | 1.78 | (±0.46) |
| 280 | 0.00 | 0.00 | 0.65 | (±0.41) | 1.4 | (±0.46) | 1.07 | (±0.43) | 0.93 | (±0.46) | 0.68 | (±0.4) |
| 281 | 0.00 | 0.00 | 1.24 | (±0.39) | 2.15 | (±0.5) | 1.81 | (±0.54) | 1.54 | (±0.56) | 1.47 | (±0.48) |
| 282 | 0.00 | 0.00 | 1.09 | (±0.61) | 2.05 | (±0.61) | 1.63 | (±0.62) | 1.61 | (±0.61) | 1.32 | (±0.73) |
| 283 | 0.01 | 0.04 | 0.59 | (±0.49) | 0.93 | (±0.43) | 0.79 | (±0.4) | 0.63 | (±0.37) | 0.5 | (±0.46) |
| 284 | 0.00 | 0.01 | 0.74 | (±0.63) | 1.42 | (±0.63) | 1.03 | (±0.5) | 0.85 | (±0.53) | 0.7 | (±0.55) |
| 285 | 0.00 | 0.00 | 0.04 | (±0.47) | 0.71 | (±0.39) | 0.28 | (±0.5) | 0.25 | (±0.44) | 0.3 | (±0.4) |
| 286 | 0.01 | 0.04 | 0.04 | (±0.42) | 0.48 | (±0.32) | 0.32 | (±0.51) | 0.2 | (±0.44) | 0.11 | (±0.38) |
| 287 | 0.00 | 0.00 | 0.37 | (±0.62) | 0.63 | (±0.53) | 0.93 | (±0.45) | 0.48 | (±0.52) | 0.48 | (±0.37) |
| 288 | 0.00 | 0.00 | −0.73 | (±0.35) | −0.49 | (±0.28) | −0.46 | (±0.29) | −0.49 | (±0.27) | −0.76 | (±0.3) |
| 289 | 0.00 | 0.00 | −0.12 | (±0.43) | 0.2 | (±0.47) | 0.35 | (±0.44) | 0.13 | (±0.31) | −0.13 | (±0.31) |
| 290 | 0.00 | 0.00 | −0.12 | (±0.72) | 0.17 | (±0.75) | 0.57 | (±0.74) | 0.13 | (±0.68) | −0.18 | (±0.61) |
| 291 | 0.00 | 0.00 | 0.37 | (±0.49) | 0.79 | (±0.56) | 0.95 | (±0.58) | 0.53 | (±0.52) | 0.38 | (±0.58) |
| 292 | 0.00 | 0.00 | 0.07 | (±0.33) | 0.43 | (±0.38) | 0.59 | (±0.49) | 0.29 | (±0.37) | 0.14 | (±0.28) |
| 293 | 0.00 | 0.03 | −2.25 | (±0.4) | −1.87 | (±0.41) | −2.29 | (±0.33) | −2.25 | (±0.4) | −2.26 | (±0.32) |
| 294 | 0.01 | 0.03 | −1.01 | (±0.41) | −0.5 | (±0.36) | −0.98 | (±0.43) | −0.82 | (±0.4) | −1.12 | (±0.4) |
| 295 | 0.00 | 0.00 | −2.31 | (±0.46) | −1.8 | (±0.29) | −2.12 | (±0.33) | −2.21 | (±0.3) | −2.45 | (±0.34) |
| 296 | 0.00 | 0.01 | −0.7 | (±0.54) | −0.27 | (±0.43) | −0.6 | (±0.3) | −0.67 | (±0.36) | −0.95 | (±0.47) |
| 297 | 0.00 | 0.01 | −0.77 | (±0.64) | −0.2 | (±0.47) | −0.63 | (±0.36) | −0.73 | (±0.47) | −0.83 | (±0.49) |
| 298 | 0.00 | 0.01 | −0.98 | (±0.64) | −0.36 | (±0.41) | −0.76 | (±0.43) | −0.68 | (±0.36) | −1.01 | (±0.44) |
| 299 | 0.00 | 0.01 | −1.52 | (±0.59) | −1 | (±0.49) | −1.42 | (±0.54) | −1.33 | (±0.35) | −1.69 | (±0.33) |
| 300 | 0.01 | 0.04 | 0.05 | (±0.75) | 0.43 | (±0.56) | 0.25 | (±0.55) | 0.18 | (±0.34) | −0.13 | (±0.48) |
| 301 | 0.00 | 0.01 | −1.89 | (±0.46) | −2.02 | (±0.31) | −2.29 | (±0.33) | −1.98 | (±0.36) | −2.32 | (±0.39) |
| 302 | 0.00 | 0.00 | −0.26 | (±0.48) | −0.12 | (±0.39) | −0.59 | (±0.46) | −0.41 | (±0.34) | −0.7 | (±0.41) |
| 303 | 0.00 | 0.00 | −1.22 | (±0.44) | −0.96 | (±0.4) | −1.24 | (±0.38) | −1.01 | (±0.35) | −1.47 | (±0.43) |
| 304 | 0.00 | 0.00 | −0.97 | (±0.43) | −0.97 | (±0.26) | −1.16 | (±0.4) | −1 | (±0.21) | −1.33 | (±0.43) |
| 305 | 0.01 | 0.04 | −0.33 | (±0.46) | 0.04 | (±0.44) | −0.45 | (±0.37) | −0.48 | (±0.47) | −0.84 | (±0.51) |
| 306 | 0.01 | 0.05 | −0.16 | (±0.31) | 0 | (±0.25) | −0.27 | (±0.33) | −0.16 | (±0.26) | −0.33 | (±0.35) |
| 307 | 0.00 | 0.03 | 0.91 | (±0.64) | 1.03 | (±0.45) | 0.99 | (±0.49) | 1.04 | (±0.4) | 0.6 | (±0.51) |
| 308 | 0.01 | 0.04 | −0.05 | (±0.29) | −0.05 | (±0.3) | −0.29 | (±0.35) | −0.09 | (±0.23) | −0.23 | (±0.37) |
| 309 | 0.01 | 0.04 | −2.34 | (±0.22) | −2.22 | (±0.27) | −2.42 | (±0.38) | −2.26 | (±0.43) | −2.47 | (±0.29) |
| 310 | 0.01 | 0.05 | 0.27 | (±0.38) | 0.64 | (±0.41) | 0.55 | (±0.34) | 0.4 | (±0.41) | 0.31 | (±0.41) |
| 311 | 0.00 | 0.02 | −0.49 | (±0.31) | −0.38 | (±0.28) | −0.48 | (±0.28) | −0.31 | (±0.18) | −0.58 | (±0.27) |
| 312 | 0.00 | 0.02 | −0.67 | (±0.27) | −0.58 | (±0.23) | −0.77 | (±0.22) | −0.75 | (±0.22) | −0.84 | (±0.21) |
| 313 | 0.00 | 0.03 | −0.34 | (±0.33) | −0.15 | (±0.24) | −0.25 | (±0.26) | −0.28 | (±0.24) | −0.44 | (±0.28) |
| 314 | 0.01 | 0.04 | −0.46 | (±0.25) | −0.45 | (±0.17) | −0.6 | (±0.23) | −0.54 | (±0.21) | −0.64 | (±0.26) |
| 315 | 0.00 | 0.02 | −0.34 | (±0.3) | −0.32 | (±0.18) | −0.44 | (±0.25) | −0.4 | (±0.23) | −0.59 | (±0.31) |
| 316 | 0.01 | 0.03 | −0.62 | (±0.28) | −0.51 | (±0.21) | −0.66 | (±0.26) | −0.7 | (±0.21) | −0.77 | (±0.27) |
| 317 | 0.01 | 0.05 | −0.53 | (±0.31) | −0.61 | (±0.27) | −0.6 | (±0.32) | −0.7 | (±0.25) | −0.77 | (±0.33) |
| 318 | 0.01 | 0.04 | −0.79 | (±0.28) | −0.82 | (±0.17) | −0.89 | (±0.21) | −0.83 | (±0.26) | −1.02 | (±0.28) |
| 319 | 0.00 | 0.00 | −0.81 | (±0.29) | −0.73 | (±0.31) | −0.95 | (±0.3) | −0.95 | (±0.28) | −1.1 | (±0.36) |
| 320 | 0.01 | 0.04 | −0.46 | (±0.34) | −0.41 | (±0.23) | −0.54 | (±0.27) | −0.61 | (±0.27) | −0.67 | (±0.41) |
| 321 | 0.00 | 0.01 | −0.59 | (±0.31) | −0.51 | (±0.22) | −0.68 | (±0.23) | −0.72 | (±0.3) | −0.84 | (±0.36) |
| 322 | 0.00 | 0.02 | −0.48 | (±0.46) | −0.44 | (±0.25) | −0.65 | (±0.32) | −0.72 | (±0.3) | −0.76 | (±0.41) |
| 323 | 0.01 | 0.04 | −0.58 | (±0.3) | −0.5 | (±0.27) | −0.66 | (±0.28) | −0.66 | (±0.27) | −0.76 | (±0.29) |
| 324 | 0.01 | 0.04 | −0.53 | (±0.34) | −0.37 | (±0.26) | −0.56 | (±0.36) | −0.61 | (±0.25) | −0.7 | (±0.3) |
| 325 | 0.00 | 0.01 | −0.3 | (±0.31) | −0.15 | (±0.3) | −0.4 | (±0.26) | −0.41 | (±0.26) | −0.53 | (±0.31) |
| 326 | 0.00 | 0.01 | −0.33 | (±0.24) | −0.25 | (±0.23) | −0.5 | (±0.31) | −0.36 | (±0.26) | −0.48 | (±0.32) |
| 327 | 0.00 | 0.01 | −0.41 | (±0.25) | −0.33 | (±0.2) | −0.5 | (±0.29) | −0.45 | (±0.2) | −0.63 | (±0.28) |
| 328 | 0.00 | 0.00 | −0.75 | (±0.28) | −0.56 | (±0.16) | −0.86 | (±0.26) | −0.85 | (±0.23) | −0.99 | (±0.29) |
| 329 | 0.01 | 0.03 | −0.56 | (±0.21) | −0.45 | (±0.16) | −0.65 | (±0.22) | −0.65 | (±0.27) | −0.7 | (±0.32) |
| 330 | 0.00 | 0.01 | −0.3 | (±0.27) | 0.08 | (±0.26) | −0.21 | (±0.25) | −0.3 | (±0.24) | −0.37 | (±0.28) |
| 331 | 0.00 | 0.00 | −0.45 | (±0.33) | −0.14 | (±0.3) | −0.38 | (±0.28) | −0.47 | (±0.24) | −0.48 | (±0.28) |
| 332 | 0.01 | 0.04 | −0.54 | (±0.41) | −0.06 | (±0.47) | −0.47 | (±0.29) | −0.4 | (±0.36) | −0.42 | (±0.41) |
| 333 | 0.00 | 0.00 | −0.63 | (±0.25) | −0.27 | (±0.25) | −0.41 | (±0.23) | −0.48 | (±0.28) | −0.57 | (±0.24) |
| 334 | 0.00 | 0.02 | 0.15 | (±0.38) | 0.43 | (±0.27) | 0.34 | (±0.31) | 0.13 | (±0.23) | 0.3 | (±0.25) |
| 335 | 0.01 | 0.05 | 0.27 | (±0.27) | 0.45 | (±0.25) | 0.32 | (±0.19) | 0.25 | (±0.2) | 0.24 | (±0.18) |
| 336 | 0.00 | 0.02 | −0.11 | (±0.37) | 0.1 | (±0.29) | 0 | (±0.17) | −0.16 | (±0.19) | −0.17 | (±0.28) |
| 337 | 0.01 | 0.05 | 0.07 | (±0.3) | 0.15 | (±0.33) | 0.12 | (±0.2) | −0.05 | (±0.29) | −0.07 | (±0.26) |
| 338 | 0.00 | 0.02 | −0.25 | (±0.48) | −0.09 | (±0.39) | −0.27 | (±0.29) | −0.35 | (±0.27) | −0.47 | (±0.3) |
| 339 | 0.00 | 0.01 | −0.5 | (±0.35) | −0.29 | (±0.35) | −0.53 | (±0.31) | −0.62 | (±0.35) | −0.65 | (±0.27) |
| 340 | 0.00 | 0.00 | −0.06 | (±0.2) | 0.1 | (±0.2) | −0.08 | (±0.2) | −0.15 | (±0.21) | −0.09 | (±0.2) |
| 341 | 0.01 | 0.04 | −0.22 | (±0.13) | −0.03 | (±0.21) | −0.11 | (±0.27) | −0.23 | (±0.21) | −0.21 | (±0.18) |
| 342 | 0.00 | 0.01 | 0.17 | (±0.41) | 0.53 | (±0.31) | 0.4 | (±0.43) | 0.15 | (±0.46) | 0.09 | (±0.41) |
| 343 | 0.00 | 0.02 | −0.36 | (±0.16) | −0.23 | (±0.17) | −0.34 | (±0.15) | −0.41 | (±0.23) | −0.47 | (±0.23) |
| 344 | 0.00 | 0.02 | 0.36 | (±0.41) | 0.73 | (±0.21) | 0.48 | (±0.53) | 0.15 | (±0.46) | 0.42 | (±0.63) |
| 345 | 0.00 | 0.00 | 0.12 | (±0.24) | 0.38 | (±0.21) | 0.15 | (±0.24) | 0.23 | (±0.23) | 0.08 | (±0.24) |
| 346 | 0.00 | 0.00 | −0.65 | (±0.38) | −0.02 | (±0.42) | −0.48 | (±0.4) | −0.28 | (±0.45) | −0.42 | (±0.52) |
| 347 | 0.00 | 0.03 | −0.66 | (±0.32) | −0.34 | (±0.21) | −0.54 | (±0.2) | −0.6 | (±0.26) | −0.49 | (±0.28) |
| 348 | 0.00 | 0.03 | −0.48 | (±0.42) | −0.14 | (±0.35) | −0.31 | (±0.32) | −0.38 | (±0.36) | −0.44 | (±0.39) |
| 349 | 0.00 | 0.01 | −0.23 | (±0.24) | 0.01 | (±0.24) | −0.04 | (±0.26) | −0.21 | (±0.24) | −0.19 | (±0.27) |
| 350 | 0.00 | 0.02 | −0.07 | (±0.22) | 0.17 | (±0.33) | −0.04 | (±0.23) | −0.24 | (±0.26) | −0.13 | (±0.24) |
| 351 | 0.00 | 0.02 | −0.96 | (±0.26) | −0.82 | (±0.26) | −0.99 | (±0.24) | −1.08 | (±0.28) | −1.04 | (±0.23) |
| 352 | 0.01 | 0.05 | 0.06 | (±0.31) | 0.18 | (±0.33) | 0.08 | (±0.3) | −0.12 | (±0.29) | 0 | (±0.25) |
| 353 | 0.01 | 0.04 | −0.07 | (±0.26) | 0.19 | (±0.35) | 0 | (±0.26) | −0.06 | (±0.28) | −0.02 | (±0.25) |

TABLE 8
Cluster 3

| Sequence | PC-Test | | Median (±Standard deviation) | | | | | | | | | |
|---|---|---|---|---|---|---|---|---|---|---|---|---|
| No. | p-value | q-value | t0 | | t0 post | | t3 post | | t7 post | | t14 post | |
| 354 | 0.01 | 0.05 | −0.37 | (±0.3) | −0.32 | (±0.26) | −0.49 | (±0.2) | −0.54 | (±0.27) | −0.48 | (±0.25) |
| 355 | 0.01 | 0.04 | −0.24 | (±0.29) | −0.07 | (±0.26) | −0.18 | (±0.23) | −0.33 | (±0.33) | −0.27 | (±0.25) |
| 356 | 0.00 | 0.01 | 0.18 | (±0.32) | 0.3 | (±0.5) | 0 | (±0.27) | 0 | (±0.28) | −0.04 | (±0.34) |
| 357 | 0.00 | 0.01 | −0.39 | (±0.32) | −0.47 | (±0.31) | −0.58 | (±0.32) | −0.44 | (±0.3) | −0.7 | (±0.29) |
| 358 | 0.00 | 0.02 | −0.16 | (±0.44) | −0.08 | (±0.41) | −0.41 | (±0.46) | −0.44 | (±0.42) | −0.42 | (±0.38) |
| 359 | 0.01 | 0.04 | −0.37 | (±0.43) | −0.26 | (±0.44) | −0.51 | (±0.35) | −0.55 | (±0.31) | −0.61 | (±0.35) |
| 360 | 0.00 | 0.01 | 0.05 | (±0.29) | −0.31 | (±0.22) | −0.27 | (±0.21) | −0.22 | (±0.39) | −0.33 | (±0.46) |
| 361 | 0.00 | 0.03 | −0.45 | (±0.27) | −0.4 | (±0.43) | −0.42 | (±0.4) | −0.15 | (±0.42) | −0.58 | (±0.38) |
| 362 | 0.01 | 0.04 | −0.4 | (±0.32) | −0.41 | (±0.38) | −0.73 | (±0.5) | −0.43 | (±0.28) | −0.57 | (±0.34) |
| 363 | 0.00 | 0.01 | −0.47 | (±0.35) | −0.55 | (±0.36) | −0.76 | (±0.35) | −0.29 | (±0.4) | −0.68 | (±0.5) |
| 364 | 0.00 | 0.03 | −0.13 | (±0.23) | −0.04 | (±0.24) | −0.14 | (±0.27) | 0.06 | (±0.27) | −0.23 | (±0.35) |
| 365 | 0.01 | 0.05 | 0.01 | (±0.26) | 0.34 | (±0.47) | −0.06 | (±0.5) | 0.12 | (±0.4) | −0.08 | (±0.5) |
| 366 | 0.01 | 0.03 | 0.04 | (±0.34) | 0.2 | (±0.64) | −0.04 | (±0.43) | −0.28 | (±0.43) | −0.12 | (±0.33) |
| 367 | 0.00 | 0.02 | −0.33 | (±0.25) | −0.61 | (±0.27) | −0.51 | (±0.24) | −0.41 | (±0.43) | −0.38 | (±0.3) |
| 368 | 0.00 | 0.01 | 0.25 | (±0.4) | 0.32 | (±0.3) | 0.07 | (±0.22) | 0.1 | (±0.36) | 0.35 | (±0.21) |
| 369 | 0.01 | 0.05 | −0.09 | (±0.33) | 0.39 | (±0.38) | 0.08 | (±0.43) | 0.14 | (±0.51) | 0.2 | (±0.51) |
| 370 | 0.00 | 0.00 | −0.88 | (±0.38) | −0.49 | (±0.3) | −0.78 | (±0.3) | −0.62 | (±0.3) | −0.84 | (±0.3) |
| 371 | 0.00 | 0.01 | −0.33 | (±0.35) | −0.05 | (±0.55) | −0.56 | (±0.51) | −0.32 | (±0.42) | −0.51 | (±0.55) |
| 372 | 0.00 | 0.00 | −0.03 | (±0.42) | 0.31 | (±0.32) | 0.09 | (±0.36) | −0.1 | (±0.44) | −0.14 | (±0.3) |
| 373 | 0.01 | 0.05 | −0.09 | (±0.31) | −0.03 | (±0.35) | −0.13 | (±0.35) | −0.27 | (±0.37) | −0.23 | (±0.35) |
| 374 | 0.01 | 0.04 | 0.21 | (±0.4) | 0.24 | (±0.45) | 0.09 | (±0.52) | −0.06 | (±0.47) | 0.19 | (±0.37) |
| 375 | 0.00 | 0.03 | −0.25 | (±0.34) | −0.14 | (±0.22) | −0.22 | (±0.24) | −0.42 | (±0.38) | −0.42 | (±0.28) |
| 376 | 0.00 | 0.00 | −0.02 | (±0.28) | −0.07 | (±0.2) | −0.24 | (±0.19) | −0.23 | (±0.22) | −0.28 | (±0.21) |
| 377 | 0.01 | 0.04 | −1.01 | (±0.37) | −0.82 | (±0.29) | −1.11 | (±0.29) | −1 | (±0.4) | −1.22 | (±0.28) |
| 378 | 0.00 | 0.01 | −0.67 | (±0.27) | −0.63 | (±0.22) | −0.86 | (±0.19) | −0.79 | (±0.24) | −0.87 | (±0.28) |
| 379 | 0.01 | 0.04 | −0.83 | (±0.27) | −0.83 | (±0.23) | −0.93 | (±0.3) | −1 | (±0.32) | −1 | (±0.25) |
| 380 | 0.01 | 0.04 | −0.22 | (±0.37) | −0.51 | (±0.28) | −0.42 | (±0.38) | −0.54 | (±0.28) | −0.54 | (±0.3) |
| 381 | 0.00 | 0.01 | −1.2 | (±0.24) | −1.04 | (±0.24) | −1.2 | (±0.25) | −1.29 | (±0.24) | −1.3 | (±0.28) |
| 382 | 0.00 | 0.02 | −0.81 | (±0.29) | −0.66 | (±0.27) | −0.97 | (±0.24) | −0.94 | (±0.32) | −0.98 | (±0.34) |
| 383 | 0.01 | 0.05 | −0.46 | (±0.24) | −0.34 | (±0.26) | −0.56 | (±0.31) | −0.55 | (±0.33) | −0.46 | (±0.21) |
| 384 | 0.00 | 0.02 | −0.04 | (±0.4) | −0.01 | (±0.2) | −0.16 | (±0.27) | −0.33 | (±0.41) | −0.26 | (±0.35) |
| 385 | 0.00 | 0.01 | −0.49 | (±0.46) | −0.44 | (±0.5) | −0.59 | (±0.55) | −0.9 | (±0.55) | −1.04 | (±0.56) |
| 386 | 0.00 | 0.03 | −0.76 | (±0.21) | −0.72 | (±0.18) | −0.93 | (±0.23) | −0.88 | (±0.25) | −0.91 | (±0.27) |
| 387 | 0.01 | 0.04 | −1.01 | (±0.57) | −0.65 | (±0.46) | −0.59 | (±0.39) | −0.73 | (±0.53) | −1.01 | (±0.68) |
| 388 | 0.01 | 0.04 | −1.58 | (±0.41) | −1.37 | (±0.26) | −1.5 | (±0.46) | −1.39 | (±0.44) | −1.8 | (±0.36) |
| 389 | 0.00 | 0.02 | −0.15 | (±0.33) | −0.03 | (±0.36) | −0.27 | (±0.38) | −0.24 | (±0.39) | −0.42 | (±0.33) |
| 390 | 0.00 | 0.00 | −0.82 | (±0.47) | −0.8 | (±0.42) | −1.1 | (±0.41) | −1.09 | (±0.44) | −1.36 | (±0.42) |
| 391 | 0.01 | 0.04 | −0.6 | (±0.5) | −0.49 | (±0.29) | −0.81 | (±0.54) | −0.73 | (±0.44) | −0.98 | (±0.41) |
| 392 | 0.00 | 0.00 | −0.83 | (±0.26) | −1.08 | (±0.43) | −1.23 | (±0.38) | −0.92 | (±0.3) | −1.2 | (±0.26) |
| 393 | 0.01 | 0.04 | −0.17 | (±0.36) | −0.06 | (±0.26) | −0.14 | (±0.27) | −0.02 | (±0.28) | −0.31 | (±0.35) |
| 394 | 0.00 | 0.00 | −0.72 | (±0.34) | −0.73 | (±0.43) | −1.09 | (±0.47) | −0.81 | (±0.41) | −1.15 | (±0.5) |
| 395 | 0.01 | 0.04 | −0.25 | (±0.33) | −0.51 | (±0.47) | −0.49 | (±0.57) | −0.36 | (±0.47) | −0.78 | (±0.46) |
| 396 | 0.00 | 0.02 | −0.51 | (±0.2) | −0.3 | (±0.33) | −0.49 | (±0.25) | −0.49 | (±0.27) | −0.6 | (±0.3) |
| 397 | 0.00 | 0.01 | −1.16 | (±0.33) | −1.22 | (±0.49) | −1.34 | (±0.5) | −1.61 | (±0.6) | −1.62 | (±0.45) |
| 398 | 0.00 | 0.01 | −0.19 | (±0.33) | −0.11 | (±0.33) | −0.31 | (±0.28) | −0.35 | (±0.41) | −0.46 | (±0.35) |
| 399 | 0.01 | 0.03 | −1.63 | (±0.25) | −1.77 | (±0.37) | −1.84 | (±0.36) | −1.81 | (±0.42) | −1.94 | (±0.31) |
| 400 | 0.00 | 0.01 | −0.35 | (±0.41) | −0.52 | (±0.42) | −0.94 | (±0.58) | −0.76 | (±0.46) | −0.81 | (±0.44) |
| 401 | 0.00 | 0.02 | −0.54 | (±0.65) | −0.56 | (±0.61) | −0.94 | (±0.55) | −0.72 | (±0.73) | −1 | (±0.53) |
| 402 | 0.01 | 0.05 | −0.55 | (±0.24) | −0.45 | (±0.28) | −0.64 | (±0.31) | −0.49 | (±0.21) | −0.61 | (±0.24) |
| 403 | 0.00 | 0.00 | −0.32 | (±0.44) | −0.73 | (±0.34) | −0.5 | (±0.3) | −0.69 | (±0.31) | −0.71 | (±0.36) |
| 404 | 0.01 | 0.04 | −0.23 | (±0.38) | −0.65 | (±0.34) | −0.53 | (±0.41) | −0.66 | (±0.53) | −0.62 | (±0.37) |
| 405 | 0.01 | 0.05 | 0.12 | (±0.42) | 0.04 | (±0.41) | −0.07 | (±0.38) | −0.18 | (±0.41) | −0.11 | (±0.3) |
| 406 | 0.01 | 0.05 | −0.76 | (±0.36) | −0.81 | (±0.4) | −1 | (±0.34) | −0.98 | (±0.45) | −0.99 | (±0.4) |
| 407 | 0.01 | 0.04 | −0.69 | (±0.58) | −0.93 | (±0.52) | −1.18 | (±0.61) | −0.8 | (±0.72) | −1.22 | (±0.65) |
| 408 | 0.01 | 0.05 | −0.78 | (±0.22) | −0.62 | (±0.26) | −0.77 | (±0.34) | −0.83 | (±0.28) | −0.87 | (±0.24) |
| 409 | 0.01 | 0.04 | −0.33 | (±0.37) | −0.1 | (±0.37) | −0.41 | (±0.42) | −0.38 | (±0.34) | −0.31 | (±0.34) |
| 410 | 0.00 | 0.00 | −0.06 | (±0.44) | 0.09 | (±0.17) | −0.22 | (±0.49) | −0.34 | (±0.36) | 0.05 | (±0.38) |

TABLE 9
Cluster 4

| Sequence | PC-Test | | Median (±Standard deviation) | | | | | | | | | |
|---|---|---|---|---|---|---|---|---|---|---|---|---|
| No. | p-value | q-value | t0 | | t0 post | | t3 post | | t7 post | | t14 post | |
| 411 | 0.00 | 0.02 | 0.27 | (±0.33) | −0.04 | (±0.44) | −0.16 | (±0.46) | 0.02 | (±0.49) | 0.24 | (±0.44) |
| 412 | 0.01 | 0.05 | −0.3 | (±0.33) | −0.43 | (±0.42) | 0.01 | (±0.5) | −0.14 | (±0.48) | −0.13 | (±0.38) |
| 413 | 0.00 | 0.03 | −0.35 | (±0.41) | −0.66 | (±0.44) | −0.47 | (±0.44) | −0.24 | (±0.32) | −0.22 | (±0.43) |
| 414 | 0.01 | 0.05 | −0.08 | (±0.43) | −0.32 | (±0.41) | −0.4 | (±0.42) | −0.22 | (±0.31) | −0.08 | (±0.37) |
| 415 | 0.01 | 0.03 | 0.01 | (±0.26) | −0.16 | (±0.29) | −0.34 | (±0.34) | −0.13 | (±0.22) | −0.18 | (±0.28) |
| 416 | 0.01 | 0.05 | 0 | (±0.31) | −0.01 | (±0.56) | −0.19 | (±0.4) | 0.14 | (±0.36) | −0.16 | (±0.45) |
| 417 | 0.00 | 0.02 | 0.57 | (±0.61) | 0.17 | (±0.46) | 0.19 | (±0.47) | 0.53 | (±0.46) | 0.23 | (±0.49) |
| 418 | 0.00 | 0.01 | −0.19 | (±0.5) | −0.4 | (±0.41) | −0.57 | (±0.48) | −0.19 | (±0.34) | −0.56 | (±0.54) |
| 419 | 0.00 | 0.02 | −0.31 | (±0.43) | −0.69 | (±0.5) | −0.8 | (±0.51) | −0.41 | (±0.4) | −0.51 | (±0.43) |
| 420 | 0.01 | 0.05 | 0.35 | (±0.46) | 0.25 | (±0.44) | −0.05 | (±0.58) | 0.28 | (±0.45) | −0.04 | (±0.36) |
| 421 | 0.00 | 0.01 | −0.08 | (±0.43) | −0.39 | (±0.34) | −0.56 | (±0.54) | −0.07 | (±0.52) | −0.37 | (±0.4) |
| 422 | 0.01 | 0.04 | −0.17 | (±0.39) | −0.48 | (±0.37) | −0.4 | (±0.37) | −0.33 | (±0.31) | −0.48 | (±0.28) |
| 423 | 0.00 | 0.00 | 0.35 | (±0.4) | −0.35 | (±0.39) | −0.23 | (±0.43) | 0.22 | (±0.48) | −0.15 | (±0.63) |
| 424 | 0.00 | 0.03 | −0.35 | (±0.22) | −0.56 | (±0.24) | −0.44 | (±0.2) | −0.36 | (±0.2) | −0.36 | (±0.23) |
| 425 | 0.01 | 0.04 | −0.41 | (±0.35) | −0.73 | (±0.25) | −0.72 | (±0.26) | −0.7 | (±0.31) | −0.68 | (±0.35) |
| 426 | 0.01 | 0.03 | −0.02 | (±0.35) | −0.34 | (±0.34) | −0.27 | (±0.39) | −0.16 | (±0.33) | −0.26 | (±0.37) |
| 427 | 0.01 | 0.05 | −0.45 | (±0.33) | −0.74 | (±0.3) | −0.78 | (±0.36) | −0.58 | (±0.4) | −0.58 | (±0.37) |
| 428 | 0.00 | 0.01 | 0.33 | (±0.39) | −0.23 | (±0.37) | 0.14 | (±0.43) | 0.24 | (±0.46) | 0.01 | (±0.49) |
| 429 | 0.01 | 0.05 | −0.4 | (±0.41) | −0.59 | (±0.38) | −0.82 | (±0.56) | −0.76 | (±0.58) | −0.77 | (±0.41) |
| 430 | 0.00 | 0.01 | −0.27 | (±0.3) | −0.39 | (±0.27) | −0.54 | (±0.25) | −0.49 | (±0.3) | −0.58 | (±0.29) |
| 431 | 0.01 | 0.04 | −1.4 | (±0.36) | −1.72 | (±0.26) | −1.75 | (±0.34) | −1.6 | (±0.35) | −1.7 | (±0.4) |
| 432 | 0.00 | 0.00 | −0.76 | (±0.28) | −1.04 | (±0.21) | −1.07 | (±0.24) | −0.91 | (±0.31) | −1.06 | (±0.2) |
| 433 | 0.00 | 0.00 | −0.91 | (±0.36) | −1.26 | (±0.34) | −1.41 | (±0.25) | −1.3 | (±0.42) | −1.32 | (±0.35) |
| 434 | 0.01 | 0.04 | −1.88 | (±0.27) | −2.04 | (±0.21) | −2.08 | (±0.19) | −2 | (±0.26) | −2.08 | (±0.18) |
| 435 | 0.00 | 0.01 | −0.61 | (±0.53) | −0.73 | (±0.38) | −1.06 | (±0.45) | −0.96 | (±0.55) | −1.07 | (±0.45) |
| 436 | 0.01 | 0.05 | −0.54 | (±0.27) | −0.56 | (±0.23) | −0.79 | (±0.3) | −0.75 | (±0.35) | −0.77 | (±0.31) |
| 437 | 0.00 | 0.02 | −2.12 | (±0.38) | −2.32 | (±0.78) | −2.43 | (±0.43) | −2.15 | (±0.51) | −2.45 | (±0.3) |
| 438 | 0.00 | 0.00 | −2.01 | (±0.36) | −2.53 | (±0.5) | −2.34 | (±0.34) | −2.07 | (±0.36) | −2.36 | (±0.36) |
| 439 | 0.00 | 0.01 | −2.75 | (±0.27) | −3.13 | (±0.4) | −3.22 | (±0.36) | −3.01 | (±0.51) | −3.23 | (±0.3) |
| 440 | 0.00 | 0.02 | −1.56 | (±0.4) | −1.95 | (±0.38) | −1.94 | (±0.41) | −1.65 | (±0.42) | −1.83 | (±0.52) |
| 441 | 0.00 | 0.00 | −1.45 | (±0.35) | −1.92 | (±0.3) | −1.95 | (±0.42) | −1.52 | (±0.45) | −1.69 | (±0.35) |
| 442 | 0.00 | 0.01 | −0.92 | (±0.43) | −1.24 | (±0.29) | −1.23 | (±0.33) | −0.97 | (±0.46) | −1.16 | (±0.39) |
| 443 | 0.00 | 0.03 | −1.17 | (±0.35) | −1.37 | (±0.26) | −1.46 | (±0.32) | −1.21 | (±0.33) | −1.42 | (±0.34) |
| 444 | 0.01 | 0.04 | −2 | (±0.45) | −2.28 | (±0.35) | −2.05 | (±0.46) | −2.03 | (±0.47) | −2.33 | (±0.38) |
| 445 | 0.00 | 0.02 | −1.55 | (±0.52) | −1.94 | (±0.34) | −1.9 | (±0.41) | −1.7 | (±0.46) | −1.92 | (±0.45) |
| 446 | 0.00 | 0.01 | −0.91 | (±0.25) | −1.2 | (±0.17) | −1.27 | (±0.27) | −1.13 | (±0.26) | −1.14 | (±0.24) |
| 447 | 0.00 | 0.00 | −0.32 | (±0.35) | −0.73 | (±0.25) | −0.62 | (±0.38) | −0.39 | (±0.27) | −0.41 | (±0.36) |
| 448 | 0.00 | 0.00 | 1.57 | (±0.41) | 0.9 | (±0.41) | 1.01 | (±0.49) | 1.31 | (±0.36) | 1.42 | (±0.47) |
| 449 | 0.00 | 0.00 | −0.68 | (±0.35) | −1.22 | (±0.36) | −1.04 | (±0.41) | −0.84 | (±0.32) | −0.89 | (±0.4) |
| 450 | 0.01 | 0.05 | 0.54 | (±0.39) | 0.28 | (±0.31) | 0.36 | (±0.5) | 0.49 | (±0.48) | 0.53 | (±0.52) |
| 451 | 0.00 | 0.00 | −1.38 | (±0.42) | −1.92 | (±0.33) | −1.59 | (±0.51) | −1.19 | (±0.48) | −1.42 | (±0.51) |
| 452 | 0.00 | 0.00 | 0.14 | (±0.35) | −0.12 | (±0.26) | −0.05 | (±0.31) | 0.17 | (±0.27) | 0.12 | (±0.31) |
| 453 | 0.00 | 0.00 | 0.2 | (±0.48) | −0.13 | (±0.42) | −0.21 | (±0.46) | 0.31 | (±0.52) | 0.14 | (±0.5) |
| 454 | 0.00 | 0.01 | −0.55 | (±0.33) | −0.83 | (±0.48) | −0.76 | (±0.47) | −0.31 | (±0.5) | −0.52 | (±0.47) |
| 455 | 0.00 | 0.00 | 0.03 | (±0.42) | −0.28 | (±0.4) | −0.36 | (±0.4) | 0.06 | (±0.61) | −0.18 | (±0.44) |
| 456 | 0.00 | 0.02 | −0.4 | (±0.4) | −0.85 | (±0.38) | −0.72 | (±0.39) | −0.6 | (±0.36) | −0.69 | (±0.48) |
| 457 | 0.00 | 0.00 | −0.01 | (±0.32) | −0.39 | (±0.31) | −0.35 | (±0.36) | −0.19 | (±0.31) | −0.4 | (±0.38) |
| 458 | 0.01 | 0.04 | −0.16 | (±0.31) | −0.31 | (±0.23) | −0.19 | (±0.27) | −0.05 | (±0.26) | −0.25 | (±0.29) |
| 459 | 0.00 | 0.01 | −0.49 | (±0.46) | −0.86 | (±0.27) | −0.81 | (±0.51) | −0.65 | (±0.48) | −0.93 | (±0.33) |
| 460 | 0.01 | 0.03 | −1.43 | (±0.39) | −1.62 | (±0.21) | −1.52 | (±0.29) | −1.37 | (±0.26) | −1.49 | (±0.37) |
| 461 | 0.01 | 0.05 | −0.46 | (±0.4) | −0.52 | (±0.34) | −0.59 | (±0.34) | −0.4 | (±0.28) | −0.63 | (±0.3) |
| 462 | 0.01 | 0.05 | −0.02 | (±0.4) | −0.48 | (±0.31) | −0.36 | (±0.31) | −0.4 | (±0.32) | −0.39 | (±0.39) |
| 463 | 0.00 | 0.02 | 0.45 | (±0.38) | −0.04 | (±0.3) | 0.3 | (±0.41) | 0.16 | (±0.39) | 0.18 | (±0.37) |
| 464 | 0.00 | 0.01 | 0.24 | (±0.34) | −0.07 | (±0.23) | 0.09 | (±0.33) | 0.22 | (±0.2) | −0.04 | (±0.28) |
| 465 | 0.00 | 0.01 | 0.3 | (±0.4) | −0.02 | (±0.22) | 0.02 | (±0.26) | 0.13 | (±0.25) | 0.01 | (±0.31) |
| 466 | 0.00 | 0.00 | 0 | (±0.42) | −0.31 | (±0.27) | −0.24 | (±0.4) | 0.02 | (±0.42) | −0.35 | (±0.4) |
| 467 | 0.00 | 0.01 | −0.23 | (±0.4) | −0.6 | (±0.36) | −0.61 | (±0.29) | −0.43 | (±0.36) | −0.61 | (±0.34) |
| 468 | 0.00 | 0.00 | 1.16 | (±0.53) | 0.53 | (±0.4) | 0.7 | (±0.28) | 0.92 | (±0.48) | 0.77 | (±0.43) |
| 469 | 0.00 | 0.02 | 0 | (±0.67) | −0.43 | (±0.69) | −0.42 | (±0.74) | 0.39 | (±0.8) | −0.32 | (±0.65) |
| 470 | 0.00 | 0.00 | 2.13 | (±0.91) | 1.46 | (±1) | 1.46 | (±1.14) | 2.57 | (±1.08) | 1.41 | (±0.95) |
| 471 | 0.00 | 0.00 | 0.96 | (±0.58) | −0.01 | (±0.64) | 0.06 | (±0.4) | 0.09 | (±0.45) | 0.29 | (±0.45) |
| 472 | 0.00 | 0.00 | 1.48 | (±1.05) | 0.52 | (±0.46) | 0.76 | (±0.51) | 0.94 | (±0.7) | 1.32 | (±0.64) |
| 473 | 0.00 | 0.00 | 0.57 | (±0.66) | −0.51 | (±0.52) | −0.5 | (±0.56) | 0.3 | (±0.67) | 0.36 | (±0.56) |
| 474 | 0.00 | 0.00 | 1.09 | (±0.63) | 0.06 | (±0.43) | 0.07 | (±0.75) | 0.6 | (±0.6) | 0.53 | (±0.69) |
| 475 | 0.00 | 0.00 | 1.64 | (±0.76) | 0.38 | (±0.34) | 0.68 | (±0.64) | 1.14 | (±0.65) | 1.13 | (±0.62) |
| 476 | 0.00 | 0.01 | 1.1 | (±0.77) | 0.03 | (±0.62) | 0.13 | (±0.53) | 0.44 | (±0.51) | 0.43 | (±0.64) |
| 477 | 0.00 | 0.00 | 1.94 | (±0.66) | 0.74 | (±0.57) | 0.81 | (±0.74) | 1.1 | (±0.75) | 1.18 | (±0.68) |
| 478 | 0.00 | 0.00 | 1.62 | (±0.69) | 0.53 | (±0.53) | 0.68 | (±0.69) | 0.97 | (±0.74) | 1.04 | (±0.56) |
| 479 | 0.00 | 0.01 | −1.67 | (±0.56) | −2.13 | (±0.36) | −1.94 | (±0.28) | −1.69 | (±0.36) | −1.9 | (±0.37) |
| 480 | 0.00 | 0.01 | −2.4 | (±0.25) | −2.6 | (±0.34) | −2.47 | (±0.35) | −2.22 | (±0.37) | −2.67 | (±0.32) |
| 481 | 0.01 | 0.05 | −2.16 | (±0.34) | −2.35 | (±0.3) | −2.25 | (±0.32) | −2.01 | (±0.28) | −2.19 | (±0.26) |
| 482 | 0.00 | 0.00 | −1.39 | (±0.33) | −1.95 | (±0.3) | −1.75 | (±0.29) | −1.6 | (±0.3) | −1.71 | (±0.34) |
| 483 | 0.00 | 0.01 | −0.7 | (±0.62) | −1.28 | (±0.47) | −1.15 | (±0.59) | −0.74 | (±0.35) | −0.96 | (±0.5) |
| 484 | 0.00 | 0.00 | −0.76 | (±0.34) | −1.13 | (±0.26) | −1.1 | (±0.19) | −0.85 | (±0.17) | −1.02 | (±0.25) |
| 485 | 0.00 | 0.00 | −0.35 | (±0.24) | −0.66 | (±0.29) | −0.56 | (±0.18) | −0.4 | (±0.22) | −0.6 | (±0.26) |
| 486 | 0.00 | 0.00 | −0.31 | (±0.29) | −0.62 | (±0.23) | −0.57 | (±0.24) | −0.31 | (±0.27) | −0.33 | (±0.27) |
| 487 | 0.00 | 0.00 | −1.16 | (±0.44) | −1.46 | (±0.3) | −1.47 | (±0.33) | −1.16 | (±0.28) | −1.38 | (±0.38) |
| 488 | 0.00 | 0.00 | −1.51 | (±0.53) | −1.93 | (±0.26) | −1.83 | (±0.34) | −1.55 | (±0.36) | −1.77 | (±0.4) |
| 489 | 0.00 | 0.02 | −0.81 | (±0.27) | −0.97 | (±0.14) | −0.97 | (±0.22) | −0.85 | (±0.18) | −0.9 | (±0.24) |
| 490 | 0.00 | 0.00 | 1.37 | (±0.63) | 0.13 | (±0.7) | 0.47 | (±0.66) | 0.9 | (±0.52) | 0.88 | (±0.64) |
| 491 | 0.00 | 0.00 | 0.65 | (±0.58) | −0.03 | (±0.56) | 0 | (±0.4) | 0.03 | (±0.42) | 0.15 | (±0.58) |
| 492 | 0.00 | 0.02 | 1.08 | (±0.81) | 0.11 | (±0.51) | 0.14 | (±0.54) | 0.37 | (±0.41) | 0.35 | (±0.59) |
| 493 | 0.00 | 0.00 | 2.94 | (±0.51) | 1.41 | (±0.47) | 1.46 | (±0.59) | 1.85 | (±0.69) | 1.84 | (±0.62) |
| 494 | 0.00 | 0.00 | −0.42 | (±0.62) | −1.14 | (±0.4) | −1.13 | (±0.35) | −0.73 | (±0.36) | −0.92 | (±0.47) |
| 495 | 0.00 | 0.00 | −0.47 | (±0.48) | −1.16 | (±0.39) | −1.04 | (±0.32) | −0.86 | (±0.38) | −0.89 | (±0.51) |
| 496 | 0.00 | 0.00 | −0.54 | (±0.44) | −1.16 | (±0.26) | −1.14 | (±0.29) | −0.93 | (±0.43) | −0.94 | (±0.33) |
| 497 | 0.00 | 0.01 | −0.47 | (±0.36) | −0.84 | (±0.24) | −0.69 | (±0.23) | −0.53 | (±0.24) | −0.63 | (±0.19) |
| 498 | 0.00 | 0.00 | −0.12 | (±0.57) | −0.93 | (±0.44) | −0.7 | (±0.4) | −0.35 | (±0.4) | −0.52 | (±0.38) |
| 499 | 0.00 | 0.00 | −2.33 | (±0.43) | −2.78 | (±0.35) | −2.54 | (±0.34) | −2.25 | (±0.35) | −2.5 | (±0.31) |
| 500 | 0.00 | 0.00 | −2.16 | (±0.41) | −2.85 | (±0.37) | −2.62 | (±0.25) | −2.33 | (±0.33) | −2.4 | (±0.35) |
| 501 | 0.00 | 0.00 | −2.04 | (±0.44) | −2.87 | (±0.45) | −2.6 | (±0.36) | −2.35 | (±0.33) | −2.64 | (±0.43) |
| 502 | 0.00 | 0.00 | −2.25 | (±0.46) | −3.03 | (±0.46) | −2.84 | (±0.37) | −2.47 | (±0.34) | −2.68 | (±0.46) |
| 503 | 0.00 | 0.00 | −0.06 | (±0.48) | −0.74 | (±0.35) | −0.63 | (±0.42) | −0.48 | (±0.33) | −0.69 | (±0.32) |
| 504 | 0.00 | 0.00 | 0.06 | (±0.42) | −0.52 | (±0.36) | −0.41 | (±0.46) | −0.17 | (±0.35) | −0.3 | (±0.34) |
| 505 | 0.00 | 0.00 | −1.07 | (±0.56) | −1.77 | (±0.55) | −1.59 | (±0.47) | −1.29 | (±0.53) | −1.55 | (±0.58) |
| 506 | 0.00 | 0.00 | 0.72 | (±0.47) | 0.1 | (±0.38) | 0 | (±0.35) | 0 | (±0.38) | 0.15 | (±0.41) |
| 507 | 0.01 | 0.04 | −1.34 | (±0.5) | −1.88 | (±0.39) | −1.69 | (±0.33) | −1.54 | (±0.38) | −1.62 | (±0.39) |
| 508 | 0.00 | 0.00 | −0.42 | (±0.39) | −0.81 | (±0.33) | −0.85 | (±0.34) | −0.61 | (±0.33) | −0.73 | (±0.3) |
| 509 | 0.01 | 0.05 | 0.46 | (±0.86) | 0.15 | (±0.84) | 0.32 | (±0.86) | 0.14 | (±0.87) | 0.22 | (±0.73) |
| 510 | 0.00 | 0.00 | 2.58 | (±0.42) | 2.12 | (±0.59) | 2.57 | (±0.54) | 2.46 | (±0.66) | 2.27 | (±0.51) |
| 511 | 0.00 | 0.02 | 0.77 | (±0.47) | 0.22 | (±0.34) | 0.37 | (±0.42) | 0.59 | (±0.49) | 0.43 | (±0.42) |
| 512 | 0.00 | 0.00 | −0.2 | (±0.38) | −0.69 | (±0.23) | −0.57 | (±0.28) | −0.33 | (±0.26) | −0.38 | (±0.31) |
| 513 | 0.00 | 0.00 | 0.01 | (±0.47) | −0.55 | (±0.28) | −0.51 | (±0.36) | −0.33 | (±0.39) | −0.43 | (±0.39) |
| 514 | 0.00 | 0.00 | 0.82 | (±0.39) | −0.09 | (±0.26) | 0.07 | (±0.37) | 0.25 | (±0.37) | 0.22 | (±0.4) |
| 515 | 0.00 | 0.01 | −1.54 | (±0.51) | −1.79 | (±0.32) | −1.74 | (±0.34) | −1.59 | (±0.42) | −2.01 | (±0.34) |
| 516 | 0.00 | 0.01 | 0.27 | (±0.55) | −0.18 | (±0.28) | 0.06 | (±0.32) | 0.17 | (±0.41) | 0.04 | (±0.45) |
| 517 | 0.00 | 0.00 | −0.79 | (±0.41) | −1.17 | (±0.38) | −0.95 | (±0.35) | −0.77 | (±0.37) | −0.97 | (±0.39) |
| 518 | 0.00 | 0.00 | 0.35 | (±0.44) | −0.23 | (±0.4) | 0.31 | (±0.37) | 0.29 | (±0.36) | 0.04 | (±0.53) |
| 519 | 0.00 | 0.00 | 1.25 | (±0.66) | 0.54 | (±0.56) | 1.04 | (±0.65) | 1.15 | (±0.71) | 0.71 | (±0.61) |
| 520 | 0.00 | 0.00 | 1.71 | (±0.91) | 0.62 | (±0.7) | 1.12 | (±0.7) | 1.21 | (±0.82) | 1.15 | (±0.64) |
| 521 | 0.00 | 0.00 | 0.52 | (±0.63) | −0.38 | (±0.36) | −0.08 | (±0.41) | 0.12 | (±0.36) | −0.18 | (±0.38) |
| 522 | 0.00 | 0.00 | 0.34 | (±0.54) | −0.28 | (±0.34) | −0.12 | (±0.42) | 0.09 | (±0.46) | −0.1 | (±0.43) |
| 523 | 0.00 | 0.00 | 1.55 | (±0.76) | 0.14 | (±0.46) | 0.54 | (±0.53) | 0.79 | (±0.67) | 0.54 | (±0.7) |
| 524 | 0.01 | 0.04 | 1.46 | (±0.52) | 0.75 | (±0.63) | 1.24 | (±0.57) | 1.37 | (±0.52) | 1.42 | (±0.5) |
| 525 | 0.01 | 0.04 | 0.61 | (±0.63) | 0.04 | (±0.41) | 0.18 | (±0.43) | 0.49 | (±0.48) | 0.36 | (±0.5) |
| 526 | 0.00 | 0.00 | 2.53 | (±0.77) | 1.61 | (±0.61) | 2.07 | (±0.65) | 2.31 | (±0.91) | 2.1 | (±0.66) |
| 527 | 0.00 | 0.00 | 3.03 | (±0.7) | 2.03 | (±0.41) | 2.47 | (±0.46) | 2.49 | (±0.59) | 2.44 | (±0.58) |
| 528 | 0.00 | 0.00 | 3.18 | (±0.73) | 2.27 | (±0.57) | 2.77 | (±0.6) | 2.87 | (±0.64) | 2.65 | (±0.61) |
| 529 | 0.00 | 0.00 | 3.12 | (±0.72) | 2.15 | (±0.56) | 2.71 | (±0.6) | 2.86 | (±0.7) | 2.64 | (±0.63) |
| 530 | 0.01 | 0.04 | 1.39 | (±0.61) | 0.63 | (±0.49) | 1.04 | (±0.47) | 1.15 | (±0.5) | 0.93 | (±0.57) |
| 531 | 0.00 | 0.00 | 2.24 | (±0.68) | 1.32 | (±0.44) | 1.73 | (±0.48) | 1.87 | (±0.52) | 1.66 | (±0.5) |
| 532 | 0.00 | 0.00 | 2.3 | (±0.69) | 1.33 | (±0.49) | 1.78 | (±0.53) | 1.95 | (±0.56) | 1.68 | (±0.57) |

The inventive determined gene activity and the distribution in specific gene clusters is thus characteristic for operative intervention in a liver transplantation and usable for the in vitro monitoring of liver transplantations.

TABLE 10

| | | GenBank Accession | GenBank | RefSeq | | UniGene Accession | UniGene | UniGene |
|---|---|---|---|---|---|---|---|---|
| Sequence-ID | GenBank clone ID | No. | Description | Accession No. | Best RefSeq Description | No. | Gen | Description |
| 1 | | BC063685.1 | Homo sapiens | NM_005429.2 | Homo sapiens vascular | Hs.435215 | Vascular | VEGFC |
| | | | vascular endothelial | | endothelial growth factor C | | endothelial | |
| | | | growth factor C, | | (VEGFC), mRNA | | growth factor C | |
| | | | mRNA (cDNA clone | | Length = 2076 | | | |
| | | | MGC: 74703 | | | | | |
| | | | IMAGE: 4513934), | | | | | |
| | | | complete cds | | | | | |
| | | | Length = 2076 | | | | | |
| 2 | | | | | | Hs.528087 | Inositol | ITPKB |
| | | | | | | | 1,4,5- | |
| | | | | | | | trisphosphate | |
| | | | | | | | 3-kinase B | |
| 3 | | | | | | Hs.162868 | D4, zinc and | DPF3 |
| | | | | | | | double PHD | |
| | | | | | | | fingers, | |
| | | | | | | | family 3 | |
| 4 | | AC011418.5 | Homo sapiens | NM_002187.2 | Homo sapiens interleukin | Hs.674 | Interleukin | IL12B |
| | | | chromosome 5 clone | | 12B (natural killer cell | | 12B (natural | |
| | | | CTB-9P16, complete | | stimulatory factor 2, | | killer cell | |
| | | | sequence | | cytotoxic lymphocyte | | stimulatory | |
| | | | Length = 108511 | | maturation factor 2, p40) | | factor 2, | |
| | | | | | (IL12B), mRNA | | cytotoxic | |
| | | | | | Length = 2347 | | lymphocyte | |
| | | | | | | | maturation | |
| | | | | | | | factor 2, p40) | |
| 5 | | BC014661.1 | Homo sapiens | NM_022895.1 | Homo sapiens hypothetical | Hs.528671 | Hypothetical | FLJ12448 |
| | | | hypothetical protein | | protein FLJ12448 | | protein | |
| | | | FLJ12448, mRNA | | (FLJ12448), mRNA | | FLJ12448 | |
| | | | (cDNA clone | | Length = 1920 | | | |
| | | | MGC: 22955 | | | | | |
| | | | IMAGE: 4860511), | | | | | |
| | | | complete cds | | | | | |
| | | | Length = 1921 | | | | | |
| 6 | | AY720441.1 | Homo sapiens | NM_000758.2 | Homo sapiens colony | Hs.1349 | Colony | CSF2 |
| | | | granulocyte- | | stimulating factor 2 | | stimulating | |
| | | | macrophage colony | | (granulocyte-macrophage) | | factor 2 | |
| | | | stimulating factor 2 | | (CSF2), mRNA Length = 781 | | (granulocyte- | |
| | | | mRNA, complete cds | | | | macrophage) | |
| | | | Length = 459 | | | | | |
| 7 | | BC063474.1 | Homo sapiens G | NM_018040.1 | Homo sapiens G patch | Hs.420757 | G patch | GPATC2 |
| | | | patch domain | | domain containing 2 | | domain | |
| | | | containing 2, mRNA | | (GPATC2), mRNA | | containing 2 | |
| | | | (cDNA clone | | Length = 2338 | | | |
| | | | MGC: 74998 | | | | | |
| | | | IMAGE: 5206348), | | | | | |
| | | | complete cds | | | | | |
| | | | Length = 3250 | | | | | |
| 8 | IMAGE: 1707196 | | | | | | | |
| 9 | | BC008628.1 | Homo sapiens | NM_003851.2 | Homo sapiens cellular | Hs.5710 | Cellular | CREG1 |
| | | | cellular repressor of | | repressor of E1A- | | repressor of | |
| | | | E1A-stimulated | | stimulated genes 1 | | E1A- | |
| | | | genes 1, mRNA | | (CREG1), mRNA | | stimulated | |
| | | | (cDNA clone | | Length = 2048 | | genes 1 | |
| | | | MGC: 17147 | | | | | |
| | | | IMAGE: 4339480), | | | | | |
| | | | complete cds | | | | | |
| | | | Length = 1969 | | | | | |
| 10 | | AY545477.1 | Homo sapiens | NM_001752.2 | Homo sapiens catalase | Hs.502302 | Catalase | CAT |
| | | | catalase (CAT) gene, | | (CAT), mRNA Length = 2305 | | | |
| | | | complete cds | | | | | |
| | | | Length = 37116 | | | | | |
| 11 | | | | NM_012450 | Homo sapiens solute | | solute carrier | SLC13A4 |
| | | | | | carrier family 13 | | family 13 | |
| | | | | | (sodium/sulfate | | (sodium/sulfate | |
| | | | | | symporters), member 4 | | symporters), | |
| | | | | | (SLC13A4), mRNA | | member 4 | |
| 12 | | | | | | Hs.561807 | Transcribed | |
| | | | | | | | locus | |
| 13 | IMAGE: 2244531 | | | | | | | |
| 14 | | AC147090.3 | Pan troglodytes BAC | | | Hs.559750 | Transcribed | |
| | | | clone RP43-58D11 | | | | locus | |
| | | | from 7, complete | | | | | |
| | | | sequence | | | | | |
| | | | Length = 200913 | | | | | |
| 15 | | | | | | Hs.522463 | Eukaryotic | EEF1A1 |
| | | | | | | | translation | |
| | | | | | | | elongation | |
| | | | | | | | factor 1 | |
| | | | | | | | alpha 1 | |
| 16 | IMAGE: 460157 | | | | | | | |
| 17 | | AC012360.7 | Homo sapiens BAC | | | Hs.446350 | Transforming | TGFBRAP1 |
| | | | clone RP11-332H14 | | | | growth | |
| | | | from 2, complete | | | | factor, beta | |
| | | | sequence | | | | receptor | |
| | | | Length = 200918 | | | | associated | |
| | | | Features flanking | | | | protein 1 | |
| | | | this part of subject | | | | | |
| | | | sequence: 2755 bp | | | | | |
| | | | at 5 side: unknown | | | | | |
| | | | 21585 bp at 3 side: | | | | | |
| | | | unknown | | | | | |
| 18 | | AC002076.2 | Homo sapiens BAC | | | | | |
| | | | clone GS1-345D13 | | | | | |
| | | | from 7, complete | | | | | |
| | | | sequence | | | | | |
| | | | Length = 149764 | | | | | |
| 19 | | AC011200.9 | AC011200 Homo | | | Hs.374023 | Potassium | KCNQ3 |
| | | | sapiens | | | | voltage- | |
| | | | chromosome 8 clone | | | | gated | |
| | | | RP11-71N3, | | | | channel, | |
| | | | complete sequence | | | | KQT-like | |
| | | | Length = 161271 | | | | subfamily, | |
| | | | | | | | member 3 | |
| 20 | | AC137055.12 | Homo sapiens 12 | | | Hs.76253 | Ataxin 2 | ATXN2 |
| | | | BAC RP11-686G8 | | | | | |
| | | | (Roswell Park | | | | | |
| | | | Cancer Institute | | | | | |
| | | | Human BAC Library) | | | | | |
| | | | complete sequence | | | | | |
| | | | Length = 107717 | | | | | |
| 21 | | BC038295.2 | Homo sapiens | NM_145185.2 | Homo sapiens mitogen- | Hs.531754 | Mitogen- | MAP2K7 |
| | | | mitogen-activated | | activated protein kinase | | activated | |
| | | | protein kinase kinase | | kinase 7 (MAP2K7), mRNA | | protein | |
| | | | 7, mRNA (cDNA | | Length = 3386 | | kinase | |
| | | | clone MGC: 33283 | | | | kinase 7 | |
| | | | IMAGE: 5269063), | | | | | |
| | | | complete cds | | | | | |
| | | | Length = 3446 | | | | | |
| 22 | | BC017065.1 | Homo sapiens tumor | NM_032957.2 | Homo sapiens regulator of | Hs.434878 | Tumor | TNFRSF6B |
| | | | necrosis factor | | telomere elongation | | necrosis | |
| | | | receptor superfamily, | | helicase 1 (RTEL1), | | factor | |
| | | | member 6b, decoy, | | transcript variant 2, mRNA | | receptor | |
| | | | transcript variant | | Length = 5288 | | superfamily, | |
| | | | M68E, mRNA (cDNA | | | | member 6b, | |
| | | | clone MGC: 9587 | | | | decoy | |
| | | | IMAGE: 3886635), | | | | | |
| | | | complete cds | | | | | |
| | | | Length = 1125 | | | | | |
| 23 | | | | | | Hs.163301 | CDNA | |
| | | | | | | | FLJ35132 | |
| | | | | | | | fis, clone | |
| | | | | | | | PLACE6008989 | |
| 24 | | BC082233.1 | Homo sapiens cDNA | | | Hs.187337 | Zinc finger | LOC148213 |
| | | | clone | | | | protein 681 | |
| | | | IMAGE: 3833639 | | | | | |
| | | | Length = 1196 | | | | | |
| 25 | | BC038289.1 | Homo sapiens | NM_032964.2 | Homo sapiens chemokine | Hs.272493 | Chemokine | CCL15 |
| | | | chemokine (C-C | | (C-C motif) ligand 15 | | (C-C motif) | |
| | | | motif) ligand 14, | | (CCL15), transcript variant | | ligand 15 | |
| | | | transcript variant 3, | | 1, mRNA Length = 1344 | | | |
| | | | mRNA (cDNA clone | | | | | |
| | | | MGC: 47546 | | | | | |
| | | | IMAGE: 5222914), | | | | | |
| | | | complete cds | | | | | |
| | | | Length = 533 | | | | | |
| 26 | | AY463360.1 | Homo sapiens | | | Hs.476018 | Catenin | CTNNB1 |
| | | | catenin (cadherin- | | | | (cadherin- | |
| | | | associated protein), | | | | associated | |
| | | | beta 1, 88 kDa | | | | protein), beta | |
| | | | (CTNNB1) gene, | | | | 1, 88 kDa | |
| | | | complete cds | | | | | |
| | | | Length = 44505 | | | | | |
| 27 | | BC001830.2 | Homo sapiens | NM_015927.3 | Homo sapiens transforming | Hs.513530 | Transforming | TGFB1I1 |
| | | | transforming growth | | growth factor beta 1 | | growth factor | |
| | | | factor beta 1 induced | | induced transcript 1 | | beta 1 | |
| | | | transcript 1, mRNA | | (TGFB1I1), mRNA | | induced | |
| | | | (cDNA clone | | Length = 1824 | | transcript 1 | |
| | | | MGC: 4078 | | | | | |
| | | | IMAGE: 3030477), | | | | | |
| | | | complete cds | | | | | |
| | | | Length = 1824 | | | | | |
| 28 | | AC004076.1 | AC004076 Homo | | | Hs.147471 | Similar to | |
| | | | sapiens | | | | R30217_1 | |
| | | | chromosome 19, | | | | | |
| | | | cosmid R30217, | | | | | |
| | | | complete sequence | | | | | |
| | | | Length = 41322 | | | | | |
| 29 | | BC038289.1 | Homo sapiens | NM_032964.2 | Homo sapiens chemokine | Hs.272493 | Chemokine | CCL15 |
| | | | chemokine (C-C | | (C-C motif) ligand 15 | | (C-C motif) | |
| | | | motif) ligand 14, | | (CCL15), transcript variant | | ligand 15 | |
| | | | transcript variant 3, | | 1, mRNA Length = 1344 | | | |
| | | | mRNA (cDNA clone | | | | | |
| | | | MGC: 47546 | | | | | |
| | | | IMAGE: 5222914), | | | | | |
| | | | complete cds | | | | | |
| | | | Length = 533 | | | | | |
| 30 | | | | | | Hs.546743 | CDNA clone | |
| | | | | | | | IMAGE: 5272066 | |
| 31 | | AC006020.2 | AC006020 Homo | | | Hs.119807 | Transcribed | |
| | | | sapiens PAC clone | | | | locus | |
| | | | RP5-1049N15 from | | | | | |
| | | | 7q31.2-7q32, | | | | | |
| | | | complete sequence | | | | | |
| | | | Length = 152811 | | | | | |
| 32 | | AC069387.8 | Homo sapiens BAC | NM_138938.1 | Homo sapiens regenerating | Hs.503388 | Regenerating | PAP |
| | | | clone RP11-295G11 | | islet-derived 3 alpha | | islet- | |
| | | | from 2, complete | | (REG3A), transcript variant | | derived 3 | |
| | | | sequence | | 2, mRNA Length = 1002 | | alpha | |
| | | | Length = 47340 | | | | | |
| 33 | | | | | | Hs.283844 | Sideroflexin 3 | SFXN3 |
| 34 | | BC023640.1 | Homo sapiens, clone | XM_520711.1 | PREDICTED: Pan | Hs.494538 | Patched | PTCH |
| | | | IMAGE: 4890344, | | troglodytes similar to | | homolog | |
| | | | mRNA Length = 1429 | | patched; patched | | (Drosophila) | |
| | | | | | (Drosophila) homolog | | | |
| | | | | | (LOC465254), mRNA | | | |
| | | | | | Length = 5538 | | | |
| 35 | | BC077719.1 | Homo sapiens | NM_152718.1 | Homo sapiens hypothetical | Hs.60640 | Hypothetical | FLJ32009 |
| | | | hypothetical protein | | protein FLJ32009 | | protein | |
| | | | FLJ32009, mRNA | | (FLJ32009), mRNA | | FLJ32009 | |
| | | | (cDNA clone | | Length = 3292 | | | |
| | | | IMAGE: 6305035), | | | | | |
| | | | complete cds | | | | | |
| | | | Length = 3385 | | | | | |
| 36 | | BC073768.1 | Homo sapiens heat | NM_001540.2 | Homo sapiens heat shock | Hs.521554 | Transcribed | |
| | | | shock 27 kDa protein | | 27 kDa protein 1 (HSPB1), | | locus, | |
| | | | 1, mRNA (cDNA | | mRNA Length = 865 | | strongly | |
| | | | clone MGC: 88776 | | | | similar to | |
| | | | IMAGE: 5444848), | | | | NP_001531.1 | |
| | | | complete cds | | | | heat shock | |
| | | | Length = 794 | | | | 27 kDa | |
| | | | | | | | protein 1; | |
| | | | | | | | heat shock | |
| | | | | | | | 27 kD protein | |
| | | | | | | | 1 [Homo | |
| | | | | | | | sapiens] | |
| 37 | | | | | | Hs.399800 | A kinase | AKAP8L |
| | | | | | | | (PRKA) | |
| | | | | | | | anchor | |
| | | | | | | | protein 8-like | |
| 38 | | AC091564.12 | Homo sapiens | | | | | |
| | | | chromosome 11, | | | | | |
| | | | clone RP11-732A19, | | | | | |
| | | | complete sequence | | | | | |
| | | | Length = 211735 | | | | | |
| 39 | | AC005808.1 | AC005808 Homo | | | | | |
| | | | sapiens | | | | | |
| | | | chromosome 20, P1 | | | | | |
| | | | clone 185 (LBNL | | | | | |
| | | | H136), complete | | | | | |
| | | | sequence | | | | | |
| | | | Length = 57434 | | | | | |
| 40 | | BC015542.1 | Homo sapiens | NM_006505.2 | Homo sapiens poliovirus | Hs.171844 | Poliovirus | PVR |
| | | | poliovirus receptor, | | receptor (PVR), mRNA | | receptor | |
| | | | mRNA (cDNA clone | | Length = 3215 | | | |
| | | | MGC: 9603 | | | | | |
| | | | IMAGE: 3902226), | | | | | |
| | | | complete cds | | | | | |
| | | | Length = 3215 | | | | | |
| 41 | | | | NM_173582.3 | Homo sapiens | Hs.26612 | Phosphoglucomutase | PGM2L1 |
| | | | | | phosphoglucomutase 2-like | | 2- | |
| | | | | | 1 (PGM2L1), mRNA | | like 1 | |
| | | | | | Length = 8519 | | | |
| 42 | | BC023518.2 | Homo sapiens heat | NM_002157.1 | Homo sapiens heat shock | Hs.558338 | Heat shock | HSPE1 |
| | | | shock 10 kDa protein | | 10 kDa protein 1 | | 10 kDa | |
| | | | 1 (chaperonin 10), | | (chaperonin 10) (HSPE1), | | protein 1 | |
| | | | mRNA (cDNA clone | | mRNA Length = 538 | | (chaperonin | |
| | | | MGC: 12734 | | | | 10) | |
| | | | IMAGE: 4304224), | | | | | |
| | | | complete cds | | | | | |
| | | | Length = 556 | | | | | |
| 43 | | AC139705.4 | Homo sapiens X | | | | | |
| | | | BAC RP11-102M2 | | | | | |
| | | | (Roswell Park | | | | | |
| | | | Cancer Institute | | | | | |
| | | | Human BAC Library) | | | | | |
| | | | complete sequence | | | | | |
| | | | Length = 148316 | | | | | |
| 44 | | BC040709.2 | Homo sapiens cDNA | | | Hs.190394 | Hypothetical | LOC91661 |
| | | | clone | | | | protein | |
| | | | IMAGE: 6017695, | | | | BC001610 | |
| | | | partial cds | | | | | |
| | | | Length = 608 | | | | | |
| 45 | IMAGE: 2179769 | | | | | | | |
| 46 | | BC026356.1 | Homo sapiens Rho | NM_014470.2 | Homo sapiens Rho family | Hs.124940 | Rho family | RND1 |
| | | | family GTPase 1, | | GTPase 1 (RND1), mRNA | | GTPase 1 | |
| | | | mRNA (cDNA clone | | Length = 1681 | | | |
| | | | MGC: 26932 | | | | | |
| | | | IMAGE: 4793442), | | | | | |
| | | | complete cds | | | | | |
| | | | Length = 1681 | | | | | |
| 47 | | | | | | Hs.523732 | Secretoglobin, | SCGB1A1 |
| | | | | | | | family 1A, | |
| | | | | | | | member 1 | |
| | | | | | | | (uteroglobin) | |
| 48 | | | | | | Hs.120058 | CDNA | |
| | | | | | | | FLJ34446 | |
| | | | | | | | fis, clone | |
| | | | | | | | HLUNG2002050 | |
| 49 | | AC026230.5 | AC026230 Homo | NG_002793.1 | Homo sapiens | Hs.519304 | Pelota | PELO |
| | | | sapiens | | pelota/integrin, alpha 1 | | homolog | |
| | | | chromosome 5 clone | | region (PELO/ITGA1@) on | | (Drosophila) | |
| | | | CTD-2288O8, | | chromosome 5 | | | |
| | | | complete sequence | | Length = 166541 | | | |
| | | | Length = 114802 | | | | | |
| 50 | | AC026634.11 | Homo sapiens | | | Hs.445511 | RIO kinase 3 | RIOK3 |
| | | | chromosome, clone | | | | (yeast) | |
| | | | RP11-639E23, | | | | | |
| | | | complete sequence | | | | | |
| | | | Length = 164858 | | | | | |
| 51 | | | | | | Hs.177275 | Hypothetical | DJ122O8.2 |
| | | | | | | | protein | |
| | | | | | | | dJ122O8.2 | |
| 52 | | U95367.1 | HSU95367 Human | NM_014211.1 | Homo sapiens gamma- | Hs.26225 | Gamma- | GABRP |
| | | | GABA-A receptor pi | | aminobutyric acid (GABA) | | aminobutyric | |
| | | | subunit mRNA, | | A receptor, pi (GABRP), | | acid (GABA) | |
| | | | complete cds | | mRNA Length = 3282 | | A receptor, pi | |
| | | | Length = 3282 | | | | | |
| 53 | IMAGE: 1563026 | | | | | | | |
| 54 | | BC009421.1 | Homo sapiens | NM_153639.1 | Homo sapiens | Hs.516859 | Pantothenate | PANK2 |
| | | | pantothenate kinase | | pantothenate kinase 2 | | kinase 2 | |
| | | | 2 (Hallervorden- | | (Hallervorden-Spatz | | (Hallervorde | |
| | | | Spatz syndrome), | | syndrome) (PANK2), | | n-Spatz | |
| | | | mRNA (cDNA clone | | transcript variant 2, mRNA | | syndrome) | |
| | | | IMAGE: 3528235) | | Length = 1816 | | | |
| | | | Length = 1774 | | | | | |
| 55 | | BC007433.2 | Homo sapiens | NM_000366.5 | Homo sapiens tropomyosin | Hs.133892 | Tropomyosin | TPM1 |
| | | | tropomyosin 1 | | 1 (alpha) (TPM1), transcript | | 1 (alpha) | |
| | | | (alpha), mRNA | | variant 5, mRNA | | | |
| | | | (cDNA clone | | Length = 1294 | | | |
| | | | MGC: 4021 | | | | | |
| | | | IMAGE: 2962925), | | | | | |
| | | | complete cds | | | | | |
| | | | Length = 1256 | | | | | |
| 56 | | BC028614.1 | Homo sapiens Ras | NM_017805.2 | Homo sapiens Ras | Hs.233955 | Ras | RASIP1 |
| | | | interacting protein 1, | | interacting protein 1 | | interacting | |
| | | | mRNA (cDNA clone | | (RASIP1), mRNA | | protein 1 | |
| | | | MGC: 33504 | | Length = 3311 | | | |
| | | | IMAGE: 4815880), | | | | | |
| | | | complete cds | | | | | |
| | | | Length = 3190 | | | | | |
| 57 | | AY703984.1 | Homo sapiens SP-A | NM_004740.3 | Homo sapiens TGFB1- | Hs.462590 | TGFB1- | TIAF1 |
| | | | receptor subunit SP- | | induced anti-apoptotic | | induced anti- | |
| | | | R210 alphaS | | factor 1 (TIAF1), mRNA | | apoptotic | |
| | | | (MYO18A) mRNA, | | Length = 2110 | | factor 1 | |
| | | | complete cds, | | | | | |
| | | | alternatively spliced | | | | | |
| | | | Length = 6479 | | | | | |
| 58 | IMAGE: 1882829 | | | | | | | |
| 59 | | | | | | Hs.444975 | Pleiomorphic | PLAGL1 |
| | | | | | | | adenoma | |
| | | | | | | | gene-like 1 | |
| 60 | | AC009230.3 | Homo sapiens BAC | | | Hs.357567 | Hypothetical | LOC130576 |
| | | | clone RP11-343B21 | | | | protein | |
| | | | from 2, complete | | | | LOC130576 | |
| | | | sequence | | | | | |
| | | | Length = 173993 | | | | | |
| 61 | | | | | | Hs.292580 | Hypothetical | LOC283551 |
| | | | | | | | protein | |
| | | | | | | | LOC283551 | |
| 62 | | BC026340.1 | Homo sapiens | NM_000674.1 | Homo sapiens adenosine | Hs.77867 | Adenosine | ADORA1 |
| | | | adenosine A1 | | A1 receptor (ADORA1), | | A1 receptor | |
| | | | receptor, mRNA | | mRNA Length = 2900 | | | |
| | | | (cDNA clone | | | | | |
| | | | MGC: 26536 | | | | | |
| | | | IMAGE: 4797116), | | | | | |
| | | | complete cds | | | | | |
| | | | Length = 2756 | | | | | |
| 63 | | AC108080.2 | Homo sapiens | | | Hs.268939 | Matrin 3 | MATR3 |
| | | | chromosome 5 clone | | | | | |
| | | | CTC-315N8, | | | | | |
| | | | complete sequence | | | | | |
| | | | Length = 160494 | | | | | |
| 64 | | | | | | Hs.484678 | TBC1 | TBC1D7 |
| | | | | | | | domain | |
| | | | | | | | family, | |
| | | | | | | | member 7 | |
| 65 | | AY795482.1 | Homo sapiens DnaJ | NM_006260.2 | Homo sapiens DnaJ | Hs.59214 | Hypothetical | DNAJC3 |
| | | | (Hsp40) homolog, | | (Hsp40) homolog, | | protein | |
| | | | subfamily C, member | | subfamily C, member 3 | | LOC144871 | |
| | | | 3 (DNAJC3) gene, | | (DNAJC3), mRNA | | | |
| | | | complete cds | | Length = 1542 | | | |
| | | | Length = 117695 | | | | | |
| 66 | | BC042972.1 | Homo sapiens | NM_001003406.1 | Homo sapiens calcium | Hs.125116 | Calcium | CACNA1I |
| | | | calcium channel, | | channel, voltage- | | channel, | |
| | | | voltage-dependent, | | dependent, alpha 1I subunit | | voltage- | |
| | | | alpha 1I subunit, | | (CACNA1I), transcript | | dependent, | |
| | | | mRNA (cDNA clone | | variant 2, mRNA | | alpha 1I | |
| | | | IMAGE: 5294701) | | Length = 9902 | | subunit | |
| | | | Length = 2465 | | | | | |
| 67 | | AC020917.4 | AC020917 Homo | NM_016270.2 | Homo sapiens kruppel-like | Hs.107740 | Kruppel-like | KLF2 |
| | | | sapiens | | factor 2 (lung) (KLF2), | | factor 2 | |
| | | | chromosome 19 | | mRNA Length = 1655 | | (lung) | |
| | | | clone CTD-2013N17, | | | | | |
| | | | complete sequence | | | | | |
| | | | Length = 151088 | | | | | |
| 68 | IMAGE: 2018147 | | | | | | | |
| 69 | | | | | | Hs.100431 | Chemokine | CXCL13 |
| | | | | | | | (C—X—C motif) | |
| | | | | | | | ligand 13 (B- | |
| | | | | | | | cell | |
| | | | | | | | chemoattractant) | |
| 70 | | AC116552.2 | Homo sapiens | | | Hs.507185 | Zinc finger | ZFPM1 |
| | | | chromosome 16 | | | | protein, | |
| | | | clone RP11-21B21, | | | | multitype 1 | |
| | | | complete sequence | | | | | |
| | | | Length = 176797 | | | | | |
| 71 | | | | | | Hs.473218 | Phosphatase | PHACTR3 |
| | | | | | | | and actin | |
| | | | | | | | regulator 3 | |
| 72 | | AY358388.1 | Homo sapiens clone | NM_030945.2 | Homo sapiens C1q and | Hs.171929 | C1q and | C1QTNF3 |
| | | | DNA44686 CTRP3 | | tumor necrosis factor | | tumor | |
| | | | (UNQ753) mRNA, | | related protein 3 | | necrosis | |
| | | | complete cds | | (C1QTNF3), transcript | | factor related | |
| | | | Length = 1712 | | variant 1, mRNA | | protein 3 | |
| | | | | | Length = 3570 | | | |
| 73 | | | | | | Hs.280881 | Cyclin- | CDKL1 |
| | | | | | | | dependent | |
| | | | | | | | kinase-like 1 | |
| | | | | | | | (CDC2- | |
| | | | | | | | related | |
| | | | | | | | kinase) | |
| 74 | | AC012236.12 | Homo sapiens | | | Hs.558722 | Hypothetical | |
| | | | chromosome 15, | | | | LOC283710 | |
| | | | clone RP11-16E12, | | | | | |
| | | | complete sequence | | | | | |
| | | | Length = 173729 | | | | | |
| 75 | | AC006230.11 | AC006230 Homo | | | | | |
| | | | sapiens | | | | | |
| | | | chromosome 4 clone | | | | | |
| | | | C0287J14 map | | | | | |
| | | | 4p16, complete | | | | | |
| | | | sequence | | | | | |
| | | | Length = 166998 | | | | | |
| 76 | | AC112504.6 | Homo sapiens 3 | | | Hs.477789 | ATPase, | ATP1B3 |
| | | | BAC RP11-271K21 | | | | Na+/K+ | |
| | | | (Roswell Park | | | | transporting, | |
| | | | Cancer Institute | | | | beta 3 | |
| | | | Human BAC Library) | | | | polypeptide | |
| | | | complete sequence | | | | | |
| | | | Length = 215077 | | | | | |
| 77 | | | | | | Hs.170726 | Transcribed | |
| | | | | | | | locus | |
| 78 | | AF028823.2 | AF028823 Homo | NM_014604.2 | Homo sapiens Tax1 | Hs.12956 | Tax1 (human | TAX1BP3 |
| | | | sapiens Tax | | (human T-cell leukemia | | T-cell | |
| | | | interaction protein 1 | | virus type I) binding protein | | leukemia | |
| | | | mRNA, complete cds | | 3 (TAX1BP3), mRNA | | virus type I) | |
| | | | Length = 1451 | | Length = 1421 | | binding | |
| | | | | | | | protein 3 | |
| 79 | | BC008062.2 | Homo sapiens basic | NM_001207.3 | Homo sapiens basic | Hs.560854 | Transcribed | |
| | | | transcription factor 3, | | transcription factor 3 | | locus | |
| | | | mRNA (cDNA clone | | (BTF3), mRNA Length = 895 | | | |
| | | | MGC: 2209 | | | | | |
| | | | IMAGE: 2966788), | | | | | |
| | | | complete cds | | | | | |
| | | | Length = 865 | | | | | |
| 80 | | BC104562.1 | Bos taurus cDNA | NM_003295.1 | Homo sapiens tumor | Hs.527980 | DUTP | DUT |
| | | | clone MGC: 128232 | | protein, translationally- | | pyrophosphatase | |
| | | | IMAGE: 7948168, | | controlled 1 (TPT1), mRNA | | | |
| | | | complete cds | | Length = 830 | | | |
| | | | Length = 861 | | | | | |
| 81 | IMAGE: 1915051 | | | | | | | |
| 82 | | BC080645.1 | Homo sapiens ring | | | Hs.436922 | Ret finger | RFP2 |
| | | | finger protein 187, | | | | protein 2 | |
| | | | mRNA (cDNA clone | | | | | |
| | | | IMAGE: 5093646) | | | | | |
| | | | Length = 1516 | | | | | |
| 83 | | BC001936.1 | Homo sapiens | NM_004323.3 | Homo sapiens BCL2- | Hs.377484 | BCL2- | BAG1 |
| | | | BCL2-associated | | associated athanogene | | associated | |
| | | | athanogene, mRNA | | (BAG1), mRNA | | athanogene | |
| | | | (cDNA clone | | Length = 3858 | | | |
| | | | MGC: 4060 | | | | | |
| | | | IMAGE: 2823774), | | | | | |
| | | | complete cds | | | | | |
| | | | Length = 1307 | | | | | |
| 84 | | BC005831.2 | Homo sapiens T-cell | NM_021966.1 | Homo sapiens T-cell | Hs.2484 | T-cell | TCL1A |
| | | | leukemia/lymphoma | | leukemia/lymphoma 1A | | leukemia/lymphoma | |
| | | | 1A, mRNA (cDNA | | (TCL1A), mRNA | | 1A | |
| | | | clone MGC: 2170 | | Length = 1324 | | | |
| | | | IMAGE: 2966563), | | | | | |
| | | | complete cds | | | | | |
| | | | Length = 1231 | | | | | |
| 85 | | | | | | Hs.200413 | Transmembrane | TMED8 |
| | | | | | | | emp24 | |
| | | | | | | | protein | |
| | | | | | | | transport | |
| | | | | | | | domain | |
| | | | | | | | containing 8 | |
| 86 | | AC004854.3 | Homo sapiens PAC | | | | | |
| | | | clone RP4-673M15 | | | | | |
| | | | from 7, complete | | | | | |
| | | | sequence | | | | | |
| | | | Length = 98697 | | | | | |
| 87 | | L29065.1 | HUMBPA02 Human | XM_612661.2 | PREDICTED: Bos taurus | Hs.558424 | Cold shock | CSDA |
| | | | DNA-binding protein | | similar to muscle Y-box | | domain | |
| | | | A gene, exon 2 | | protein YB2 (LOC533297), | | protein A | |
| | | | Length = 459 | | mRNA Length = 2010 | | | |
| 88 | | BC035128.1 | Homo sapiens cDNA | NM_005962.4 | Homo sapiens MAX | Hs.501023 | MAX | MXI1 |
| | | | clone | | interactor 1 (MXI1), | | interactor 1 | |
| | | | IMAGE: 5263647, | | transcript variant 1, mRNA | | | |
| | | | partial cds | | Length = 3272 | | | |
| | | | Length = 2756 | | | | | |
| 89 | | AC013272.5 | Homo sapiens BAC | XM_530502.1 | PREDICTED: Pan | Hs.546387 | Hypothetical | DKFZp434N062 |
| | | | clone RP11-401C13 | | troglodytes LOC459556 | | protein | |
| | | | from 2, complete | | (LOC459556), mRNA | | DKFZp434N062 | |
| | | | sequence | | Length = 644 | | | |
| | | | Length = 172705 | | | | | |
| 90 | | | | | | Hs.213065 | Hypothetical | LOC158257 |
| | | | | | | | protein | |
| | | | | | | | LOC158257 | |
| 91 | | BC053070.1 | Mus musculus ring | NM_052916.1 | Homo sapiens ring finger | Hs.500643 | Ring finger | RNF157 |
| | | | finger protein 157, | | protein 157 (RNF157), | | protein 157 | |
| | | | mRNA (cDNA clone | | mRNA Length = 3570 | | | |
| | | | IMAGE: 6401088) | | | | | |
| | | | Length = 4436 | | | | | |
| 92 | | | | | | Hs.2316 | SRY (sex | SOX9 |
| | | | | | | | determining | |
| | | | | | | | region Y)- | |
| | | | | | | | box 9 | |
| | | | | | | | (campomelic | |
| | | | | | | | dysplasia, | |
| | | | | | | | autosomal | |
| | | | | | | | sex-reversal) | |
| 93 | | | | | | Hs.397705 | Calmodulin | CAMTA1 |
| | | | | | | | binding | |
| | | | | | | | transcription | |
| | | | | | | | activator 1 | |
| 94 | | | | | | Hs.349656 | Scavenger | SCARB2 |
| | | | | | | | receptor | |
| | | | | | | | class B, | |
| | | | | | | | member 2 | |
| 95 | | AC098616.2 | Homo sapiens | NM_022461.2 | Homo sapiens 5- | Hs.444724 | 5-azacytidine | AZI2 |
| | | | chromosome 3 clone | | azacytidine induced 2 | | induced 2 | |
| | | | RP11-279I18, | | (AZI2), mRNA | | | |
| | | | complete sequence | | Length = 3113 | | | |
| | | | Length = 159629 | | | | | |
| 96 | | BC016755.1 | Homo sapiens | NM_000186.2 | Homo sapiens complement | Hs.363396 | Complement | CFH |
| | | | complement factor | | factor H (CFH), transcript | | factor H | |
| | | | H-related 1, mRNA | | variant 1, mRNA | | | |
| | | | (cDNA clone | | Length = 4004 | | | |
| | | | MGC: 13525 | | | | | |
| | | | IMAGE: 3934474), | | | | | |
| | | | complete cds | | | | | |
| | | | Length = 1296 | | | | | |
| 97 | | AC146430.4 | Pan troglodytes BAC | | | Hs.490817 | Hypothetical | VIPR2 |
| | | | clone RP43-21F8 | | | | protein | |
| | | | from 7, complete | | | | LOC154822 | |
| | | | sequence | | | | | |
| | | | Length = 179251 | | | | | |
| 98 | | BC016003.1 | Homo sapiens | NM_001009567.1 | Homo sapiens mannose | Hs.461247 | Mannose | MRC1L1 |
| | | | mannose receptor, C | | receptor, C type 1-like 1 | | receptor, C | |
| | | | type 1, mRNA (cDNA | | (MRC1L1), mRNA | | type 1 | |
| | | | clone | | Length = 5171 | | | |
| | | | IMAGE: 4618280), | | | | | |
| | | | with apparent | | | | | |
| | | | retained intron | | | | | |
| | | | Length = 1618 | | | | | |
| 99 | | BC000954.1 | Homo sapiens | NM_007276.3 | Homo sapiens chromobox | Hs.381189 | Chromobox | CBX3 |
| | | | chromobox homolog | | homolog 3 (HP1 gamma | | homolog 3 | |
| | | | 3 (HP1 gamma | | homolog, Drosophila) | | (HP1 gamma | |
| | | | homolog, | | (CBX3), transcript variant 1, | | homolog, | |
| | | | Drosophila), | | mRNA Length = 1837 | | Drosophila) | |
| | | | transcript variant 1, | | | | | |
| | | | mRNA (cDNA clone | | | | | |
| | | | MGC: 4912 | | | | | |
| | | | IMAGE: 3450099), | | | | | |
| | | | complete cds | | | | | |
| | | | Length = 1833 | | | | | |
| 100 | | BC040494.1 | Homo sapiens DnaJ | NM_006736.4 | Homo sapiens DnaJ | Hs.77768 | DnaJ | DNAJB2 |
| | | | (Hsp40) homolog, | | (Hsp40) homolog, | | (Hsp40) | |
| | | | subfamily B, member | | subfamily B, member 2 | | homolog, | |
| | | | 2, mRNA (cDNA | | (DNAJB2), mRNA | | subfamily B, | |
| | | | clone | | Length = 3041 | | member 2 | |
| | | | IMAGE: 5297011), | | | | | |
| | | | with apparent | | | | | |
| | | | retained intron | | | | | |
| | | | Length = 4397 | | | | | |
| 101 | | U21049.1 | HSU21049 Homo | NM_005764.3 | Homo sapiens PDZK1 | Hs.431099 | PDZK1 | MAP17 |
| | | | sapiens DD96 | | interacting protein 1 | | interacting | |
| | | | mRNA, complete cds | | (PDZK1IP1), mRNA | | protein 1 | |
| | | | Length = 927 | | Length = 894 | | | |
| 102 | | BC012133.1 | Homo sapiens | NM_006058.2 | Homo sapiens TNFAIP3 | Hs.355141 | TNFAIP3 | TNIP1 |
| | | | TNFAIP3 interacting | | interacting protein 1 | | interacting | |
| | | | protein 1, mRNA | | (TNIP1), mRNA | | protein 1 | |
| | | | (cDNA clone | | Length = 2921 | | | |
| | | | MGC: 20223 | | | | | |
| | | | IMAGE: 4549542), | | | | | |
| | | | complete cds | | | | | |
| | | | Length = 2842 | | | | | |
| 103 | | BC000312.2 | Homo sapiens cyclin- | NM_078467.1 | Homo sapiens cyclin- | Hs.370771 | Cyclin- | CDKN1A |
| | | | dependent kinase | | dependent kinase inhibitor | | dependent | |
| | | | inhibitor 1A (p21, | | 1A (p21, Cip1) (CDKN1A), | | kinase | |
| | | | Cip1), transcript | | transcript variant 2, mRNA | | inhibitor 1A | |
| | | | variant 2, mRNA | | Length = 2281 | | (p21, Cip1) | |
| | | | (cDNA clone | | | | | |
| | | | MGC: 8428 | | | | | |
| | | | IMAGE: 2821049), | | | | | |
| | | | complete cds | | | | | |
| | | | Length = 2265 | | | | | |
| 104 | | BC014460.1 | Homo sapiens | NM_001911.2 | Homo sapiens cathepsin G | Hs.421724 | Cathepsin G | CTSG |
| | | | cathepsin G, mRNA | | (CTSG), mRNA | | | |
| | | | (cDNA clone | | Length = 924 | | | |
| | | | MGC: 23078 | | | | | |
| | | | IMAGE: 4859911), | | | | | |
| | | | complete cds | | | | | |
| | | | Length = 901 | | | | | |
| 105 | | BC074817.2 | Homo sapiens | NM_001972.2 | Homo sapiens elastase 2, | Hs.99863 | Elastase 2, | ELA2 |
| | | | elastase 2, | | neutrophil (ELA2), mRNA | | neutrophil | |
| | | | neutrophil, mRNA | | Length = 938 | | | |
| | | | (cDNA clone | | | | | |
| | | | MGC: 103928 | | | | | |
| | | | IMAGE: 30915320), | | | | | |
| | | | complete cds | | | | | |
| | | | Length = 901 | | | | | |
| 106 | | L33930.1 | HUMCD24B Homo | NM_013230.2 | Homo sapiens CD24 | Hs.375108 | CD24 | CD24 |
| | | | sapiens CD24 signal | | antigen (small cell lung | | antigen | |
| | | | transducer mRNA, | | carcinoma cluster 4 | | (small cell | |
| | | | complete cds and 3 | | antigen) (CD24), mRNA | | lung | |
| | | | region Length = 2116 | | Length = 2194 | | carcinoma | |
| | | | | | | | cluster 4 | |
| | | | | | | | antigen) | |
| 107 | | BC056250.1 | Homo sapiens leptin | NM_017526.2 | Homo sapiens leptin | Hs.23581 | Leptin | LEPR |
| | | | receptor overlapping | | receptor overlapping | | receptor | |
| | | | transcript, mRNA | | transcript (LEPROT), | | | |
| | | | (cDNA clone | | mRNA Length = 1147 | | | |
| | | | MGC: 61988 | | | | | |
| | | | IMAGE: 4328021), | | | | | |
| | | | complete cds | | | | | |
| | | | Length = 1092 | | | | | |
| 108 | | AF176043.1 | Homo sapiens | NM_181526.1 | Homo sapiens myosin, light | Hs.504687 | Elongation | MYL9 |
| | | | regulatory myosin | | polypeptide 9, regulatory | | factor Tu | |
| | | | light chain short | | (MYL9), transcript variant 2, | | family protein | |
| | | | version mRNA, | | mRNA Length = 1044 | | | |
| | | | alternatively spliced, | | | | | |
| | | | complete cds | | | | | |
| | | | Length = 971 | | | | | |
| 109 | | BC093965.1 | Homo sapiens | NM_002620.1 | Homo sapiens platelet | Hs.72933 | Platelet | PF4V1 |
| | | | platelet factor 4 | | factor 4 variant 1 (PF4V1), | | factor 4 | |
| | | | (chemokine (C—X—C | | mRNA Length = 755 | | variant 1 | |
| | | | motif) ligand 4), | | | | | |
| | | | mRNA (cDNA clone | | | | | |
| | | | MGC: 121000 | | | | | |
| | | | IMAGE: 7939810), | | | | | |
| | | | complete cds | | | | | |
| | | | Length = 338 | | | | | |
| 110 | | BC093965.1 | Homo sapiens | NM_002619.1 | Homo sapiens platelet | Hs.81564 | Platelet | PF4 |
| | | | platelet factor 4 | | factor 4 (chemokine (C—X—C | | factor 4 | |
| | | | (chemokine (C—X—C | | motif) ligand 4) (PF4), | | (chemokine | |
| | | | motif) ligand 4), | | mRNA Length = 439 | | (C—X—C motif) | |
| | | | mRNA (cDNA clone | | | | ligand 4) | |
| | | | MGC: 121000 | | | | | |
| | | | IMAGE: 7939810), | | | | | |
| | | | complete cds | | | | | |
| | | | Length = 338 | | | | | |
| 111 | | BC028217.1 | Homo sapiens pro- | NM_002704.2 | Homo sapiens pro-platelet | Hs.2164 | Pro-platelet | PPBP |
| | | | platelet basic protein | | basic protein (chemokine | | basic protein | |
| | | | (chemokine (C—X—C | | (C—X—C motif) ligand 7) | | (chemokine | |
| | | | motif) ligand 7), | | (PPBP), mRNA | | (C—X—C motif) | |
| | | | mRNA (cDNA clone | | Length = 715 | | ligand 7) | |
| | | | MGC: 40008 | | | | | |
| | | | IMAGE: 5214257), | | | | | |
| | | | complete cds | | | | | |
| | | | Length = 703 | | | | | |
| 112 | | BC028217.1 | Homo sapiens pro- | NM_002704.2 | Homo sapiens pro-platelet | Hs.2164 | Pro-platelet | PPBP |
| | | | platelet basic protein | | basic protein (chemokine | | basic protein | |
| | | | (chemokine (C—X—C | | (C—X—C motif) ligand 7) | | (chemokine | |
| | | | motif) ligand 7), | | (PPBP), mRNA | | (C—X—C motif) | |
| | | | mRNA (cDNA clone | | Length = 715 | | ligand 7) | |
| | | | MGC: 40008 | | | | | |
| | | | IMAGE: 5214257), | | | | | |
| | | | complete cds | | | | | |
| | | | Length = 703 | | | | | |
| 113 | | AC003043.1 | AC003043 Homo | NM_000419.2 | Homo sapiens integrin, | Hs.411312 | Integrin, | ITGA2B |
| | | | sapiens | | alpha 2b (platelet | | alpha 2b | |
| | | | chromosome 17, | | glycoprotein IIb of IIb/IIIa | | (platelet | |
| | | | clone HRPC1067M6, | | complex, antigen CD41B) | | glycoprotein | |
| | | | complete sequence | | (ITGA2B), mRNA | | IIb of IIb/IIIa | |
| | | | Length = 139488 | | Length = 3334 | | complex, | |
| | | | | | | | antigen | |
| | | | | | | | CD41B) | |
| 114 | | BC093965.1 | Homo sapiens | NM_002619.1 | Homo sapiens platelet | Hs.81564 | Platelet | PF4 |
| | | | platelet factor 4 | | factor 4 (chemokine (C—X—C | | factor 4 | |
| | | | (chemokine (C—X—C | | motif) ligand 4) (PF4), | | (chemokine | |
| | | | motif) ligand 4), | | mRNA Length = 439 | | (C—X—C motif) | |
| | | | mRNA (cDNA clone | | | | ligand 4) | |
| | | | MGC: 121000 | | | | | |
| | | | IMAGE: 7939810), | | | | | |
| | | | complete cds | | | | | |
| | | | Length = 338 | | | | | |
| 115 | | BC013221.1 | Homo sapiens | NM_203339.1 | Homo sapiens clusterin | Hs.436657 | Clusterin | CLU |
| | | | clusterin | | (complement lysis inhibitor, | | (complement | |
| | | | (complement lysis | | SP-40,40, sulfated | | lysis | |
| | | | inhibitor, SP-40,40, | | glycoprotein 2, | | inhibitor, SP- | |
| | | | sulfated glycoprotein | | testosterone-repressed | | 40,40, | |
| | | | 2, testosterone- | | prostate message 2, | | sulfated | |
| | | | repressed prostate | | apolipoprotein J) (CLU), | | glycoprotein | |
| | | | message 2, | | transcript variant 2, mRNA | | 2, | |
| | | | apolipoprotein J), | | Length = 2979 | | testosterone- | |
| | | | mRNA (cDNA clone | | | | repressed | |
| | | | IMAGE: 4149765) | | | | prostate | |
| | | | Length = 1620 | | | | message 2, | |
| | | | | | | | apolipoprotein | |
| | | | | | | | J) | |
| 116 | | BC029972.1 | Homo sapiens | NM_203339.1 | Homo sapiens clusterin | Hs.559474 | RPB11b2alpha | |
| | | | clusterin | | (complement lysis inhibitor, | | protein | |
| | | | (complement lysis | | SP-40,40, sulfated | | | |
| | | | inhibitor, SP-40,40, | | glycoprotein 2, | | | |
| | | | sulfated glycoprotein | | testosterone-repressed | | | |
| | | | 2, testosterone- | | prostate message 2, | | | |
| | | | repressed prostate | | apolipoprotein J) (CLU), | | | |
| | | | message 2, | | transcript variant 2, mRNA | | | |
| | | | apolipoprotein J), | | Length = 2979 | | | |
| | | | mRNA (cDNA clone | | | | | |
| | | | IMAGE: 4939961) | | | | | |
| | | | Length = 2821 | | | | | |
| 117 | | BC013221.1 | Homo sapiens | NM_203339.1 | Homo sapiens clusterin | Hs.436657 | Clusterin | CLU |
| | | | clusterin | | (complement lysis inhibitor, | | (complement | |
| | | | (complement lysis | | SP-40,40, sulfated | | lysis | |
| | | | inhibitor, SP-40,40, | | glycoprotein 2, | | inhibitor, SP- | |
| | | | sulfated glycoprotein | | testosterone-repressed | | 40,40, | |
| | | | 2, testosterone- | | prostate message 2, | | sulfated | |
| | | | repressed prostate | | apolipoprotein J) (CLU), | | glycoprotein | |
| | | | message 2, | | transcript variant 2, mRNA | | 2, | |
| | | | apolipoprotein J), | | Length = 2979 | | testosterone- | |
| | | | mRNA (cDNA clone | | | | repressed | |
| | | | IMAGE: 4149765) | | | | prostate | |
| | | | Length = 1620 | | | | message 2, | |
| | | | | | | | apolipoprotein | |
| | | | | | | | J) | |
| 118 | | | | | | Hs.144925 | Transcribed | |
| | | | | | | | locus | |
| 119 | | AC092959.6 | Homo sapiens 3 | XM_586391.2 | PREDICTED: Bos taurus | Hs.415766 | Zic family | ZIC4 |
| | | | BAC RP11-649A16 | | similar to zinc finger protein | | member 4 | |
| | | | (Roswell Park | | of the cerebellum 4 | | | |
| | | | Cancer Institute | | (LOC539312), mRNA | | | |
| | | | Human BAC Library) | | Length = 2941 | | | |
| | | | complete sequence | | | | | |
| | | | Length = 179714 | | | | | |
| 120 | | BC012293.1 | Homo sapiens | NM_020070.2 | Homo sapiens | Hs.546463 | Similar to | |
| | | | immunoglobulin | | immunoglobulin lambda- | | omega | |
| | | | lambda-like | | like polypeptide 1 (IGLL1), | | protein | |
| | | | polypeptide 1, | | transcript variant 1, mRNA | | | |
| | | | transcript variant 1, | | Length = 901 | | | |
| | | | mRNA (cDNA clone | | | | | |
| | | | MGC: 21361 | | | | | |
| | | | IMAGE: 4508892), | | | | | |
| | | | complete cds | | | | | |
| | | | Length = 853 | | | | | |
| 121 | | AY358309.1 | Homo sapiens clone | NM_148967.1 | Homo sapiens tumor | Hs.462529 | Tumor | TNFRSF25 |
| | | | DNA58801 DR3 | | necrosis factor receptor | | necrosis | |
| | | | (UNQ455) mRNA, | | superfamily, member 25 | | factor | |
| | | | complete cds | | (TNFRSF25), transcript | | receptor | |
| | | | Length = 1662 | | variant 4, mRNA | | superfamily, | |
| | | | | | Length = 1503 | | member 25 | |
| 122 | | | | | | Hs.179646 | Hypothetical | MGC35118 |
| | | | | | | | protein | |
| | | | | | | | MGC35118 | |
| 123 | | | | | | Hs.561568 | Transcribed | |
| | | | | | | | locus | |
| 124 | | AY422212.1 | Homo sapiens | NM_181806.2 | Homo sapiens 2- | Hs.104347 | 2- | AASDH |
| | | | putative non- | | aminoadipic 6- | | aminoadipic | |
| | | | ribosomal peptide | | semialdehyde | | 6- | |
| | | | synthetase | | dehydrogenase (AASDH), | | semialdehyde | |
| | | | NRPS1098 mRNA, | | mRNA Length = 3573 | | dehydrogenase | |
| | | | complete cds | | | | | |
| | | | Length = 3528 | | | | | |
| 125 | | BC012506.1 | Homo sapiens | NM_000641.2 | Homo sapiens interleukin | Hs.467304 | Interleukin | IL11 |
| | | | interleukin 11, mRNA | | 11 (IL11), mRNA | | 11 | |
| | | | (cDNA clone | | Length = 2354 | | | |
| | | | MGC: 21360 | | | | | |
| | | | IMAGE: 4476678), | | | | | |
| | | | complete cds | | | | | |
| | | | Length = 1201 | | | | | |
| 126 | | AC080162.7 | Homo sapiens BAC | | | Hs.404914 | ADAM | ADAM17 |
| | | | clone RP11-400L8 | | | | metallopeptidase | |
| | | | from 2, complete | | | | domain | |
| | | | sequence | | | | 17 (tumor | |
| | | | Length = 171653 | | | | necrosis | |
| | | | | | | | factor, alpha, | |
| | | | | | | | converting | |
| | | | | | | | enzyme) | |
| 127 | | | | | | Hs.518614 | Transcriptional | MGC21874 |
| | | | | | | | adaptor 2 | |
| | | | | | | | (ADA2 | |
| | | | | | | | homolog, | |
| | | | | | | | yeast)-beta | |
| 128 | | BC091477.1 | Homo sapiens Yip1 | NM_001031731.1 | Homo sapiens Yip1 | Hs.280741 | Yip1 | LOC90522 |
| | | | interacting factor | | interacting factor homolog | | interacting | |
| | | | homolog B (S. cerevisiae), | | B (S. cerevisiae) (YIF1B), | | factor | |
| | | | mRNA | | transcript variant 1, mRNA | | homolog B | |
| | | | (cDNA clone | | Length = 956 | | (S. cerevisiae) | |
| | | | MGC: 110904 | | | | | |
| | | | IMAGE: 30344388), | | | | | |
| | | | complete cds | | | | | |
| | | | Length = 1217 | | | | | |
| 129 | 1393148 | | | | | | | |
| 130 | | AF521134.1 | Homo sapiens tumor | | | Hs.492555 | Enhancer of | e(y)2 |
| | | | antigen CML66 | | | | yellow 2 | |
| | | | gene, short isoform | | | | homolog | |
| | | | promoter region and | | | | (Drosophila) | |
| | | | partial cds | | | | | |
| | | | Length = 4283 | | | | | |
| 131 | | | | | | Hs.567598 | Transcribed | |
| | | | | | | | locus | |
| 132 | | AC093415.2 | Homo sapiens | | | | | |
| | | | chromosome 3 clone | | | | | |
| | | | RP11-586N22, | | | | | |
| | | | complete sequence | | | | | |
| | | | Length = 196641 | | | | | |
| 133 | | BC019323.1 | Homo sapiens | NM_001024382.1 | Homo sapiens | Hs.82609 | Hydroxymethylbilane | HMBS |
| | | | hydroxymethylbilane | | hydroxymethylbilane | | synthase | |
| | | | synthase, mRNA | | synthase (HMBS), | | | |
| | | | (cDNA clone | | transcript variant 2, mRNA | | | |
| | | | MGC: 4369 | | Length = 1428 | | | |
| | | | IMAGE: 2822949), | | | | | |
| | | | complete cds | | | | | |
| | | | Length = 1536 | | | | | |
| 134 | | BC007928.1 | Homo sapiens | NM_032910.2 | Homo sapiens | Hs.58149 | Chromosome | C21orf119 |
| | | | chromosome 21 | | chromosome 21 open | | 21 open | |
| | | | open reading frame | | reading frame 119 | | reading | |
| | | | 119, mRNA (cDNA | | (C21orf119), mRNA | | frame 119 | |
| | | | clone MGC: 14136 | | Length = 838 | | | |
| | | | IMAGE: 3945901), | | | | | |
| | | | complete cds | | | | | |
| | | | Length = 844 | | | | | |
| 135 | | | | | | Hs.549607 | CDNA clone | |
| | | | | | | | IMAGE: 5757380 | |
| 136 | | AC093635.4 | Homo sapiens BAC | | | | | |
| | | | clone RP11-191L17 | | | | | |
| | | | from 2, complete | | | | | |
| | | | sequence | | | | | |
| | | | Length = 132032 | | | | | |
| 137 | | AC073082.6 | Homo sapiens BAC | NM_006873.2 | Homo sapiens stoned B- | Hs.44385 | TFIIA- | ALF |
| | | | clone RP11-310N16 | | like factor (SBLF), mRNA | | alpha/beta- | |
| | | | from 2, complete | | Length = 5822 | | like factor | |
| | | | sequence | | | | | |
| | | | Length = 177047 | | | | | |
| 138 | | AC018545.14 | Homo sapiens 3 | | | Hs.52526 | TSC22 | KIAA0669 |
| | | | BAC RP11-145F16 | | | | domain | |
| | | | (Roswell Park | | | | family, | |
| | | | Cancer Institute | | | | member 2 | |
| | | | Human BAC Library) | | | | | |
| | | | complete sequence | | | | | |
| | | | Length = 170359 | | | | | |
| 139 | | AC005858.1 | Homo sapiens | | | Hs.386211 | Transcribed | |
| | | | 12q24.2 PAC RPCI3- | | | | locus | |
| | | | 446N13 (Roswell | | | | | |
| | | | Park Cancer Institute | | | | | |
| | | | Human PAC Library) | | | | | |
| | | | complete sequence | | | | | |
| | | | Length = 135521 | | | | | |
| 140 | | AY077696.1 | Homo sapiens clone | | | Hs.352631 | Chromosome | C21orf74 |
| | | | qd65g07 PRED16 | | | | 21 open | |
| | | | protein (PRED16) | | | | reading | |
| | | | mRNA, complete cds | | | | frame 74 | |
| | | | Length = 726 | | | | | |
| 141 | | U19251.1 | HSU19251 Homo | NM_004536.1 | Homo sapiens baculoviral | Hs.558359 | Baculoviral | BIRC1 |
| | | | sapiens neuronal | | IAP repeat-containing 1 | | IAP repeat- | |
| | | | apoptosis inhibitory | | (BIRC1), mRNA | | containing 1 | |
| | | | protein mRNA, | | Length = 6133 | | | |
| | | | complete cds | | | | | |
| | | | Length = 6124 | | | | | |
| 142 | | | | | | Hs.196578 | Chromosome | C10orf85 |
| | | | | | | | 10 open | |
| | | | | | | | reading | |
| | | | | | | | frame 85 | |
| 143 | | | | XM_513171.1 | PREDICTED: Pan | Hs.558906 | Hypothetical | LOC400745 |
| | | | | | troglodytes similar to | | protein | |
| | | | | | BC036961 protein | | LOC149194 | |
| | | | | | (LOC456593), mRNA | | | |
| | | | | | Length = 2715 | | | |
| 144 | | AC064836.7 | Homo sapiens BAC | | | Hs.541555 | Transcribed | |
| | | | clone RP11-686O6 | | | | locus | |
| | | | from 2, complete | | | | | |
| | | | sequence | | | | | |
| | | | Length = 154754 | | | | | |
| 145 | | | | | | Hs.127851 | Chromosome | |
| | | | | | | | 1 open | |
| | | | | | | | reading | |
| | | | | | | | frame 137 | |
| 146 | | BC001017.1 | Homo sapiens PDZ | | | Hs.85862 | PDZ and LIM | PDLIM3 |
| | | | and LIM domain 3, | | | | domain 3 | |
| | | | mRNA (cDNA clone | | | | | |
| | | | MGC: 2005 | | | | | |
| | | | IMAGE: 3345880), | | | | | |
| | | | complete cds | | | | | |
| | | | Length = 2819 | | | | | |
| 147 | | AC005300.10 | Homo sapiens | NM_014339.3 | Homo sapiens interleukin | Hs.129751 | Interleukin | IL17R |
| | | | chromosome 22 | | 17 receptor (IL17R), mRNA | | 17 receptor | |
| | | | clone p143i13 map | | Length = 3429 | | | |
| | | | 22q11, complete | | | | | |
| | | | sequence | | | | | |
| | | | Length = 101269 | | | | | |
| 148 | | AC110926.5 | Homo sapiens BAC | NM_006609.2 | Homo sapiens mitogen- | Hs.145605 | Mitogen- | MAP3K2 |
| | | | clone RP11-313N8 | | activated protein kinase | | activated | |
| | | | from 2, complete | | kinase kinase 2 (MAP3K2), | | protein | |
| | | | sequence | | mRNA Length = 3336 | | kinase | |
| | | | Length = 193648 | | | | kinase | |
| | | | | | | | kinase 2 | |
| 149 | | AF327353.1 | AF327353 Homo | NM_018367.3 | Homo sapiens | Hs.23862 | Phytoceramidase, | PHCA |
| | | | sapiens alkaline | | phytoceramidase, alkaline | | alkaline | |
| | | | dihydroceramidase | | (PHCA), mRNA | | | |
| | | | SB89 mRNA, | | Length = 3473 | | | |
| | | | complete cds | | | | | |
| | | | Length = 3473 | | | | | |
| 150 | | AC022898.11 | Homo sapiens | | | Hs.135183 | Centaurin, | CENTA1 |
| | | | chromosome 15, | | | | alpha 1 | |
| | | | clone RP11-554D20, | | | | | |
| | | | complete sequence | | | | | |
| | | | Length = 200380 | | | | | |
| 151 | | AF005887.1 | AF005887 Homo | NM_007348.2 | Homo sapiens activating | Hs.533771 | STIP1 | STUB1 |
| | | | sapiens ATF family | | transcription factor 6 | | homology | |
| | | | member ATF6 | | (ATF6), mRNA | | and U-box | |
| | | | (ATF6) mRNA, | | Length = 2488 | | containing | |
| | | | complete cds | | | | protein 1 | |
| | | | Length = 2474 | | | | | |
| 152 | | | | | | Hs.147880 | LOC440132 | |
| 153 | | AC067945.4 | Homo sapiens BAC | NM_139266.1 | Homo sapiens signal | Hs.470943 | Signal | STAT1 |
| | | | clone RP11-629B4 | | transducer and activator of | | transducer | |
| | | | from 2, complete | | transcription 1, 91 kDa | | and activator | |
| | | | sequence | | (STAT1), transcript variant | | of | |
| | | | Length = 162471 | | beta, mRNA Length = 2762 | | transcription | |
| | | | | | | | 1, 91 kDa | |
| 154 | | BC035716.2 | Homo sapiens | NM_006084.3 | Homo sapiens interferon- | Hs.1706 | Interferon- | ISGF3G |
| | | | interferon-stimulated | | stimulated transcription | | stimulated | |
| | | | transcription factor 3, | | factor 3, gamma 48 kDa | | transcription | |
| | | | gamma 48 kDa, | | (ISGF3G), mRNA | | factor 3, | |
| | | | mRNA (cDNA clone | | Length = 1584 | | gamma | |
| | | | MGC: 46151 | | | | 48 kDa | |
| | | | IMAGE: 5588434), | | | | | |
| | | | complete cds | | | | | |
| | | | Length = 1593 | | | | | |
| 155 | | AC138374.2 | Homo sapiens | NM_001572.2 | Homo sapiens interferon | Hs.166120 | Interferon | IRF7 |
| | | | chromosome 11, | | regulatory factor 7 (IRF7), | | regulatory | |
| | | | clone CTD-2647G13, | | transcript variant a, mRNA | | factor 7 | |
| | | | complete sequence | | Length = 1890 | | | |
| | | | Length = 196424 | | | | | |
| 156 | | BC008060.2 | Homo sapiens cDNA | NM_033307.2 | Homo sapiens caspase 4, | Hs.138378 | Caspase 4, | CASP4 |
| | | | clone | | apoptosis-related cysteine | | apoptosis- | |
| | | | IMAGE: 2988427, | | peptidase (CASP4), | | related | |
| | | | containing frame- | | transcript variant delta, | | cysteine | |
| | | | shift errors | | mRNA Length = 1327 | | peptidase | |
| | | | Length = 1291 | | | | | |
| 157 | | AC093798.3 | Homo sapiens BAC | XM_498892.1 | PREDICTED: Homo | Hs.158920 | LOC440852 | |
| | | | clone RP11-306N14 | | sapiens LOC440852 | | | |
| | | | from 2, complete | | (LOC440852), mRNA | | | |
| | | | sequence | | Length = 618 | | | |
| | | | Length = 233877 | | | | | |
| 158 | | BC001079.2 | Homo sapiens | NM_024068.2 | Homo sapiens hypothetical | Hs.240170 | Hypothetical | MGC2731 |
| | | | hypothetical protein | | protein MGC2731 | | protein | |
| | | | MGC2731, mRNA | | (MGC2731), mRNA | | MGC2731 | |
| | | | (cDNA clone | | Length = 1332 | | | |
| | | | MGC: 2731 | | | | | |
| | | | IMAGE: 2822460), | | | | | |
| | | | complete cds | | | | | |
| | | | Length = 1332 | | | | | |
| 159 | | BC011589.1 | Homo sapiens | NM_020530.3 | Homo sapiens oncostatin M | Hs.248156 | Oncostatin M | OSM |
| | | | oncostatin M, mRNA | | (OSM), mRNA | | | |
| | | | (cDNA clone | | Length = 1880 | | | |
| | | | MGC: 20461 | | | | | |
| | | | IMAGE: 4548943), | | | | | |
| | | | complete cds | | | | | |
| | | | Length = 1869 | | | | | |
| 160 | | | | | | Hs.310536 | Histone | HDAC8 |
| | | | | | | | deacetylase 8 | |
| 161 | | AF109146.1 | Homo sapiens C- | NM_194447.1 | Homo sapiens C-type lectin | Hs.504657 | C-type lectin | CLECSF6 |
| | | | type lectin | | domain family 4, member A | | domain | |
| | | | superfamily 6 | | (CLEC4A), transcript | | family 4, | |
| | | | (CLECSF6) mRNA, | | variant 3, mRNA | | member A | |
| | | | complete cds | | Length = 1171 | | | |
| | | | Length = 1133 | | | | | |
| 162 | | BC070085.1 | Homo sapiens | NM_000395.1 | Homo sapiens colony | Hs.285401 | Colony | CSF2RB |
| | | | colony stimulating | | stimulating factor 2 | | stimulating | |
| | | | factor 2 receptor, | | receptor, beta, low-affinity | | factor 2 | |
| | | | beta, low-affinity | | (granulocyte-macrophage) | | receptor, | |
| | | | (granulocyte- | | (CSF2RB), mRNA | | beta, low- | |
| | | | macrophage), mRNA | | Length = 2996 | | affinity | |
| | | | (cDNA clone | | | | (granulocyte- | |
| | | | MGC: 87425 | | | | macrophage) | |
| | | | IMAGE: 30344148), | | | | | |
| | | | complete cds | | | | | |
| | | | Length = 4116 | | | | | |
| 163 | | BC008678.1 | Homo sapiens | NM_000576.2 | Homo sapiens interleukin 1, | Hs.126256 | Interleukin 1, | IL1B |
| | | | interleukin 1, beta, | | beta (IL1B), mRNA | | beta | |
| | | | mRNA (cDNA clone | | Length = 1498 | | | |
| | | | MGC: 9216 | | | | | |
| | | | IMAGE: 3875593), | | | | | |
| | | | complete cds | | | | | |
| | | | Length = 1522 | | | | | |
| 164 | | BC008678.1 | Homo sapiens | NM_000576.2 | Homo sapiens interleukin 1, | Hs.126256 | Interleukin 1, | IL1B |
| | | | interleukin 1, beta, | | beta (IL1B), mRNA | | beta | |
| | | | mRNA (cDNA clone | | Length = 1498 | | | |
| | | | MGC: 9216 | | | | | |
| | | | IMAGE: 3875593), | | | | | |
| | | | complete cds | | | | | |
| | | | Length = 1522 | | | | | |
| 165 | | BC012181.1 | Homo sapiens furin | NM_002569.2 | Homo sapiens furin (paired | Hs.513153 | Furin (paired | FURIN |
| | | | (paired basic amino | | basic amino acid cleaving | | basic amino | |
| | | | acid cleaving | | enzyme) (FURIN), mRNA | | acid cleaving | |
| | | | enzyme), mRNA | | Length = 4180 | | enzyme) | |
| | | | (cDNA clone | | | | | |
| | | | MGC: 20463 | | | | | |
| | | | IMAGE: 4550620), | | | | | |
| | | | complete cds | | | | | |
| | | | Length = 4033 | | | | | |
| 166 | | BC001506.2 | Homo sapiens CD59 | NM_203329.1 | Homo sapiens CD59 | Hs.278573 | CD59 | CD59 |
| | | | antigen p18-20 | | antigen p18-20 (antigen | | antigen p18-20 | |
| | | | (antigen identified by | | identified by monoclonal | | (antigen | |
| | | | monoclonal | | antibodies 16.3A5, EJ16, | | identified by | |
| | | | antibodies 16.3A5, | | EJ30, EL32 and G344) | | monoclonal | |
| | | | EJ16, EJ30, EL32 | | (CD59), transcript variant 3, | | antibodies | |
| | | | and G344), transcript | | mRNA Length = 7682 | | 16.3A5, | |
| | | | variant 2, mRNA | | | | EJ16, EJ30, | |
| | | | (cDNA clone | | | | EL32 and | |
| | | | MGC: 2354 | | | | G344) | |
| | | | IMAGE: 2988140), | | | | | |
| | | | complete cds | | | | | |
| | | | Length = 1937 | | | | | |
| 167 | | BC106764.1 | Homo sapiens cDNA | NM_003853.2 | Homo sapiens interleukin | Hs.158315 | Interleukin | IL18RAP |
| | | | clone MGC: 120589 | | 18 receptor accessory | | 18 receptor | |
| | | | IMAGE: 40026248, | | protein (IL18RAP), mRNA | | accessory | |
| | | | complete cds | | Length = 2668 | | protein | |
| | | | Length = 1891 | | | | | |
| 168 | | AC007248.4 | Homo sapiens BAC | NM_003855.2 | Homo sapiens interleukin | Hs.469521 | Interleukin | IL18R1 |
| | | | clone RP11-315O22 | | 18 receptor 1 (IL18R1), | | 18 receptor 1 | |
| | | | from 2, complete | | mRNA Length = 3522 | | | |
| | | | sequence | | | | | |
| | | | Length = 201535 | | | | | |
| | | | Features flanking | | | | | |
| | | | this part of subject | | | | | |
| | | | sequence: 1411 bp | | | | | |
| | | | at 5 side: unknown | | | | | |
| | | | 24927 bp at 3 side: | | | | | |
| | | | unknown | | | | | |
| 169 | | AC007248.4 | Homo sapiens BAC | NM_003855.2 | Homo sapiens interleukin | Hs.469521 | Interleukin | IL18R1 |
| | | | clone RP11-315O22 | | 18 receptor 1 (IL18R1), | | 18 receptor 1 | |
| | | | from 2, complete | | mRNA Length = 3522 | | | |
| | | | sequence | | | | | |
| | | | Length = 201535 | | | | | |
| | | | Features flanking | | | | | |
| | | | this part of subject | | | | | |
| | | | sequence: 1401 bp | | | | | |
| | | | at 5 side: unknown | | | | | |
| | | | 24927 bp at 3 side: | | | | | |
| | | | unknown | | | | | |
| 170 | | BC016281.1 | Homo sapiens | NM_004049.2 | Homo sapiens BCL2- | Hs.227817 | BCL2-related | BCL2A1 |
| | | | BCL2-related protein | | related protein A1 | | protein A1 | |
| | | | A1, mRNA (cDNA | | (BCL2A1), mRNA | | | |
| | | | clone MGC: 8991 | | Length = 885 | | | |
| | | | IMAGE: 3920808), | | | | | |
| | | | complete cds | | | | | |
| | | | Length = 829 | | | | | |
| 171 | | M28585.1 | HUMIFNN Human | NM_002173.1 | Homo sapiens interferon, | Hs.56303 | Interferon, | IFNA16 |
| | | | leukocyte interferon- | | alpha 16 (IFNA16), mRNA | | alpha 16 | |
| | | | alpha mRNA, | | Length = 941 | | | |
| | | | complete cds, clone | | | | | |
| | | | pIFN105 Length = 941 | | | | | |
| 172 | | BC030205.1 | Homo sapiens tumor | NM_007115.2 | Homo sapiens tumor | Hs.437322 | Tumor | TNFAIP6 |
| | | | necrosis factor, | | necrosis factor, alpha- | | necrosis | |
| | | | alpha-induced | | induced protein 6 | | factor, alpha- | |
| | | | protein 6, mRNA | | (TNFAIP6), mRNA | | induced | |
| | | | (cDNA clone | | Length = 1440 | | protein 6 | |
| | | | MGC: 34404 | | | | | |
| | | | IMAGE: 5208773), | | | | | |
| | | | complete cds | | | | | |
| | | | Length = 1430 | | | | | |
| 173 | | AC007198.6 | AC007198 Homo | NM_003268.3 | Homo sapiens toll-like | Hs.114408 | Toll-like | TLR5 |
| | | | sapiens, clone | | receptor 5 (TLR5), mRNA | | receptor 5 | |
| | | | 18_A_13, complete | | Length = 3369 | | | |
| | | | sequence | | | | | |
| | | | Length = 166977 | | | | | |
| 174 | | BC026025.1 | Homo sapiens | NM_017817.1 | Homo sapiens RAB20, | Hs.508720 | RAB20, | RAB20 |
| | | | RAB20, member | | member RAS oncogene | | member | |
| | | | RAS oncogene | | family (RAB20), mRNA | | RAS | |
| | | | family, mRNA (cDNA | | Length = 1530 | | oncogene | |
| | | | clone MGC: 39156 | | | | family | |
| | | | IMAGE: 4636491), | | | | | |
| | | | complete cds | | | | | |
| | | | Length = 1541 | | | | | |
| 175 | | BC063827.1 | Homo sapiens | NM_002957.3 | Homo sapiens retinoid X | Hs.20084 | Retinoid X | RXRA |
| | | | retinoid X receptor, | | receptor, alpha (RXRA), | | receptor, | |
| | | | alpha, mRNA (cDNA | | mRNA Length = 5449 | | alpha | |
| | | | clone | | | | | |
| | | | IMAGE: 6528306), | | | | | |
| | | | partial cds | | | | | |
| | | | Length = 5534 | | | | | |
| 176 | | BC018058.1 | Homo sapiens tumor | NM_172014.1 | Homo sapiens tumor | Hs.129708 | Tumor | TNFSF14 |
| | | | necrosis factor | | necrosis factor (ligand) | | necrosis | |
| | | | (ligand) superfamily, | | superfamily, member 14 | | factor | |
| | | | member 14, mRNA | | (TNFSF14), transcript | | (ligand) | |
| | | | (cDNA clone | | variant 2, mRNA | | superfamily, | |
| | | | MGC: 26477 | | Length = 1395 | | member 14 | |
| | | | IMAGE: 4793299), | | | | | |
| | | | complete cds | | | | | |
| | | | Length = 1211 | | | | | |
| 177 | | AF205589.5 | Homo sapiens | NM_024531.3 | Homo sapiens G protein- | Hs.6459 | G protein- | GPR172A |
| | | | chromosome 8 clone | | coupled receptor 172A | | coupled | |
| | | | CTA-393G12 map | | (GPR172A), mRNA | | receptor | |
| | | | qtel, complete | | Length = 1952 | | 172A | |
| | | | sequence | | | | | |
| | | | Length = 143847 | | | | | |
| 178 | | BC065291.1 | Homo sapiens ras | NM_012249.2 | Homo sapiens ras homolog | Hs.549125 | Ras homolog | RHOQ |
| | | | homolog gene family, | | gene family, member Q | | gene family, | |
| | | | member Q, mRNA | | (RHOQ), mRNA | | member Q | |
| | | | (cDNA clone | | Length = 2127 | | | |
| | | | IMAGE: 6061807), | | | | | |
| | | | partial cds | | | | | |
| | | | Length = 1922 | | | | | |
| 179 | | AY005151.1 | Homo sapiens BID S | NM_197967.1 | Homo sapiens BH3 | Hs.474150 | BH3 | BID |
| | | | (BID) mRNA, | | interacting domain death | | interacting | |
| | | | complete cds | | agonist (BID), transcript | | domain | |
| | | | Length = 854 | | variant 3, mRNA | | death | |
| | | | | | Length = 2144 | | agonist | |
| 180 | | BC010931.1 | Homo sapiens | | | Hs.153629 | MADS box | MEF2B |
| | | | MADS box | | | | transcription | |
| | | | transcription | | | | enhancer | |
| | | | enhancer factor 2, | | | | factor 2, | |
| | | | polypeptide B | | | | polypeptide | |
| | | | (myocyte enhancer | | | | B (myocyte | |
| | | | factor 2B), mRNA | | | | enhancer | |
| | | | (cDNA clone | | | | factor 2B) | |
| | | | IMAGE: 4280324), | | | | | |
| | | | complete cds | | | | | |
| | | | Length = 1020 | | | | | |
| 181 | | BC017197.2 | Homo sapiens | NM_182763.1 | Homo sapiens myeloid cell | Hs.532826 | Myeloid cell | MCL1 |
| | | | myeloid cell leukemia | | leukemia sequence 1 | | leukemia | |
| | | | sequence 1 (BCL2- | | (BCL2-related) (MCL1), | | sequence 1 | |
| | | | related), transcript | | transcript variant 2, mRNA | | (BCL2- | |
| | | | variant 1, mRNA | | Length = 3772 | | related) | |
| | | | (cDNA clone | | | | | |
| | | | MGC: 1839 | | | | | |
| | | | IMAGE: 3138465), | | | | | |
| | | | complete cds | | | | | |
| | | | Length = 3944 | | | | | |
| 182 | | | | NM_001560.2 | Homo sapiens interleukin | Hs.496646 | Interleukin | IL13RA1 |
| | | | | | 13 receptor, alpha 1 | | 13 receptor, | |
| | | | | | (IL13RA1), mRNA | | alpha 1 | |
| | | | | | Length = 4006 | | | |
| 183 | | BC072446.1 | Homo sapiens | NM_198057.2 | Homo sapiens TSC22 | Hs.522074 | TSC22 | DSIPI |
| | | | TSC22 domain | | domain family, member 3 | | domain | |
| | | | family, member 3, | | (TSC22D3), transcript | | family, | |
| | | | transcript variant 2, | | variant 1, mRNA | | member 3 | |
| | | | mRNA (cDNA clone | | Length = 2312 | | | |
| | | | MGC: 90321 | | | | | |
| | | | IMAGE: 6165765), | | | | | |
| | | | complete cds | | | | | |
| | | | Length = 1959 | | | | | |
| 184 | | AC004962.1 | AC004962 Homo | | | Hs.298858 | CDNA | |
| | | | sapiens PAC clone | | | | FLJ42664 | |
| | | | RP5-1099N7 from 1, | | | | fis, clone | |
| | | | complete sequence | | | | BRAMY2019985 | |
| | | | Length = 143749 | | | | | |
| 185 | IMAGE: 2210177 | | | | | | | |
| 186 | | AC090827.11 | Homo sapiens | | | Hs.301974 | Low density | LRP12 |
| | | | chromosome 8, | | | | lipoprotein- | |
| | | | clone CTD-2594K21, | | | | related | |
| | | | complete sequence | | | | protein 12 | |
| | | | Length = 135010 | | | | | |
| 187 | IMAGE: 1371638 | | | | | | | |
| 188 | | BC005821.2 | Homo sapiens | NM_000314.3 | Homo sapiens | Hs.500466 | Phosphatase | PTEN |
| | | | phosphatase and | | phosphatase and tensin | | and tensin | |
| | | | tensin homolog | | homolog (mutated in | | homolog | |
| | | | (mutated in multiple | | multiple advanced cancers | | (mutated in | |
| | | | advanced cancers | | 1) (PTEN), mRNA | | multiple | |
| | | | 1), mRNA (cDNA | | Length = 3416 | | advanced | |
| | | | clone MGC: 11227 | | | | cancers 1) | |
| | | | IMAGE: 3937787), | | | | | |
| | | | complete cds | | | | | |
| | | | Length = 2357 | | | | | |
| 189 | | AY455929.1 | Homo sapiens | NM_004054.2 | Homo sapiens complement | Hs.527839 | Complement | C3AR1 |
| | | | complement | | component 3a receptor 1 | | component | |
| | | | component 3a | | (C3AR1), mRNA | | 3a receptor 1 | |
| | | | receptor 1 (C3AR1) | | Length = 1985 | | | |
| | | | gene, complete cds | | | | | |
| | | | Length = 12053 | | | | | |
| 190 | | BC003091.1 | Homo sapiens | NM_002856.1 | Homo sapiens poliovirus | Hs.326371 | Poliovirus | PVRL2 |
| | | | poliovirus receptor- | | receptor-related 2 | | receptor- | |
| | | | related 2 | | (herpesvirus entry mediator | | related 2 | |
| | | | (herpesvirus entry | | B) (PVRL2), mRNA | | (herpesvirus | |
| | | | mediator B), mRNA | | Length = 1928 | | entry | |
| | | | (cDNA clone | | | | mediator B) | |
| | | | MGC: 1349 | | | | | |
| | | | IMAGE: 3503222), | | | | | |
| | | | complete cds | | | | | |
| | | | Length = 1968 | | | | | |
| 191 | | AY834276.1 | Homo sapiens | NM_145686.2 | Homo sapiens mitogen- | Hs.431550 | Mitogen- | MAP4K4 |
| | | | mitogen-activated | | activated protein kinase | | activated | |
| | | | protein kinase kinase | | kinase kinase kinase 4 | | protein | |
| | | | kinase kinase 4 | | (MAP4K4), transcript | | kinase | |
| | | | isoform (MAP4K4) | | variant 2, mRNA | | kinase | |
| | | | mRNA, complete | | Length = 7495 | | kinase | |
| | | | cds, alternatively | | | | kinase 4 | |
| | | | spliced Length = 3129 | | | | | |
| 192 | | AC158549.8 | Mus musculus | NM_000684.1 | Homo sapiens adrenergic, | Hs.99913 | Adrenergic, | ADRB1 |
| | | | chromosome 19, | | beta-1-, receptor (ADRB1), | | beta-1-, | |
| | | | clone RP24-216K4, | | mRNA Length = 1723 | | receptor | |
| | | | complete sequence | | | | | |
| | | | Length = 195500 | | | | | |
| 193 | | BC038711.1 | Homo sapiens UDP- | XM_592193.2 | PREDICTED: Bos taurus | Hs.304249 | UDP-glucose | UGCG |
| | | | glucose ceramide | | similar to Ceramide | | ceramide | |
| | | | glucosyltransferase, | | glucosyltransferase | | glucosyltransferase | |
| | | | mRNA (cDNA clone | | (Glucosylceramide | | | |
| | | | MGC: 33797 | | synthase) (GCS) (UDP- | | | |
| | | | IMAGE: 5295561), | | glucose: N-acylsphingosine | | | |
| | | | complete cds | | D-glucosyltransferase) | | | |
| | | | Length = 1730 | | (UDP-glucose ceramide | | | |
| | | | | | glucosyltransferase) | | | |
| | | | | | (GLCT-1) (LOC514357), | | | |
| | | | | | mRNA Length = 1433 | | | |
| 194 | | AC021754.6 | Homo sapiens | NG_005156.1 | Homo sapiens creatine | Hs.253260 | Cation | |
| | | | chromosome 15 | | kinase, mitochondrial 1 | | channel, | |
| | | | clone RP11-263I19 | | duplicated region | | sperm | |
| | | | map 15q15, | | (CKMT1@) on | | associated 2 | |
| | | | complete sequence | | chromosome 15 | | pseudogene | |
| | | | Length = 172474 | | Length = 239654 Features | | | |
| | | | | | flanking this part of subject | | | |
| | | | | | sequence: 5819 bp at 5 | | | |
| | | | | | side: creatine kinase, | | | |
| | | | | | mitochondrial 1A precursor | | | |
| | | | | | 41564 bp at 3 side: protein | | | |
| | | | | | disulfide | | | |
| 195 | | | | | | Hs.421340 | Chromosome | C9orf98 |
| | | | | | | | 9 open | |
| | | | | | | | reading | |
| | | | | | | | frame 98 | |
| 196 | IMAGE: 144802 | | | | | | | |
| 197 | | AC012653.8 | AC012653 Homo | | | Hs.104792 | Transcribed | |
| | | | sapiens | | | | locus | |
| | | | chromosome 15 | | | | | |
| | | | clone RP11-86K22 | | | | | |
| | | | map 15q21.3, | | | | | |
| | | | complete sequence | | | | | |
| | | | Length = 190420 | | | | | |
| 198 | | BC064689.1 | Homo sapiens tumor | NM_006290.2 | Homo sapiens tumor | Hs.211600 | Tumor | TNFAIP3 |
| | | | necrosis factor, | | necrosis factor, alpha- | | necrosis | |
| | | | alpha-induced | | induced protein 3 | | factor, alpha- | |
| | | | protein 3, mRNA | | (TNFAIP3), mRNA | | induced | |
| | | | (cDNA clone | | Length = 4446 | | protein 3 | |
| | | | IMAGE: 30345989), | | | | | |
| | | | complete cds | | | | | |
| | | | Length = 5457 | | | | | |
| 199 | | BC005080.1 | Homo sapiens | NM_052815.1 | Homo sapiens immediate | Hs.76095 | Immediate | IER3 |
| | | | immediate early | | early response 3 (IER3), | | early | |
| | | | response 3, | | transcript variant long, | | response 3 | |
| | | | transcript variant | | mRNA Length = 1345 | | | |
| | | | short, mRNA (cDNA | | | | | |
| | | | clone MGC: 13037 | | | | | |
| | | | IMAGE: 3617088), | | | | | |
| | | | complete cds | | | | | |
| | | | Length = 1240 | | | | | |
| 200 | | BC009015.1 | Homo sapiens serpin | NM_030666.2 | Homo sapiens serpin | Hs.381167 | Serpin | SERPINB1 |
| | | | peptidase inhibitor, | | peptidase inhibitor, clade B | | peptidase | |
| | | | clade B (ovalbumin), | | (ovalbumin), member 1 | | inhibitor, | |
| | | | member 1, mRNA | | (SERPINB1), mRNA | | clade B | |
| | | | (cDNA clone | | Length = 1310 | | (ovalbumin), | |
| | | | MGC: 9340 | | | | member 1 | |
| | | | IMAGE: 3456154), | | | | | |
| | | | complete cds | | | | | |
| | | | Length = 1310 | | | | | |
| 201 | | AF531102.1 | Homo sapiens | NM_000877.2 | Homo sapiens interleukin 1 | Hs.557403 | Interleukin 1 | IL1R1 |
| | | | interleukin 1 | | receptor, type I (IL1R1), | | receptor, | |
| | | | receptor, type I | | mRNA Length = 4909 | | type I | |
| | | | (IL1R1) gene, | | | | | |
| | | | complete cds | | | | | |
| | | | Length = 27864 | | | | | |
| 202 | | AF531102.1 | Homo sapiens | NM_000877.2 | Homo sapiens interleukin 1 | Hs.557403 | Interleukin 1 | IL1R1 |
| | | | interleukin 1 | | receptor, type I (IL1R1), | | receptor, | |
| | | | receptor, type I | | mRNA Length = 4909 | | type I | |
| | | | (IL1R1) gene, | | | | | |
| | | | complete cds | | | | | |
| | | | Length = 27864 | | | | | |
| 203 | | BC105285.1 | Homo sapiens | NM_004607.1 | Homo sapiens tubulin- | Hs.495912 | Dystrophin | DMD |
| | | | tubulin-specific | | specific chaperone a | | (muscular | |
| | | | chaperone a, mRNA | | (TBCA), mRNA | | dystrophy, | |
| | | | (cDNA clone | | Length = 574 | | Duchenne | |
| | | | MGC: 104402 | | | | and Becker | |
| | | | IMAGE: 6702573), | | | | types) | |
| | | | complete cds | | | | | |
| | | | Length = 590 | | | | | |
| 204 | | BC000866.1 | Homo sapiens TIMP | NM_003254.2 | Homo sapiens TIMP | Hs.522632 | TIMP | TIMP1 |
| | | | metallopeptidase | | metallopeptidase inhibitor 1 | | metallopeptidase | |
| | | | inhibitor 1, mRNA | | (TIMP1), mRNA | | inhibitor 1 | |
| | | | (cDNA clone | | Length = 931 | | | |
| | | | MGC: 5065 | | | | | |
| | | | IMAGE: 3460153), | | | | | |
| | | | complete cds | | | | | |
| | | | Length = 881 | | | | | |
| 205 | | BC025733.1 | Homo sapiens Fas | NM_003824.2 | Homo sapiens Fas | Hs.86131 | Fas | FADD |
| | | | (TNFRSF6)- | | (TNFRSF6)-associated via | | (TNFRSF6)- | |
| | | | associated via death | | death domain (FADD), | | associated | |
| | | | domain, mRNA | | mRNA Length = 1873 | | via death | |
| | | | (cDNA clone | | | | domain | |
| | | | IMAGE: 5203692) | | | | | |
| | | | Length = 1689 | | | | | |
| 206 | | BT019443.1 | Homo sapiens ras- | NM_005052.2 | Homo sapiens ras-related | Hs.45002 | Ras-related | RAC3 |
| | | | related C3 botulinum | | C3 botulinum toxin | | C3 botulinum | |
| | | | toxin substrate 3 (rho | | substrate 3 (rho family, | | toxin | |
| | | | family, small GTP | | small GTP binding protein | | substrate 3 | |
| | | | binding protein Rac3) | | Rac3) (RAC3), mRNA | | (rho family, | |
| | | | mRNA, complete cds | | Length = 1077 | | small GTP | |
| | | | Length = 579 | | | | binding | |
| | | | | | | | protein | |
| | | | | | | | Rac3) | |
| 207 | | AF517525.1 | Homo sapiens cyclin | NM_001760.2 | Homo sapiens cyclin D3 | Hs.534307 | Cyclin D3 | CCND3 |
| | | | D3 (CCND3) gene, | | (CCND3), mRNA | | | |
| | | | complete cds | | Length = 2095 | | | |
| | | | Length = 8987 | | | | | |
| 208 | | AF111168.2 | AF111168 Homo | NM_004863.2 | Homo sapiens serine | Hs.435661 | Serine | SPTLC2 |
| | | | sapiens serine | | palmitoyltransferase, long | | palmitoyltransferase, | |
| | | | palmitoyl transferase, | | chain base subunit 2 | | long | |
| | | | subunit II gene, | | (SPTLC2), mRNA | | chain base | |
| | | | complete cds; and | | Length = 7250 | | subunit 2 | |
| | | | unknown genes | | | | | |
| | | | Length = 231464 | | | | | |
| | | | Features flanking | | | | | |
| | | | this part of subject | | | | | |
| | | | sequence: 8949 bp | | | | | |
| | | | at 5 side: unknown | | | | | |
| | | | 4488 bp at 3 side: | | | | | |
| | | | serine palmitoyl | | | | | |
| | | | transferase, subunit | | | | | |
| | | | II | | | | | |
| 209 | | BC051281.1 | Homo sapiens | NM_203416.1 | Homo sapiens CD163 | Hs.504641 | CD163 | CD163 |
| | | | CD163 antigen, | | antigen (CD163), transcript | | antigen | |
| | | | transcript variant 1, | | variant 2, mRNA | | | |
| | | | mRNA (cDNA clone | | Length = 3728 | | | |
| | | | MGC: 59680 | | | | | |
| | | | IMAGE: 6280265), | | | | | |
| | | | complete cds | | | | | |
| | | | Length = 3780 | | | | | |
| 210 | | AC068946.4 | Homo sapiens BAC | NM_024293.2 | Homo sapiens | Hs.516707 | Chromosome | C2orf17 |
| | | | clone RP11-803J6 | | chromosome 2 open | | 2 open | |
| | | | from 2, complete | | reading frame 17 | | reading | |
| | | | sequence | | (C2orf17), mRNA | | frame 17 | |
| | | | Length = 172260 | | Length = 2615 | | | |
| 211 | | AY525612.1 | Macaca mulatta Toll- | NM_003264.3 | Homo sapiens toll-like | Hs.519033 | Toll-like | TLR2 |
| | | | like receptor 2 | | receptor 2 (TLR2), mRNA | | receptor 2 | |
| | | | (TLR2) mRNA, | | Length = 3417 | | | |
| | | | partial cds | | | | | |
| | | | Length = 434 | | | | | |
| 212 | | BC033756.1 | Homo sapiens toll- | NM_003264.3 | Homo sapiens toll-like | Hs.519033 | Toll-like | TLR2 |
| | | | like receptor 2, | | receptor 2 (TLR2), mRNA | | receptor 2 | |
| | | | mRNA (cDNA clone | | Length = 3417 | | | |
| | | | MGC: 45301 | | | | | |
| | | | IMAGE: 5213439), | | | | | |
| | | | complete cds | | | | | |
| | | | Length = 2756 | | | | | |
| 213 | | AY124010.1 | Homo sapiens | | | | | |
| | | | interleukin 1 | | | | | |
| | | | receptor, type II | | | | | |
| | | | (IL1R2) gene, | | | | | |
| | | | complete cds | | | | | |
| | | | Length = 23160 | | | | | |
| 214 | | BC022463.1 | Homo sapiens dual | NM_004417.2 | Homo sapiens dual | Hs.171695 | Dual | DUSP1 |
| | | | specificity | | specificity phosphatase 1 | | specificity | |
| | | | phosphatase 1, | | (DUSP1), mRNA | | phosphatase 1 | |
| | | | mRNA (cDNA clone | | Length = 2015 | | | |
| | | | MGC: 26153 | | | | | |
| | | | IMAGE: 4794895), | | | | | |
| | | | complete cds | | | | | |
| | | | Length = 2026 | | | | | |
| 215 | | BC012346.1 | Homo sapiens cDNA | NM_173343.1 | Homo sapiens interleukin 1 | Hs.25333 | Interleukin 1 | IL1R2 |
| | | | clone | | receptor, type II (IL1R2), | | receptor, | |
| | | | IMAGE: 4562827, | | transcript variant 2, mRNA | | type II | |
| | | | containing frame- | | Length = 1436 | | | |
| | | | shift errors | | | | | |
| | | | Length = 1430 | | | | | |
| 216 | | | | | | Hs.511486 | Similar to | |
| | | | | | | | 60S | |
| | | | | | | | ribosomal | |
| | | | | | | | protein L37a | |
| 217 | | J05070.1 | HUM4COLA Human | NM_004994.2 | Homo sapiens matrix | Hs.297413 | Matrix | MMP9 |
| | | | type IV collagenase | | metallopeptidase 9 | | metallopeptidase 9 | |
| | | | mRNA, complete cds | | (gelatinase B, 92 kDa | | (gelatinase | |
| | | | Length = 2334 | | gelatinase, 92 kDa type IV | | B, 92 kDa | |
| | | | | | collagenase) (MMP9), | | gelatinase, | |
| | | | | | mRNA Length = 2387 | | 92 kDa type | |
| | | | | | | | IV | |
| | | | | | | | collagenase) | |
| 218 | | AF134726.1 | Homo sapiens BAC | NM_005345.4 | Homo sapiens heat shock | Hs.520028 | Heat shock | HSPA1A |
| | | | clone 215O12 NG35, | | 70 kDa protein 1A | | 70 kDa | |
| | | | NG36, G9A, NG22, | | (HSPA1A), mRNA | | protein 1A | |
| | | | G9, HSP70-2, | | Length = 2383 | | | |
| | | | HSP70-1, HSP70- | | | | | |
| | | | HOM, snRNP, G7A, | | | | | |
| | | | NG37, NG23, and | | | | | |
| | | | MutSH5 genes, | | | | | |
| | | | complete cds | | | | | |
| | | | Length = 180283 | | | | | |
| 219 | | AY135686.1 | Homo sapiens | NM_001924.2 | Homo sapiens growth | Hs.80409 | Growth | GADD45A |
| | | | growth arrest and | | arrest and DNA-damage- | | arrest and | |
| | | | DNA-damage- | | inducible, alpha | | DNA- | |
| | | | inducible, alpha | | (GADD45A), mRNA | | damage- | |
| | | | (GADD45A) gene, | | Length = 1355 | | inducible, | |
| | | | complete cds | | | | alpha | |
| | | | Length = 4417 | | | | | |
| 220 | | BC002755.2 | Homo sapiens MAP | NM_003684.3 | Homo sapiens MAP kinase | Hs.371594 | MAP kinase | MKNK1 |
| | | | kinase interacting | | interacting serine/threonine | | interacting | |
| | | | serine/threonine | | kinase 1 (MKNK1), | | serine/threonine | |
| | | | kinase 1, mRNA | | transcript variant 1, mRNA | | kinase 1 | |
| | | | (cDNA clone | | Length = 2736 | | | |
| | | | MGC: 3690 | | | | | |
| | | | IMAGE: 3629765), | | | | | |
| | | | complete cds | | | | | |
| | | | Length = 2736 | | | | | |
| 221 | | AC004525.1 | HUAC004525 Homo | NM_000418.2 | Homo sapiens interleukin 4 | Hs.513457 | Interleukin 4 | IL4R |
| | | | sapiens | | receptor (IL4R), transcript | | receptor | |
| | | | Chromosome 16 | | variant 1, mRNA | | | |
| | | | BAC clone | | Length = 3678 | | | |
| | | | CIT987SK-582J2, | | | | | |
| | | | complete sequence | | | | | |
| | | | Length = 130020 | | | | | |
| 222 | | BC053871.2 | Homo sapiens | NM_005587.1 | Homo sapiens MADS box | Hs.268675 | MADS box | MEF2A |
| | | | MADS box | | transcription enhancer | | transcription | |
| | | | transcription | | factor 2, polypeptide A | | enhancer | |
| | | | enhancer factor 2, | | (myocyte enhancer factor | | factor 2, | |
| | | | polypeptide A | | 2A) (MEF2A), mRNA | | polypeptide | |
| | | | (myocyte enhancer | | Length = 2975 | | A (myocyte | |
| | | | factor 2A), mRNA | | | | enhancer | |
| | | | (cDNA clone | | | | factor 2A) | |
| | | | MGC: 61461 | | | | | |
| | | | IMAGE: 5555678), | | | | | |
| | | | complete cds | | | | | |
| | | | Length = 5054 | | | | | |
| 223 | | BC101075.2 | Homo sapiens cDNA | NM_016610.2 | Homo sapiens toll-like | Hs.272410 | Toll-like | TLR8 |
| | | | clone MGC: 119599 | | receptor 8 (TLR8), | | receptor 8 | |
| | | | IMAGE: 40008788, | | transcript variant 1, mRNA | | | |
| | | | complete cds | | Length = 3468 | | | |
| | | | Length = 3500 | | | | | |
| 224 | | BC017292.1 | Homo sapiens flotillin | NM_004475.1 | Homo sapiens flotillin 2 | Hs.514038 | Flotillin 2 | FLOT2 |
| | | | 2, mRNA (cDNA | | (FLOT2), mRNA | | | |
| | | | clone MGC: 29558 | | Length = 2488 | | | |
| | | | IMAGE: 4907083), | | | | | |
| | | | complete cds | | | | | |
| | | | Length = 2481 | | | | | |
| 225 | | AY619687.1 | Homo sapiens | NM_001629.2 | Homo sapiens | Hs.507658 | Arachidonate | ALOX5AP |
| | | | arachidonate 5- | | arachidonate 5- | | 5- | |
| | | | lipoxygenase- | | lipoxygenase-activating | | lipoxygenase- | |
| | | | activating protein | | protein (ALOX5AP), mRNA | | activating | |
| | | | (ALOX5AP) gene, | | Length = 873 | | protein | |
| | | | complete cds | | | | | |
| | | | Length = 32877 | | | | | |
| 226 | | BC001146.1 | Homo sapiens flotillin | NM_005803.2 | Homo sapiens flotillin 1 | Hs.179986 | Flotillin 1 | FLOT1 |
| | | | 1, mRNA (cDNA | | (FLOT1), mRNA | | | |
| | | | clone MGC: 1199 | | Length = 1839 | | | |
| | | | IMAGE: 3534771), | | | | | |
| | | | complete cds | | | | | |
| | | | Length = 1610 | | | | | |
| 227 | | AF177765.1 | AF177765 Homo | NM_138556.1 | Homo sapiens toll-like | Hs.174312 | Toll-like | TLR4 |
| | | | sapiens toll-like | | receptor 4 (TLR4), | | receptor 4 | |
| | | | receptor 4 (TLR4) | | transcript variant 2, mRNA | | | |
| | | | gene, TLR4A allele, | | Length = 3767 | | | |
| | | | complete cds | | | | | |
| | | | Length = 18974 | | | | | |
| 228 | | L35263.1 | HUMCSBP1 Human | NM_139014.1 | Homo sapiens mitogen- | Hs.485233 | Mitogen- | MAPK14 |
| | | | CSaids binding | | activated protein kinase 14 | | activated | |
| | | | protein (CSBP1) | | (MAPK14), transcript | | protein | |
| | | | mRNA, complete cds | | variant 4, mRNA | | kinase 14 | |
| | | | Length = 3757 | | Length = 3679 | | | |
| 229 | | L35263.1 | HUMCSBP1 Human | NM_139014.1 | Homo sapiens mitogen- | Hs.485233 | Mitogen- | MAPK14 |
| | | | CSaids binding | | activated protein kinase 14 | | activated | |
| | | | protein (CSBP1) | | (MAPK14), transcript | | protein | |
| | | | mRNA, complete cds | | variant 4, mRNA | | kinase 14 | |
| | | | Length = 3757 | | Length = 3679 | | | |
| 230 | | U19775.1 | HSU19775 Human | NM_139013.1 | Homo sapiens mitogen- | Hs.485233 | Mitogen- | MAPK14 |
| | | | MAP kinase Mxi2 | | activated protein kinase 14 | | activated | |
| | | | (MXI2) mRNA, | | (MAPK14), transcript | | protein | |
| | | | complete cds | | variant 3, mRNA | | kinase 14 | |
| | | | Length = 993 | | Length = 1312 | | | |
| 231 | | BC031574.1 | Homo sapiens | NM_139014.1 | Homo sapiens mitogen- | Hs.485233 | Mitogen- | MAPK14 |
| | | | mitogen-activated | | activated protein kinase 14 | | activated | |
| | | | protein kinase 14, | | (MAPK14), transcript | | protein | |
| | | | transcript variant 2, | | variant 4, mRNA | | kinase 14 | |
| | | | mRNA (cDNA clone | | Length = 3679 | | | |
| | | | MGC: 34610 | | | | | |
| | | | IMAGE: 5181064), | | | | | |
| | | | complete cds | | | | | |
| | | | Length = 3751 | | | | | |
| 232 | | M69043.1 | HUMMAD3A Homo | NM_020529.1 | Homo sapiens nuclear | Hs.81328 | Nuclear | NFKBIA |
| | | | sapiens MAD-3 | | factor of kappa light | | factor of | |
| | | | mRNA encoding lkB- | | polypeptide gene enhancer | | kappa light | |
| | | | like activity, complete | | in B-cells inhibitor, alpha | | polypeptide | |
| | | | cds Length = 1550 | | (NFKBIA), mRNA | | gene | |
| | | | | | Length = 1550 | | enhancer in | |
| | | | | | | | B-cells | |
| | | | | | | | inhibitor, | |
| | | | | | | | alpha | |
| 233 | | BC004983.1 | Homo sapiens | NM_020529.1 | Homo sapiens nuclear | Hs.81328 | Nuclear | NFKBIA |
| | | | nuclear factor of | | factor of kappa light | | factor of | |
| | | | kappa light | | polypeptide gene enhancer | | kappa light | |
| | | | polypeptide gene | | in B-cells inhibitor, alpha | | polypeptide | |
| | | | enhancer in B-cells | | (NFKBIA), mRNA | | gene | |
| | | | inhibitor, alpha, | | Length = 1550 | | enhancer in | |
| | | | mRNA (cDNA clone | | | | B-cells | |
| | | | MGC: 3942 | | | | inhibitor, | |
| | | | IMAGE: 2957970), | | | | alpha | |
| | | | complete cds | | | | | |
| | | | Length = 1566 | | | | | |
| 234 | | BC101075.2 | Homo sapiens cDNA | NM_016610.2 | Homo sapiens toll-like | Hs.272410 | Toll-like | TLR8 |
| | | | clone MGC: 119599 | | receptor 8 (TLR8), | | receptor 8 | |
| | | | IMAGE: 40008788, | | transcript variant 1, mRNA | | | |
| | | | complete cds | | Length = 3468 | | | |
| | | | Length = 3500 | | | | | |
| 235 | | BC068476.1 | Homo sapiens numb | NM_001005745.1 | Homo sapiens numb | Hs.509909 | Numb | NUMB |
| | | | homolog | | homolog (Drosophila) | | homolog | |
| | | | (Drosophila), | | (NUMB), transcript variant | | (Drosophila) | |
| | | | transcript variant 4, | | 4, mRNA Length = 3470 | | | |
| | | | mRNA (cDNA clone | | | | | |
| | | | MGC: 87117 | | | | | |
| | | | IMAGE: 4822300), | | | | | |
| | | | complete cds | | | | | |
| | | | Length = 3509 | | | | | |
| 236 | | BC012527.2 | Homo sapiens | NM_181054.1 | Homo sapiens hypoxia- | Hs.509554 | Hypoxia- | HIF1A |
| | | | hypoxia-inducible | | inducible factor 1, alpha | | inducible | |
| | | | factor 1, alpha | | subunit (basic helix-loop- | | factor 1, | |
| | | | subunit (basic helix- | | helix transcription factor) | | alpha subunit | |
| | | | loop-helix | | (HIF1A), transcript variant | | (basic helix- | |
| | | | transcription factor), | | 2, mRNA Length = 3812 | | loop-helix | |
| | | | transcript variant 1, | | | | transcription | |
| | | | mRNA (cDNA clone | | | | factor) | |
| | | | MGC: 10483 | | | | | |
| | | | IMAGE: 3842146), | | | | | |
| | | | complete cds | | | | | |
| | | | Length = 3933 | | | | | |
| 237 | | AY851161.1 | Homo sapiens decay | NM_000574.2 | Homo sapiens decay | Hs.527653 | Decay | DAF |
| | | | accelerating factor | | accelerating factor for | | accelerating | |
| | | | for complement | | complement (CD55, | | factor for | |
| | | | (CD55, Cromer | | Cromer blood group | | complement | |
| | | | blood group system) | | system) (DAF), mRNA | | (CD55, | |
| | | | (DAF) gene, | | Length = 2308 | | Cromer | |
| | | | complete cds | | | | blood group | |
| | | | Length = 43741 | | | | system) | |
| 238 | | AY323826.1 | Homo sapiens | NM_000628.3 | Homo sapiens interleukin | Hs.418291 | Interleukin | IL10RB |
| | | | interleukin 10 | | 10 receptor, beta (IL10RB), | | 10 receptor, | |
| | | | receptor, beta | | mRNA Length = 1935 | | beta | |
| | | | (IL10RB) gene, | | | | | |
| | | | complete cds | | | | | |
| | | | Length = 36210 | | | | | |
| 239 | | AC061992.11 | Homo sapiens | | | Hs.464217 | Dynein, | PGS1 |
| | | | chromosome 17, | | | | axonemal, | |
| | | | clone RP11-806H10, | | | | heavy | |
| | | | complete sequence | | | | polypeptide | |
| | | | Length = 100190 | | | | 17 | |
| 240 | | L21950.1 | HUMBENZA Human | NM_007311.3 | Homo sapiens | Hs.202 | Benzodiazapine | BZRP |
| | | | peripheral | | benzodiazapine receptor | | receptor | |
| | | | benzodiazepine | | (peripheral) (BZRP), | | (peripheral) | |
| | | | receptor related | | transcript variant PBR-S, | | | |
| | | | mRNA sequence | | mRNA Length = 665 | | | |
| | | | Length = 639 | | | | | |
| 241 | | BC001110.2 | Homo sapiens | NM_000714.4 | Homo sapiens | Hs.202 | Benzodiazapine | BZRP |
| | | | benzodiazapine | | benzodiazapine receptor | | receptor | |
| | | | receptor (peripheral), | | (peripheral) (BZRP), | | (peripheral) | |
| | | | transcript variant | | transcript variant PBR, | | | |
| | | | PBR, mRNA (cDNA | | mRNA Length = 876 | | | |
| | | | clone MGC: 1184 | | | | | |
| | | | IMAGE: 2989070), | | | | | |
| | | | complete cds | | | | | |
| | | | Length = 871 | | | | | |
| 242 | | BC013008.2 | Homo sapiens | NM_002795.2 | Homo sapiens proteasome | Hs.550875 | Transcribed | |
| | | | proteasome | | (prosome, macropain) | | locus, | |
| | | | (prosome, | | subunit, beta type, 3 | | strongly | |
| | | | macropain) subunit, | | (PSMB3), mRNA | | similar to | |
| | | | beta type, 3, mRNA | | Length = 784 | | NP_002786.2 | |
| | | | (cDNA clone | | | | proteasome | |
| | | | MGC: 4147 | | | | beta 3 | |
| | | | IMAGE: 3010175), | | | | subunit; | |
| | | | complete cds | | | | proteasome | |
| | | | Length = 783 | | | | theta chain; | |
| | | | | | | | proteasome | |
| | | | | | | | chain 13; | |
| | | | | | | | proteasome | |
| | | | | | | | component | |
| | | | | | | | C10-II | |
| | | | | | | | [Homo | |
| | | | | | | | sapiens] | |
| 243 | | AC025471.6 | Homo sapiens | NM_001737.2 | Homo sapiens complement | Hs.1290 | Complement | C9 |
| | | | chromosome 5 clone | | component 9 (C9), mRNA | | component 9 | |
| | | | RP11-138C1, | | Length = 2094 | | | |
| | | | complete sequence | | | | | |
| | | | Length = 148607 | | | | | |
| 244 | | BT019502.1 | Homo sapiens bone | NM_004334.1 | Homo sapiens bone | Hs.169998 | Bone | BST1 |
| | | | marrow stromal cell | | marrow stromal cell antigen | | marrow | |
| | | | antigen 1 mRNA, | | 1 (BST1), mRNA | | stromal cell | |
| | | | complete cds | | Length = 1411 | | antigen 1 | |
| | | | Length = 957 | | | | | |
| 245 | | BC013589.1 | Homo sapiens | NM_002468.2 | Homo sapiens myeloid | Hs.82116 | Myeloid | MYD88 |
| | | | myeloid | | differentiation primary | | differentiation | |
| | | | differentiation | | response gene (88) | | primary | |
| | | | primary response | | (MYD88), mRNA | | response | |
| | | | gene (88), mRNA | | Length = 2678 | | gene (88) | |
| | | | (cDNA clone | | | | | |
| | | | MGC: 9601 | | | | | |
| | | | IMAGE: 3900951), | | | | | |
| | | | complete cds | | | | | |
| | | | Length = 2678 | | | | | |
| 246 | | BC013589.1 | Homo sapiens | NM_002468.2 | Homo sapiens myeloid | Hs.82116 | Myeloid | MYD88 |
| | | | myeloid | | differentiation primary | | differentiation | |
| | | | differentiation | | response gene (88) | | primary | |
| | | | primary response | | (MYD88), mRNA | | response | |
| | | | gene (88), mRNA | | Length = 2678 | | gene (88) | |
| | | | (cDNA clone | | | | | |
| | | | MGC: 9601 | | | | | |
| | | | IMAGE: 3900951), | | | | | |
| | | | complete cds | | | | | |
| | | | Length = 2678 | | | | | |
| 247 | | AY279384.1 | Homo sapiens | NM_198829.1 | Homo sapiens ras-related | Hs.558376 | Ras-related | RAC1 |
| | | | migration-inducing | | C3 botulinum toxin | | C3 botulinum | |
| | | | protein 5 (MIG5) | | substrate 1 (rho family, | | toxin | |
| | | | mRNA, complete cds | | small GTP binding protein | | substrate 1 | |
| | | | Length = 833 | | Rac1) (RAC1), transcript | | (rho family, | |
| | | | | | variant Rac1c, mRNA | | small GTP | |
| | | | | | Length = 2413 | | binding | |
| | | | | | | | protein | |
| | | | | | | | Rac1) | |
| 248 | | AC007655.1 | Homo sapiens 12p13 | NM_001175.4 | Homo sapiens Rho GDP | Hs.504877 | Rho GDP | ARHGDIB |
| | | | BAC RPCI11-233G1 | | dissociation inhibitor (GDI) | | dissociation | |
| | | | (Roswell Park | | beta (ARHGDIB), mRNA | | inhibitor | |
| | | | Cancer Institute | | Length = 1216 | | (GDI) beta | |
| | | | Human BAC Library) | | | | | |
| | | | complete sequence | | | | | |
| | | | Length = 180069 | | | | | |
| 249 | | BC001410.2 | Homo sapiens S100 | NM_005620.1 | Homo sapiens S100 | Hs.547382 | CDNA clone | |
| | | | calcium binding | | calcium binding protein A11 | | IMAGE: 30389268 | |
| | | | protein A11 | | (calgizzarin) (S100A11), | | | |
| | | | (calgizzarin), mRNA | | mRNA Length = 595 | | | |
| | | | (cDNA clone | | | | | |
| | | | MGC: 2149 | | | | | |
| | | | IMAGE: 3140092), | | | | | |
| | | | complete cds | | | | | |
| | | | Length = 568 | | | | | |
| 250 | | DQ214322.1 | Taeniopygia guttata | NM_002107.3 | Homo sapiens H3 histone, | Hs.546259 | Similar to | |
| | | | clone | | family 3A (H3F3A), mRNA | | Histone H3.3 | |
| | | | 0058P0025E11 H3 | | Length = 1117 | | | |
| | | | histone family 3A-like | | | | | |
| | | | mRNA, complete | | | | | |
| | | | sequence | | | | | |
| | | | Length = 1109 | | | | | |
| 251 | | AF129756.1 | DJ201G24 Homo | NM_001288.4 | Homo sapiens chloride | Hs.414565 | Chloride | CLIC1 |
| | | | sapiens MSH55 | | intracellular channel 1 | | intracellular | |
| | | | gene, partial cds; | | (CLIC1), mRNA | | channel 1 | |
| | | | and CLIC1, DDAH, | | Length = 1265 | | | |
| | | | G6b, G6c, G5b, G6d, | | | | | |
| | | | G6e, G6f, BAT5, | | | | | |
| | | | G5b, CSK2B, BAT4, | | | | | |
| | | | G4, Apo M, BAT3, | | | | | |
| | | | BAT2, AIF-1, 1C7, | | | | | |
| | | | LST-1, LTB, TNF, | | | | | |
| | | | and LTA genes, | | | | | |
| | | | complete cds | | | | | |
| | | | Length = 184666 | | | | | |
| 252 | | M90727.1 | HUMFCGRA07 | NM_021642.2 | Homo sapiens Fc fragment | Hs.352642 | Fc fragment | FCGR2A |
| | | | Homo sapiens Fc- | | of IgG, low affinity IIa, | | of IgG, low | |
| | | | gamma-receptor IIA | | receptor (CD32) | | affinity IIa, | |
| | | | (FCGR2A) gene, | | (FCGR2A), mRNA | | receptor | |
| | | | exon 8 Length = 1659 | | Length = 2411 | | (CD32) | |
| 253 | | M90727.1 | HUMFCGRA07 | NM_021642.2 | Homo sapiens Fc fragment | Hs.352642 | Fc fragment | FCGR2A |
| | | | Homo sapiens Fc- | | of IgG, low affinity IIa, | | of IgG, low | |
| | | | gamma-receptor IIA | | receptor (CD32) | | affinity IIa, | |
| | | | (FCGR2A) gene, | | (FCGR2A), mRNA | | receptor | |
| | | | exon 8 Length = 1659 | | Length = 2411 | | (CD32) | |
| 254 | | U87575.1 | HSU87575 Human | NM_001002273.1 | Homo sapiens Fc fragment | Hs.492655 | Fc fragment | FCGR2B |
| | | | Fc-gamma-RIIb2 | | of IgG, low affinity IIb, | | of IgG, low | |
| | | | (Fc-gamma-RIIB) | | receptor (CD32) | | affinity IIc, | |
| | | | mRNA, partial cds | | (FCGR2B), transcript | | receptor for | |
| | | | Length = 873 | | variant 2, mRNA | | (CD32) | |
| | | | | | Length = 1573 | | | |
| 255 | | AC108044.5 | Homo sapiens BAC | NM_006068.2 | Homo sapiens toll-like | Hs.366986 | Toll-like | TLR6 |
| | | | clone RP11-280E3 | | receptor 6 (TLR6), mRNA | | receptor 6 | |
| | | | from 4, complete | | Length = 2753 | | | |
| | | | sequence | | | | | |
| | | | Length = 173834 | | | | | |
| 256 | | AY651785.1 | Homo sapiens | NM_000634.2 | Homo sapiens interleukin 8 | Hs.194778 | Interleukin 8 | IL8RA |
| | | | interleukin 8 | | receptor, alpha (IL8RA), | | receptor, | |
| | | | receptor, alpha | | mRNA Length = 2502 | | alpha | |
| | | | (IL8RA) gene, | | | | | |
| | | | complete cds | | | | | |
| | | | Length = 19092 | | | | | |
| 257 | | AY714242.1 | Homo sapiens | NM_001557.2 | Homo sapiens interleukin 8 | Hs.846 | Interleukin 8 | IL8RB |
| | | | interleukin 8 | | receptor, beta (IL8RB), | | receptor, | |
| | | | receptor, beta | | mRNA Length = 2859 | | beta | |
| | | | (IL8RB) gene, | | | | | |
| | | | complete cds | | | | | |
| | | | Length = 25733 | | | | | |
| 258 | | AY771351.1 | Homo sapiens zinc | NM_003407.1 | Homo sapiens zinc finger | Hs.534052 | Zinc finger | ZFP36 |
| | | | finger protein 36, | | protein 36, C3H type, | | protein 36, | |
| | | | C3H type, homolog | | homolog (mouse) (ZFP36), | | C3H type, | |
| | | | (mouse) (ZFP36) | | mRNA Length = 1746 | | homolog | |
| | | | gene, complete cds | | | | (mouse) | |
| | | | Length = 5413 | | | | | |
| 259 | | BC065293.1 | Homo sapiens cDNA | NM_004862.2 | Homo sapiens | Hs.459940 | Lipopolysaccharide- | LITAF |
| | | | clone | | lipopolysaccharide-induced | | induced TNF | |
| | | | IMAGE: 6060454, | | TNF factor (LITAF), mRNA | | factor | |
| | | | containing frame- | | Length = 2368 | | | |
| | | | shift errors | | | | | |
| | | | Length = 2238 | | | | | |
| 260 | | BC010140.2 | Homo sapiens tumor | NM_001065.2 | Homo sapiens tumor | Hs.279594 | Tumor | TNFRSF1A |
| | | | necrosis factor | | necrosis factor receptor | | necrosis | |
| | | | receptor superfamily, | | superfamily, member 1A | | factor | |
| | | | member 1A, mRNA | | (TNFRSF1A), mRNA | | receptor | |
| | | | (cDNA clone | | Length = 2236 | | superfamily, | |
| | | | MGC: 19588 | | | | member 1A | |
| | | | IMAGE: 4131360), | | | | | |
| | | | complete cds | | | | | |
| | | | Length = 2185 | | | | | |
| 261 | | BC010140.2 | Homo sapiens tumor | NM_001065.2 | Homo sapiens tumor | Hs.279594 | Tumor | TNFRSF1A |
| | | | necrosis factor | | necrosis factor receptor | | necrosis | |
| | | | receptor superfamily, | | superfamily, member 1A | | factor | |
| | | | member 1A, mRNA | | (TNFRSF1A), mRNA | | receptor | |
| | | | (cDNA clone | | Length = 2236 | | superfamily, | |
| | | | MGC: 19588 | | | | member 1A | |
| | | | IMAGE: 4131360), | | | | | |
| | | | complete cds | | | | | |
| | | | Length = 2185 | | | | | |
| 262 | | BC001602.1 | Homo sapiens | NM_003879.3 | Homo sapiens CASP8 and | Hs.447489 | Similar to | IFITM3 |
| | | | CASP8 and FADD- | | FADD-like apoptosis | | Interferon- | |
| | | | like apoptosis | | regulator (CFLAR), mRNA | | induced | |
| | | | regulator, mRNA | | Length = 2243 | | transmembrane | |
| | | | (cDNA clone | | | | protein 3 | |
| | | | MGC: 2044 | | | | (Interferon- | |
| | | | IMAGE: 3542993), | | | | inducible | |
| | | | complete cds | | | | protein 1-8U) | |
| | | | Length = 2025 | | | | | |
| 263 | | BC001602.1 | Homo sapiens | NM_003879.3 | Homo sapiens CASP8 and | Hs.390736 | CASP8 and | CFLAR |
| | | | CASP8 and FADD- | | FADD-like apoptosis | | FADD-like | |
| | | | like apoptosis | | regulator (CFLAR), mRNA | | apoptosis | |
| | | | regulator, mRNA | | Length = 2243 | | regulator | |
| | | | (cDNA clone | | | | | |
| | | | MGC: 2044 | | | | | |
| | | | IMAGE: 3542993), | | | | | |
| | | | complete cds | | | | | |
| | | | Length = 2025 | | | | | |
| 264 | | AC005300.10 | Homo sapiens | NM_014339.3 | Homo sapiens interleukin | Hs.129751 | Interleukin | IL17R |
| | | | chromosome 22 | | 17 receptor (IL17R), mRNA | | 17 receptor | |
| | | | clone p143i13 map | | Length = 3429 | | | |
| | | | 22q11, complete | | | | | |
| | | | sequence | | | | | |
| | | | Length = 101269 | | | | | |
| 265 | | BC014966.1 | Homo sapiens | NM_001006665.1 | Homo sapiens ribosomal | Hs.149957 | Ribosomal | RPS6KA1 |
| | | | ribosomal protein S6 | | protein S6 kinase, 90 kDa, | | protein S6 | |
| | | | kinase, 90 kDa, | | polypeptide 1 (RPS6KA1), | | kinase, | |
| | | | polypeptide 1, mRNA | | transcript variant 2, mRNA | | 90 kDa, | |
| | | | (cDNA clone | | Length = 3112 | | polypeptide 1 | |
| | | | MGC: 23062 | | | | | |
| | | | IMAGE: 4867373), | | | | | |
| | | | complete cds | | | | | |
| | | | Length = 3206 | | | | | |
| 266 | | BC029782.1 | Homo sapiens | NM_003006.3 | Homo sapiens selectin P | Hs.127346 | Selectin P | SELPLG |
| | | | selectin P ligand, | | ligand (SELPLG), mRNA | | ligand | |
| | | | mRNA (cDNA clone | | Length = 2550 | | | |
| | | | MGC: 34401 | | | | | |
| | | | IMAGE: 5180824), | | | | | |
| | | | complete cds | | | | | |
| | | | Length = 2173 | | | | | |
| 267 | | BC064500.1 | Homosapiens | NM_001109.2 | Homo sapiens ADAM | Hs.501574 | ADAM | ADAM8 |
| | | | ADAM | | metallopeptidase domain 8 | | metallopeptidase | |
| | | | metallopeptidase | | (ADAM8), mRNA | | domain 8 | |
| | | | domain 8, mRNA | | Length = 3275 | | | |
| | | | (cDNA clone | | | | | |
| | | | IMAGE: 6247789) | | | | | |
| | | | Length = 3115 | | | | | |
| 268 | | BC014117.2 | Homo sapiens | NM_030984.1 | Homo sapiens | Hs.520757 | Thromboxane | TBXAS1 |
| | | | thromboxane A | | thromboxane A synthase 1 | | A synthase | |
| | | | synthase 1 (platelet, | | (platelet, cytochrome P450, | | 1 (platelet, | |
| | | | cytochrome P450, | | family 5, subfamily A) | | cytochrome | |
| | | | family 5, subfamily | | (TBXAS1), transcript | | P450, family | |
| | | | A), mRNA (cDNA | | variant TXS-II, mRNA | | 5, subfamily | |
| | | | clone MGC: 20885 | | Length = 1783 | | A) | |
| | | | IMAGE: 4548935), | | | | | |
| | | | complete cds | | | | | |
| | | | Length = 2119 | | | | | |
| 269 | | BC017412.1 | Homo sapiens | NM_006866.1 | Homo sapiens leukocyte | Hs.534394 | Leukocyte | LILRA2 |
| | | | leukocyte | | immunoglobulin-like | | immunoglobulin- | |
| | | | immunoglobulin-like | | receptor, subfamily A (with | | like | |
| | | | receptor, subfamily A | | TM domain), member 2 | | receptor, | |
| | | | (with TM domain), | | (LILRA2), mRNA | | subfamily A | |
| | | | member 2, mRNA | | Length = 1696 | | (with TM | |
| | | | (cDNA clone | | | | domain), | |
| | | | MGC: 27265 | | | | member 2 | |
| | | | IMAGE: 4618777), | | | | | |
| | | | complete cds | | | | | |
| | | | Length = 1682 | | | | | |
| 270 | | AY651785.1 | Homo sapiens | NM_000634.2 | Homo sapiens interleukin 8 | Hs.194778 | Interleukin 8 | IL8RA |
| | | | interleukin 8 | | receptor, alpha (IL8RA), | | receptor, | |
| | | | receptor, alpha | | mRNA Length = 2502 | | alpha | |
| | | | (IL8RA) gene, | | | | | |
| | | | complete cds | | | | | |
| | | | Length = 19092 | | | | | |
| 271 | | BC011991.1 | Homo sapiens serpin | NM_001002236.1 | Homo sapiens serpin | Hs.525557 | Serpin | SERPINA1 |
| | | | peptidase inhibitor, | | peptidase inhibitor, clade A | | peptidase | |
| | | | clade A (alpha-1 | | (alpha-1 antiproteinase, | | inhibitor, | |
| | | | antiproteinase, | | antitrypsin), member 1 | | clade A | |
| | | | antitrypsin), member | | (SERPINA1), transcript | | (alpha-1 | |
| | | | 1, transcript variant | | variant 2, mRNA | | antiproteinase, | |
| | | | 1, mRNA (cDNA | | Length = 1902 | | antitrypsin), | |
| | | | clone MGC: 9222 | | | | member 1 | |
| | | | IMAGE: 3859644), | | | | | |
| | | | complete cds | | | | | |
| | | | Length = 1584 | | | | | |
| 272 | | AF543826.1 | Homo sapiens IgG | NM_001002273.1 | Homo sapiens Fc fragment | Hs.492655 | Fc fragment | FCGR2B |
| | | | receptor IIB | | of IgG, low affinity IIb, | | of IgG, low | |
| | | | (FCGR2B) mRNA, | | receptor (CD32) | | affinity IIc, | |
| | | | complete cds | | (FCGR2B), transcript | | receptor for | |
| | | | Length = 1509 | | variant 2, mRNA | | (CD32) | |
| | | | | | Length = 1573 | | | |
| 273 | | AC068657.6 | Homo sapiens | NM_000679.3 | Homo sapiens adrenergic, | Hs.368632 | Adrenergic, | ADRA1B |
| | | | chromosome 5 clone | | alpha-1B-, receptor | | alpha-1B-, | |
| | | | CTB-67I13, complete | | (ADRA1B), mRNA | | receptor | |
| | | | sequence | | Length = 2272 | | | |
| | | | Length = 128332 | | | | | |
| 274 | | AC004849.1 | Homo sapiens PAC | XM_376680.2 | PREDICTED: Homo | Hs.308710 | KIAA1718 | KIAA1718 |
| | | | clone RP4-659J6 | | sapiens KIAA1718 protein | | protein | |
| | | | from 7, complete | | (KIAA1718), mRNA | | | |
| | | | sequence | | Length = 9910 | | | |
| | | | Length = 126801 | | | | | |
| 275 | | BC066116.1 | Homo sapiens | NM_000478.2 | Homo sapiens alkaline | Hs.75431 | Alkaline | ALPL |
| | | | alkaline | | phosphatase, | | phosphatase, | |
| | | | phosphatase, | | liver/bone/kidney (ALPL), | | liver/bone/kidney | |
| | | | liver/bone/kidney, | | mRNA Length = 2580 | | | |
| | | | mRNA (cDNA clone | | | | | |
| | | | IMAGE: 5754297), | | | | | |
| | | | partial cds | | | | | |
| | | | Length = 2561 | | | | | |
| 276 | | AC072022.19 | Homo sapiens 3 | NM_138931.1 | Homo sapiens B-cell | Hs.478588 | B-cell | BCL6 |
| | | | BAC RP11-211G3 | | CLL/lymphoma 6 (zinc | | CLL/lymphoma | |
| | | | (Roswell Park | | finger protein 51) (BCL6), | | 6 (zinc | |
| | | | Cancer Institute | | transcript variant 2, mRNA | | finger protein | |
| | | | Human BAC Library) | | Length = 3630 | | 51) | |
| | | | complete sequence | | | | | |
| | | | Length = 125395 | | | | | |
| 277 | | J03925.1 | HUMMAC1A Human | NM_000632.2 | Homo sapiens integrin, | Hs.172631 | Integrin, | ITGAM |
| | | | Mac-1 gene | | alpha M (complement | | alpha M | |
| | | | encoding | | component receptor 3, | | (complement | |
| | | | complement receptor | | alpha; also known as | | component | |
| | | | type 3, CD11b, | | CD11b (p170), | | receptor 3, | |
| | | | complete cds | | macrophage antigen alpha | | alpha; also | |
| | | | Length = 4740 | | polypeptide) (ITGAM), | | known as | |
| | | | | | mRNA Length = 4740 | | CD11b | |
| | | | | | | | (p170), | |
| | | | | | | | macrophage | |
| | | | | | | | antigen | |
| | | | | | | | alpha | |
| | | | | | | | polypeptide) | |
| 278 | | AY233976.1 | Homo sapiens | NM_000655.2 | Homo sapiens selectin L | Hs.82848 | Selectin L | SELL |
| | | | selectin L | | (lymphocyte adhesion | | (lymphocyte | |
| | | | (lymphocyte | | molecule 1) (SELL), mRNA | | adhesion | |
| | | | adhesion molecule | | Length = 2324 | | molecule 1) | |
| | | | 1) (SELL) gene, | | | | | |
| | | | complete cds | | | | | |
| | | | Length = 24822 | | | | | |
| 279 | | AC108044.5 | Homo sapiens BAC | NM_003263.3 | Homo sapiens toll-like | Hs.111805 | Toll-like | TLR1 |
| | | | clone RP11-280E3 | | receptor 1 (TLR1), mRNA | | receptor 1 | |
| | | | from 4, complete | | Length = 2867 | | | |
| | | | sequence | | | | | |
| | | | Length = 173834 | | | | | |
| 280 | | J03571.1 | HUMLOX5A Human | NM_000698.2 | Homo sapiens | Hs.89499 | Arachidonate | ALOX5 |
| | | | 5-lipoxygenase | | arachidonate 5- | | 5- | |
| | | | mRNA, complete cds | | lipoxygenase (ALOX5), | | lipoxygenase | |
| | | | Length = 2484 | | mRNA Length = 2568 | | | |
| 281 | | BC053585.1 | Homo sapiens | NM_172313.1 | Homo sapiens colony | Hs.524517 | Colony | CSF3R |
| | | | colony stimulating | | stimulating factor 3 | | stimulating | |
| | | | factor 3 receptor | | receptor (granulocyte) | | factor 3 | |
| | | | (granulocyte), | | (CSF3R), transcript variant | | receptor | |
| | | | transcript variant 1, | | 4, mRNA Length = 2583 | | (granulocyte) | |
| | | | mRNA (cDNA clone | | | | | |
| | | | MGC: 61541 | | | | | |
| | | | IMAGE: 5217305), | | | | | |
| | | | complete cds | | | | | |
| | | | Length = 3029 | | | | | |
| 282 | | | | | | Hs.89499 | Arachidonate | ALOX5 |
| | | | | | | | 5- | |
| | | | | | | | lipoxygenase | |
| 283 | | AY358712.1 | Homo sapiens clone | NM_024308.2 | Homo sapiens short-chain | Hs.462859 | Short-chain | MGC4172 |
| | | | DNA77626 | | dehydrogenase/reductase | | dehydrogenase/ | |
| | | | ARPG836 (UNQ836) | | (MGC4172), mRNA | | reductase | |
| | | | mRNA, complete cds | | Length = 1501 | | | |
| | | | Length = 1505 | | | | | |
| 284 | | | | | | Hs.501853 | TMEM9 | C11orf15 |
| | | | | | | | domain | |
| | | | | | | | family, | |
| | | | | | | | member B | |
| 285 | | AC011241.7 | Homo sapiens BAC | | | Hs.146657 | Transcribed | |
| | | | clone RP11-467P13 | | | | locus | |
| | | | from 2, complete | | | | | |
| | | | sequence | | | | | |
| | | | Length = 104891 | | | | | |
| 286 | | AC093752.2 | Homo sapiens BAC | | | Hs.535008 | Full length | |
| | | | clone RP11-33B1 | | | | insert cDNA | |
| | | | from 4, complete | | | | clone | |
| | | | sequence | | | | YU26H08 | |
| | | | Length = 130879 | | | | | |
| 287 | | AF131806.1 | AF131806 Homo | | | Hs.494875 | Regulator of | RGS3 |
| | | | sapiens clone 24863 | | | | G-protein | |
| | | | mRNA sequence | | | | signalling 3 | |
| | | | Length = 1841 | | | | | |
| 288 | | AC016735.10 | Homo sapiens BAC | | | | | |
| | | | clone RP11-391M15 | | | | | |
| | | | from 2, complete | | | | | |
| | | | sequence | | | | | |
| | | | Length = 182730 | | | | | |
| 289 | | AC013472.8 | Homo sapiens BAC | XM_515349.1 | PREDICTED: Pan | Hs.132725 | Transcribed | |
| | | | clone RP11-503P10 | | troglodytes LOC459090 | | locus, | |
| | | | from 2, complete | | (LOC459090), mRNA | | strongly | |
| | | | sequence | | Length = 1791 | | similar to | |
| | | | Length = 149481 | | | | XP_515349.1 | |
| | | | | | | | PREDICTED: | |
| | | | | | | | hypothetical | |
| | | | | | | | protein | |
| | | | | | | | XP_515349 | |
| | | | | | | | [Pan | |
| | | | | | | | troglodytes] | |
| 290 | | BC073839.1 | Homo sapiens tumor | NM_152942.2 | Homo sapiens tumor | Hs.1314 | Tumor | TNFRSF8 |
| | | | necrosis factor | | necrosis factor receptor | | necrosis | |
| | | | receptor superfamily, | | superfamily, member 8 | | factor | |
| | | | member 8, mRNA | | (TNFRSF8), transcript | | receptor | |
| | | | (cDNA clone | | variant 2, mRNA | | superfamily, | |
| | | | IMAGE: 5219187), | | Length = 2361 | | member 8 | |
| | | | partial cds | | | | | |
| | | | Length = 2179 | | | | | |
| 291 | | | | | | Hs.527684 | Transcribed | |
| | | | | | | | locus | |
| 292 | | BC073999.1 | Homo sapiens | NM_004121.1 | Homo sapiens gamma- | Hs.437156 | Gamma- | GGTLA1 |
| | | | gamma- | | glutamyltransferase-like | | glutamyltransferase- | |
| | | | glutamyltransferase- | | activity 1 (GGTLA1), mRNA | | like | |
| | | | like activity 1, mRNA | | Length = 2414 | | activity 1 | |
| | | | (cDNA clone | | | | | |
| | | | MGC: 87996 | | | | | |
| | | | IMAGE: 6198113), | | | | | |
| | | | complete cds | | | | | |
| | | | Length = 2481 | | | | | |
| 293 | | BC004490.2 | Homo sapiens v-fos | NM_005252.2 | Homo sapiens v-fos FBJ | Hs.25647 | V-fos FBJ | FOS |
| | | | FBJ murine | | murine osteosarcoma viral | | murine | |
| | | | osteosarcoma viral | | oncogene homolog (FOS), | | osteosarcoma | |
| | | | oncogene homolog, | | mRNA Length = 2084 | | viral | |
| | | | mRNA (cDNA clone | | | | oncogene | |
| | | | MGC: 11074 | | | | homolog | |
| | | | IMAGE: 3688670), | | | | | |
| | | | complete cds | | | | | |
| | | | Length = 1814 | | | | | |
| 294 | | BC099908.1 | Homo sapiens E2F | NM_001949.2 | Homo sapiens E2F | Hs.269408 | E2F | E2F3 |
| | | | transcription factor 3, | | transcription factor 3 | | transcription | |
| | | | mRNA (cDNA clone | | (E2F3), mRNA | | factor 3 | |
| | | | IMAGE: 30531209), | | Length = 4744 | | | |
| | | | complete cds | | | | | |
| | | | Length = 4128 | | | | | |
| 295 | | S75476.1 | PGK1 = phosphoglycerate | NM_000291.2 | Homo sapiens | Hs.78771 | Phosphoglycerate | PGK1 |
| | | | kinase 1 {3 | | phosphoglycerate kinase 1 | | kinase 1 | |
| | | | nuclease-sensitive | | (PGK1), mRNA | | | |
| | | | region} [human, | | Length = 2338 | | | |
| | | | Genomic, 3571 nt] | | | | | |
| | | | Length = 3571 | | | | | |
| 296 | | AC152891.2 | Pan troglodytes | NM_022977.1 | Homo sapiens acyl-CoA | Hs.268785 | Acyl-CoA | ACSL4 |
| | | | chromosome X clone | | synthetase long-chain | | synthetase | |
| | | | RP43-038O03 map | | family member 4 (ACSL4), | | long-chain | |
| | | | human ortholog | | transcript variant 2, mRNA | | family | |
| | | | Xq23, complete | | Length = 5356 | | member 4 | |
| | | | sequence | | | | | |
| | | | Length = 197819 | | | | | |
| 297 | | AC152891.2 | Pan troglodytes | NM_022977.1 | Homo sapiens acyl-CoA | Hs.268785 | Acyl-CoA | ACSL4 |
| | | | chromosome X clone | | synthetase long-chain | | synthetase | |
| | | | RP43-038O03 map | | family member 4 (ACSL4), | | long-chain | |
| | | | human ortholog | | transcript variant 2, mRNA | | family | |
| | | | Xq23, complete | | Length = 5356 | | member 4 | |
| | | | sequence | | | | | |
| | | | Length = 197819 | | | | | |
| 298 | | BC067779.1 | Homo sapiens TRAF | NM_004180.2 | Homo sapiens TRAF family | Hs.132257 | TRAF family | TANK |
| | | | family member- | | member-associated NFKB | | member- | |
| | | | associated NFKB | | activator (TANK), transcript | | associated | |
| | | | activator, mRNA | | variant 1, mRNA | | NFKB | |
| | | | (cDNA clone | | Length = 2089 | | activator | |
| | | | MGC: 87157 | | | | | |
| | | | IMAGE: 5296558), | | | | | |
| | | | complete cds | | | | | |
| | | | Length = 2052 | | | | | |
| 299 | | BC002461.2 | Homo sapiens | NM_004330.1 | Homo sapiens | Hs.283454 | BCL2/adeno | BNIP2 |
| | | | BCL2/adenovirus | | BCL2/adenovirus E1B | | virus E1B | |
| | | | E1B 19 kDa | | 19 kDa interacting protein 2 | | 19 kDa | |
| | | | interacting protein 2, | | (BNIP2), mRNA | | interacting | |
| | | | mRNA (cDNA clone | | Length = 2382 | | protein 2 | |
| | | | MGC: 1529 | | | | | |
| | | | IMAGE: 3343559), | | | | | |
| | | | complete cds | | | | | |
| | | | Length = 2409 | | | | | |
| 300 | | M23492.1 | HUMLCA35 Homo | NM_080922.1 | Homo sapiens protein | Hs.192039 | Protein | PTPRC |
| | | | sapiens T200 | | tyrosine phosphatase, | | tyrosine | |
| | | | leukocyte common | | receptor type, C (PTPRC), | | phosphatase, | |
| | | | antigen precursor | | transcript variant 3, mRNA | | receptor | |
| | | | (PTPRC) gene, exon | | Length = 4882 | | type, C | |
| | | | 33 and partial cds | | | | | |
| | | | Length = 1375 | | | | | |
| 301 | | BC019816.2 | Homo sapiens heat | NM_153201.1 | Homo sapiens heat shock | Hs.535026 | Similar to | |
| | | | shock 70 kDa protein | | 70 kDa protein 8 (HSPA8), | | Heat shock | |
| | | | 8, transcript variant | | transcript variant 2, mRNA | | cognate 71 kDa | |
| | | | 1, mRNA (cDNA | | Length = 1817 | | protein | |
| | | | clone MGC: 29929 | | | | | |
| | | | IMAGE: 2899894), | | | | | |
| | | | complete cds | | | | | |
| | | | Length = 2250 | | | | | |
| 302 | | AC116913.6 | Homo sapiens | NM_002755.2 | Homo sapiens mitogen- | Hs.145442 | Mitogen- | MAP2K1 |
| | | | chromosome 15, | | activated protein kinase | | activated | |
| | | | clone CTD-3185P2, | | kinase 1 (MAP2K1), mRNA | | protein | |
| | | | complete sequence | | Length = 2222 | | kinase | |
| | | | Length = 152561 | | | | kinase 1 | |
| 303 | | BC18569.2 | Homo sapiens | NM_005389.1 | Homo sapiens protein-L- | Hs.279257 | Protein-L- | PCMT1 |
| | | | protein-L- | | isoaspartate (D-aspartate) | | isoaspartate | |
| | | | isoaspartate (D- | | O-methyltransferase | | (D-aspartate) | |
| | | | aspartate) O- | | (PCMT1), mRNA | | O- | |
| | | | methyltransferase, | | Length = 1599 | | methyltransferase | |
| | | | mRNA (cDNA clone | | | | | |
| | | | IMAGE: 3446814) | | | | | |
| | | | Length = 1237 | | | | | |
| 304 | | AC098587.5 | Homo sapiens BAC | NM_006206.3 | Homo sapiens platelet- | Hs.74615 | Platelet- | PDGFRA |
| | | | clone RP11-231C18 | | derived growth factor | | derived | |
| | | | from 4, complete | | receptor, alpha polypeptide | | growth factor | |
| | | | sequence | | (PDGFRA), mRNA | | receptor, | |
| | | | Length = 132164 | | Length = 6405 | | alpha | |
| | | | | | | | polypeptide | |
| 305 | | AY101192.1 | Homo sapiens CD44 | NM_001001392.1 | Homo sapiens CD44 | Hs.502328 | CD44 | CD44 |
| | | | antigen (CD44) | | antigen (homing function | | antigen | |
| | | | mRNA, complete cds | | and Indian blood group | | (homing | |
| | | | Length = 2292 | | system) (CD44), transcript | | function and | |
| | | | | | variant 5, mRNA | | Indian blood | |
| | | | | | Length = 3985 | | group | |
| | | | | | | | system) | |
| 306 | | AF277315.6 | Homo sapiens | NM_003639.2 | Homo sapiens inhibitor of | Hs.559535 | Transcribed | |
| | | | chromosome X clone | | kappa light polypeptide | | locus, | |
| | | | RP5-865E18, RP5- | | gene enhancer in B-cells, | | strongly | |
| | | | 1087L19 map q28, | | kinase gamma (IKBKG), | | similar to | |
| | | | complete sequence | | mRNA Length = 2121 | | NP_003630.1 | |
| | | | Length = 147234 | | | | inhibitor of | |
| | | | | | | | kappa light | |
| | | | | | | | polypeptide | |
| | | | | | | | gene | |
| | | | | | | | enhancer in | |
| | | | | | | | B-cells, | |
| | | | | | | | kinase | |
| | | | | | | | gamma | |
| | | | | | | | [Homo | |
| | | | | | | | sapiens] | |
| 307 | | BT019600.1 | Synthetic construct | NM_172088.1 | Homo sapiens tumor | Hs.54673 | Tumor | TNFSF13 |
| | | | Homo sapiens tumor | | necrosis factor (ligand) | | necrosis | |
| | | | necrosis factor | | superfamily, member 13 | | factor | |
| | | | (ligand) superfamily, | | (TNFSF13), transcript | | (ligand) | |
| | | | member 13 mRNA, | | variant gamma, mRNA | | superfamily, | |
| | | | partial cds | | Length = 2095 | | member 12 | |
| | | | Length = 753 | | | | | |
| 308 | | BC030815.1 | Homo sapiens | NM_181524.1 | Homo sapiens | Hs.191911 | Nuclear | NFIA |
| | | | phosphoinositide-3- | | phosphoinositide-3-kinase, | | factor I/A | |
| | | | kinase, regulatory | | regulatory subunit 1 (p85 | | | |
| | | | subunit 1 (p85 | | alpha) (PIK3R1), transcript | | | |
| | | | alpha), transcript | | variant 3, mRNA | | | |
| | | | variant 2, mRNA | | Length = 5823 | | | |
| | | | (cDNA clone | | | | | |
| | | | MGC: 22760 | | | | | |
| | | | IMAGE: 4290954), | | | | | |
| | | | complete cds | | | | | |
| | | | Length = 2427 | | | | | |
| 309 | 1240372 | | | | | | | |
| 310 | | U39657.1 | HSU39657 Human | NM_031988.1 | Homo sapiens mitogen- | Hs.463978 | Mitogen- | MAP2K6 |
| | | | MAP kinase kinase 6 | | activated protein kinase | | activated | |
| | | | (MKK6) mRNA, | | kinase 6 (MAP2K6), | | protein | |
| | | | complete cds | | transcript variant 2, mRNA | | kinase | |
| | | | Length = 2924 | | Length = 2820 | | kinase 6 | |
| 311 | IMAGE: 114030 | | | | | | | |
| 312 | | AC073336.6 | Homo sapiens BAC | | | Hs.434944 | Transcribed | |
| | | | clone RP11-476H24 | | | | locus | |
| | | | from 7, complete | | | | | |
| | | | sequence | | | | | |
| | | | Length = 166261 | | | | | |
| 313 | | AC023818.5 | Homo sapiens | | | Hs.295923 | Seven in | SIAH1 |
| | | | chromosome 16 | | | | absentia | |
| | | | clone CTD-2600H12, | | | | homolog 1 | |
| | | | complete sequence | | | | (Drosophila) | |
| | | | Length = 173304 | | | | | |
| 314 | | AC005856.1 | AC005856 Homo | | | Hs.517949 | Microtubule- | MAP4 |
| | | | sapiens | | | | associated | |
| | | | chromosome 17, | | | | protein 4 | |
| | | | clone | | | | | |
| | | | hRPK.136_H_19, | | | | | |
| | | | complete sequence | | | | | |
| | | | Length = 126910 | | | | | |
| 315 | | AC005881.3 | AC005881 | | | Hs.567261 | Single | SSBP3 |
| | | | citb_79_e_16, | | | | stranded | |
| | | | complete sequence | | | | DNA binding | |
| | | | Length = 181773 | | | | protein 3 | |
| 316 | | AC025423.32 | Homo sapiens 12 | NM_006878.2 | Homo sapiens Mdm2, | Hs.369849 | Mdm2, | MDM2 |
| | | | BAC RP11-611O2 | | transformed 3T3 cell | | transformed | |
| | | | (Roswell Park | | double minute 2, p53 | | 3T3 cell | |
| | | | Cancer Institute | | binding protein (mouse) | | double | |
| | | | Human BAC Library) | | (MDM2), transcript variant | | minute 2, | |
| | | | complete sequence | | MDM2a, mRNA | | p53 binding | |
| | | | Length = 150579 | | Length = 1613 | | protein | |
| | | | | | | | (mouse) | |
| 317 | | | | | | Hs.127386 | MAM domain | MAMDC2 |
| | | | | | | | containing 2 | |
| 318 | | | | | | Hs.293928 | Hypothetical | |
| | | | | | | | gene | |
| | | | | | | | supported by | |
| | | | | | | | AK024293 | |
| 319 | | AC011092.4 | Homo sapiens, clone | | | Hs.438794 | SET binding | SBF2 |
| | | | RP11-1H15, | | | | factor 2 | |
| | | | complete sequence | | | | | |
| | | | Length = 192318 | | | | | |
| 320 | | AY789109.1 | Homo sapiens | | | | | |
| | | | interleukin 3 | | | | | |
| | | | receptor, alpha (low | | | | | |
| | | | affinity) (IL3RA) | | | | | |
| | | | gene, complete cds | | | | | |
| | | | Length = 49973 | | | | | |
| 321 | | AC007878.2 | Homo sapiens BAC | | | Hs.434401 | Zinc finger | ZNF638 |
| | | | clone RP11-236P2 | | | | protein 638 | |
| | | | from 2, complete | | | | | |
| | | | sequence | | | | | |
| | | | Length = 185134 | | | | | |
| 322 | | | | | | Hs.123070 | Homeo box | HOXD10 |
| | | | | | | | D10 | |
| 323 | | AY162833.1 | Homo sapiens E2F | | | Hs.445758 | E2F | E2F5 |
| | | | transcription factor 5, | | | | transcription | |
| | | | p130-binding (E2F5) | | | | factor 5, | |
| | | | gene, complete cds | | | | p130-binding | |
| | | | Length = 38504 | | | | | |
| 324 | | AC084794.3 | Homo sapiens | | | Hs.547315 | Hypothetical | |
| | | | chromosome 16 | | | | protein | |
| | | | clone CTA-154B9, | | | | LOC440345 | |
| | | | complete sequence | | | | | |
| | | | Length = 230510 | | | | | |
| 325 | | AC005532.2 | Homo sapiens PAC | | | | | |
| | | | clone RP4-733B9 | | | | | |
| | | | from 7, complete | | | | | |
| | | | sequence | | | | | |
| | | | Length = 115954 | | | | | |
| 326 | IMAGE: 2336269 | | | | | | | |
| 327 | | | | | | Hs.513706 | MARVEL | MARVELD3 |
| | | | | | | | domain | |
| | | | | | | | containing 3 | |
| 328 | | | | | | Hs.22930 | Transcribed | |
| | | | | | | | locus | |
| 329 | | BC039424.1 | Homo sapiens cDNA | | | Hs.378808 | Eukaryotic | eIF2A |
| | | | clone | | | | translation | |
| | | | IMAGE: 5302722 | | | | initiation | |
| | | | Length = 1526 | | | | factor (eIF) | |
| | | | | | | | 2A | |
| 330 | | AF377331.2 | AF377331 Homo | NM_002188.2 | Homo sapiens interleukin | Hs.845 | Interleukin | IL13 |
| | | | sapiens interleukin | | 13 (IL13), mRNA | | 13 | |
| | | | 13 (IL13) gene, | | Length = 1282 | | | |
| | | | complete cds | | | | | |
| | | | Length = 6919 | | | | | |
| 331 | | | | | | Hs.147531 | Transcribed | |
| | | | | | | | locus | |
| 332 | | AC126283.3 | Homo sapiens BAC | NM_000204.1 | Homo sapiens I factor | Hs.312485 | I factor | IF |
| | | | clone RP11-602N24 | | (complement) (IF), mRNA | | (complement) | |
| | | | from 4, complete | | Length = 1963 | | | |
| | | | sequence | | | | | |
| | | | Length = 118642 | | | | | |
| 333 | | BC046362.1 | Homo sapiens | NM_183240.1 | Homo sapiens | Hs.26216 | Transmembrane | TMEM37 |
| | | | transmembrane | | transmembrane protein 37 | | protein | |
| | | | protein 37, mRNA | | (TMEM37), mRNA | | 37 | |
| | | | (cDNA clone | | Length = 579 | | | |
| | | | MGC: 50757 | | | | | |
| | | | IMAGE: 5221396), | | | | | |
| | | | complete cds | | | | | |
| | | | Length = 1850 | | | | | |
| 334 | | AF313041.4 | Homo sapiens | | | Hs.336994 | Metastasis | MTSS1 |
| | | | chromosome 8 clone | | | | suppressor 1 | |
| | | | RP11-158K1 map | | | | | |
| | | | q24.13, complete | | | | | |
| | | | sequence | | | | | |
| | | | Length = 193882 | | | | | |
| 335 | | | | | | Hs.159827 | Transcribed | |
| | | | | | | | locus | |
| 336 | | AC009265.10 | Homo sapiens, clone | | | Hs.558316 | Chitobiase, | CTBS |
| | | | RP11-45B18, | | | | di-N-acetyl- | |
| | | | complete sequence | | | | | |
| | | | Length = 179844 | | | | | |
| 337 | | AY714878.1 | Homo sapiens anti- | NM_020547.1 | Homo sapiens anti- | Hs.437877 | Anti- | AMHR2 |
| | | | Mullerian hormone | | Mullerian hormone | | Mullerian | |
| | | | receptor type II | | receptor, type II (AMHR2), | | hormone | |
| | | | (AMHR2) mRNA, | | mRNA Length = 1855 | | receptor, | |
| | | | complete cds, | | | | type II | |
| | | | alternatively spliced | | | | | |
| | | | Length = 1437 | | | | | |
| 338 | | AF235103.5 | Homo sapiens | | | | | |
| | | | chromosome 8 | | | | | |
| | | | multiple clones map | | | | | |
| | | | q24.3, complete | | | | | |
| | | | sequence | | | | | |
| | | | Length = 345524 | | | | | |
| 339 | | AY509906.1 | Homo sapiens | NM_018402.1 | Homo sapiens interleukin | Hs.272350 | Interleukin | IL26 |
| | | | interleukin 26 (IL26) | | 26 (IL26), mRNA | | 26 | |
| | | | gene, complete cds | | Length = 1047 | | | |
| | | | Length = 33991 | | | | | |
| 340 | | | | | | Hs.148360 | Transcribed | |
| | | | | | | | locus | |
| 341 | | XM_499571 | PREDICTED: Homo | XM_499571 | PREDICTED: Homo | | Hs.558664 | PREDICTED: |
| | | | sapiens hypothetical | | sapiens hypothetical protein | | | Homo sapiens |
| | | | protein FLJ30672 | | FLJ30672 (FLJ30672) | | | hypothetical |
| | | | (FLJ30672), mRNA | | | | | protein |
| | | | | | | | | FLJ30672 |
| | | | | | | | | (FLJ30672) |
| 342 | | AC008870.8 | Homo sapiens | | | Hs.560464 | Transcribed | |
| | | | chromosome 16 | | | | locus | |
| | | | clone CTD-2196E14, | | | | | |
| | | | complete sequence | | | | | |
| | | | Length = 150522 | | | | | |
| 343 | | AC131097.6 | Homo sapiens BAC | | | Hs.372194 | CDNA | |
| | | | clone RP11-789L24 | | | | FLJ40615 | |
| | | | from 2, complete | | | | fis, clone | |
| | | | sequence | | | | THYMU2012882 | |
| | | | Length = 187030 | | | | | |
| 344 | | AC002395.1 | Homo sapiens 12 | | | Hs.76253 | Ataxin 2 | ATXN2 |
| | | | PAC RPCI1-315L5 | | | | | |
| | | | (Roswell Park | | | | | |
| | | | Cancer Institute | | | | | |
| | | | Human PAC library) | | | | | |
| | | | complete sequence | | | | | |
| | | | Length = 155022 | | | | | |
| 345 | | AC107909.5 | Homo sapiens | | | | | |
| | | | chromosome 8, | | | | | |
| | | | clone CTD-2340D6, | | | | | |
| | | | complete sequence | | | | | |
| | | | Length = 149001 | | | | | |
| 346 | | BC058922.1 | Homo sapiens cDNA | NM_001017402.1 | Homo sapiens laminin, beta | Hs.497636 | Laminin, | LAMB3 |
| | | | clone | | 3 (LAMB3), transcript | | beta 3 | |
| | | | IMAGE: 5175186, | | variant 2, mRNA | | | |
| | | | containing frame- | | Length = 4339 | | | |
| | | | shift errors | | | | | |
| | | | Length = 4119 | | | | | |
| 347 | | AC124833.1 | Homo sapiens BAC | | | Hs.134634 | Transcribed | |
| | | | clone RP11-324E12 | | | | locus | |
| | | | from 7, complete | | | | | |
| | | | sequence | | | | | |
| | | | Length = 80050 | | | | | |
| 348 | | BC007032.1 | Homo sapiens cDNA | NM_000607.1 | Homo sapiens | Hs.522356 | Orosomucoid 1 | ORM1 |
| | | | clone | | orosomucoid 1 (ORM1), | | | |
| | | | IMAGE: 4071534, | | mRNA Length = 803 | | | |
| | | | containing frame- | | | | | |
| | | | shift errors | | | | | |
| | | | Length = 845 | | | | | |
| 349 | | | | | | Hs.522520 | Chromosome | C9orf37 |
| | | | | | | | 9 open | |
| | | | | | | | reading | |
| | | | | | | | frame 37 | |
| 350 | | AB008193 | Homo sapiens genes | AB008193 | Homo sapiens genes for | Hs.567248 | leukotriene | LTB4R |
| | | | for leukotriene B4 | | leukotriene B4 receptor | | B4 receptor | |
| | | | receptor BLT2, | | BLT2, leukotriene B4 | | BLT2, | |
| | | | leukotriene B4 | | | | leukotriene | |
| | | | | | | | B4 | |
| 351 | | BC101754.1 | Homo sapiens cDNA | XM_514191.1 | PREDICTED: Pan | Hs.19281 | Protein | PTPN14 |
| | | | clone MGC: 126803 | | troglodytes similar to | | tyrosine | |
| | | | IMAGE: 8069260, | | protein tyrosine | | phosphatase, | |
| | | | complete cds | | phosphatase, non-receptor | | non- | |
| | | | Length = 3645 | | type 14; cytoskeletal- | | receptor type | |
| | | | | | associated protein tyrosine | | 14 | |
| | | | | | phosphatase (LOC457730), | | | |
| | | | | | mRNA Length = 3218 | | | |
| 352 | | AC067881.5 | Homo sapiens | | | Hs.332422 | Aspartate | ASPH |
| | | | chromosome 8, | | | | beta- | |
| | | | clone RP11-687K23, | | | | hydroxylase | |
| | | | complete sequence | | | | | |
| | | | Length = 186676 | | | | | |
| 353 | | | | | | Hs.476706 | Mitochondrial | MRPL37 |
| | | | | | | | ribosomal | |
| | | | | | | | protein L37 | |
| 354 | | | | | | Hs.86080 | Transcribed | |
| | | | | | | | locus, | |
| | | | | | | | moderately | |
| | | | | | | | similar to | |
| | | | | | | | NP_777603.1 | |
| | | | | | | | hypothetical | |
| | | | | | | | protein | |
| | | | | | | | FLJ25976 | |
| | | | | | | | [Homo | |
| | | | | | | | sapiens] | |
| 355 | | BC034406.1 | Homo sapiens | NM_022141.4 | Homo sapiens parvin, | Hs.437857 | Parvin, | PARVG |
| | | | parvin, gamma, | | gamma (PARVG), mRNA | | gamma | |
| | | | mRNA (cDNA clone | | Length = 1629 | | | |
| | | | MGC: 35319 | | | | | |
| | | | IMAGE: 5178529), | | | | | |
| | | | complete cds | | | | | |
| | | | Length = 1684 | | | | | |
| 356 | | | | | | Hs.529643 | Transcribed | |
| | | | | | | | locus | |
| 357 | | BC034761.1 | Homo sapiens | NM_007264.3 | Homo sapiens | Hs.483909 | Adrenomedullin | ADMR |
| | | | adrenomedullin | | adrenomedullin receptor | | receptor | |
| | | | receptor, mRNA | | (ADMR), mRNA | | | |
| | | | (cDNA clone | | Length = 1620 | | | |
| | | | MGC: 34399 | | | | | |
| | | | IMAGE: 5185932), | | | | | |
| | | | complete cds | | | | | |
| | | | Length = 1745 | | | | | |
| 358 | IMAGE: 1713245 | | | | | | | |
| 359 | | | | | | Hs.287537 | B cell RAG | GALNAC4S- |
| | | | | | | | associated | 6ST |
| | | | | | | | protein | |
| 360 | | BC072681.1 | Homo sapiens zinc | NM_020205.1 | Homo sapiens zinc finger, | Hs.98322 | Zinc finger, | ZA20D1 |
| | | | finger, A20 domain | | A20 domain containing 1 | | A20 domain | |
| | | | containing 1, mRNA | | (ZA20D1), mRNA | | containing 1 | |
| | | | (cDNA clone | | Length = 3159 | | | |
| | | | MGC: 87939 | | | | | |
| | | | IMAGE: 6170513), | | | | | |
| | | | complete cds | | | | | |
| | | | Length = 3618 | | | | | |
| 361 | | | | | | Hs.558924 | FLJ42177 | FLJ42177 |
| | | | | | | | protein | |
| 362 | | AC006531.1 | AC006531 Homo | | | Hs.411472 | Glutamate | GRIN2A |
| | | | sapiens | | | | receptor, | |
| | | | chromosome 16 | | | | ionotropic, N- | |
| | | | clone 113K5, | | | | methyl D- | |
| | | | complete sequence | | | | aspartate 2A | |
| | | | Length = 167525 | | | | | |
| 363 | | AC019227.4 | Homo sapiens BAC | | | Hs.178499 | CDNA | |
| | | | clone RP11-567O18 | | | | FLJ39357 | |
| | | | from 11, complete | | | | fis, clone | |
| | | | sequence | | | | PEBLM2003914 | |
| | | | Length = 190314 | | | | | |
| 364 | | AC055711.18 | Homo sapiens 3 | | | | | |
| | | | BAC RP11-67E18 | | | | | |
| | | | (Roswell Park | | | | | |
| | | | Cancer Institute | | | | | |
| | | | Human BAC Library) | | | | | |
| | | | complete sequence | | | | | |
| | | | Length = 154850 | | | | | |
| 365 | | BC074964.2 | Homo sapiens ciliary | NM_170768.1 | Homo sapiens zinc finger | Hs.524920 | Zinc finger | ZFP91 |
| | | | neurotrophic factor, | | protein 91 homolog | | protein 91 | |
| | | | mRNA (cDNA clone | | (mouse) (ZFP91), transcript | | homolog | |
| | | | MGC: 103900 | | variant 2, mRNA | | (mouse) | |
| | | | IMAGE: 30915287), | | Length = 3478 | | | |
| | | | complete cds | | | | | |
| | | | Length = 665 | | | | | |
| 366 | | AC091974.3 | Homo sapiens | | | Hs.444390 | Homo | |
| | | | chromosome 5 clone | | | | sapiens, | |
| | | | RP11-51P10, | | | | clone | |
| | | | complete sequence | | | | IMAGE: 4479080 | |
| | | | Length = 130853 | | | | | |
| 367 | | AC010412.9 | Homo sapiens | NM_003573.1 | Homo sapiens latent | Hs.466766 | Latent | LTBP4 |
| | | | chromosome 19 | | transforming growth factor | | transforming | |
| | | | clone CTD-2166J9, | | beta binding protein 4 | | growth factor | |
| | | | complete sequence | | (LTBP4), mRNA | | beta binding | |
| | | | Length = 155085 | | Length = 5054 | | protein 4 | |
| 368 | | BC022283.1 | Homo sapiens | NM_005666.2 | Homo sapiens complement | Hs.154224 | Complement | CFHL1 |
| | | | complement factor | | factor H-related 2 (CFHL2), | | factor H- | |
| | | | H-related 2, mRNA | | mRNA Length = 1043 | | related 1 | |
| | | | (cDNA clone | | | | pseudogene | |
| | | | MGC: 22434 | | | | | |
| | | | IMAGE: 4734331), | | | | | |
| | | | complete cds | | | | | |
| | | | Length = 1075 | | | | | |
| 369 | | BC015663.2 | Homo sapiens serpin | NM_006216.2 | Homo sapiens serpin | Hs.38449 | Serpin | SERPINE2 |
| | | | peptidase inhibitor, | | peptidase inhibitor, clade E | | peptidase | |
| | | | clade E (nexin, | | (nexin, plasminogen | | inhibitor, | |
| | | | plasminogen | | activator inhibitor type 1), | | clade E | |
| | | | activator inhibitor | | member 2 (SERPINE2), | | (nexin, | |
| | | | type 1), member 2, | | mRNA Length = 2120 | | plasminogen | |
| | | | mRNA (cDNA clone | | | | activator | |
| | | | MGC: 23129 | | | | inhibitor type | |
| | | | IMAGE: 4578406), | | | | 1), member 2 | |
| | | | complete cds | | | | | |
| | | | Length = 2120 | | | | | |
| 370 | IMAGE: 2329442 | | | | | | | |
| 371 | | AC002395.1 | Homo sapiens 12 | | | Hs.76253 | Ataxin 2 | ATXN2 |
| | | | PAC RPCI1-315L5 | | | | | |
| | | | (Roswell Park | | | | | |
| | | | Cancer Institute | | | | | |
| | | | Human PAC library) | | | | | |
| | | | complete sequence | | | | | |
| | | | Length = 155022 | | | | | |
| 372 | | AC026975.5 | Homo sapiens | NM_002843.2 | Homo sapiens protein | Hs.318547 | Protein | PTPRJ |
| | | | chromosome 11, | | tyrosine phosphatase, | | tyrosine | |
| | | | clone RP11-709C9, | | receptor type, J (PTPRJ), | | phosphatase, | |
| | | | complete sequence | | mRNA Length = 5119 | | receptor | |
| | | | Length = 200105 | | | | type, J | |
| 373 | | BC041943.1 | Homo sapiens | | | Hs.479954 | Methylenetetrahydrofolate | MTHFD2L |
| | | | methylenetetrahydrofolate | | | | dehydrogenase | |
| | | | dehydrogenase | | | | (NADP+ | |
| | | | (NADP+ dependent) | | | | dependent) | |
| | | | 2-like, mRNA (cDNA | | | | 2-like | |
| | | | clone | | | | | |
| | | | IMAGE: 5301277) | | | | | |
| | | | Length = 2377 | | | | | |
| 374 | | AC007653.5 | AC007653 Homo | | | Hs.478393 | Peroxisomal | PEX5L |
| | | | sapiens | | | | biogenesis | |
| | | | chromosome, clone | | | | factor 5-like | |
| | | | RP11-51A1, | | | | | |
| | | | complete sequence | | | | | |
| | | | Length = 159926 | | | | | |
| 375 | | AC026250.16 | Homo sapiens | | | Hs.552269 | MRNA; | |
| | | | chromosome 11, | | | | cDNA | |
| | | | clone RP11-540A21, | | | | DKFZp686C19106 | |
| | | | complete sequence | | | | (from | |
| | | | Length = 160681 | | | | clone | |
| | | | | | | | DKFZp686C19106) | |
| 376 | | | | | | Hs.190342 | Transcribed | |
| | | | | | | | locus | |
| 377 | | BC068537.1 | Homo sapiens serine | NM_006415.2 | Homo sapiens serine | Hs.124503 | Serine | TCF8 |
| | | | palmitoyltransferase, | | palmitoyltransferase, long | | palmitoyltransferase, | |
| | | | long chain base | | chain base subunit 1 | | long | |
| | | | subunit 1, mRNA | | (SPTLC1), transcript | | chain base | |
| | | | (cDNA clone | | variant 1, mRNA | | subunit 1 | |
| | | | MGC: 87384 | | Length = 2780 | | | |
| | | | IMAGE: 5259635), | | | | | |
| | | | complete cds | | | | | |
| | | | Length = 2742 | | | | | |
| 378 | | BC063588.1 | Homo sapiens | NM_201274.2 | Homo sapiens myosin | Hs.462341 | Myosin | M-RIP |
| | | | myosin phosphatase- | | phosphatase-Rho | | phosphatase- | |
| | | | Rho interacting | | interacting protein (M-RIP), | | Rho | |
| | | | protein, mRNA | | mRNA Length = 3923 | | interacting | |
| | | | (cDNA clone | | | | protein | |
| | | | IMAGE: 4441347), | | | | | |
| | | | partial cds | | | | | |
| | | | Length = 2072 | | | | | |
| 379 | | AY779023.1 | Homo sapiens | NM_181310.1 | Homo sapiens interleukin | Hs.126891 | Interleukin | IL22RA2 |
| | | | interleukin 22 | | 22 receptor, alpha 2 | | 22 receptor, | |
| | | | receptor, alpha 2 | | (IL22RA2), transcript | | alpha 2 | |
| | | | (IL22RA2) gene, | | variant 3, mRNA | | | |
| | | | complete cds | | Length = 2644 | | | |
| | | | Length = 32866 | | | | | |
| 380 | | AC008770.7 | Homo sapiens | | | | | |
| | | | chromosome 19 | | | | | |
| | | | clone CTD-2006C1, | | | | | |
| | | | complete sequence | | | | | |
| | | | Length = 178184 | | | | | |
| 381 | | AC011452.6 | AC011452 Homo | NM_012068.2 | Homo sapiens activating | Hs.9754 | Activating | ATF5 |
| | | | sapiens | | transcription factor 5 | | transcription | |
| | | | chromosome 19 | | (ATF5), mRNA | | factor 5 | |
| | | | clone CTC-326K19, | | Length = 2010 | | | |
| | | | complete sequence | | | | | |
| | | | Length = 146741 | | | | | |
| 382 | | AC117395.5 | Homo sapiens 3 | | | Hs.12333 | Ring finger | RNF13 |
| | | | BAC RP11-651P23 | | | | protein 13 | |
| | | | (Roswell Park | | | | | |
| | | | Cancer Institute | | | | | |
| | | | Human BAC Library) | | | | | |
| | | | complete sequence | | | | | |
| | | | Length = 172715 | | | | | |
| 383 | | | | | | Hs.562136 | Transcribed | |
| | | | | | | | locus | |
| 384 | | BC033989.2 | Homo sapiens cDNA | | | Hs.369473 | CDNA clone | |
| | | | clone | | | | IMAGE: 5296164 | |
| | | | IMAGE: 5296164 | | | | | |
| | | | Length = 911 | | | | | |
| 385 | | AF220188.1 | AF220188 Homo | | | Hs.479766 | TPA | TPARL |
| | | | sapiens | | | | regulated | |
| | | | uncharacterized | | | | locus | |
| | | | hypothalamus | | | | | |
| | | | protein HTMP | | | | | |
| | | | mRNA, complete cds | | | | | |
| | | | Length = 1913 | | | | | |
| 386 | | AY603772.1 | Homo sapiens UDP | NG_002601.1 | Homo sapiens UDP | Hs.554822 | UDP | UGT1A6 |
| | | | glycosyltransferase 1 | | glucuronosyltransferase 1 | | glucuronosyltransferase 1 | |
| | | | family, polypeptide | | family, polypeptide A | | family, | |
| | | | A1 (UGT1A1) gene, | | cluster (UGT1A@) on | | polypeptide | |
| | | | complete cds | | chromosome 2 | | A9 | |
| | | | Length = 16944 | | Length = 189056 | | | |
| 387 | | BC067511.1 | Homo sapiens | NM_016584.2 | Homo sapiens interleukin | Hs.98309 | Interleukin | IL23A |
| | | | interleukin 23, alpha | | 23, alpha subunit p19 | | 23, alpha | |
| | | | subunit p19, mRNA | | (IL23A), mRNA | | subunit p19 | |
| | | | (cDNA clone | | Length = 1049 | | | |
| | | | MGC: 79390 | | | | | |
| | | | IMAGE: 6971850), | | | | | |
| | | | complete cds | | | | | |
| | | | Length = 618 | | | | | |
| 388 | | AF305083.1 | AF305083 Homo | NM_002033.2 | Homo sapiens | Hs.568106 | Transcribed | |
| | | | sapiens alpha(1,3)- | | fucosyltransferase 4 (alpha | | locus | |
| | | | fucosyltransferase IV | | (1,3) fucosyltransferase, | | | |
| | | | (FUTIV) gene, 3 | | myeloid-specific) (FUT4), | | | |
| | | | UTR Length = 4192 | | mRNA Length = 5937 | | | |
| 389 | | K01500.1 | HUMA1ACM Human | NM_001085.4 | Homo sapiens serpin | Hs.534293 | Serpin | SERPINA3 |
| | | | alpha-1- | | peptidase inhibitor, clade A | | peptidase | |
| | | | antichymotrypsin | | (alpha-1 antiproteinase, | | inhibitor, | |
| | | | (AACT) mRNA, | | antitrypsin), member 3 | | clade A | |
| | | | complete cds | | (SERPINA3), mRNA | | (alpha-1 | |
| | | | Length = 1520 | | Length = 1629 | | antiproteinase, | |
| | | | | | | | antitrypsin), | |
| | | | | | | | member 3 | |
| 390 | | BC027933.1 | Homo sapiens | NM_002751.5 | Homo sapiens mitogen- | Hs.57732 | Mitogen- | MAPK11 |
| | | | mitogen-activated | | activated protein kinase 11 | | activated | |
| | | | protein kinase 11, | | (MAPK11), transcript | | protein | |
| | | | transcript variant 1, | | variant 1, mRNA | | kinase 11 | |
| | | | mRNA (cDNA clone | | Length = 2420 | | | |
| | | | MGC: 34566 | | | | | |
| | | | IMAGE: 5199628), | | | | | |
| | | | complete cds | | | | | |
| | | | Length = 2480 | | | | | |
| 391 | | | | | | Hs.562012 | Transcribed | |
| | | | | | | | locus | |
| 392 | | BC027933.1 | Homo sapiens | NM_002751.5 | Homo sapiens mitogen- | Hs.57732 | Mitogen- | MAPK11 |
| | | | mitogen-activated | | activated protein kinase 11 | | activated | |
| | | | protein kinase 11, | | (MAPK11), transcript | | protein | |
| | | | transcript variant 1, | | variant 1, mRNA | | kinase 11 | |
| | | | mRNA (cDNA clone | | Length = 2420 | | | |
| | | | MGC: 34566 | | | | | |
| | | | IMAGE: 5199628), | | | | | |
| | | | complete cds | | | | | |
| | | | Length = 2480 | | | | | |
| 393 | | AF284095.1 | AF284095 Homo | NM_000681.2 | Homo sapiens adrenergic, | Hs.249159 | Adrenergic, | ADRA2A |
| | | | sapiens alpha-2A | | alpha-2A-, receptor | | alpha-2A-, | |
| | | | adrenergic receptor | | (ADRA2A), mRNA | | receptor | |
| | | | mRNA, complete cds | | Length = 3653 | | | |
| | | | Length = 3653 | | | | | |
| 394 | | BC033694.1 | Homo sapiens | NM_006538.2 | Homo sapiens BCL2-like | Hs.469658 | BCL2-like 11 | BCL2L11 |
| | | | BCL2-like 11 | | 11 (apoptosis facilitator) | | (apoptosis | |
| | | | (apoptosis | | (BCL2L11), transcript | | facilitator) | |
| | | | facilitator), transcript | | variant 6, mRNA | | | |
| | | | variant 1, mRNA | | Length = 3242 | | | |
| | | | (cDNA clone | | | | | |
| | | | MGC: 45206 | | | | | |
| | | | IMAGE: 5213713), | | | | | |
| | | | complete cds | | | | | |
| | | | Length = 3679 | | | | | |
| 395 | | AC104850.2 | Homo sapiens | NM_005201.2 | Homo sapiens chemokine | Hs.113222 | Chemokine | CCR8 |
| | | | chromosome 3 clone | | (C-C motif) receptor 8 | | (C-C motif) | |
| | | | RP11-241K18, | | (CCR8), mRNA | | receptor 8 | |
| | | | complete sequence | | Length = 2370 | | | |
| | | | Length = 173580 | | | | | |
| 396 | | | | | | Hs.6874 | Receptor | RIPK5 |
| | | | | | | | interacting | |
| | | | | | | | protein | |
| | | | | | | | kinase 5 | |
| 397 | | | | XM_514542.1 | PREDICTED: Pan | Hs.176013 | Chromosome | C20orf26 |
| | | | | | troglodytes similar to | | 20 open | |
| | | | | | hypothetical protein | | reading | |
| | | | | | (LOC458126), mRNA | | frame 26 | |
| | | | | | Length = 3904 | | | |
| 398 | | AC026458.8 | Homo sapiens | | | Hs.512856 | SEC14-like 5 | KIAA0420 |
| | | | chromosome 16 | | | | (S. cerevisiae) | |
| | | | clone RP11-10K17, | | | | | |
| | | | complete sequence | | | | | |
| | | | Length = 153873 | | | | | |
| 399 | | BC030514.1 | Homo sapiens tudor | XM_509806.1 | PREDICTED: Pan | Hs.525061 | Tudor | TDRD3 |
| | | | domain containing 3, | | troglodytes similar to tudor | | domain | |
| | | | mRNA (cDNA clone | | domain containing 3 | | containing 3 | |
| | | | MGC: 40253 | | (LOC452750), mRNA | | | |
| | | | IMAGE: 4998330), | | Length = 2260 | | | |
| | | | complete cds | | | | | |
| | | | Length = 2550 | | | | | |
| 400 | | AC015604.5 | AC015604 Homo | | | Hs.97313 | Transcribed | |
| | | | sapiens, clone RP11- | | | | locus | |
| | | | 45D18, complete | | | | | |
| | | | sequence | | | | | |
| | | | Length = 172914 | | | | | |
| 401 | | BC033094.1 | Homo sapiens | NM_003640.2 | Homo sapiens inhibitor of | Hs.494738 | Inhibitor of | IKBKAP |
| | | | inhibitor of kappa | | kappa light polypeptide | | kappa light | |
| | | | light polypeptide | | gene enhancer in B-cells, | | polypeptide | |
| | | | gene enhancer in B- | | kinase complex-associated | | gene | |
| | | | cells, kinase | | protein (IKBKAP), mRNA | | enhancer in | |
| | | | complex-associated | | Length = 5917 | | B-cells, | |
| | | | protein, mRNA | | | | kinase | |
| | | | (cDNA clone | | | | complex- | |
| | | | MGC: 45667 | | | | associated | |
| | | | IMAGE: 4562340), | | | | protein | |
| | | | complete cds | | | | | |
| | | | Length = 5047 | | | | | |
| 402 | | | | | | Hs.485489 | Chloride | CLIC5 |
| | | | | | | | intracellular | |
| | | | | | | | channel 5 | |
| 403 | | BC005168.1 | Homo sapiens | NM_032646.5 | Homo sapiens tweety | Hs.27935 | Tweety | TTYH2 |
| | | | tweety homolog 2 | | homolog 2 (Drosophila) | | homolog 2 | |
| | | | (Drosophila), mRNA | | (TTYH2), transcript variant | | (Drosophila) | |
| | | | (cDNA clone | | 1, mRNA Length = 3468 | | | |
| | | | IMAGE: 3543080), | | | | | |
| | | | partial cds | | | | | |
| | | | Length = 1775 | | | | | |
| 404 | | BC040503.1 | Homo sapiens | NM_152332.2 | Homo sapiens membrane | Hs.510262 | Membrane | MTAC2D1 |
| | | | membrane targeting | | targeting (tandem) C2 | | targeting | |
| | | | (tandem) C2 domain | | domain containing 1 | | (tandem) C2 | |
| | | | containing 1, mRNA | | (MTAC2D1), mRNA | | domain | |
| | | | (cDNA clone | | Length = 4620 | | containing 1 | |
| | | | MGC: 33891 | | | | | |
| | | | IMAGE: 5298787), | | | | | |
| | | | complete cds | | | | | |
| | | | Length = 4620 | | | | | |
| 405 | | AC012307.8 | Homo sapiens BAC | NM_017849.1 | Homo sapiens hypothetical | Hs.355708 | Hypothetical | FLJ20507 |
| | | | clone RP11-574O17 | | protein FLJ20507 | | protein | |
| | | | from 2, complete | | (FLJ20507), mRNA | | FLJ20507 | |
| | | | sequence | | Length = 4223 | | | |
| | | | Length = 145918 | | | | | |
| 406 | | AC010542.7 | Homo sapiens | | | Hs.560416 | Transcribed | |
| | | | chromosome 16 | | | | locus | |
| | | | clone RP11-403P17, | | | | | |
| | | | complete sequence | | | | | |
| | | | Length = 188460 | | | | | |
| 407 | | | | | | Hs.370100 | Chromosome | CXorf26 |
| | | | | | | | X open | |
| | | | | | | | reading | |
| | | | | | | | frame 26 | |
| 408 | | AC004080.2 | Homo sapiens PAC | | | Hs.414467 | Homeo box | HOXA13 |
| | | | clone RP1-170O19 | | | | A13 | |
| | | | from 7p15-p21, | | | | | |
| | | | complete sequence | | | | | |
| | | | Length = 129355 | | | | | |
| 409 | | | | | | Hs.470943 | Signal | STAT1 |
| | | | | | | | transducer | |
| | | | | | | | and activator | |
| | | | | | | | of | |
| | | | | | | | transcription | |
| | | | | | | | 1, 91 kDa | |
| 410 | | AC091529.1 | AC091529 Homo | | | Hs.445574 | Full-length | |
| | | | sapiens | | | | cDNA clone | |
| | | | chromosome 17 | | | | CS0DK005YO12 | |
| | | | clone 1005, | | | | of HeLa | |
| | | | complete sequence | | | | cells Cot 25- | |
| | | | Length = 157463 | | | | normalized | |
| | | | | | | | of Homo | |
| | | | | | | | sapiens | |
| | | | | | | | (human) | |
| 411 | | | | XM_374767.1 | PREDICTED: Homo | Hs.146539 | Hypothetical | DKFZp667F0711 |
| | | | | | sapiens hypothetical protein | | protein | |
| | | | | | DKFZp667F0711 | | DKFZp667F0711 | |
| | | | | | (DKFZp667F0711), mRNA | | | |
| | | | | | Length = 2447 | | | |
| 412 | | BC052667.1 | Mus musculus | NM_008110.1 | Mus musculus growth | Mm.9714 | Growth | Gdf9 |
| | | | growth differentiation | | differentiation factor 9 | | differentiation | |
| | | | factor 9, mRNA | | (Gdf9), mRNA | | factor 9 | |
| | | | (cDNA clone | | Length = 1712 | | (Gdf9), | |
| | | | MGC: 60651 | | | | mRNA | |
| | | | IMAGE: 30054181), | | | | | |
| | | | complete cds | | | | | |
| | | | Length = 1752 | | | | | |
| 413 | | BC051316.1 | Homo sapiens | NM_018029.3 | Homo sapiens hypothetical | Hs.534462 | Hypothetical | FLJ10213 |
| | | | hypothetical protein | | protein FLJ10213 | | protein | |
| | | | FLJ10213, mRNA | | (FLJ10213), mRNA | | FLJ10213 | |
| | | | (cDNA clone | | Length = 1679 | | | |
| | | | IMAGE: 6214587) | | | | | |
| | | | Length = 1679 | | | | | |
| 414 | | BC033026.1 | Homo sapiens | NM_024899.2 | Homo sapiens | Hs.236940 | Chromosome | C18orf9 |
| | | | chromosome 18 | | chromosome 18 open | | 18 open | |
| | | | open reading frame | | reading frame 9 (C18orf9), | | reading | |
| | | | 9, mRNA (cDNA | | mRNA Length = 2884 | | frame 9 | |
| | | | clone | | | | | |
| | | | IMAGE: 5267551) | | | | | |
| | | | Length = 2267 | | | | | |
| 415 | | AC105206.8 | Homo sapiens | | | Hs.491223 | Retinoic acid | RAI16 |
| | | | chromosome 8, | | | | induced 16 | |
| | | | clone CTD-2530N21, | | | | | |
| | | | complete sequence | | | | | |
| | | | Length = 218931 | | | | | |
| 416 | | AC111196.4 | Homo sapiens BAC | | | Hs.128199 | Septin 11 | Sep 11 |
| | | | clone RP11-585K17 | | | | | |
| | | | from 4, complete | | | | | |
| | | | sequence | | | | | |
| | | | Length = 35191 | | | | | |
| 417 | | BC070091.1 | Homo sapiens | NM_052813.2 | Homo sapiens caspase | Hs.558530 | Caspase | CARD9 |
| | | | caspase recruitment | | recruitment domain family, | | recruitment | |
| | | | domain family, | | member 9 (CARD9), | | domain | |
| | | | member 9, mRNA | | mRNA Length = 2136 | | family, | |
| | | | (cDNA clone | | | | member 9 | |
| | | | MGC: 87491 | | | | | |
| | | | IMAGE: 30343821), | | | | | |
| | | | complete cds | | | | | |
| | | | Length = 4078 | | | | | |
| 418 | | AC118535.1 | Homo sapiens | | | Hs.408456 | Protein | PTPRS |
| | | | chromosome 19 | | | | tyrosine | |
| | | | clone LLNLR-287H1, | | | | phosphatase, | |
| | | | complete sequence | | | | receptor | |
| | | | Length = 32887 | | | | type, S | |
| 419 | | BC107044.1 | Homo sapiens cDNA | NM_138294.2 | Homo sapiens expressed | Hs.148565 | Expressed in | PATE |
| | | | clone MGC: 129554 | | in prostate and testis | | prostate and | |
| | | | IMAGE: 40002653, | | (PATE), mRNA | | testis | |
| | | | complete cds | | Length = 1576 | | | |
| | | | Length = 465 | | | | | |
| 420 | | BC047698.1 | Homo sapiens | NM_000880.2 | Homo sapiens interleukin 7 | Hs.536926 | Interleukin 7 | IL7 |
| | | | interleukin 7, mRNA | | (IL7), mRNA Length = 2116 | | | |
| | | | (cDNA clone | | | | | |
| | | | MGC: 51943 | | | | | |
| | | | IMAGE: 5748841), | | | | | |
| | | | complete cds | | | | | |
| | | | Length = 2125 | | | | | |
| 421 | | AC108451.9 | Homo sapiens | | | Hs.459070 | Aryl- | ARNT2 |
| | | | chromosome 15, | | | | hydrocarbon | |
| | | | clone RP11-379K22, | | | | receptor | |
| | | | complete sequence | | | | nuclear | |
| | | | Length = 198707 | | | | translocator 2 | |
| 422 | | BC077726.1 | Homo sapiens sema | NM_001031702.1 | Homo sapiens sema | Hs.210870 | Sema | SEMA5B |
| | | | domain, seven | | domain, seven | | domain, | |
| | | | thrombospondin | | thrombospondin repeats | | seven | |
| | | | repeats (type 1 and | | (type 1 and type 1-like), | | thrombospondin | |
| | | | type 1-like), | | transmembrane domain | | repeats | |
| | | | transmembrane | | (TM) and short cytoplasmic | | (type 1 and | |
| | | | domain (TM) and | | domain, (semaphorin) 5B | | type 1-like), | |
| | | | short cytoplasmic | | (SEMA5B), transcript | | transmembrane | |
| | | | domain, | | variant 1, mRNA | | domain | |
| | | | (semaphorin) 5B, | | Length = 4494 | | (TM) and | |
| | | | transcript variant 1, | | | | short | |
| | | | mRNA (cDNA clone | | | | cytoplasmic | |
| | | | MGC: 78583 | | | | domain, | |
| | | | IMAGE: 6597493), | | | | (semaphorin) | |
| | | | complete cds | | | | 5B | |
| | | | Length = 4494 | | | | | |
| 423 | | BC027920.1 | Homo sapiens, clone | NM_002348.2 | Homo sapiens lymphocyte | Hs.403857 | Lymphocyte | LY9 |
| | | | IMAGE: 5225850, | | antigen 9 (LY9), transcript | | antigen 9 | |
| | | | mRNA Length = 2131 | | variant 1, mRNA | | | |
| | | | | | Length = 2486 | | | |
| 424 | | AF207550.3 | Homo sapiens | NM_017602.2 | Homo sapiens hypothetical | Hs.496098 | OTU domain | DKFZp761A052 |
| | | | chromosome X | | protein DKFZp761A052 | | containing 5 | |
| | | | multiple clones map | | (DKFZp761A052), mRNA | | | |
| | | | p11.23, complete | | Length = 2759 | | | |
| | | | sequence | | | | | |
| | | | Length = 128713 | | | | | |
| 425 | | BC010284.1 | Homo sapiens | NM_004109.3 | Homo sapiens ferredoxin 1 | Hs.744 | Ferredoxin 1 | FDX1 |
| | | | ferredoxin 1, mRNA | | (FDX1), nuclear gene | | | |
| | | | (cDNA clone | | encoding mitochondrial | | | |
| | | | MGC: 5249 | | protein, mRNA | | | |
| | | | IMAGE: 3048743), | | Length = 3236 | | | |
| | | | complete cds | | | | | |
| | | | Length = 3138 | | | | | |
| 426 | | | | | | Hs.517717 | Special AT- | SATB1 |
| | | | | | | | rich | |
| | | | | | | | sequence | |
| | | | | | | | binding | |
| | | | | | | | protein 1 | |
| | | | | | | | (binds to | |
| | | | | | | | nuclear | |
| | | | | | | | matrix/scaffold- | |
| | | | | | | | associating | |
| | | | | | | | DNA's) | |
| 427 | | S49852.1 | plasma membrane | NM_001001323.1 | Homo sapiens ATPase, | Hs.506276 | ATPase, | ATP2B1 |
| | | | Ca2+ pump isoform | | Ca++ transporting, plasma | | Ca++ | |
| | | | 1a {alternatively | | membrane 1 (ATP2B1), | | transporting, | |
| | | | spliced} [human, | | transcript variant 1, mRNA | | plasma | |
| | | | fetal brain, mRNA | | Length = 6950 | | membrane 1 | |
| | | | Partial, 769 nt] | | | | | |
| | | | Length = 769 | | | | | |
| 428 | | BC020063.1 | Homo sapiens CD84 | NM_003874.1 | Homo sapiens CD84 | Hs.398093 | CD84 | CD84 |
| | | | antigen (leukocyte | | antigen (leukocyte antigen) | | antigen | |
| | | | antigen), mRNA | | (CD84), mRNA | | (leukocyte | |
| | | | (cDNA clone | | Length = 3299 | | antigen) | |
| | | | MGC: 21324 | | | | | |
| | | | IMAGE: 4385965), | | | | | |
| | | | complete cds | | | | | |
| | | | Length = 3278 | | | | | |
| 429 | | AY162833.1 | Homo sapiens E2F | NM_001951.2 | Homo sapiens E2F | Hs.445758 | E2F | E2F5 |
| | | | transcription factor 5, | | transcription factor 5, p130- | | transcription | |
| | | | p130-binding (E2F5) | | binding (E2F5), mRNA | | factor 5, | |
| | | | gene, complete cds | | Length = 1752 | | p130-binding | |
| | | | Length = 38504 | | | | | |
| 430 | | BC050585.1 | Homo sapiens | NM_000210.1 | Homo sapiens integrin, | Hs.133397 | Integrin, | ITGA6 |
| | | | integrin, alpha 6, | | alpha 6 (ITGA6), mRNA | | alpha 6 | |
| | | | mRNA (cDNA clone | | Length = 5611 | | | |
| | | | IMAGE: 6150124), | | | | | |
| | | | partial cds | | | | | |
| | | | Length = 4267 | | | | | |
| 431 | | BC007721.2 | Homo sapiens DNA | NM_213566.1 | Homo sapiens DNA | Hs.484782 | DNA | DFFA |
| | | | fragmentation factor, | | fragmentation factor, | | fragmentation | |
| | | | 45 kDa, alpha | | 45 kDa, alpha polypeptide | | factor, | |
| | | | polypeptide, | | (DFFA), transcript variant 2, | | 45 kDa, alpha | |
| | | | transcript variant 1, | | mRNA Length = 3408 | | polypeptide | |
| | | | mRNA (cDNA clone | | | | | |
| | | | MGC: 12704 | | | | | |
| | | | IMAGE: 4299734), | | | | | |
| | | | complete cds | | | | | |
| | | | Length = 1985 | | | | | |
| 432 | | AC146440.5 | Pan troglodytes BAC | NM_033015.2 | Homo sapiens Fas- | Hs.75087 | Fas- | FASTK |
| | | | clone RP43-11P11 | | activated serine/threonine | | activated | |
| | | | from 7, complete | | kinase (FASTK), transcript | | serine/threonine | |
| | | | sequence | | variant 4, mRNA | | kinase | |
| | | | Length = 181369 | | Length = 1443 | | | |
| 433 | | NM_007177 | family with sequence | NM_007177 | family with sequence | | Hs.8022 | family with |
| | | | similarity 107, | | similarity 107, member A | | | sequence |
| | | | member A | | | | | similarity 107, |
| | | | | | | | | member A |
| 434 | | | | | | Hs.126573 | Transcribed | |
| | | | | | | | locus | |
| 435 | | BC019903.1 | Homo sapiens | XM_519249.1 | PREDICTED: Pan | Hs.369867 | Hypothetical | MGC40499 |
| | | | hypothetical protein | | troglodytes similar to | | protein | |
| | | | MGC40499, mRNA | | hypothetical protein | | MGC40499 | |
| | | | (cDNA clone | | MGC40499 (LOC463589), | | | |
| | | | IMAGE: 4991480), | | mRNA Length = 1122 | | | |
| | | | partial cds | | | | | |
| | | | Length = 1413 | | | | | |
| 436 | | | | | | Hs.304233 | Similar to | |
| | | | | | | | MGC52970 | |
| | | | | | | | protein | |
| 437 | | BC004192.2 | Homo sapiens | NM_014754.1 | Homo sapiens | Hs.292579 | Phosphatidylserine | PTDSS1 |
| | | | phosphatidylserine | | phosphatidylserine | | synthase 1 | |
| | | | synthase 1, mRNA | | synthase 1 (PTDSS1), | | | |
| | | | (cDNA clone | | mRNA Length = 2504 | | | |
| | | | MGC: 3305 | | | | | |
| | | | IMAGE: 3509418), | | | | | |
| | | | complete cds | | | | | |
| | | | Length = 2528 | | | | | |
| 438 | | BC015519.2 | Homo sapiens | NM_004708.2 | Homo sapiens | Hs.504942 | Transcribed | |
| | | | programmed cell | | programmed cell death 5 | | locus, | |
| | | | death 5, mRNA | | (PDCD5), mRNA | | strongly | |
| | | | (cDNA clone | | Length = 599 | | similar to | |
| | | | MGC: 9294 | | | | XP_512563.1 | |
| | | | IMAGE: 3888459), | | | | PREDICTED: | |
| | | | complete cds | | | | similar to | |
| | | | Length = 590 | | | | programmed | |
| | | | | | | | cell death 5; | |
| | | | | | | | TFAR19 | |
| | | | | | | | novel | |
| | | | | | | | apoptosis- | |
| | | | | | | | related gene; | |
| | | | | | | | TF1 cell | |
| | | | | | | | apoptosis- | |
| | | | | | | | related gene | |
| | | | | | | | 19 [Pan | |
| | | | | | | | troglodytes] | |
| 439 | | BC024034.2 | Homo sapiens heat | NM_004134.4 | Homo sapiens heat shock | Hs.560739 | Heat shock | HSPA9B |
| | | | shock 70 kDa protein | | 70 kDa protein 9B (mortalin- | | 70 kDa | |
| | | | 9B (mortalin-2), | | 2) (HSPA9B), nuclear gene | | protein 9B | |
| | | | mRNA (cDNA clone | | encoding mitochondrial | | (mortalin-2) | |
| | | | MGC: 4500 | | protein, mRNA | | | |
| | | | IMAGE: 2964588), | | Length = 2883 | | | |
| | | | complete cds | | | | | |
| | | | Length = 2802 | | | | | |
| 440 | | BC016288.1 | Homo sapiens | NM_002013.2 | Homo sapiens FK506 | Hs.509226 | FK506 | FKBP3 |
| | | | FK506 binding | | binding protein 3, 25 kDa | | binding | |
| | | | protein 3, 25 kDa, | | (FKBP3), mRNA | | protein 3, | |
| | | | mRNA (cDNA clone | | Length = 1420 | | 25 kDa | |
| | | | MGC: 9482 | | | | | |
| | | | IMAGE: 3919642), | | | | | |
| | | | complete cds | | | | | |
| | | | Length = 1420 | | | | | |
| 441 | | BC023616.2 | Homo sapiens | NM_024026.4 | Homo sapiens | Hs.458367 | Mitochondrial | MRP63 |
| | | | mitochondrial | | mitochondrial ribosomal | | ribosomal | |
| | | | ribosomal protein 63, | | protein 63 (MRP63), | | protein 63 | |
| | | | mRNA (cDNA clone | | nuclear gene encoding | | | |
| | | | MGC: 23340 | | mitochondrial protein, | | | |
| | | | IMAGE: 4645641), | | mRNA Length = 2671 | | | |
| | | | complete cds | | | | | |
| | | | Length = 1160 | | | | | |
| 442 | | AC008649.8 | Homo sapiens | NM_000540.1 | Homo sapiens ryanodine | Hs.466664 | Ryanodine | RYR1 |
| | | | chromosome 19 | | receptor 1 (skeletal) | | receptor 1 | |
| | | | clone CTB-186G2, | | (RYR1), mRNA | | (skeletal) | |
| | | | complete sequence | | Length = 15359 | | | |
| | | | Length = 126682 | | | | | |
| 443 | | | | | | Hs.444414 | AF4/FMR2 | LAF4 |
| | | | | | | | family, | |
| | | | | | | | member 3 | |
| 444 | | BC001169.1 | Homo sapiens | NM_001984.1 | Homo sapiens esterase | Hs.432491 | Esterase | ESD |
| | | | esterase | | D/formylglutathione | | D/formylglutathione | |
| | | | D/formylglutathione | | hydrolase (ESD), mRNA | | hydrolase | |
| | | | hydrolase, mRNA | | Length = 1208 | | | |
| | | | (cDNA clone | | | | | |
| | | | MGC: 1873 | | | | | |
| | | | IMAGE: 3528593), | | | | | |
| | | | complete cds | | | | | |
| | | | Length = 1032 | | | | | |
| 445 | | | | | | Hs.309763 | G-rich RNA | GRSF1 |
| | | | | | | | sequence | |
| | | | | | | | binding | |
| | | | | | | | factor 1 | |
| 446 | | U66464.1 | HSU66464 Human | NM_007181.3 | Homo sapiens mitogen- | Hs.95424 | Mitogen- | MAP4K1 |
| | | | hematopoietic | | activated protein kinase | | activated | |
| | | | progenitor kinase | | kinase kinase kinase 1 | | protein | |
| | | | (HPK1) mRNA, | | (MAP4K1), mRNA | | kinase | |
| | | | complete cds | | Length = 2732 | | kinase | |
| | | | Length = 2732 | | | | kinase | |
| | | | | | | | kinase 1 | |
| 447 | | AC025457.5 | AC025457 Homo | NM_019846.3 | Homo sapiens chemokine | Hs.334633 | Chemokine | CCL28 |
| | | | sapiens | | (C-C motif) ligand 28 | | (C-C motif) | |
| | | | chromosome 5 clone | | (CCL28), transcript variant | | ligand 28 | |
| | | | CTD-2202K16, | | 1, mRNA Length = 1349 | | | |
| | | | complete sequence | | | | | |
| | | | Length = 140734 | | | | | |
| 448 | | BC009401.2 | Homo sapiens | NM_001012636.1 | Homo sapiens interleukin | Hs.943 | Interleukin | NK4 |
| | | | interleukin 32, mRNA | | 32 (IL32), transcript variant | | 32 | |
| | | | (cDNA clone | | 7, mRNA Length = 880 | | | |
| | | | MGC: 15353 | | | | | |
| | | | IMAGE: 4300407), | | | | | |
| | | | complete cds | | | | | |
| | | | Length = 1159 | | | | | |
| 449 | | AC013274.6 | Homo sapiens BAC | | | Hs.413434 | Solute carrier | SLC39A10 |
| | | | clone RP11-449J2 | | | | family 39 | |
| | | | from 2, complete | | | | (zinc | |
| | | | sequence | | | | transporter), | |
| | | | Length = 125133 | | | | member 10 | |
| 450 | | | | | | Hs.516807 | Serine/threonine | STK25 |
| | | | | | | | kinase | |
| | | | | | | | 25 (STE20 | |
| | | | | | | | homolog, | |
| | | | | | | | yeast) | |
| 451 | | AF034970.1 | AF034970 Homo | NM_003974.2 | Homo sapiens docking | Hs.71215 | Docking | DOK2 |
| | | | sapiens docking | | protein 2, 56 kDa (DOK2), | | protein 2, | |
| | | | protein (DOK-2) | | transcript variant 1, mRNA | | 56 kDa | |
| | | | mRNA, complete cds | | Length = 1870 | | | |
| | | | Length = 1239 | | | | | |
| 452 | | BC034403.1 | Homo sapiens | NM_001504.1 | Homo sapiens chemokine | Hs.198252 | Chemokine | CXCR3 |
| | | | chemokine (C—X—C | | (C—X—C motif) receptor 3 | | (C—X—C motif) | |
| | | | motif) receptor 3, | | (CXCR3), mRNA | | receptor 3 | |
| | | | mRNA (cDNA clone | | Length = 1670 | | | |
| | | | MGC: 34593 | | | | | |
| | | | IMAGE: 5176136), | | | | | |
| | | | complete cds | | | | | |
| | | | Length = 1626 | | | | | |
| 453 | | | | | | Hs.525015 | Transcribed | |
| | | | | | | | locus, | |
| | | | | | | | moderately | |
| | | | | | | | similar to | |
| | | | | | | | NP_005496.3 | |
| | | | | | | | scavenger | |
| | | | | | | | receptor | |
| | | | | | | | class B, | |
| | | | | | | | member 1; | |
| | | | | | | | CD36 | |
| | | | | | | | antigen-like | |
| | | | | | | | 1; CD36 | |
| | | | | | | | antigen | |
| | | | | | | | (collagen | |
| | | | | | | | type I | |
| | | | | | | | receptor, | |
| | | | | | | | thrombospondin | |
| | | | | | | | receptor)-like | |
| | | | | | | | 1; scavenger | |
| | | | | | | | receptor | |
| | | | | | | | class B type | |
| | | | | | | | 1; scavenger | |
| | | | | | | | receptor | |
| | | | | | | | class B type | |
| | | | | | | | III | |
| 454 | | AC023670.6 | Homo sapiens BAC | NG_002811.1 | Homo sapiens glycogenin 2 | Hs.541209 | Glycogenin 2 | GYG2 |
| | | | clone RP11-457M7 | | pseudogene (GYG2P) on | | | |
| | | | from Xq23, complete | | chromosome Y | | | |
| | | | sequence | | Length = 15111 | | | |
| | | | Length = 59565 | | | | | |
| 455 | | BC010856.2 | Homo sapiens | NM_001014842.1 | Homo sapiens | Hs.91586 | Transmembrane 9 | TM9SF1 |
| | | | transmembrane 9 | | transmembrane 9 | | superfamily | |
| | | | superfamily member | | superfamily member 1 | | member 1 | |
| | | | 1, transcript variant | | (TM9SF1), transcript | | | |
| | | | 1, mRNA (cDNA | | variant 2, mRNA | | | |
| | | | clone MGC: 9160 | | Length = 2124 | | | |
| | | | IMAGE: 3899246), | | | | | |
| | | | complete cds | | | | | |
| | | | Length = 2121 | | | | | |
| 456 | | BC038289.1 | Homo sapiens | NM_032962.2 | Homo sapiens chemokine | Hs.272493 | Chemokine | CCL15 |
| | | | chemokine (C-C | | (C-C motif) ligand 14 | | (C-C motif) | |
| | | | motif) ligand 14, | | (CCL14), transcript variant | | ligand 15 | |
| | | | transcript variant 3, | | 2, mRNA Length = 1392 | | | |
| | | | mRNA (cDNA clone | | | | | |
| | | | MGC: 47546 | | | | | |
| | | | IMAGE: 5222914), | | | | | |
| | | | complete cds | | | | | |
| | | | Length = 533 | | | | | |
| 457 | | U19261.1 | HSU19261 Homo | | | Hs.531251 | TNF | TRAF1 |
| | | | sapiens Epstein-Barr | | | | receptor- | |
| | | | virus-induced protein | | | | associated | |
| | | | mRNA, complete cds | | | | factor 1 | |
| | | | Length = 2380 | | | | | |
| 458 | | BC000635.2 | Homo sapiens | NM_001607.2 | Homo sapiens acetyl- | Hs.166160 | Acetyl- | ACAA1 |
| | | | acetyl-Coenzyme A | | Coenzyme A | | Coenzyme A | |
| | | | acyltransferase 1 | | acyltransferase 1 | | acyltransferase 1 | |
| | | | (peroxisomal 3- | | (peroxisomal 3-oxoacyl- | | (peroxisomal | |
| | | | oxoacyl-Coenzyme A | | Coenzyme A thiolase) | | 3-oxoacyl- | |
| | | | thiolase), mRNA | | (ACAA1), nuclear gene | | Coenzyme A | |
| | | | (cDNA clone | | encoding mitochondrial | | thiolase) | |
| | | | MGC: 1165 | | protein, mRNA | | | |
| | | | IMAGE: 3345271), | | Length = 1695 | | | |
| | | | complete cds | | | | | |
| | | | Length = 1720 | | | | | |
| 459 | | BC027968.1 | Homo sapiens | NM_006274.2 | Homo sapiens chemokine | Hs.50002 | Chemokine | CCL19 |
| | | | chemokine (C-C | | (C-C motif) ligand 19 | | (C-C motif) | |
| | | | motif) ligand 19, | | (CCL19), mRNA | | ligand 19 | |
| | | | mRNA (cDNA clone | | Length = 684 | | | |
| | | | MGC: 34433 | | | | | |
| | | | IMAGE: 5225849), | | | | | |
| | | | complete cds | | | | | |
| | | | Length = 694 | | | | | |
| 460 | | BC067735.1 | Homo sapiens cDNA | | | Hs.106432 | Hypothetical | DKFZp761B107 |
| | | | clone MGC: 86968 | | | | protein | |
| | | | IMAGE: 4792861, | | | | DKFZp761B107 | |
| | | | complete cds | | | | | |
| | | | Length = 1096 | | | | | |
| 461 | | AC016939.8 | Homo sapiens X | NM_005365.4 | Homo sapiens melanoma | Hs.512582 | Melanoma | MAGEA9 |
| | | | BAC RP5-1014O16 | | antigen family A, 9 | | antigen | |
| | | | (Roswell Park | | (MAGEA9), mRNA | | family A, 9 | |
| | | | Cancer Institute | | Length = 1824 | | | |
| | | | Human BAC Library) | | | | | |
| | | | complete sequence | | | | | |
| | | | Length = 118440 | | | | | |
| 462 | | | | | | Hs.567676 | Transcribed | |
| | | | | | | | locus, | |
| | | | | | | | strongly | |
| | | | | | | | similar to | |
| | | | | | | | XP_509380.1 | |
| | | | | | | | PREDICTED: | |
| | | | | | | | similar to | |
| | | | | | | | fertilin alpha- | |
| | | | | | | | I [Pan | |
| | | | | | | | troglodytes] | |
| 463 | | AC017104.8 | Homo sapiens BAC | | | Hs.470765 | Zinc finger | ARCH |
| | | | clone RP11-562I5 | | | | and BTB | |
| | | | from 2, complete | | | | domain | |
| | | | sequence | | | | containing 8 | |
| | | | Length = 168880 | | | | opposite | |
| | | | | | | | strand | |
| 464 | | BC070309.1 | Homo sapiens | NM_003175.2 | Homo sapiens chemokine | Hs.546295 | Chemokine | XCL1 |
| | | | chemokine (C motif) | | (C motif) ligand 2 (XCL2), | | (C motif) | |
| | | | ligand 1, mRNA | | mRNA Length = 552 | | ligand 1 | |
| | | | (cDNA clone | | | | | |
| | | | MGC: 88311 | | | | | |
| | | | IMAGE: 30416260), | | | | | |
| | | | complete cds | | | | | |
| | | | Length = 597 | | | | | |
| 465 | | BC003582.1 | Homo sapiens | NM_021974.2 | Homo sapiens polymerase | Hs.436578 | Polymerase | POLR2F |
| | | | polymerase (RNA) II | | (RNA) II (DNA directed) | | (RNA) II | |
| | | | (DNA directed) | | polypeptide F (POLR2F), | | (DNA | |
| | | | polypeptide F, mRNA | | mRNA Length = 546 | | directed) | |
| | | | (cDNA clone | | | | polypeptide F | |
| | | | MGC: 2669 | | | | | |
| | | | IMAGE: 3546712), | | | | | |
| | | | complete cds | | | | | |
| | | | Length = 588 | | | | | |
| 466 | IMAGE: 2317044 | | | | | | | |
| 467 | | BT019901.1 | Homo sapiens tumor | NM_001250.3 | Homo sapiens CD40 | Hs.472860 | CD40 | CD40 |
| | | | necrosis factor | | antigen (TNF receptor | | antigen (TNF | |
| | | | receptor superfamily, | | superfamily member 5) | | receptor | |
| | | | member 5 mRNA, | | (CD40), transcript variant 1, | | superfamily | |
| | | | complete cds | | mRNA Length = 1177 | | member 5) | |
| | | | Length = 834 | | | | | |
| 468 | | BC012160.1 | Homo sapiens tumor | NM_001242.3 | Homo sapiens tumor | Hs.355307 | Tumor | TNFRSF7 |
| | | | necrosis factor | | necrosis factor receptor | | necrosis | |
| | | | receptor superfamily, | | superfamily, member 7 | | factor | |
| | | | member 7, mRNA | | (TNFRSF7), mRNA | | receptor | |
| | | | (cDNA clone | | Length = 1323 | | superfamily, | |
| | | | MGC: 20393 | | | | member 7 | |
| | | | IMAGE: 4575359), | | | | | |
| | | | complete cds | | | | | |
| | | | Length = 1323 | | | | | |
| 469 | | BC067226.1 | Homo sapiens | NG_000834.1 | Homo sapiens | Hs.560836 | Ig kappa | |
| | | | immunoglobulin | | immunoglobulin kappa | | {clone | |
| | | | kappa constant, | | locus, proximal V-cluster | | cYF.kappa} | |
| | | | mRNA (cDNA clone | | and J-C cluster (IGK- | | [human, | |
| | | | MGC: 72070 | | proximal) on chromosome | | mRNA | |
| | | | IMAGE: 30349629), | | 2 Length = 515442 Features | | Partial, 1209 | |
| | | | complete cds | | in this part of subject | | nt] | |
| | | | Length = 969 | | sequence: CDS | | | |
| 470 | | BC018761.1 | Homo sapiens cDNA | XM_515609.1 | PREDICTED: Pan | Hs.551722 | Ig | |
| | | | clone | | troglodytes similar to | | rearranged | |
| | | | IMAGE: 4565991 | | [Human Ig rearranged | | kappa-chain | |
| | | | Length = 944 | | gamma chain mRNA, V-J- | | mRNA V-J1- | |
| | | | | | C region and complete | | region, | |
| | | | | | cds.], gene product | | hybridoma | |
| | | | | | (LOC459391), mRNA | | AE6-5,5′ | |
| | | | | | Length = 1867 | | end | |
| 471 | | AC099334.2 | Homo sapiens | NM_003243.2 | Homo sapiens transforming | Hs.482390 | Transforming | TGFBR3 |
| | | | chromosome 1 clone | | growth factor, beta receptor | | growth | |
| | | | RP5-865M20, | | III (betaglycan, 300 kDa) | | factor, beta | |
| | | | complete sequence | | (TGFBR3), mRNA | | receptor III | |
| | | | Length = 112562 | | Length = 4199 | | (betaglycan, | |
| | | | | | | | 300 kDa) | |
| 472 | | BC047695.1 | Homo sapiens | NM_005041.3 | Homo sapiens perforin 1 | Hs.2200 | Perforin 1 | PRF1 |
| | | | perforin 1 (pore | | (pore forming protein) | | (pore | |
| | | | forming protein), | | (PRF1), mRNA | | forming | |
| | | | mRNA (cDNA clone | | Length = 2512 | | protein) | |
| | | | IMAGE: 5752084), | | | | | |
| | | | partial cds | | | | | |
| | | | Length = 2502 | | | | | |
| 473 | | DQ017060.1 | Homo sapiens | NM_002985.2 | Homo sapiens chemokine | Hs.514821 | Chemokine | CCL5 |
| | | | chemokine (C-C | | (C-C motif) ligand 5 | | (C-C motif) | |
| | | | motif) ligand 5 | | (CCL5), mRNA | | ligand 5 | |
| | | | (CCL5) gene, | | Length = 1237 | | | |
| | | | complete cds | | | | | |
| | | | Length = 12438 | | | | | |
| 474 | | AY372494.1 | Homo sapiens | NM_004131.3 | Homo sapiens granzyme B | Hs.1051 | Granzyme B | GZMB |
| | | | endogenous | | (granzyme 2, cytotoxic T- | | (granzyme 2, | |
| | | | granzyme B | | lymphocyte-associated | | cytotoxic T- | |
| | | | precursor, mRNA, | | serine esterase 1) (GZMB), | | lymphocyte- | |
| | | | complete cds | | mRNA Length = 955 | | associated | |
| | | | Length = 951 | | | | serine | |
| | | | | | | | esterase 1) | |
| 475 | | M17016.1 | HUMSP13E Human | NM_004131.3 | Homo sapiens granzyme B | Hs.1051 | Granzyme B | GZMB |
| | | | serine protease-like | | (granzyme 2, cytotoxic T- | | (granzyme 2, | |
| | | | protein mRNA, | | lymphocyte-associated | | cytotoxic T- | |
| | | | complete cds | | serine esterase 1) (GZMB), | | lymphocyte- | |
| | | | Length = 934 | | mRNA Length = 955 | | associated | |
| | | | | | | | serine | |
| | | | | | | | esterase 1) | |
| 476 | | L78838.1 | L78837S2 Homo | NM_006725.2 | Homo sapiens CD6 antigen | Hs.502710 | CD6 antigen | CD6 |
| | | | sapiens T cell | | (CD6), mRNA Length = 3248 | | | |
| | | | surface glycoprotein | | | | | |
| | | | CD6 isoform (CD6) | | | | | |
| | | | gene, exons 2-13, | | | | | |
| | | | and complete cds, | | | | | |
| | | | alternatively spliced | | | | | |
| | | | Length = 17763 | | | | | |
| 477 | | BC025691.1 | Homo sapiens | NM_000878.2 | Homo sapiens interleukin 2 | Hs.474787 | Interleukin 2 | IL2RB |
| | | | interleukin 2 | | receptor, beta (IL2RB), | | receptor, | |
| | | | receptor, beta, | | mRNA Length = 4045 | | beta | |
| | | | mRNA (cDNA clone | | | | | |
| | | | MGC: 34584 | | | | | |
| | | | IMAGE: 5207833), | | | | | |
| | | | complete cds | | | | | |
| | | | Length = 4035 | | | | | |
| 478 | | BC033583.1 | Homo sapiens CD2 | NM_001767.2 | Homo sapiens CD2 antigen | Hs.523500 | CD2 antigen | CD2 |
| | | | antigen (p50), sheep | | (p50), sheep red blood cell | | (p50), sheep | |
| | | | red blood cell | | receptor (CD2), mRNA | | red blood cell | |
| | | | receptor, mRNA | | Length = 1579 | | receptor | |
| | | | (cDNA clone | | | | | |
| | | | MGC: 34621 | | | | | |
| | | | IMAGE: 5227138), | | | | | |
| | | | complete cds | | | | | |
| | | | Length = 1579 | | | | | |
| 479 | | BC014142.2 | Homo sapiens | NM_000181.1 | Homo sapiens | Hs.255230 | Glucuronidase, | GUSB |
| | | | glucuronidase, beta, | | glucuronidase, beta | | beta | |
| | | | mRNA (cDNA clone | | (GUSB), mRNA | | | |
| | | | MGC: 20659 | | Length = 2191 | | | |
| | | | IMAGE: 4662011), | | | | | |
| | | | complete cds | | | | | |
| | | | Length = 2182 | | | | | |
| 480 | | L10284.1 | HUMIP90 Homo | NM_001024649.1 | Homo sapiens calnexin | Hs.529890 | Calnexin | CANX |
| | | | sapiens integral | | (CANX), transcript variant | | | |
| | | | membrane protein, | | 2, mRNA Length = 4900 | | | |
| | | | calnexin, (IP90) | | | | | |
| | | | mRNA, complete cds | | | | | |
| | | | Length = 4117 | | | | | |
| 481 | | BC073754.1 | Homo sapiens | NM_182810.1 | Homo sapiens activating | Hs.496487 | Activating | ATF4 |
| | | | activating | | transcription factor 4 (tax- | | transcription | |
| | | | transcription factor 4 | | responsive enhancer | | factor 4 (tax- | |
| | | | (tax-responsive | | element B67) (ATF4), | | responsive | |
| | | | enhancer element | | transcript variant 2, mRNA | | enhancer | |
| | | | B67), transcript | | Length = 1420 | | element | |
| | | | variant 2, mRNA | | | | B67) | |
| | | | (cDNA clone | | | | | |
| | | | MGC: 88758 | | | | | |
| | | | IMAGE: 6580199), | | | | | |
| | | | complete cds | | | | | |
| | | | Length = 1417 | | | | | |
| 482 | | BC018857.2 | Homo sapiens | NM_001404.3 | Homo sapiens eukaryotic | Hs.557802 | Transcribed | |
| | | | eukaryotic translation | | translation elongation factor | | locus | |
| | | | elongation factor 1 | | 1 gamma (EEF1G), mRNA | | | |
| | | | gamma, mRNA | | Length = 1429 | | | |
| | | | (cDNA clone | | | | | |
| | | | IMAGE: 3141750) | | | | | |
| | | | Length = 1414 | | | | | |
| 483 | | BC020057.1 | Homo sapiens | NM_002211.2 | Homo sapiens integrin, | Hs.429052 | Integrin, beta | ITGB1 |
| | | | integrin, beta 1 | | beta 1 (fibronectin receptor, | | 1 (fibronectin | |
| | | | (fibronectin receptor, | | beta polypeptide, antigen | | receptor, | |
| | | | beta polypeptide, | | CD29 includes MDF2, | | beta | |
| | | | antigen CD29 | | MSK12) (ITGB1), transcript | | polypeptide, | |
| | | | includes MDF2, | | variant 1A, mRNA | | antigen | |
| | | | MSK12), transcript | | Length = 3700 | | CD29 | |
| | | | variant 1A, mRNA | | | | includes | |
| | | | (cDNA clone | | | | MDF2, | |
| | | | MGC: 17220 | | | | MSK12) | |
| | | | IMAGE: 3924411), | | | | | |
| | | | complete cds | | | | | |
| | | | Length = 3656 | | | | | |
| 484 | | BC068201.1 | Homo sapiens cDNA | NM_012224.1 | Homo sapiens NIMA (never | Hs.481181 | NIMA (never | NEK1 |
| | | | clone | | in mitosis gene a)-related | | in mitosis | |
| | | | IMAGE: 4771714, | | kinase 1 (NEK1), mRNA | | gene a)- | |
| | | | partial cds | | Length = 5501 | | related | |
| | | | Length = 1745 | | | | kinase 1 | |
| 485 | | AF395009.1 | AF395009 Homo | | | | | |
| | | | sapiens glycoprotein | | | | | |
| | | | lb (platelet), alpha | | | | | |
| | | | polypeptide (GP1BA) | | | | | |
| | | | gene, complete cds | | | | | |
| | | | Length = 6241 | | | | | |
| 486 | | BC039593.1 | Homo sapiens | NM_006676.4 | Homo sapiens ubiquitin | Hs.5452 | Ubiquitin | USP20 |
| | | | ubiquitin specific | | specific peptidase 20 | | specific | |
| | | | peptidase 20, | | (USP20), transcript variant | | peptidase 20 | |
| | | | transcript variant 2, | | 2, mRNA Length = 4490 | | | |
| | | | mRNA (cDNA clone | | | | | |
| | | | MGC: 48817 | | | | | |
| | | | IMAGE: 5588842), | | | | | |
| | | | complete cds | | | | | |
| | | | Length = 4490 | | | | | |
| 487 | | BC004491.2 | Homo sapiens | NM_153425.1 | Homo sapiens TNFRSF1A- | Hs.460996 | TNFRSF1A- | TRADD |
| | | | TNFRSF1A- | | associated via death | | associated | |
| | | | associated via death | | domain (TRADD), | | via death | |
| | | | domain, transcript | | transcript variant 2, mRNA | | domain | |
| | | | variant 1, mRNA | | Length = 1959 | | | |
| | | | (cDNA clone | | | | | |
| | | | MGC: 11078 | | | | | |
| | | | IMAGE: 3689007), | | | | | |
| | | | complete cds | | | | | |
| | | | Length = 1466 | | | | | |
| 488 | | AC083899.6 | Homo sapiens BAC | | | Hs.135043 | Transcribed | |
| | | | clone RP11-223J6 | | | | locus | |
| | | | from 2, complete | | | | | |
| | | | sequence | | | | | |
| | | | Length = 153666 | | | | | |
| 489 | | | | | | Hs.126007 | Hypothetical | LOC375295 |
| | | | | | | | gene | |
| | | | | | | | supported by | |
| | | | | | | | BC013438 | |
| 490 | | BC012160.1 | Homo sapiens tumor | NM_001242.3 | Homo sapiens tumor | Hs.355307 | Tumor | TNFRSF7 |
| | | | necrosis factor | | necrosis factor receptor | | necrosis | |
| | | | receptor superfamily, | | superfamily, member 7 | | factor | |
| | | | member 7, mRNA | | (TNFRSF7), mRNA | | receptor | |
| | | | (cDNA clone | | Length = 1323 | | superfamily, | |
| | | | MGC: 20393 | | | | member 7 | |
| | | | IMAGE: 4575359), | | | | | |
| | | | complete cds | | | | | |
| | | | Length = 1323 | | | | | |
| 491 | | BC035343.1 | Homo sapiens | NM_001838.2 | Homo sapiens chemokine | Hs.370036 | Chemokine | CCR7 |
| | | | chemokine (C-C | | (C-C motif) receptor 7 | | (C-C motif) | |
| | | | motif) receptor 7, | | (CCR7), mRNA | | receptor 7 | |
| | | | mRNA (cDNA clone | | Length = 2188 | | | |
| | | | MGC: 34623 | | | | | |
| | | | IMAGE: 5180758), | | | | | |
| | | | complete cds | | | | | |
| | | | Length = 2173 | | | | | |
| 492 | | BC067540.1 | Homo sapiens | NM_002185.2 | Homo sapiens interleukin 7 | Hs.362807 | Interleukin 7 | IL7R |
| | | | interleukin 7 | | receptor (IL7R), mRNA | | receptor | |
| | | | receptor, mRNA | | Length = 1809 | | | |
| | | | (cDNA clone | | | | | |
| | | | MGC: 79450 | | | | | |
| | | | IMAGE: 6971846), | | | | | |
| | | | complete cds | | | | | |
| | | | Length = 1503 | | | | | |
| 493 | | BC049847.1 | Homo sapiens CD3E | NM_000733.2 | Homo sapiens CD3E | Hs.3003 | CD3E | CD3E |
| | | | antigen, epsilon | | antigen, epsilon polypeptide | | antigen, | |
| | | | polypeptide (TiT3 | | (TiT3 complex) (CD3E), | | epsilon | |
| | | | complex), mRNA | | mRNA Length = 1377 | | polypeptide | |
| | | | (cDNA clone | | | | (TiT3 | |
| | | | MGC: 51948 | | | | complex) | |
| | | | IMAGE: 5214563), | | | | | |
| | | | complete cds | | | | | |
| | | | Length = 1382 | | | | | |
| 494 | | BC029273.1 | Homo sapiens | NM_021258.2 | Homo sapiens interleukin | Hs.110915 | Interleukin | IL22RA1 |
| | | | interleukin 22 | | 22 receptor, alpha 1 | | 22 receptor, | |
| | | | receptor, alpha 1, | | (IL22RA1), mRNA | | alpha 1 | |
| | | | mRNA (cDNA clone | | Length = 2811 | | | |
| | | | MGC: 34232 | | | | | |
| | | | IMAGE: 4347189), | | | | | |
| | | | complete cds | | | | | |
| | | | Length = 2824 | | | | | |
| 495 | | AC013468.12 | Homo sapiens BAC | | | Hs.60772 | O- | OSGEPL1 |
| | | | clone RP11-455J20 | | | | sialoglycoprotein | |
| | | | from 2, complete | | | | endopeptidase- | |
| | | | sequence | | | | like 1 | |
| | | | Length = 84115 | | | | | |
| 496 | | AC084880.12 | Homo sapiens 12 | NM_014365.2 | Homo sapiens heat shock | Hs.400095 | Heat shock | HSPB8 |
| | | | BAC RP11-64B16 | | 22 kDa protein 8 (HSPB8), | | 22 kDa | |
| | | | (Roswell Park | | mRNA Length = 2056 | | protein 8 | |
| | | | Cancer Institute | | | | | |
| | | | Human BAC Library) | | | | | |
| | | | complete sequence | | | | | |
| | | | Length = 110001 | | | | | |
| 497 | | BT007890.1 | Synthetic construct | NM_001803.2 | Homo sapiens CD52 | Hs.276770 | CD52 | CDW52 |
| | | | Homo sapiens | | antigen (CAMPATH-1 | | antigen | |
| | | | CDW52 antigen | | antigen) (CD52), mRNA | | (CAMPATH- | |
| | | | (CAMPATH-1 | | Length = 523 | | 1 antigen) | |
| | | | antigen) mRNA, | | | | | |
| | | | partial cds | | | | | |
| | | | Length = 186 | | | | | |
| 498 | | BC069685.1 | Homo sapiens cDNA | NM_000073.1 | Homo sapiens CD3G | Hs.2259 | CD3G antigen, | CD3G |
| | | | clone | | antigen, gamma | | gamma | |
| | | | IMAGE: 7262517, | | polypeptide (TiT3 complex) | | polypeptide (TiT3 | |
| | | | containing frame- | | (CD3G), mRNA | | complex) | |
| | | | shift errors | | Length = 822 | | | |
| | | | Length = 737 | | | | | |
| 499 | | BC018843.2 | Homo sapiens | NM_203431.1 | Homo sapiens | Hs.567674 | Transcribed locus, | |
| | | | peptidylprolyl | | peptidylprolyl isomerase A | | moderately similar | |
| | | | isomerase A | | (cyclophilin A) (PPIA), | | to XP_526904.1 | |
| | | | (cyclophilin A), | | transcript variant 3, mRNA | | PREDICTED: | |
| | | | transcript variant 1, | | Length = 1707 | | similar to | |
| | | | mRNA (cDNA clone | | | | peptidylprolyl | |
| | | | IMAGE: 3139693) | | | | isomerase A | |
| | | | Length = 811 | | | | isoform 1; | |
| | | | | | | | cyclophilin A; | |
| | | | | | | | peptidyl-prolyl cis- | |
| | | | | | | | trans isomerase A; | |
| | | | | | | | T cell cyclophilin; | |
| | | | | | | | rotamase; | |
| | | | | | | | cyclosporin A- | |
| | | | | | | | binding protein | |
| | | | | | | | [Pan troglodytes] | |
| 500 | | BC018840.2 | Homo sapiens | NM_000968.2 | Homo sapiens ribosomal | Hs.447798 | Similar to | |
| | | | ribosomal protein L4, | | protein L4 (RPL4), mRNA | | ribosomal | |
| | | | mRNA (cDNA clone | | Length = 1449 | | protein L4; | |
| | | | IMAGE: 3051153) | | | | 60S | |
| | | | Length = 1439 | | | | ribosomal | |
| | | | | | | | protein L4; | |
| | | | | | | | homologue | |
| | | | | | | | of Xenopus | |
| | | | | | | | ribosomal | |
| | | | | | | | protein L1 | |
| 501 | | BC032107.2 | Homo sapiens | NM_012423.2 | Homo sapiens ribosomal | Hs.561045 | Transcribed | |
| | | | ribosomal protein | | protein L13a (RPL13A), | | locus, | |
| | | | L13a, mRNA (cDNA | | mRNA Length = 1142 | | strongly | |
| | | | clone | | | | similar to | |
| | | | IMAGE: 4644246) | | | | XP_512821.1 | |
| | | | Length = 1139 | | | | PREDICTED: | |
| | | | | | | | similar to | |
| | | | | | | | ribosomal | |
| | | | | | | | protein L13a; | |
| | | | | | | | 60S | |
| | | | | | | | ribosomal | |
| | | | | | | | protein L13a; | |
| | | | | | | | 23 kD highly | |
| | | | | | | | basic protein | |
| | | | | | | | [Pan | |
| | | | | | | | troglodytes] | |
| 502 | | BC018867.2 | Homo sapiens | NM_002295.4 | Homo sapiens ribosomal | Hs.563823 | Transcribed | |
| | | | ribosomal protein | | protein SA (RPSA), | | locus | |
| | | | SA, transcript variant | | transcript variant 1, mRNA | | | |
| | | | 1, mRNA (cDNA | | Length = 1155 | | | |
| | | | clone | | | | | |
| | | | IMAGE: 3141130) | | | | | |
| | | | Length = 1035 | | | | | |
| 503 | | BC028082.1 | Homo sapiens | NM_001558.2 | Homo sapiens interleukin | Hs.504035 | Interleukin | IL10RA |
| | | | interleukin 10 | | 10 receptor, alpha | | 10 receptor, | |
| | | | receptor, alpha, | | (IL10RA), mRNA | | alpha | |
| | | | mRNA (cDNA clone | | Length = 3649 | | | |
| | | | MGC: 40017 | | | | | |
| | | | IMAGE: 5216986), | | | | | |
| | | | complete cds | | | | | |
| | | | Length = 3691 | | | | | |
| 504 | | BC106007.1 | Homo sapiens single | NM_021805.1 | Homo sapiens single Ig IL- | Hs.501624 | Single Ig IL- | SIGIRR |
| | | | Ig IL-1R-related | | 1R-related molecule | | 1R-related | |
| | | | molecule, mRNA | | (SIGIRR), mRNA | | molecule | |
| | | | (cDNA clone | | Length = 1695 | | | |
| | | | MGC: 110992 | | | | | |
| | | | IMAGE: 6092503), | | | | | |
| | | | complete cds | | | | | |
| | | | Length = 1735 | | | | | |
| 505 | | AY324320.1 | Homo sapiens | NM_032808.5 | Homo sapiens leucine rich | Hs.132344 | Hypothetical | LRRN6A |
| | | | leucine-rich repeat | | repeat neuronal 6A | | protein | |
| | | | neuronal 6A | | (LRRN6A), mRNA | | LOC253044 | |
| | | | (LRRN6A) mRNA, | | Length = 2932 | | | |
| | | | complete cds | | | | | |
| | | | Length = 2940 | | | | | |
| 506 | | BC025703.1 | Homo sapiens CD3Z | NM_198053.1 | Homo sapiens CD3Z | Hs.156445 | CD3Z | CD3Z |
| | | | antigen, zeta | | antigen, zeta polypeptide | | antigen, zeta | |
| | | | polypeptide (TiT3 | | (TiT3 complex) (CD3Z), | | polypeptide | |
| | | | complex), transcript | | transcript variant 1, mRNA | | (TiT3 | |
| | | | variant 1, mRNA | | Length = 1677 | | complex) | |
| | | | (cDNA clone | | | | | |
| | | | MGC: 34513 | | | | | |
| | | | IMAGE: 5222574), | | | | | |
| | | | complete cds | | | | | |
| | | | Length = 1614 | | | | | |
| 507 | | U07802.1 | HSU07802 Human | | | Hs.503093 | Zinc finger | ZFP36L2 |
| | | | Tis11d gene, | | | | protein 36, | |
| | | | complete cds | | | | C3H type- | |
| | | | Length = 5504 | | | | like 2 | |
| 508 | | AF033347.1 | AF033347 Homo | NM_004519.2 | Homo sapiens potassium | Hs.374023 | Potassium | KCNQ3 |
| | | | sapiens potassium | | voltage-gated channel, | | voltage- | |
| | | | channel homolog | | KQT-like subfamily, | | gated | |
| | | | (KCNQ3) mRNA, | | member 3 (KCNQ3), | | channel, | |
| | | | partial cds | | mRNA Length = 3097 | | KQT-like | |
| | | | Length = 2755 | | | | subfamily, | |
| | | | | | | | member 3 | |
| 509 | | BC029848.1 | Homo sapiens tumor | NM_003820.2 | Homo sapiens tumor | Hs.512898 | Tumor | TNFRSF14 |
| | | | necrosis factor | | necrosis factor receptor | | necrosis | |
| | | | receptor superfamily, | | superfamily, member 14 | | factor | |
| | | | member 14 | | (herpesvirus entry | | receptor | |
| | | | (herpesvirus entry | | mediator) (TNFRSF14), | | superfamily, | |
| | | | mediator), mRNA | | mRNA Length = 1730 | | member 14 | |
| | | | (cDNA clone | | | | (herpesvirus | |
| | | | MGC: 35320 | | | | entry | |
| | | | IMAGE: 5178561), | | | | mediator) | |
| | | | complete cds | | | | | |
| | | | Length = 1756 | | | | | |
| 510 | | AF129756.1 | DJ201G24 Homo | NM_009588.1 | Homo sapiens lymphotoxin | Hs.376208 | Lymphotoxin | LTB |
| | | | sapiens MSH55 | | beta (TNF superfamily, | | beta (TNF | |
| | | | gene, partial cds; | | member 3) (LTB), transcript | | superfamily, | |
| | | | and CLIC1, DDAH, | | variant 2, mRNA | | member 3) | |
| | | | G6b, G6c, G5b, G6d, | | Length = 848 | | | |
| | | | G6e, G6f, BAT5, | | | | | |
| | | | G5b, CSK2B, BAT4, | | | | | |
| | | | G4, Apo M, BAT3, | | | | | |
| | | | BAT2, AIF-1, 1C7, | | | | | |
| | | | LST-1, LTB, TNF, | | | | | |
| | | | and LTA genes, | | | | | |
| | | | complete cds | | | | | |
| | | | Length = 184666 | | | | | |
| 511 | | BC035576.1 | Homo sapiens | NM_003954.1 | Homo sapiens mitogen- | Hs.404183 | Mitogen- | MAP3K14 |
| | | | mitogen-activated | | activated protein kinase | | activated | |
| | | | protein kinase kinase | | kinase kinase 14 | | protein | |
| | | | kinase 14, mRNA | | (MAP3K14), mRNA | | kinase | |
| | | | (cDNA clone | | Length = 4596 | | kinase | |
| | | | MGC: 45335 | | | | kinase 14 | |
| | | | IMAGE: 5497185), | | | | | |
| | | | complete cds | | | | | |
| | | | Length = 4452 | | | | | |
| 512 | | AF340053.1 | Homo sapiens | NM_000873.2 | Homo sapiens intercellular | Hs.431460 | Intercellular | ICAM2 |
| | | | isolate H#8 | | adhesion molecule 2 | | adhesion | |
| | | | intercellular adhesion | | (ICAM2), mRNA | | molecule 2 | |
| | | | molecule 2 (ICAM2) | | Length = 1035 | | | |
| | | | mRNA, complete cds | | | | | |
| | | | Length = 1020 | | | | | |
| 513 | | AC011382.4 | Homo sapiens | NM_005211.2 | Homo sapiens colony | Hs.483829 | Colony | CSF1R |
| | | | chromosome 5 clone | | stimulating factor 1 | | stimulating | |
| | | | CTB-13H5, complete | | receptor, formerly | | factor 1 | |
| | | | sequence | | McDonough feline sarcoma | | receptor, | |
| | | | Length = 86829 | | viral (v-fms) oncogene | | formerly | |
| | | | | | homolog (CSF1R), mRNA | | McDonough | |
| | | | | | Length = 3985 | | feline | |
| | | | | | | | sarcoma | |
| | | | | | | | viral (v-fms) | |
| | | | | | | | oncogene | |
| | | | | | | | homolog | |
| 514 | | BC025782.1 | Homo sapiens CD4 | NM_000616.2 | Homo sapiens CD4 antigen | Hs.17483 | CD4 antigen | CD4 |
| | | | antigen (p55), mRNA | | (p55) (CD4), mRNA | | (p55) | |
| | | | (cDNA clone | | Length = 3084 | | | |
| | | | MGC: 34241 | | | | | |
| | | | IMAGE: 5226427), | | | | | |
| | | | complete cds | | | | | |
| | | | Length = 3084 | | | | | |
| 515 | | BC098564.1 | Homo sapiens serpin | NM_004568.4 | Homo sapiens serpin | Hs.519523 | Serpin | SERPINB6 |
| | | | peptidase inhibitor, | | peptidase inhibitor, clade B | | peptidase | |
| | | | clade B (ovalbumin), | | (ovalbumin), member 6 | | inhibitor, | |
| | | | member 6, mRNA | | (SERPINB6), mRNA | | clade B | |
| | | | (cDNA clone | | Length = 1665 | | (ovalbumin), | |
| | | | MGC: 111370 | | | | member 6 | |
| | | | IMAGE: 30563105), | | | | | |
| | | | complete cds | | | | | |
| | | | Length = 1421 | | | | | |
| 516 | | AC129492.6 | Homo sapiens | NM_014232.1 | Homo sapiens vesicle- | Hs.25348 | Vesicle- | VAMP2 |
| | | | chromosome 17, | | associated membrane | | associated | |
| | | | clone RP11-599B13, | | protein 2 (synaptobrevin 2) | | membrane | |
| | | | complete sequence | | (VAMP2), mRNA | | protein 2 | |
| | | | Length = 196037 | | Length = 2159 | | (synaptobrevin | |
| | | | | | | | 2) | |
| 517 | | BC084538.1 | Homo sapiens v-akt | NM_001014432.1 | Homo sapiens v-akt murine | Hs.525622 | V-akt murine | AKT1 |
| | | | murine thymoma | | thymoma viral oncogene | | thymoma | |
| | | | viral oncogene | | homolog 1 (AKT1), | | viral | |
| | | | homolog 1, transcript | | transcript variant 2, mRNA | | oncogene | |
| | | | variant 2, mRNA | | Length = 2878 | | homolog 1 | |
| | | | (cDNA clone | | | | | |
| | | | MGC: 99656 | | | | | |
| | | | IMAGE: 6645486), | | | | | |
| | | | complete cds | | | | | |
| | | | Length = 2868 | | | | | |
| 518 | | BC022512.1 | Homo sapiens | NM_000442.2 | Homo sapiens | Hs.514412 | Platelet/endothelial | PECAM1 |
| | | | platelet/endothelial | | platelet/endothelial cell | | cell | |
| | | | cell adhesion | | adhesion molecule (CD31 | | adhesion | |
| | | | molecule (CD31 | | antigen) (PECAM1), mRNA | | molecule | |
| | | | antigen), mRNA | | Length = 3189 | | (CD31 | |
| | | | (cDNA clone | | | | antigen) | |
| | | | MGC: 26550 | | | | | |
| | | | IMAGE: 4816878), | | | | | |
| | | | complete cds | | | | | |
| | | | Length = 3189 | | | | | |
| 519 | | BC016838.2 | Homo sapiens | NM_031311.2 | Homo sapiens | Hs.233389 | Carboxypeptidase, | CPVL |
| | | | carboxypeptidase, | | carboxypeptidase, | | vitellogenic- | |
| | | | vitellogenic-like, | | vitellogenic-like (CPVL), | | like | |
| | | | transcript variant 2, | | transcript variant 1, mRNA | | | |
| | | | mRNA (cDNA clone | | Length = 1638 | | | |
| | | | MGC: 10029 | | | | | |
| | | | IMAGE: 3888647), | | | | | |
| | | | complete cds | | | | | |
| | | | Length = 1772 | | | | | |
| 520 | | M20431.1 | HUMMHDQAR | NM_002122.3 | Homo sapiens major | Hs.555872 | Major | HLA-DQA1 |
| | | | Human MHC class II | | histocompatibility complex, | | histocompatibility | |
| | | | HLA-DQ-alpha | | class II, DQ alpha 1 (HLA- | | complex, | |
| | | | (DR2-DQw1/DR4 | | DQA1), mRNA | | class II, DQ | |
| | | | DQw3) mRNA, | | Length = 1542 | | alpha 1 | |
| | | | partial cds, clone | | | | | |
| | | | ROF1.1-alpha | | | | | |
| | | | Length = 1030 | | | | | |
| 521 | | M29615.1 | HUMMHDXAR2 | NM_020056.2 | Homo sapiens major | Hs.387679 | Major | HLA-DQA1 |
| | | | Human MHC class II | | histocompatibility complex, | | histocompatibility | |
| | | | HLA-DX-alpha chain | | class II, DQ alpha 2 (HLA- | | complex, | |
| | | | gene, exons 2 | | DQA2), mRNA | | class II, DQ | |
| | | | through 4 | | Length = 1709 | | alpha 1 | |
| | | | Length = 2522 | | | | | |
| 522 | | M89957.1 | Homo sapiens cell | NM_021602.1 | Homo sapiens CD79B | Hs.89575 | CD79B | CD79B |
| | | | surface glycoprotein | | antigen (immunoglobulin- | | antigen | |
| | | | (IGB) mRNA, | | associated beta) (CD79B), | | (immunoglobulin- | |
| | | | complete cds | | transcript variant 2, mRNA | | associated | |
| | | | Length = 1270 | | Length = 958 | | beta) | |
| 523 | | BC028078.1 | Homo sapiens | NM_001337.3 | Homo sapiens chemokine | Hs.78913 | Chemokine | CX3CR1 |
| | | | chemokine (C—X3—C | | (C—X3—C motif) receptor 1 | | (C—X3—C | |
| | | | motif) receptor 1, | | (CX3CR1), mRNA | | motif) | |
| | | | mRNA (cDNA clone | | Length = 3108 | | receptor 1 | |
| | | | MGC: 40024 | | | | | |
| | | | IMAGE: 5216452), | | | | | |
| | | | complete cds | | | | | |
| | | | Length = 3119 | | | | | |
| 524 | | BC073986.1 | Homo sapiens | NM_006682.1 | Homo sapiens fibrinogen- | Hs.557207 | Transcribed | |
| | | | fibrinogen-like 2, | | like 2 (FGL2), mRNA | | locus, weakly | |
| | | | mRNA (cDNA clone | | Length = 1496 | | similar to | |
| | | | MGC: 87984 | | | | NP_006673.1 | |
| | | | IMAGE: 5748616), | | | | fibrinogen- | |
| | | | complete cds | | | | like 2; | |
| | | | Length = 1533 | | | | fibrinogen- | |
| | | | | | | | like protein 2; | |
| | | | | | | | fibroleukin | |
| | | | | | | | [Homo | |
| | | | | | | | sapiens] | |
| 525 | | BC037236.1 | Homo sapiens dual | NM_001946.2 | Homo sapiens dual | Hs.298654 | Dual | DUSP6 |
| | | | specificity | | specificity phosphatase 6 | | specificity | |
| | | | phosphatase 6, | | (DUSP6), transcript variant | | phosphatase 6 | |
| | | | transcript variant 1, | | 1, mRNA Length = 2842 | | | |
| | | | mRNA (cDNA clone | | | | | |
| | | | MGC: 35368 | | | | | |
| | | | IMAGE: 5183133), | | | | | |
| | | | complete cds | | | | | |
| | | | Length = 2874 | | | | | |
| 526 | | AF522250.1 | Homo sapiens MHC | NM_019111.3 | Homo sapiens major | Hs.555872 | Major | HLA-DQA1 |
| | | | class II antigen | | histocompatibility complex, | | histocompatibility | |
| | | | (HLA-DQA1/DRA) | | class II, DR alpha (HLA- | | complex, | |
| | | | mRNA, complete cds | | DRA), mRNA Length = 1267 | | class II, DQ | |
| | | | Length = 811 | | | | alpha 1 | |
| 527 | | M23905.1 | HUMMHDRC03 | NM_033554.2 | Homo sapiens major | Hs.347270 | Major | HLA-DPA1 |
| | | | Human MHC class II | | histocompatibility complex, | | histocompatibility | |
| | | | lymphocyte antigen | | class II, DP alpha 1 (HLA- | | complex, | |
| | | | (DPw4-alpha-1) gene | | DPA1), mRNA | | class II, DP | |
| | | | Length = 627 | | Length = 1157 | | alpha 1 | |
| 528 | | AY663400.1 | Homo sapiens | NM_002124.1 | Homo sapiens major | Hs.520049 | Major | HLA-DRB1 |
| | | | voucher Coriell Cell | | histocompatibility complex, | | histocompatibility | |
| | | | Repository DNA | | class II, DR beta 1 (HLA- | | complex, | |
| | | | sample NA01018 | | DRB1), mRNA | | class II, DR | |
| | | | MHC class II antigen | | Length = 1182 | | beta 4 | |
| | | | (HLA-DQB1) gene, | | | | | |
| | | | HLA-DQB1- | | | | | |
| | | | DQB1*050101 allele, | | | | | |
| | | | MHC class II antigen | | | | | |
| | | | (HLA-DQA1) gene, | | | | | |
| | | | HLA-DQA1- | | | | | |
| | | | DQA1*010102 allele, | | | | | |
| | | | and MHC class II | | | | | |
| | | | antigen (HLA-DRB1) | | | | | |
| | | | gene, complete cds | | | | | |
| | | | Le | | | | | |
| 529 | | BC007920.2 | Homo sapiens major | NM_022555.3 | Homo sapiens major | Hs.545653 | Major | HLA-DRB6 |
| | | | histocompatibility | | histocompatibility complex, | | histocompatibility | |
| | | | complex, class II, DR | | class II, DR beta 3 (HLA- | | complex, | |
| | | | beta 1, mRNA | | DRB3), mRNA | | class II, DR | |
| | | | (cDNA clone | | Length = 1158 | | beta 6 | |
| | | | MGC: 14111 | | | | (pseudogene) | |
| | | | IMAGE: 4309837), | | | | | |
| | | | complete cds | | | | | |
| | | | Length = 1218 | | | | | |
| 530 | | BC018726.1 | Homo sapiens CD74 | NM_001025158.1 | Homo sapiens CD74 | Hs.533282 | Non-POU | NONO |
| | | | antigen (invariant | | antigen (invariant | | domain | |
| | | | polypeptide of major | | polypeptide of major | | containing, | |
| | | | histocompatibility | | histocompatibility complex, | | octamer- | |
| | | | complex, class II | | class II antigen-associated) | | binding | |
| | | | antigen-associated), | | (CD74), transcript variant 3, | | | |
| | | | transcript variant 2, | | mRNA Length = 1155 | | | |
| | | | mRNA (cDNA clone | | | | | |
| | | | MGC: 31825 | | | | | |
| | | | IMAGE: 4853578), | | | | | |
| | | | complete cds | | | | | |
| | | | Length = 1327 | | | | | |
| 531 | | M16942.1 | HUMMHDRBU | NM_021983.4 | Homo sapiens major | Hs.545653 | Major | HLA-DRB6 |
| | | | Human MHC class II | | histocompatibility complex, | | histocompatibility | |
| | | | HLA-DRw53- | | class II, DR beta 4 (HLA- | | complex, | |
| | | | associated | | DRB4), mRNA | | class II, DR | |
| | | | glycoprotein beta- | | Length = 1193 | | beta 6 | |
| | | | chain mRNA, | | | | (pseudogene) | |
| | | | complete cds | | | | | |
| | | | Length = 1141 | | | | | |
| 532 | | BC009234.2 | Homo sapiens major | NM_002125.3 | Homo sapiens major | Hs.534322 | Major | HLA-DRB5 |
| | | | histocompatibility | | histocompatibility complex, | | histocompatibility | |
| | | | complex, class II, DR | | class II, DR beta 5 (HLA- | | complex, | |
| | | | beta 5, mRNA | | DRB5), mRNA | | class II, DR | |
| | | | (cDNA clone | | Length = 1171 | | beta 3 | |
| | | | MGC: 16550 | | | | | |
| | | | IMAGE: 4054398), | | | | | |
| | | | complete cds | | | | | |
| | | | Length = 1185 | | | | | |

### LITERARY REFERENCES

1. Jecklin, E., Arbeitsbuch Anatomie und Physiologie, (2004), Elsevier Urban und Fisher Verlag München, 12. Auflage, S. 294-299.
2. Trebsdorf, M., Gebhardt P., Biologie, Anatomie, Physiologie, Lehrbuch und Atlas, Lauverlag, 4. Auflage, S250-251.
3. Penko, M. E., Tirbaso D., (1999), An overview of liver transplantation, AACN clinical issues, 10 (2), 176-184.
4. Bauer, M., Winning J.; Kortgen A., (2005), Liver failure, Curr Opin Anaesthesiol., 18, 11-116.
5. Gao, L. H., Zheng, S. S., (2004), Cytomegalovirus and chronic allograft rejection in liver transplantation, World J. Gastroenterol., 10 (13), 1857-1861.
6. Shimizu T, Tajiri T, Akimaru K, Yoshida H, Yokomuro S, Mamada Y, Taniai N, Kawano Y, Mizuguchi Y, Takahashi T, Mizuta K, Kawarasaki H., (2003), Postoperative management and complications in living-related liver transplantation, J. Nippon Med. School, 70 (6), 522-527.
7. Müller A. R., Neuhaus P., (2004), Dünndarmtransplantation, Deutsches Ärtzeblatt, Jg. 101, Heft 1-2, A38-A43.
8. Huber W, Heydebreck A, Sueltmann H, et al. (2003) Parameter estimation for the calibration and variance stabilization of microarray data. Stat. Appl. in Gen. and Mol. Biol. Vol. 2, Issue 1, Article 3
9. Tomic V., et. al., Transcriptomic and proteomic patterns of systemic inflammation in on- and off-pump coronary artery bypass grafting. Circulation 2005, 112:2912-2920.
10. Troyanskaya O., et. al., Missing value estimation methods for DNA Microarrays. Bioinformatics 2001, 17 (6): 520-525.
11. Läuter J., Exact t and F tests for analyzing studies with multiple endpoints. Biometrics 1996, 52: 964-970.
Storey J. D., Tibshirani R., Statistical Significance for Genome-Wide Experiments. PNAS 2003, 100 (16): 9440-9445.

1-34. (canceled)

35. A method for detection of possible postoperative tissue incompatibility reactions following a liver transplantation, comprising:
obtaining a gene expression profile from in vitro samples obtained from patients, including genes with characteristic expression associated with patient condition following liver transplantation, and
monitoring the expression of characteristic genes as indicators of possible postoperative occurring tissue incompatibility reactions.

36. The method of claim 35, wherein the patient has received a liver transplant and is monitored for risk of a rejection due to tissue incompatibility.

37. The method of claim 35, wherein the expression profile includes at least 4 polynucleotides selected from SEQ-IDs 1 through 532.

38. The method of claim 35, wherein gene activities of polynucleotides with SEQ-IDs No 1 through 532 are compared and these expression behaviors are compiled into gene activity clusters.

39. The method of claim 38, wherein the individual clusters are composed as follows:
Cluster 1: Gene activities of SEQ-IDs No. 1 through 132;
Cluster 2: Gene activity data of SEQ-IDs No. 133 through 353;
Cluster 3: Gene activity data of SEQ-IDs No. 354 through 410; and
Cluster 4: Gene activity data of SEQ-IDs No 411 through 532.

40. The method of claim 35, wherein the method is used as criteria for inclusion or exclusion of liver transplant patients in clinical studies of phases 2-4.

41. The method of claim 35, wherein the method is used for establishing gene activity data for electronic further processing.

42. The method of claim 35, wherein gene activity data is obtained and employed for production of software for the description of the individual prognosis of the patient, for diagnostic purposes and/or patient data management systems.

43. The method of claim 35, wherein the in vitro expression profiles obtained from a patient sample are employed for production of clinical expert systems and/or for modeling of cellular signal transmission pathways.

44. The method of claim 35, wherein for establishment of the gene expression profile a specific gene and/or gene fragment is employed, which is selected from the group comprised of SEQ-ID No. 1 through SEQ-ID No. 532 as well as gene fragments thereof with at least 5-2000 nucleotides.

45. The method of claim 44, wherein the gene fragment includes 20-200, preferably 20-80 nucleotides.

46. The method of claim 35, wherein the gene expression profile is determined using hybridization processes.

47. The method of claim 46, wherein the hybridization process is based on microarrays.

48. A process for in-vitro measurement of (a) gene expression profiles and/or (b) at least one gene activity cluster, for detection of possible postoperative tissue incompatibility reactions following a liver transplantation, comprising
identifying the gene activity of a plurality of particular genes in a patient sample which are associated with a liver transplantation,
monitoring the gene activity in a patient to be diagnosed for possible postoperative tissue incompatibility reactions following a liver transplantation,
wherein the genes and/or gene fragments selected for the monitoring of the postoperative condition are selected from the group comprised of: SEQ-ID No. 1 through SEQ-ID No. 532 as well as gene fragments thereof with at least 5-2000 nucleotides.

49. The process according to claim 48, wherein the gene fragments include 20-200, preferably 20-80 nucleotides.

50. The process according to claim 48, wherein at least 4 to 100 different genes and/or gene fragments are employed.

51. The process according to claim 48, wherein at least 200 different genes and/or gene fragments are employed.

52. The process according to claim 48, wherein at least 200 to 500 different genes and/or gene fragments are employed.

53. The process according to claim 48, wherein at least 500 to 1000 different genes and/or gene fragments are employed.

54. The process according to claim 48, wherein at least 1000 to 2000 different genes and/or gene fragments are employed.

55. The process according to claim 48, wherein the genes or gene fragments of SEQ-ID No. 1 through SEQ-ID No. 532 and/or the sequences derived from their RNA are replaced by one or more of: synthetic analogs, aptamers, spiegelmeres as well as peptido- and morpholinonucleic acids.

56. The process according to claim 48, wherein the synthetic analogs of the genes include 5-100, preferably approximately 70 base pairs.

57. The process according to claim 48, wherein the gene activities are determined using a hybridization processes.

58. The process according to claim 57, wherein the gene activity is determined using microarrays.

59. The process according to claim 48, wherein the gene activity is determined by processes independent of hybridization, in particular enzymatic and/or chemical hydrolysis and/or amplification processes, preferably PCR, subsequent quantification of the nucleic acids and/or of derivatives and/or fragments thereof.

60. The process according to claim 48, wherein the sample is selected from: body fluids, in particular blood, serum, urine, ascites fluid, seminal fluid, saliva, aspirate; cellular contents or a mixture thereof.

61. The process according to claim 48, wherein sample, in particular cell sample, is subjected to a lytic treatment in order to liberate cellular contents.

62. A method for screening of active substances for usefulness against rejection reactions in patients with liver transplants and/or for evaluation of the therapy effect of active substances against rejection reactions of patients with liver transplants, the method comprising
compiling a gene expression profile from in vitro samples obtained from patients, including genes with characteristic expression associated with patient condition following liver transplantation, and/or of the therefore employed probe, which genes are selected from the group comprised of SEQ-ID No. 1 through SEQ-ID No. 532 as well as gene fragments thereof with at least 5-2000 nucleotides, the method comprising
(a) administering an active substance, and
(b) monitoring the gene profile for evidence of:
switching off a target gene,
changing the activity of a target gene and/or
determining the activity of a gene or the therefrom derived protein products.

63. The process according to claim 62, wherein hybridization capable synthetic analogs of the probes are employed.

64. The process according to claim 62, wherein the gene fragments include 20-200, preferably 20-80 nucleotides.

65. A kit, comprising:
a selection of at least 4 polynucleotides with sequences according to SEQ-ID No. 1 through SEQ-ID No. 532 and/or gene fragments thereof with at least 5-2000 nucleotides, and
printed instructions for determination of gene expression profiles in vitro in a patient sample, for a determination of possible tissue incompatibility reactions following a liver transplantation.

66. A cluster of genes useful for detection of possible postoperative tissue incompatibility reactions following a liver transplantation, the cluster comprising at least one polynucleotide selected from the group comprised of SEQ-ID No. 1 through 132.

67. A cluster of genes useful for detection of possible postoperative tissue incompatibility reactions following a liver transplantation, the cluster comprising at least one polynucleotide selected from the group comprised of SEQ-ID No. 133 through 353.

68. A cluster of genes useful for detection of possible postoperative tissue incompatibility reactions following a liver transplantation, the cluster comprising at least one polynucleotide selected from the group comprised of SEQ-ID No. 354 through 410.

69. A cluster of genes useful for detection of possible postoperative tissue incompatibility reactions following a liver transplantation, the cluster comprising at least one polynucleotide selected from the group comprised of SEQ-ID No. 411 through 532.


SEQUENCE LISTING (532 sequences)

SEQ ID NO: 1 - DNA, 69 bp, Homo Sapiens
aggaaagaagttccaccaccaaacatgcagctgttacagacggccatgtacgaaccgccagaaggcttg

SEQ ID NO: 2 - DNA, 69 bp, Homo Sapiens
aaacatgctggacaattctagtgggccacggggactgggtggaaagcctggtggtcatctggtctccat

SEQ ID NO: 3 - DNA, 66 bp, Homo Sapiens
tacttaggcctccaacttttttcactccgtggtctcctgcggcacctcttagcttctgcgtctcca

SEQ ID NO: 4 - DNA, 67 bp, Homo Sapiens
ggctaaggacatgaaattgtcagaatggcaggtggcttcttaacagccctgtgagaagcagacagat

SEQ ID NO: 5 - DNA, 67 bp, Homo Sapiens
tagaaagccaggggtgaaaaagctctcctttctcagacgattctgtgaagtctatgtgggctaagga

SEQ ID NO: 6 - DNA, 70 bp, Homo Sapiens
atctcagaaatgtttgacctccaggagccgacctgcctacagacccgcctggagctgtacaagcagggcc

SEQ ID NO: 7 - DNA, 67 bp, Homo Sapiens
tggtaggctctatcactgtccatgtcctgatctctacaaccctcaggtggctgggtcattgtccgtt

SEQ ID NO: 8 - DNA, 68 bp, Homo Sapiens
ccaataacaattcataagaagcaaaacctaagcaaactaggagcagagaggaactccctcaattcgct

SEQ ID NO: 9 - DNA, 55 bp, Homo Sapiens
gtttgtttgcttgcttgtttacttgcttgtttaccaatagagttgacctgttatt

SEQ ID NO: 10 - DNA, 66 bp, Homo Sapiens
tctcataggaaaagtacatttaatacagcagtgtcatcagaagataacttgagcaccgtcatggct

SEQ ID NO: 11 - DNA, 66 bp, Homo Sapiens
ttacttaaaaatacaaaaattaggccggggtgtgggtgggtgatggcccgctacttgagggaggct

SEQ ID NO: 12 - DNA, 70 bp, Homo Sapiens
ttattccacgccgttagctctgtgcttctattagtgtctctatctctctttctctctctcttccatctga

SEQ ID NO: 13 - DNA, 69 bp, Homo Sapiens
tccattaacccaaggggttttcccctcccccccaagggccccaatttccccttcttcaaaagccaaaat

SEQ ID NO: 14 - DNA, 65 bp, Homo Sapiens
tggttgtggtatagctctggagactagatcaagttgtcggcaccactggctccttctgagaactg

SEQ ID NO: 15 - DNA, 56 bp, Homo Sapiens
ctggagccaaaaaagcgaaaagcccgaaaaaggcgaaagcagccaagccaaaaaag

SEQ ID NO: 16 - DNA, 70 bp, Homo Sapiens
gggtgcaaaccacatacatagtgtaatctgaatgtccctggctccttgctacttagctggtctacggact

SEQ ID NO: 17 - DNA, 67 bp, Homo Sapiens
tgtgggagagcaagcaaaaagcaagggctgccccagttgcatcatgggcatccattcagagctggga

SEQ ID NO: 18 - DNA, 68 bp, Homo Sapiens
ctaatctaatctgaaggtccattgcaacgaatcccgctccgctctgacccaagaactccgcactttct

SEQ ID NO: 19 - DNA, 65 bp, Homo Sapiens
acagagccgacttcacctctccaacaggcaatctctggtccttgggcaaatggctgccccaatct

SEQ ID NO: 20 - DNA, 70 bp, Homo Sapiens
ccaccccagccgcgcacagtcaccagaaaactcttttaggtatctgaccttcatttgaacctatgttccc

SEQ ID NO: 21 - DNA, 55 bp, Homo Sapiens
agtataataagctacttgaacacagcttcatcaagcgctacgagacgctggaggt

SEQ ID NO: 22 - DNA, 55 bp, Homo Sapiens
cactgatcctggccccctcttatttattctacatccttggcaccccacttgcact

SEQ ID NO: 23 - DNA, 70 bp, Homo Sapiens
acttctcttcttctcaggctgccttggtgcagacaaatatattgtccccaacttcctgccttgtgcccca

SEQ ID NO: 24 - DNA, 65 bp, Homo Sapiens
atgagccagctctcatttaaattaacagagggtaaagtttttgattaccaagagggtgatgccaa

SEQ ID NO: 25 - DNA, 55 bp, Homo Sapiens
ccaccgctaactctcagccccagtcaccctcttggagcttccctgctttgaatta

SEQ ID NO: 26 - DNA, 70 bp, Homo Sapiens
gctgctccccatagctaaggaccatagctaaagtcaaaccatatgatccaaacttcatgtgtgccatcaa

SEQ ID NO: 27 - DNA, 70 bp, Homo Sapiens
tccttgctccctgcaccctcactgttctgtgcactttttctacctacataaacacacgcattccacctct

SEQ ID NO: 28 - DNA, 67 bp, Homo Sapiens
agcccctgagcttccacagccctccacatttattgggtaggatgaaagggaggaggaggtaacgact

SEQ ID NO: 29 - DNA, 55 bp, Homo Sapiens
cacccctaactctcagccccagtcaccctcttggagcttccctgctttgaattaa

SEQ ID NO: 30 - DNA, 69 bp, Homo Sapiens
acctggtgaacttctacccttaccataataagaaacttggtacagaggacatccacaatctcagcccct

SEQ ID NO: 31 - DNA, 69 bp, Homo Sapiens
tctcatcttttctgttggtcttccccaggttttacagcacctatttcagttactttcccagctcccatt

SEQ ID NO: 32 - DNA, 70 bp, Homo Sapiens
ccccaacctgaccacctcattcttatctttcttctgtttcttcctccccgctgtcatttcagtctcttca

SEQ ID NO: 33 - DNA, 69 bp, Homo Sapiens
ttggtttccttcctttgtcctctcctcctgcccctgcagagttccttcaccccagcccagttcattctt

SEQ ID NO: 34 - DNA, 68 bp, Homo Sapiens
aacccggcagctccgcagactcgcaccgcgaatttaaggtggcaatttgtttacaacttttcctcccc

SEQ ID NO: 35 - DNA, 55 bp, Homo Sapiens
atctcctccagctccttttaagaacgaacttgatgaaaacacagactttacctac

SEQ ID NO: 36 - DNA, 55 bp, Homo Sapiens
gtgcctcccccgccacctgtgtgttcttttgatacatttatcttctgtttttctc

SEQ ID NO: 37 - DNA, 70 bp, Homo Sapiens
cctagctccttctcctcctcggggcttgtccggtgaaaagggttctcgccctttcaagtaagcgctccaa

SEQ ID NO: 38 - DNA, 69 bp, Homo Sapiens
tctcaacatttggcttatacctgcttcttcatctttctggtcctctctctcctccgaccctccactctc

SEQ ID NO: 39 - DNA, 68 bp, Homo Sapiens
tgaggaatgacttcgtcctggttgagtggggtgaaaacagtgaaatgcgaacaaggataataagacca

SEQ ID NO: 40 - DNA, 66 bp, Homo Sapiens
cattggttgctttggtggcacgtaagctccttgtctgtctccagcacccagaatctcattaaagct

SEQ ID NO: 41 - DNA, 70 bp, Homo Sapiens
tttacaaaaagtcacttttgggctgggcgcagtggctcacgcctgtaatcccagcactttgggaggctga

SEQ ID NO: 42 - DNA, 65 bp, Homo Sapiens
acactagagcagagtacgagtctgaggcggagggagtaatggcaggacaagcgtttagaaagttt

SEQ ID NO: 43 - DNA, 65 bp, Homo Sapiens
ttgagatggctccagaacctttgtgcttactcctctcttcacggagctaagcgggctgtggcgct

SEQ ID NO: 44 - DNA, 68 bp, Homo Sapiens
aacacggtacccataaaaatccttatcacttgggctgggcgcagtggctcacgcccgcaatcccagca

SEQ ID NO: 45 - DNA, 68 bp, Homo Sapiens
aaaaattgaaaaaaaaaaaaaattttttttccccagggggcccccctcccccaagaggggaaaaaaaa

SEQ ID NO: 46 - DNA, 70 bp, Homo Sapiens
tgcaagagaagggggtctggggcttctcccttggaccctgggggacttaggtgagaagcatgtgaatgta

SEQ ID NO: 47 - DNA, 65 bp, Homo Sapiens
attaggagggtttcgaggacacgctgaaagctcgggcagatctctgcagaagcggacgggcatca

SEQ ID NO: 48 - DNA, 66 bp, Homo Sapiens
acatcaagggacgccgtgtgatatggcttggagacatgcacttgacggcagtgacaacagctctca

SEQ ID NO: 49 - DNA, 65 bp, Homo Sapiens
ccaccctcccataactagagttcataatcaaactaacactgccacctcctcaggcctaggaatgg

SEQ ID NO: 50 - DNA, 66 bp, Homo Sapiens
aactggatctctcctcttccgccctcttcgctaggacagtcgcttgcaattggccgcacgccccta

SEQ ID NO: 51 - DNA, 68 bp, Homo Sapiens
ccccactcaaggccctcctcagctcacggagttagaaaaaacacttgctttagaaaatcttaactata

SEQ ID NO: 52 - DNA, 69 bp, Homo Sapiens
taacaatgagacacgttacagaacctatgttcaggttgcgggtgagctgccctctccaaatccagccag

SEQ ID NO: 53 - DNA, 68 bp, Homo Sapiens
acatcctttatatcaactgcctcaatacgaacgtggtttgcaccccgacagcaccaccgccccttcca

SEQ ID NO: 54 - DNA, 70 bp, Homo Sapiens
tgacgcgctggcgggagaccgaggtggggccgctgtaagagcccagtcgatcccgcctcgtgccctcagc

SEQ ID NO: 55 - DNA, 68 bp, Homo Sapiens
ccacgctctcaacgatatgacttccatgtaaacgttcatccactctgcctgcttacaccctgccctca

SEQ ID NO: 56 - DNA, 70 bp, Homo Sapiens
agagcgtcgcacaaagcgaaggcgtccacgcagctgctctcgccggggccaccgccggggctgcctgcta

SEQ ID NO: 57 - DNA, 70 bp, Homo Sapiens
ccccaccctctttgtctcagcgtgttatgtgcaatgacctatttaaggtaaacccattccaactacagca

SEQ ID NO: 58 - DNA, 65 bp, Homo Sapiens
atacacaggcacgccacagtaccaggcattaccaataccagctcacgttcctctccttattcgga

SEQ ID NO: 59 - DNA, 70 bp, Homo Sapiens
aggtccacagacccgattcttacaacctggcgctctaacctcgccaacgggccaggaaaaaaacagaaca

SEQ ID NO: 60 - DNA, 65 bp, Homo Sapiens
ttacggggcacctacaatgtgtcaggcttcatgttgttcattgacgttgcctgagatcctgctcg

SEQ ID NO: 61 - DNA, 70 bp, Homo Sapiens
taagttggagtgtgtttggatgaggagggacaagaaaactgcttctgcaggggaggtggtccccactgca

SEQ ID NO: 62 - DNA, 70 bp, Homo Sapiens
tgaatccctcaatacccctagtatctggctgggttttcaggggctttggaagctctgttgcaggtgtccg

SEQ ID NO: 63 - DNA, 69 bp, Homo Sapiens
ggtgagcggtccgggaggaaacacgcggccggccaagggccagggtgcggcgggagattttgggtgaag

SEQ ID NO: 64 - DNA, 70 bp, Homo Sapiens
gctcacgggcggcaggcagaaccttccttttagtgagttgtaaagtcagagagaagctgaaaaattagag

SEQ ID NO: 65 - DNA, 67 bp, Homo Sapiens
atgagaaagaagtttgacgacggagaagatcctttggatgcagagagccagcaaggaggcggcggca

SEQ ID NO: 66 - DNA, 65 bp, Homo Sapiens
ttcattcacaccatagaaactgtacagcgactgagagtgctagagtcacggcggcggccaggcca

SEQ ID NO: 67 - DNA, 65 bp, Homo Sapiens
gcgggggcgctgtatggcgtgggcgcgccgggttcggtgtaatagaacgcaggcggcggcgcgtg

SEQ ID NO: 68 - DNA, 67 bp, Homo Sapiens
gcccagaggcagcggcggcagatctatccatactaatggaaatgtaactggaactgactctgatgat

SEQ ID NO: 69 - DNA, 55 bp, Homo Sapiens
tttttttgtgggggcggggccggggggactctggtatctaattctttaatgattc

SEQ ID NO: 70 - DNA, 70 bp, Homo Sapiens
attgtctaaatatttttgcgtctcagcgggcccctgtcgctcttggctgataggtcccagttctctcccc

SEQ ID NO: 71 - DNA, 65 bp, Homo Sapiens
gggcccatggcgcgttcggaggacgggagcggctgcctcgtgtcgcggggccgttcgcagagtga

SEQ ID NO: 72 - DNA, 67 bp, Homo Sapiens
tagtgatatgacagggctggggcaagaacaggggcactagctgccttattagctaatttagtgccct

SEQ ID NO: 73 - DNA, 68 bp, Homo Sapiens
aaaccctgttctctaccaaaagtacaaaaattaggctggggcgcggtggtggggcgcctggcaattcc

SEQ ID NO: 74 - DNA, 65 bp, Homo Sapiens
agagggctgaggagccgtggggctcacgtttcagggattagcttgtgtaacttgacttgttcccc

SEQ ID NO: 75 - DNA, 65 bp, Homo Sapiens
tgaagttatagagctaatattgcagagctgggaatctgctggttccagaacttgccctcttgggg

SEQ ID NO: 76 - DNA, 70 bp, Homo Sapiens
tttagtcgcacgcacgtttataataatagcaaaaggcgggcgtccccgcgccgcaggttgcttaggagga

SEQ ID NO: 77 - DNA, 70 bp, Homo Sapiens
tctccgggattgggagccaaaaaaaaaaaaaaagcatcaccatcatgattgagaaatggagttactggca

SEQ ID NO: 78 - DNA, 69 bp, Homo Sapiens
tttgaattccagcatttttattcagcacaccacatcggggaggggcggcagtggtttccacggtaacca

SEQ ID NO: 79 - DNA, 70 bp, Homo Sapiens
agcccaagccccttggacactgcagctcttttcagtttttgcttatacacaattcattctttgcagctaa

SEQ ID NO: 80 - DNA, 68 bp, Homo Sapiens
ctgaaggccccgagggcgaaggtaccgaaagcacagtaatcactggtgtcgatattgtcatgaaccat

SEQ ID NO: 81 - DNA, 70 bp, Homo Sapiens
tttttttttttcccaaaaaaaaaaaaaaaaaggggaacccccgggggggggaagggggggaccccccaaa

SEQ ID NO: 82 - DNA, 55 bp, Homo Sapiens
cttgctgttatctctagctctttccctcctcccatttcctttagtagttgaattt

SEQ ID NO: 83 - DNA, 57 bp, Homo Sapiens
agttgaataaagagcttactggaatccagcagggttttctgcccaaggatttgcaag

SEQ ID NO: 84 - DNA, 66 bp, Homo Sapiens
tcctgagcacagataagggctcctttcaggcctttccttgagctgcacgtgaacctgtgtggggca

SEQ ID NO: 85 - DNA, 70 bp, Homo Sapiens
gctggcccacaagaggactcattttaggctttaggaacacatacaggctggaaagtgggaggatttggaa

SEQ ID NO: 86 - DNA, 68 bp, Homo Sapiens
tgagcctcctctccttcagaccttcctggtcctactttgcctgggtctgagaaaagtgatttcttcct

SEQ ID NO: 87 - DNA, 55 bp, Homo Sapiens
aaagtccttggcactgtcaaatggttcaacgtcagaaatggatatggatttataa

SEQ ID NO: 88 - DNA, 55 bp, Homo Sapiens
tcaggagatggaacgaatacgaatggacagcattggatcaactatttcttcagat

SEQ ID NO: 89 - DNA, 68 bp, Homo Sapiens
tgttccagttcctgcaggccaccgcccaggaagaagggatcttcggtgagtcccaaccagtcagccca

SEQ ID NO: 90 - DNA, 70 bp, Homo Sapiens
cacaaacgaacaagaaagtaatgacatcagaggatgtaagccagtagtctaatactcagcccaccacggt

SEQ ID NO: 91 - DNA, 66 bp, Homo Sapiens
tggaaggaggtggacatcccgtctaattccgtgtaccgctacccgcccaagtccggaagctatttt

SEQ ID NO: 92 - DNA, 66 bp, Homo Sapiens
ctccgactctgtgagcagtttccactcggcgcccaagcgtttgctgatctccgagttgtgcatctt

SEQ ID NO: 93 - DNA, 69 bp, Homo Sapiens
tcttctccacccacctgagtttctgttgtacaatcacagccgtccggcgaagcctgccgtcttttgcca

SEQ ID NO: 94 - DNA, 65 bp, Homo Sapiens
tgggttagaaaagcctggggagcttaaaagtgggccaaagcccatttttcctgttcctgtggctt

SEQ ID NO: 95 - DNA, 70 bp, Homo Sapiens
aatatggagctagagggtgctctttcatactagaaaaccaactatgttggtttttgtactattgtacagt

SEQ ID NO: 96 - DNA, 70 bp, Homo Sapiens
cccctccacctattgacaatggggacattacttcattcccgttgtcagtatatgctccagcttcatcagt

SEQ ID NO: 97 - DNA, 65 bp, Homo Sapiens
ccgggtgagtccatgaccagactgcaacctcgaaaacgggcccggcaacatctggcacatgaagt

SEQ ID NO: 98 - DNA, 66 bp, Homo Sapiens
agataaatgcacagcaccacagcaccacatctaagcattagtgatgggtagctgatgtcagcttca

SEQ ID NO: 99 - DNA, 70 bp, Homo Sapiens
tagtctctcttcataaaaagcaattacaatttcgaggacacttcatatttgcctctttcgccagcaccaa

SEQ ID NO: 100 - DNA, 70 bp, Homo Sapiens
tgcttgtatggctttctttggcctctgaccctgctgcccattctttccaacatcacagatgaactgcctc

SEQ ID NO: 101 - DNA, 55 bp, Homo Sapiens
cacatgatcctgaccgtcggaaacaaggcagatggagtcctggtgggaacagatg

SEQ ID NO: 102 - DNA, 65 bp, Homo Sapiens
gctccacccagaaggctttcatttgtactctgctgggagtgactgggaaaaactccttccctgct

SEQ ID NO: 103 - DNA, 57 bp, Homo Sapiens
tggggttatctctgtgttaggggtatatgatgggggagtagatctttctaggaggga

SEQ ID NO: 104 - DNA, 55 bp, Homo Sapiens
gccaccctcaatataatcagcggaccatccagaatgacatcatgttattgcagct

SEQ ID NO: 105 - DNA, 55 bp, Homo Sapiens
gacatcgtgattctccagctcaacgggtcggccaccatcaacgccaacgtgcagg

SEQ ID NO: 106 - DNA, 70 bp, Homo Sapiens
aaattaagaaggcaaaatgtaaaggagtcaaactataaatcaagtatttgggaagtgaagactggaagct

SEQ ID NO: 107 - DNA, 70 bp, Homo Sapiens
gctgccaccttatgcagtgcatcgaaaccttttgcttggggatgtgcttggagaggcagataacgctgaa

SEQ ID NO: 108 - DNA, 55 bp, Homo Sapiens
caatgtcttcgcaatgtttgaccagtcccagatccaggagtttaaggaggctttc

SEQ ID NO: 109 - DNA, 57 bp, Homo Sapiens
ccactgcccaactcatagccacgctgaagaatgggaggaaaatttgcttggatctgc

SEQ ID NO: 110 - DNA, 55 bp, Homo Sapiens
cactgcccaactgatagccacgctgaagaatggaaggaaaatttgcttggacctg

SEQ ID NO: 111 - DNA, 55 bp, Homo Sapiens
tgatcgggaaaggaacccattgcaaccaagtcgaagtgatagccacactgaagga

SEQ ID NO: 112 - DNA, 66 bp, Homo Sapiens
gaacccattgcaaccaagtcgaagtgatagccacactgaaggatgggaggaaaatctgcctggacc

SEQ ID NO: 113 - DNA, 65 bp, Homo Sapiens
aggcctctggatcagtttgtgctgcagtcgcacgcatggttcaacgtgtcctccctcccctatgc

SEQ ID NO: 114 - DNA, 70 bp, Homo Sapiens
actgccccactgcccaactgatagccacgctgaagaatggaaggaaaatttgcttggacctgcaagcccc

SEQ ID NO: 115 - DNA, 70 bp, Homo Sapiens
ctaaagtcctaccagtggaagatgctcaacacctcctccttgctggagcagctgaacgagcagtttaact

SEQ ID NO: 116 - DNA, 59 bp, Homo Sapiens
ctaacactcgactctgctgctcatgggaagaacagaattgctcctgcatgcaactaatt

SEQ ID NO: 117 - DNA, 69 bp, Homo Sapiens
gaccaggaaatacaacgagctgctaaagtcctaccagtggaagatgctcaacacctcctccttgctgga

SEQ ID NO: 118 - DNA, 70 bp, Homo Sapiens
tgcttccaatcggggtgaaagtcaccctataaggcagccctggggaggatagaaggcaacgccttcccgt

SEQ ID NO: 119 - DNA, 70 bp, Homo Sapiens
aacgcggcatgctgatgcaccgtcaaactcacactttcaaccccaaaacccgactctttgaacaacccga

SEQ ID NO: 120 - DNA, 70 bp, Homo Sapiens
atccaagcataactcagtgacgcatgtgtttggcagcgggacccagctcaccgttttaagtcagcccaag

SEQ ID NO: 121 - DNA, 57 bp, Homo Sapiens
gctctggtggcccttgcagaagccctaagtacggttacttatgcgtgtagacatttt

SEQ ID NO: 122 - DNA, 67 bp, Homo Sapiens
gttgccaatctggtagtacgagatctggattgttggcagccatcttgccactaaaaggaggaggccc

SEQ ID NO: 123 - DNA, 70 bp, Homo Sapiens
ggcatgaagactacaggaggatgtgcagccagggagaacgaagacatcttggcacgttcatctgcttggt

SEQ ID NO: 124 - DNA, 70 bp, Homo Sapiens
agcagccgggacttggagaattcctaaaatccaagagggtaagtctatcccaggttggcagtagagacca

SEQ ID NO: 125 - DNA, 69 bp, Homo Sapiens
tgacccggggcccaaagccaccaccgtccttccaaagccagatcttatttatttatttatttcagtact

SEQ ID NO: 126 - DNA, 70 bp, Homo Sapiens
ccctgtatcctttagctatgatcctccaatccccccattccccacgactcaaagcaaccactaaccaccc

SEQ ID NO: 127 - DNA, 67 bp, Homo Sapiens
ggccgcttcagcagcacctagggaggttgtaagaaatgcagattcctaagccccacccctgactcag

SEQ ID NO: 128 - DNA, 70 bp, Homo Sapiens
tagtagccaatcttcccgaagagcaggcccatgaggaccccgccaatcatcccgacatatttgtagccca

SEQ ID NO: 129 - DNA, 68 bp, Homo Sapiens
tgtgagtctagggtccgcagagaaagctttgctttaggctgtgggtcttcaaggcagcaggtggagct

SEQ ID NO: 130 - DNA, 65 bp, Homo Sapiens
agtctggggatctgggaaggcgtcttggaggagttaatatctaagttgagctttgaagggtaggt

SEQ ID NO: 131 - DNA, 68 bp, Homo Sapiens
ggggatgtctcccaaatggctggaaacacgggtgttccgggctcattttaaaaaaggtgtttttaaaa

SEQ ID NO: 132 - DNA, 70 bp, Homo Sapiens
tgttataaagaagccagccaaaacccaccaaaaccaacatggctacaagagtaacttctggttcccctca

SEQ ID NO: 133 - DNA, 65 bp, Homo Sapiens
agtagccgtgcatacagctatgaaggatgggcaactgtacctgactggaggagtctggagtctag

SEQ ID NO: 134 - DNA, 70 bp, Homo Sapiens
ccatcagtttgcggaagtctggggtggaggacagtctggagtgacaggctgctaccagaccagtcgatct

SEQ ID NO: 135 - DNA, 69 bp, Homo Sapiens
agagctgagtagtcgcggcagagaccatttggccagcaaagcctaaaacacatactgtctgccggggct

SEQ ID NO: 136 - DNA, 67 bp, Homo Sapiens
ttttttttttaagaggcagggactctatcacccaggctgcagtaacagctcactgcagcctcaaact

SEQ ID NO: 137 - DNA, 69 bp, Homo Sapiens
aacagtcagccactgagtaatcaaaaatcatactggctgcggggtcacacaacgagtaacagtgccttt

SEQ ID NO: 138 - DNA, 70 bp, Homo Sapiens
ttgactgttttgtcactggtccaacatacctggtgaaggaataaatccattaggtagaaatagttgggtt

SEQ ID NO: 139 - DNA, 66 bp, Homo Sapiens
ttgtctgtggctactttcacattataagagaatctaataaccttataacagaactattgagcatga

SEQ ID NO: 140 - DNA, 66 bp, Homo Sapiens
tcctttattcagtccacttattctgcttcagggtcacaggtcgcaggagcctctcccaacagctca

SEQ ID NO: 141 - DNA, 56 bp, Homo Sapiens
tgggcctggaagccattcttacttttcagtctctcccattctgtactgttttttgt

SEQ ID NO: 142 - DNA, 69 bp, Homo Sapiens
ggataaaaatctaggatgggacaacaactttcagccatctttctctggctctggacaggggcctgcacg

SEQ ID NO: 143 - DNA, 68 bp, Homo Sapiens
gtggagagcgagggcagcctgacggagaacatctgggccttcgctggcatctccaggccctgtgccct

SEQ ID NO: 144 - DNA, 70 bp, Homo Sapiens
atagtggggcagctatgctcggggtgtgattggcgttggttgtgtcggggtctagtgggcagagaagact

SEQ ID NO: 145 - DNA, 70 bp, Homo Sapiens
tttgtggggcctgaggttacataccttcagggtcaggttggtttcctgaggcagtgagcagggaaggagg

SEQ ID NO: 146 - DNA, 68 bp, Homo Sapiens
tttatagcgaacacccctgaggctctggggatcccatccagaacactgtgacctttgagtcgggcagt

SEQ ID NO: 147 - DNA, 57 bp, Homo Sapiens
atcccaggcatccctcctaacttttctttgtgcagcggtctggttatcgtctatccc

SEQ ID NO: 148 - DNA, 69 bp, Homo Sapiens
agcgaagagttcaattagtaatgtctatgtgttatgtcaggagatgaaacccttgttcttaggatcaca

SEQ ID NO: 149 - DNA, 55 bp, Homo Sapiens
actgaggccatttagagcaatttgtttaatgagtggttcagccatcaccaccagt

SEQ ID NO: 150 - DNA, 65 bp, Homo Sapiens
aaccctctctcctcagccctgcaggccgatccctggacataaactccttgaagttcagtgggcct

SEQ ID NO: 151 - DNA, 69 bp, Homo Sapiens
tcaggtgatggacaccaggatcctccatatcaaaagttcgtcagttcctccttacctccgagatcagca

SEQ ID NO: 152 - DNA, 70 bp, Homo Sapiens
tggcctgaagcaagaatttcaaactcctctgtctggaccacatcagggactcccttcccctctggtctct

SEQ ID NO: 153 - DNA, 66 bp, Homo Sapiens
ccattgtaattgctatcgccatcacagctgaacttgttgagatccccgtgttactgcctatcagca

SEQ ID NO: 154 - DNA, 64 bp, Homo Sapiens
cgagtgcgtggagctcttcagaaccgcctacttctgcagagacttggtcaggtactttcagggc

SEQ ID NO: 155 - DNA, 64 bp, Homo Sapiens
cttccttatggagctggagcagcccgcctagaacccagtctaatgagaactccagaaagctgga

SEQ ID NO: 156 - DNA, 55 bp, Homo Sapiens
tttatgacaccatcttccagatattcaacaaccgcaactgcctcagtctgaagga

SEQ ID NO: 157 - DNA, 68 bp, Homo Sapiens
attcgggccgctctctagtcccttgtaaaagacctgaaaaaatcggacacccagggtcttcagtcctt

SEQ ID NO: 158 - DNA, 70 bp, Homo Sapiens
cgcactgcgcccagccagtaactgccatttctaaagaggaaagagagcaggcagagggtcctgactccca

SEQ ID NO: 159 - DNA, 70 bp, Homo Sapiens
ctggggcaggtcactttacctctgtggacctgttttctctttgtgaagctagggagttagaggctgtaca

SEQ ID NO: 160 - DNA, 68 bp, Homo Sapiens
acccaggcatccaggcaccagagtttttgctctaaaccactacagtatatctgcttccatgacttact

SEQ ID NO: 161 - DNA, 55 bp, Homo Sapiens
ttcttattcatgtgtaagggaggtccatagaatttaggtggtctgtcaactattc

SEQ ID NO: 162 - DNA, 55 bp, Homo Sapiens
gtaaaccttcttccccgggacccggtcctgagatcaagaacctagaccaggcttt

SEQ ID NO: 163 - DNA, 55 bp, Homo Sapiens
cacggccacatttggttctaagaaaccctctgtcattcgctcccacattctgatg

SEQ ID NO: 164 - DNA, 70 bp, Homo Sapiens
ccacggccacatttggttctaagaaaccctctgtcattcgctcccacattctgatgagcaaccgcttccc

SEQ ID NO: 165 - DNA, 67 bp, Homo Sapiens
ctcaaacctcctcttctgacgtgccttttgcacccctcccattaggacaatcagtcccctcccatct

SEQ ID NO: 166 - DNA, 70 bp, Homo Sapiens
gacatcctgagcagtgccagctttcttctgttgatgccgttccctgaacataggaaaatagaaacttgct

SEQ ID NO: 167 - DNA, 55 bp, Homo Sapiens
tgcctgtgaaaaactctcagggattcacgtggaaccagctcagaattacctctag

SEQ ID NO: 168 - DNA, 55 bp, Homo Sapiens
agaaaatcaccaagtgactgtgaaaccgtcagttcggaaggctggttagaacatg

SEQ ID NO: 169 - DNA, 65 bp, Homo Sapiens
tagctattcaagaaaatcaccaagtgactgtgaaaccgtcagttcggaaggctggttagaacatg

SEQ ID NO: 170 - DNA, 67 bp, Homo Sapiens
ttgttgcggagttcataatgaataacacaggagaatggataaggcaaaacggaggctgggaaaatgg

SEQ ID NO: 171 - DNA, 59 bp, Homo Sapiens
tcaaaatgttttcaggagtgtaaagaagcatcgtgtttacctgtgcaggcactagtcct

SEQ ID NO: 172 - DNA, 60 bp, Homo Sapiens
actttaaacgagaaaatgaaacctctcataatcccactgcatagaaataacaagcgttaa

SEQ ID NO: 173 - DNA, 70 bp, Homo Sapiens
accgcattgccaatatccaggatgccatctggaacagtagaaagatcgtttgtcttgtgagcagacactt

SEQ ID NO: 174 - DNA, 69 bp, Homo Sapiens
agatcctcaagtacaagatgctggatgagcaggatgtgccggccgctgagcaaatgtgctttgagacca

SEQ ID NO: 175 - DNA, 65 bp, Homo Sapiens
cttctagagattgaggtgaaagcttcgtccgagaaacgccaggacagacgatggcagaggagagg

SEQ ID NO: 176 - DNA, 56 bp, Homo Sapiens
ttacttcggggctttcatggtgtgaaggaaggagcgtggtgcattggacatgggtc

SEQ ID NO: 177 - DNA, 67 bp, Homo Sapiens
acaaatgttttattacgcaaaccacatgtaggtcccaggctcaggggcttaccctacagcccccact

SEQ ID NO: 178 - DNA, 69 bp, Homo Sapiens
actctatgacacggccggacaggaagactatgaccgtctgaggcctttatcttacccaatgaccgatgt

SEQ ID NO: 179 - DNA, 56 bp, Homo Sapiens
tgggatggactgaacggacagttccagaagtgtgactggctaaagcttgatgtggt

SEQ ID NO: 180 - DNA, 70 bp, Homo Sapiens
tgtggagccctggatccaactgtacctgacactagccaatttcatttcttcagtttcatggacccctaaa

SEQ ID NO: 181 - DNA, 70 bp, Homo Sapiens
ctgtggaaatacagacaaggatcttagttgatattttgggcttggggcagtgagggcttaggacacccca

SEQ ID NO: 182 - DNA, 67 bp, Homo Sapiens
ggccatgaagaggatgctgtgaaattcccaacaaacattgatgctgacagtcatgcagtctgggagt

SEQ ID NO: 183 - DNA, 70 bp, Homo Sapiens
tgaagggcctaaccagccttgggagtattgactggtcccttacctcttatggctaagtctttgactgtgt

SEQ ID NO: 184 - DNA, 68 bp, Homo Sapiens
ttgctgcacagtccctcaagcttaccatgcccatattgaactcatcccctgccccacaacctgctcct

SEQ ID NO: 185 - DNA, 70 bp, Homo Sapiens
tttcaccaaaaaaaaaaaaacggggggcccccccccccccccccccccaaaaaaaaaaggggaaaacctt

SEQ ID NO: 186 - DNA, 70 bp, Homo Sapiens
acacaaaacatttccataacccaaaaaagctctctgatgttcccttgtaatcaatcccgctcccacacct

SEQ ID NO: 187 - DNA, 70 bp, Homo Sapiens
tgagagatctgccccaatgacccaaacacctcccactaggcccacctccagtatcagagattacatttca

SEQ ID NO: 188 - DNA, 55 bp, Homo Sapiens
tcactgtaaagctggaaagggacgaactggtgtaatgatatgtgcatatttatta

SEQ ID NO: 189 - DNA, 64 bp, Homo Sapiens
ttggggaaagattttaggaagaaagcaaggcagtccattcagggaattctggaggcagccttca

SEQ ID NO: 190 - DNA, 65 bp, Homo Sapiens
ggggattctacgatccgaaagctcaggtgttgggaaatggggaccccgtcttctggacaccagta

SEQ ID NO: 191 - DNA, 55 bp, Homo Sapiens
ggggagagaaggccatagagatccgatctgtggaaactggtcacttggatggtgt

SEQ ID NO: 192 - DNA, 66 bp, Homo Sapiens
ctcttcgtcttcttcaactggctgggctacgccaactcggccttcaaccccatcatctactgccgc

SEQ ID NO: 193 - DNA, 55 bp, Homo Sapiens
tgtgtcattgcctggcatggtttatatttgactacattcaactcaggggtgtcca

SEQ ID NO: 194 - DNA, 70 bp, Homo Sapiens
tggggtcacattttaacttggggactgtggggttctgtggtcccttctcgacttggcccagctccacttg

SEQ ID NO: 195 - DNA, 69 bp, Homo Sapiens
agcctctccgggccccccagttttttccgggtaagagactcgaactgatgctctctaaggattcttcaa

SEQ ID NO: 196 - DNA, 65 bp, Homo Sapiens
ataggagctaaatttggatttttggtaccatggaaccttgtatcccatgaccctggccaaaaccg

SEQ ID NO: 197 - DNA, 67 bp, Homo Sapiens
gccaggttcaagaaccactaccctgaaggacaactattccctccctgcccaggacccctgtatctta

SEQ ID NO: 198 - DNA, 59 bp, Homo Sapiens
ggtaccctggtattgggacagcaaaagccagtaaccatgagtatgaggaaatctctttc

SEQ ID NO: 199 - DNA, 65 bp, Homo Sapiens
tgggtcgtaagtttaggaggtgactgcatcctccagcatctcaactccgtctgtctactgtgtga

SEQ ID NO: 200 - DNA, 66 bp, Homo Sapiens
ttaatgtcagcttgcccaggttcaaactggaagagagttacactctcaactccgacctcgcccgcc

SEQ ID NO: 201 - DNA, 59 bp, Homo Sapiens
tacaagtagaatggcagctggaatttaaggagggacaagaatcaatggataagcgtggg

SEQ ID NO: 202 - DNA, 70 bp, Homo Sapiens
atggcagctggaatttaaggagggacaagaatcaatggataagcgtgggtggaggaagatccaaacagaa

SEQ ID NO: 203 - DNA, 65 bp, Homo Sapiens
cgatcctcgcgtgagacagatcaagatcaagaccggcgtggtgaagcggttggtcaaagaaaaag

SEQ ID NO: 204 - DNA, 65 bp, Homo Sapiens
agctgaagcctgcacagtgtccaccctgttcccactcccatctttcttccggacaatgaaataaa

SEQ ID NO: 205 - DNA, 70 bp, Homo Sapiens
ccatgctcaaccactgtggcgttctgctgcccctgcagttggcagaaaggatgttttgtcccatttcctt

SEQ ID NO: 206 - DNA, 65 bp, Homo Sapiens
gtacatccccaccgtttttgacaactactctgccaacgtgatggtggacgggaaaccagtcaact

SEQ ID NO: 207 - DNA, 70 bp, Homo Sapiens
atagtctagatggctcctctcagtactttggaggcccctatgtagtccgtgctgacagctgctcctagag

SEQ ID NO: 208 - DNA, 55 bp, Homo Sapiens
ttttttcagtcccagaacctacagataccctgctacttgcttcacgtggatgctc

SEQ ID NO: 209 - DNA, 67 bp, Homo Sapiens
cagaaaaccccacaaaaagccacaacaggtcgctcatcccgtcagtcatcctttattgcagtcggga

SEQ ID NO: 210 - DNA, 70 bp, Homo Sapiens
tcgagtctgctctggctgtgctgtgttggctgtgttgggacactatgttccagggattatgattcctaca

SEQ ID NO: 211 - DNA, 70 bp, Homo Sapiens
ttgtttcttacagtgagcgggatgcctactgggtggagaaccttatggtccaggagctggagaacttcaa

SEQ ID NO: 212 - DNA, 70 bp, Homo Sapiens
cccattgagaaaaaagccattccccagcgcttctgcaagctgcggaagataatgaacaccaagacctacc

SEQ ID NO: 213 - DNA, 67 bp, Homo Sapiens
acgccagtaagtgggccagggtcttctgttgagaactctgtgggtttcgctcttccttttggagaca

SEQ ID NO: 214 - DNA, 65 bp, Homo Sapiens
ggctcgagagggctggtccttatttatttaacttcacccgagttcctctgggtttctaagcagtt

SEQ ID NO: 215 - DNA, 57 bp, Homo Sapiens
catttcccccctcaagaccatatcagcttctctggggtcaagactgacaatcccgtg

SEQ ID NO: 216 - DNA, 69 bp, Homo Sapiens
tacgcaggcctaaagatccaacggttcttgcctgggtctgtgtccgaaataaattctcatatagactca

SEQ ID NO: 217 - DNA, 69 bp, Homo Sapiens
cctatgacatcctgcagtgccctgaggactagggctcccgtcctgctttgcagtgccatgtaaatcccc

SEQ ID NO: 218 - DNA, 69 bp, Homo Sapiens
gagagggtgtcagccaagaacgccctggagtcctacgccttcaacatgaagagcgccgtggaggatgag

SEQ ID NO: 219 - DNA, 56 bp, Homo Sapiens
tgagttcaactacatgttctgggggcccggagatagatgactttgcagatggaaag

SEQ ID NO: 220 - DNA, 65 bp, Homo Sapiens
ttaaccatgctgttccagcaaagatgataccttaaactcccactgcaagcccatgaacttcccag

SEQ ID NO: 221 - DNA, 59 bp, Homo Sapiens
tgtctcactgaactagaagccgagcctagaaactaacacagccatcaagggaatgactt

SEQ ID NO: 222 - DNA, 65 bp, Homo Sapiens
tttcgtaaagataaccagtttttgcagagaaacgtgtacccatatataattctcccacactagct

SEQ ID NO: 223 - DNA, 64 bp, Homo Sapiens
tgactgggtgataaatgagctgcgctaccaccttgaagagagccgagacaaaaacgttctcctt

SEQ ID NO: 224 - DNA, 68 bp, Homo Sapiens
tctgagatgagggtctagccctgttggggacagaaaagtgtgtagaccttcttcctgctagggctgca

SEQ ID NO: 225 - DNA, 70 bp, Homo Sapiens
agctccgccccttgaacatgaccgtggccccaaatttgctattcccatgcattttgtttgtttcttcact

SEQ ID NO: 226 - DNA, 70 bp, Homo Sapiens
aactccagcggtcccatgtcagccttctgatgatcccactccaccccacctcaacttatttaacttccta

SEQ ID NO: 227 - DNA, 65 bp, Homo Sapiens
gcttcctggtcttatcatggacaatttgggctagaggcaggaaggaagtgggatgacctcaggag

SEQ ID NO: 228 - DNA, 57 bp, Homo Sapiens
tgtgacagaatcttgtatttgggccaaggtgtttccatttctcaatcagtgcagtga

SEQ ID NO: 229 - DNA, 55 bp, Homo Sapiens
cacttccagtgttggctgtgtgacagaatcttgtatttgggccaaggtgtttcca

SEQ ID NO: 230 - DNA, 55 bp, Homo Sapiens
gatgccgaagatgaactttgcgaatgtatttattggtgccaatcccctgggtaag

SEQ ID NO: 231 - DNA, 70 bp, Homo Sapiens
agaatcttgtatttgggccaaggtgtttccatttctcaatcagtgcagtgatacatgtactccagaggga

SEQ ID NO: 232 - DNA, 70 bp, Homo Sapiens
atccacactgcacactgcctagcccaaaacgtcttattgtggtaggatcagccctcattttgttgctttt

SEQ ID NO: 233 - DNA, 68 bp, Homo Sapiens
ccacactgcacactgcctggcccaaaacgtcttattgtggtaggatcagccctcattttgttgctttt

SEQ ID NO: 234 - DNA, 70 bp, Homo Sapiens
gatgcctctgttactgactgggtgataaatgagctgcgctaccaccttgaagagagccgagacaaaaacg

SEQ ID NO: 235 - DNA, 70 bp, Homo Sapiens
tcagtcattcattgtaagtagcacattgcaacaacaatcatgcttatgaccaatacagtcactaggttgt

SEQ ID NO: 236 - DNA, 65 bp, Homo Sapiens
tctgatgtttctatagtcactttgccagctcaaaagaaaacaataccctatgtagttgtggaagt

SEQ ID NO: 237 - DNA, 66 bp, Homo Sapiens
tgaaccacattataaagtaatctttggctgtaaggcattttcatctttccttcgggttggcaaaat

SEQ ID NO: 238 - DNA, 57 bp, Homo Sapiens
gtggtcagctcaatgctacacagagcacggacttttggattctttgcagtactttga

SEQ ID NO: 239 - DNA, 68 bp, Homo Sapiens
ggctgtcgtgttccagtccgtgttccccgtcgctggagtgcagcttaagtgcgaatcatttcttggaa

SEQ ID NO: 240 - DNA, 64 bp, Homo Sapiens
ctggtacggctcctacctggtctggaaagagctgggaggcttcacagagaaggctgtggttccc

SEQ ID NO: 241 - DNA, 64 bp, Homo Sapiens
cacgctctactcagccatggggtacggctcctacctggtctggaaagagctgggaggcttcaca

SEQ ID NO: 242 - DNA, 66 bp, Homo Sapiens
catcatcgagaaggacaaaatcaccaccaggacactgaaggcccgaatggactaaccctgttccca

SEQ ID NO: 243 - DNA, 64 bp, Homo Sapiens
ccacacatgccaaaatggaggtacagtgattctaatggatggaaagtgtttgtgtgcctgccca

SEQ ID NO: 244 - DNA, 70 bp, Homo Sapiens
ccatcctgactgtgccttaaagtcggcagcagccgctactcaaagaaaagccccaagtctttatacagaa

SEQ ID NO: 245 - DNA, 68 bp, Homo Sapiens
ttgggcactcatctattccttttctcttgtgtccctactcattgaaaccaaactctggaaaggaccca

SEQ ID NO: 246 - DNA, 70 bp, Homo Sapiens
aattgggcactcatctattccttttctcttgtgtccctactcattgaaaccaaactctggaaaggaccca

SEQ ID NO: 247 - DNA, 65 bp, Homo Sapiens
cccctatcctatccgcaaacagatgtgttcttaatttgcttttcccttgtgagtcctgcatcatt

SEQ ID NO: 248 - DNA, 70 bp, Homo Sapiens
cacagctgggtccctcttcaacactgccacatttccttattgatgcatcttttcccaccctgtcactcaa

SEQ ID NO: 249 - DNA, 70 bp, Homo Sapiens
cccctgagcacactaaccacctcatgcaggccccacctgccaatagtaataaagcaatgtcactttttta

SEQ ID NO: 250 - DNA, 70 bp, Homo Sapiens
gttgtcctacatgcaagtacatgtttttaatgttgtctgtcttctgtgctgttcctgtaagtttgctatt

SEQ ID NO: 251 - DNA, 66 bp, Homo Sapiens
tttccacattgctacccaatggacacactccaaaatggccagtgggcagggaatcctggagcactt

SEQ ID NO: 252 - DNA, 64 bp, Homo Sapiens
tgggatgaccagcatcagccccaatgtccagcctctttaacatcttctttcctatgccctctct

SEQ ID NO: 253 - DNA, 65 bp, Homo Sapiens
gaccagcatcagccccaatgtccagcctctttaacatcttctttcctatgccctctctgtggatc

SEQ ID NO: 254 - DNA, 70 bp, Homo Sapiens
ggaaaaagcggatttcagccaatcccactaatcctgatgaggctgacaaagttggggctgagaacacaat

SEQ ID NO: 255 - DNA, 65 bp, Homo Sapiens
ctctcatgacgcagcggacttatttgcagtggcccaaggagaaaagcaaacgtgggctcttttgg

SEQ ID NO: 256 - DNA, 56 bp, Homo Sapiens
gtccattgggcaggcagatgttcctaataaagcttctgttccgtgcttgtccctgt

SEQ ID NO: 257 - DNA, 57 bp, Homo Sapiens
aggggagcatgggaagtgacggtttaatgggcacagggtttatgtttaggatgttga

SEQ ID NO: 258 - DNA, 64 bp, Homo Sapiens
atcaagtaatccccttttccagaatgcattaacccactcccctgacctcacgctggggcaggtc

SEQ ID NO: 259 - DNA, 59 bp, Homo Sapiens
ttatccgtctcttcccgtccctgatgacaaagatcttgccttacagactttacaggctt

SEQ ID NO: 260 - DNA, 67 bp, Homo Sapiens
actaatagaaacttggcactcctgtgccctctgcctggacaagcacatagcaagctgaactgtccta

SEQ ID NO: 261 - DNA, 55 bp, Homo Sapiens
ctagcagccgcctacttggtgctaacccctcgatgtacatagcttttctcagctg

SEQ ID NO: 262 - DNA, 55 bp, Homo Sapiens
cccagaaactgagacaagaaagaaaacgcccactcctggatcttcacattgaact

SEQ ID NO: 263 - DNA, 70 bp, Homo Sapiens
catccctgtacctgcagtgcctctcccagaaactgagacaagaaagaaaacgcccactcctggatcttca

SEQ ID NO: 264 - DNA, 69 bp, Homo Sapiens
tctggattttaatcccaggcatccctcctaacttttctttgtgcagcggtctggttatcgtctatcccc

SEQ ID NO: 265 - DNA, 70 bp, Homo Sapiens
catcaaccaccatggatttttacaagatccatttgcctttctgggagcagaaacagccattgcggcccca

SEQ ID NO: 266 - DNA, 70 bp, Homo Sapiens
cccagccacatttcagccgaggtccaaggcaggaggccatttacttgagacagattctctcctttttcct

SEQ ID NO: 267 - DNA, 70 bp, Homo Sapiens
cagcacagacgtggggcatccccagaaagactccatcccaggaccaggttcccctccgtgctcttcgaga

SEQ ID NO: 268 - DNA, 68 bp, Homo Sapiens
aggtagacgtttttaaggagaaacacatggcccctgagttctgcagcctcgaggaaggcctgccctat

SEQ ID NO: 269 - DNA, 65 bp, Homo Sapiens
agatctgatgatcccaggaggctctggaggacaatctaggacctacattatctggactgtatgct

SEQ ID NO: 270 - DNA, 70 bp, Homo Sapiens
cctccccaaatgctttccatgagttgcagttttttcctagtctgttttccctccttggagacagggccct

SEQ ID NO: 271 - DNA, 64 bp, Homo Sapiens
gaggggaaactacagcacctggaaaatgaactcacccacgatatcatcaccaagttcctggaaa

SEQ ID NO: 272 - DNA, 70 bp, Homo Sapiens
ccacatccacacagccaatccaattaatcaaaccactgttattaacagataatagcaacttgggaaatgc

SEQ ID NO: 273 - DNA, 64 bp, Homo Sapiens
tgcctcaaccccatcatctacccatgctccagcaaggagttcaagcgcgctttcgtgcgcatcc

SEQ ID NO: 274 - DNA, 67 bp, Homo Sapiens
ggggagtgcagagggaaggtgggatataaggtaatcaacctggataacagtatgcaccttggatgtt

SEQ ID NO: 275 - DNA, 69 bp, Homo Sapiens
cctttggccaacagggtagatttctcttgggcaggcagagagtacagactgcagacattctcaaagcct

SEQ ID NO: 276 - DNA, 57 bp, Homo Sapiens
atatacccgtacaacgtgtcctcacggtgccttttttcacggaagttttcaatgatg

SEQ ID NO: 277 - DNA, 66 bp, Homo Sapiens
ctcctccacaccagcgctgatgcccaataaagatgcccactgaggaatcatgaagcttcctttctg

SEQ ID NO: 278 - DNA, 68 bp, Homo Sapiens
tatttgcactgtagcctcgccgtctgtgaattggaccatcctatttaactggcttcagcctccccacc

SEQ ID NO: 279 - DNA, 65 bp, Homo Sapiens
ctggaacccattccgcagtactccattcctagcagttatcacaagctcaaaagtctcatggccag

SEQ ID NO: 280 - DNA, 65 bp, Homo Sapiens
ccacacccagctcagcatttccacaccaagcagcaacagcaaatcacgaccactgatagatgtct

SEQ ID NO: 281 - DNA, 69 bp, Homo Sapiens
tcatgctccatccagccccacccaatggccttttgtgcttgtttcctataacttcagtattgtaaacta

SEQ ID NO: 282 - DNA, 67 bp, Homo Sapiens
agaccacgcatggcctgatctggtgttctccttcccaggtggattcatacgacgtgactgtggacga

SEQ ID NO: 283 - DNA, 70 bp, Homo Sapiens
ccaagtgcccaggggccaagaacaagttaacaagtaaaacaattagggacaccacctcctttccccagcc

SEQ ID NO: 284 - DNA, 65 bp, Homo Sapiens
aagtgcagacacaggcaggatacccaggataattacagttcaggtggcaaaagttcaaatgaggt

SEQ ID NO: 285 - DNA, 69 bp, Homo Sapiens
acaaccacccagcacatacattttataaaaggcagtagattcatgtgtttaacacaagtaagtgacatc

SEQ ID NO: 286 - DNA, 69 bp, Homo Sapiens
agagtccaaaggttcacataggtatgaattcaccatttgagagccataaaagcggccactcccaagttc

SEQ ID NO: 287 - DNA, 70 bp, Homo Sapiens
agtgcaattccatagcttgttcatcatcctggacagctaggatggactgggatgctggacagagtggcca

SEQ ID NO: 288 - DNA, 70 bp, Homo Sapiens
agctctggccctggctccaggctctgggtttaaagagctttaaaagcttattaaaaaggttccttcgaca

SEQ ID NO: 289 - DNA, 69 bp, Homo Sapiens
aagacccttccccggccctggggaacccaagatttccgagagtaagcggacctgtgcaaagacccacaa

SEQ ID NO: 290 - DNA, 69 bp, Homo Sapiens
gtttggaggtttcatcaggattttgggtttttcacatttcacgctaaggagtagtggccctgacttccg

SEQ ID NO: 291 - DNA, 67 bp, Homo Sapiens
agcagatcagtggccctgacttcccaacctcttcctgcattttagctgcctgtgaagtccagtgact

SEQ ID NO: 292 - DNA, 68 bp, Homo Sapiens
tcacagtccgcctctttaatcttggactggaggatatacagatcgagagggtgggagagtggccccag

SEQ ID NO: 293 - DNA, 60 bp, Homo Sapiens
tcccagtgacacttcagagagctggtagttagtagcatgttgagccaggcctgggtctgt

SEQ ID NO: 294 - DNA, 56 bp, Homo Sapiens
atgagaaggagggatatgaaggaagatggcttgcagagtaagtcgggtggcaattg

SEQ ID NO: 295 - DNA, 69 bp, Homo Sapiens
aggaagggtgagaatagaatcttgaggaacggatcagatgtctatattgctgaatgcaagaagtggggc

SEQ ID NO: 296 - DNA, 70 bp, Homo Sapiens
ctacaatatgtacatttctaagtcaaacacttgtgacttttgctttaattccatgaatgttcctgcctcc

SEQ ID NO: 297 - DNA, 68 bp, Homo Sapiens
tgtacatttctaagtcaaacacttgtgacttttgctttaattccatgaatgttcctgcctccttgata

SEQ ID NO: 298 - DNA, 66 bp, Homo Sapiens
agtttattgcttggatttctagtgagagctgttgaatttggtgatgtcaaatgtttctagggtttt

SEQ ID NO: 299 - DNA, 70 bp, Homo Sapiens
tcagcaagtacagatgcaagcgcattgcacactttttcctttctaaacttaaagacaagtcaaaaagcca

SEQ ID NO: 300 - DNA, 66 bp, Homo Sapiens
tcataaactgtgtgcagactcaataaaatcatgtacatttctgaaatgacctcaagatgtcctcct

SEQ ID NO: 301 - DNA, 70 bp, Homo Sapiens
cttcactacctattctgacaaccagcctggtgtgcttattcaggtttatgaaggcgagcgtgccatgaca

SEQ ID NO: 302 - DNA, 55 bp, Homo Sapiens
ttgcttttcatgtagaactcagcagttgacatccaaatctagccagagcccttca

SEQ ID NO: 303 - DNA, 70 bp, Homo Sapiens
acatgctgtgtaagctgtgtcctagttcttgaacagtttatgcagtgctgctttgccaaataaagttaaa

SEQ ID NO: 304 - DNA, 65 bp, Homo Sapiens
gtgtgtgtgttttcagcaaattccagatttgtttccttttggcctcctgcaaagtctccagaaga

SEQ ID NO: 305 - DNA, 70 bp, Homo Sapiens
aggaaatttgaatgggtccattttgcccttccatagcctaatccctgggcattgctttccactgaggttg

SEQ ID NO: 306 - DNA, 70 bp, Homo Sapiens
ctgactcgctgctctttttgttcccttctgtctgctcgaaccacttgcctcgggctaatccctccctctt

SEQ ID NO: 307 - DNA, 55 bp, Homo Sapiens
aggatgctggagtttatctgctgtatagccaggtcctgtttcaagacgtgacttt

SEQ ID NO: 308 - DNA, 68 bp, Homo Sapiens
gaggagagagaggcagaagaaccctggcctgagaaggtttggtccagcctggtttagcctggatgttg

SEQ ID NO: 309 - DNA, 69 bp, Homo Sapiens
gagaaggcccaaagtaatcaccaggatccttataaaagggaggcagaggaggatattaccacaaaagga

SEQ ID NO: 310 - DNA, 70 bp, Homo Sapiens
tgccttcttctgcgtttgtccctcctgccatgtgtaagttgtaaggattgcctttgtagttaatgtactc

SEQ ID NO: 311 - DNA, 70 bp, Homo Sapiens
aggtagctggggactatagggcgcccgctaccatgtccgggctaaattttttgtatttttaggtaggaga

SEQ ID NO: 312 - DNA, 67 bp, Homo Sapiens
aggtgtgacttccctggctggaggtgactgagatggccccacagggtcccatgacctctccaggcca

SEQ ID NO: 313 - DNA, 70 bp, Homo Sapiens
ggccaccttggctcccaaaatgctggcattacaggcgtgacacacacacccggcttacatgcatttttta

SEQ ID NO: 314 - DNA, 70 bp, Homo Sapiens
ttttatgtatttagagagatggtcttgctgtgtcacccaggctggagtgcactggtgctatcatggttca

SEQ ID NO: 315 - DNA, 66 bp, Homo Sapiens
tgaatttgtggctttaagtgatcctcccaccttggcctcccaaagtgctcgggttacaggcgtaca

SEQ ID NO: 316 - DNA, 55 bp, Homo Sapiens
ttcgcaccattctcctgcctcagcctcccaattagcttggcctacagtcatctgc

SEQ ID NO: 317 - DNA, 66 bp, Homo Sapiens
ttcaggcatgagccaccactcccggccctcaattaataacttgacttaagataatctagttcatat

SEQ ID NO: 318 - DNA, 66 bp, Homo Sapiens
aggtgggctgtgcaagtgggcgagatgagcaaccaggacttacaaacacggcatctgtatgccaga

SEQ ID NO: 319 - DNA, 68 bp, Homo Sapiens
aaaaattagccagggcatgggtgggtgtgtgcccatagtttcagctacttggggtggacgctgacgta

SEQ ID NO: 320 - DNA, 69 bp, Homo Sapiens
ccctggctaatttttatattagcggtctcgaactcctgaccttgtgatctgcccgcctctgcctcccaa

SEQ ID NO: 321 - DNA, 70 bp, Homo Sapiens
atctcctgcactcaagtgatgctaccacctcagcctcccaaatattataaccacagatgtgtaccaccat

SEQ ID NO: 322 - DNA, 70 bp, Homo Sapiens
tcatttttaaagtactgtagagcatccatccatccatccatccagcaatgtgactttttcatgtctttcc

SEQ ID NO: 323 - DNA, 69 bp, Homo Sapiens
agttgtgagccaccacgcccagccagcactaccttttctattgtgcatcctaatggtctgtagtataga

SEQ ID NO: 324 - DNA, 70 bp, Homo Sapiens
gactacaggctcacaccaccatgcccggccaacttttgtactcccagcagggacgaggttttgccatatt

SEQ ID NO: 325 - DNA, 68 bp, Homo Sapiens
agggctccggggattgggttgactaggcgcatcattcacatagctcatgaaaaagctggctcttccac

SEQ ID NO: 326 - DNA, 66 bp, Homo Sapiens
cgcaaaccaccacgcccgggtaatttttttacgtttagtagaggcggggtttcaccatgctatcca

SEQ ID NO: 327 - DNA, 70 bp, Homo Sapiens
tgagtaactggggattacaggcgtttcaccaccaagcccagatgattttttgtatttttttgagacggag

SEQ ID NO: 328 - DNA, 68 bp, Homo Sapiens
ggtgtgtgtgtgtgcgtgtgtagaggaggagaggaggaggaatatggaatgaatgaatggaatgtgaa

SEQ ID NO: 329 - DNA, 70 bp, Homo Sapiens
aacacataccttttaaatccacagctcattaagaatgggactaaaaaggccaggcgtggtggctcacgca

SEQ ID NO: 330 - DNA, 56 bp, Homo Sapiens
ttatttaaatgagtgtgtttgtcaccgttggggattggggaagactgtggctgctg

SEQ ID NO: 331 - DNA, 67 bp, Homo Sapiens
ggtgtctgcaaggttgattccttgtgagggctgtgcgagagtctgttccatgtttctctcctggctt

SEQ ID NO: 332 - DNA, 66 bp, Homo Sapiens
ccctgcctgtgtcccctggtctccttacctattccaacctaatgatacatgcatcgtttctggctg

SEQ ID NO: 333 - DNA, 70 bp, Homo Sapiens
ggtccttctttgaatccttcatccggaccctcatcatcacgtgtgtggccctggctgtggtcctgtcctc

SEQ ID NO: 334 - DNA, 68 bp, Homo Sapiens
tttgataggtggggaaactgagactgagggaggcaaagtggtctgcacagggccacaatcatgaggca

SEQ ID NO: 335 - DNA, 70 bp, Homo Sapiens
tgaagttaggaagttgatattcattgtattcatttgctgcttaggaatattgggatgaggaggggagggt

SEQ ID NO: 336 - DNA, 65 bp, Homo Sapiens
ggagagagaggggcttcccagaggaaagactggctggcagtggactcacaggattttatagtcag

SEQ ID NO: 337 - DNA, 67 bp, Homo Sapiens
gaagtggcaggcactccgctgaggcctacaggggaggatggtaggggcaggaattgaagtacagtct

SEQ ID NO: 338 - DNA, 67 bp, Homo Sapiens
atggaaactggtagtggtcctgaatgcctggctgcgctgttatttattggatacaaggggaaagggg

SEQ ID NO: 339 - DNA, 55 bp, Homo Sapiens
atgtaacctgaattaactcgtgtaatatttgtgtgtggagtgggatgtggggggt

SEQ ID NO: 340 - DNA, 68 bp, Homo Sapiens
tgaagggcacagagatgtccattgttgagttgtggcagagggaggtggggagttttgcatctgcaaca

SEQ ID NO: 341 - DNA, 66 bp, Homo Sapiens
tgaaggaacaggtttaggtgtttactaggcttttatggggtggaaaaagggatggacaggggagga

SEQ ID NO: 342 - DNA, 69 bp, Homo Sapiens
attaggacacagacacccagacagagggaccatacaaggacacagtaaggaagggcagccatctgcaag

SEQ ID NO: 343 - DNA, 69 bp, Homo Sapiens
tgaaggaggagcagaggcaggtcttacatggtggcaggcaagagagcctgtgcagggaaattcctgttt

SEQ ID NO: 344 - DNA, 70 bp, Homo Sapiens
actcgtactctgttactgatacactatcaagaccaaaaagacaaccatatgagaacacaacagcacacca

SEQ ID NO: 345 - DNA, 65 bp, Homo Sapiens
ttcacagtgctggaggtcagaagaaaatcaaggggtcagcagggctatactctctctagaggctc

SEQ ID NO: 346 - DNA, 70 bp, Homo Sapiens
agagtgtgaagacagaggcagaggagctgtttggggagaccatggagatgatggacaggatgaaagacat

SEQ ID NO: 347 - DNA, 69 bp, Homo Sapiens
aggggaatgtgcaagcaggaggggaagaaaaaaggaaggaaaaaggagagagggtaggcagtgaggcag

SEQ ID NO: 348 - DNA, 69 bp, Homo Sapiens
gattggaaaaaggataagtgtgagccactggagaagcagcacgagaaggagaggaaacaggaggagggg

SEQ ID NO: 349 - DNA, 68 bp, Homo Sapiens
atggtaaagagggaagccacaaagacactcgaaataaaggtttcagggaagggacagacagcaggggt

SEQ ID NO: 350 - DNA, 66 bp, Homo Sapiens
gaggaggtgagggagtactgtggaggggaggattgacttcatggccagaaaagcaaagtggaagga

SEQ ID NO: 351 - DNA, 65 bp, Homo Sapiens
tggcagatggtgtgggagcagggagtgaatgtgattgccatggtcactgcagaggagtgatcttt

SEQ ID NO: 352 - DNA, 70 bp, Homo Sapiens
agaggtagagggtagaaggatggttaccagaggttgggaagggtagtgaggggagaggggagcaggaggt

SEQ ID NO: 353 - DNA, 67 bp, Homo Sapiens
atgggatcagaaagggagggagtaaaacaaagaccagctacaggggagggaggaaggagaagggcat

SEQ ID NO: 354 - DNA, 66 bp, Homo Sapiens
ggctgctaagtgcagggagacaatgcagtgttggggacattgggagggcagggactatgggaggct

SEQ ID NO: 355 - DNA, 69 bp, Homo Sapiens
acaagggggttcaaagataatccaacgatggagacaggagacagggtgggagggaagcgggtcacgaat

SEQ ID NO: 356 - DNA, 68 bp, Homo Sapiens
ataaagaggggaaatttggagacacacacacacagggagagcgccgcatgaagatgaaggcagaaact

SEQ ID NO: 357 - DNA, 68 bp, Homo Sapiens
gaggtgtggagagagagacaggatcaggagcgatagagagcaggccctcagtgttgtactatttgtct

SEQ ID NO: 358 - DNA, 70 bp, Homo Sapiens
agaaattaaagaagacacacacacacacacacacacacacagtatcatgtttgcagattggaagactcaa

SEQ ID NO: 359 - DNA, 69 bp, Homo Sapiens
agacgcactcacccacacgtgcgcgtacacacacacacacgcgcgcactgtactgcaaataaatataca

SEQ ID NO: 360 - DNA, 68 bp, Homo Sapiens
ccccaactcagaccaaatgcaaacaaccgaactgcagcttctatggacaccctgagacaaacaacttc

SEQ ID NO: 361 - DNA, 66 bp, Homo Sapiens
aaatagaaaaaaacaatcaacagtgcccttcctgaggcaactaacaccctcttgactcctagaggt

SEQ ID NO: 362 - DNA, 70 bp, Homo Sapiens
gatgggatctagagcagctcaacaggaaatgcgtgggagaaagaagcactgtgagcccctttattgccca

SEQ ID NO: 363 - DNA, 70 bp, Homo Sapiens
acccagggccggcttcagctaataccaaggtttctgtgtgcataatagtttacagaggaacttaaggagt

SEQ ID NO: 364 - DNA, 70 bp, Homo Sapiens
agggcttttccttttttgcttgtctctcattctctctcctgccaccctgtgaagaggtgccttccaccat

SEQ ID NO: 365 - DNA, 55 bp, Homo Sapiens
agttaatgatactcctggaatacaagatcccccgcaatgaggctgatgggatgcc

SEQ ID NO: 366 - DNA, 65 bp, Homo Sapiens
ttctactgctttcttagctcacctgtgtcttccctgggtctttccagcaccttgtctaacatggt

SEQ ID NO: 367 - DNA, 69 bp, Homo Sapiens
cccgggcctggcccaggggcccctttacatgccctctcccttttataaaattttccattaaaaaccacc

SEQ ID NO: 368 - DNA, 65 bp, Homo Sapiens
acaacatttcatgtgtagaacggggctggtccactcctcccaaatgcaggtccactatttctgca

SEQ ID NO: 369 - DNA, 69 bp, Homo Sapiens
tgggaggtattggagggaaaaaacagcaggatggctggaacactgtactgaggaatgaatagaaaggct

SEQ ID NO: 370 - DNA, 65 bp, Homo Sapiens
agaaagactaggcagccgtgggggaccagaataagtaggaccacgagagagagtagagagaagca

SEQ ID NO: 371 - DNA, 67 bp, Homo Sapiens
acttggtttattattttggttatgaccccgtcatttgacttgtctttagttgtcttaacggggactg

SEQ ID NO: 372 - DNA, 67 bp, Homo Sapiens
tctagccgatggaaaatacctaattgtggatgatgattgcgcagggaggggtacgtggcacctcttc

SEQ ID NO: 373 - DNA, 70 bp, Homo Sapiens
acacaaaaaccacttgggagggaacattaaggggagcagcgggctaggccttttgggataatatcccgcc

SEQ ID NO: 374 - DNA, 70 bp, Homo Sapiens
caacaggactttgagttcacaagcaggggagcaggtttaaactatttttatggggggacaataaaaaact

SEQ ID NO: 375 - DNA, 69 bp, Homo Sapiens
aggagtgagcaaacaaatttacaagaaaaaaagaaacaaccccatcaaaaagtgggcgaaggacatgaa

SEQ ID NO: 376 - DNA, 70 bp, Homo Sapiens
agacgctgaaggagaagctaatgtggggagaagaaaaggtgagcttctaacagctgagggtgggagtttt

SEQ ID NO: 377 - DNA, 57 bp, Homo Sapiens
tcataaagctttacaaggcatttctggattaaaagtggtgggggagtccctttctcc

SEQ ID NO: 378 - DNA, 70 bp, Homo Sapiens
agcatttcatccgggcggagaccaatgagatcgtcagtgggtggctggagatgctcatggtctatcccct

SEQ ID NO: 379 - DNA, 68 bp, Homo Sapiens
tgccttttgtccttttgtggagagggcgatgaattaagagtagtggggtgggtggcgagtaagtaaaa

SEQ ID NO: 380 - DNA, 66 bp, Homo Sapiens
accttcctacaccactctctctgtctcactcctgctttcgccatgtgactgcctgctccccgttca

SEQ ID NO: 381 - DNA, 68 bp, Homo Sapiens
tggagaggggctgtgtctgggtgagggatggcggggtactgatttttttgggaggttatgagcaaaaa

SEQ ID NO: 382 - DNA, 66 bp, Homo Sapiens
gcaggtgggtgggtaaggagtaagggtaagggtggtaatacaaatgttatagagtcccctaacatt

SEQ ID NO: 383 - DNA, 70 bp, Homo Sapiens
tgtggagcgtgggaagtcaaagagcacggtgcatgcatatggggagggtcactcatggtgaaaggccaag

SEQ ID NO: 384 - DNA, 65 bp, Homo Sapiens
aaaggcagggtgatcatgcaggtgaaatgggtggattggtgtcaagtgcgtggaacagtactttt

SEQ ID NO: 385 - DNA, 55 bp, Homo Sapiens
atactgtattttgtaggcactggattttgtgagtttgaccccttattatgtctgg

SEQ ID NO: 386 - DNA, 65 bp, Homo Sapiens
agaagaagccgctggaaggaaggaatgggaccacaccacccaacgctctgggcacatgatgctca

SEQ ID NO: 387 - DNA, 58 bp, Homo Sapiens
tgattttttatgagaagctgctaggatcggatattttcacaggggagccttctctgct

SEQ ID NO: 388 - DNA, 65 bp, Homo Sapiens
gactttgttgtttggattcctcacagccttggctcctgagaaaggtgaggagggcagtccaagag

SEQ ID NO: 389 - DNA, 67 bp, Homo Sapiens
aggaacctagcagtctcccaggtggtccataaggtcgtgtctgatgtatttgaggagggcacagaag

SEQ ID NO: 390 - DNA, 65 bp, Homo Sapiens
atgagcgtgcactccccgtgttcatatgtcagggcacatgtgatgtggtgcgtgtgaatctgtgg

SEQ ID NO: 391 - DNA, 67 bp, Homo Sapiens
tcacacttacagcacatatcaaacagactagccatattacaagtgctcagcagccacaacgttacta

SEQ ID NO: 392 - DNA, 55 bp, Homo Sapiens
actccccgtgttcatatgtcagggcacatgtgatgtggtgcgtgtgaatctgtgg

SEQ ID NO: 393 - DNA, 64 bp, Homo Sapiens
ttgccccagtaactcactttaaaacctctctttccagtgttccctctctccctccagggccact

SEQ ID NO: 394 - DNA, 66 bp, Homo Sapiens
aaattctgctgtgtggtgatgaacagaaaggtagtcccatccagctcggtgtcttctgaaacgtca

SEQ ID NO: 395 - DNA, 57 bp, Homo Sapiens
aagagacgtttaaaagccatcaaacacaatgcctcatcttccctggaggacccactt

SEQ ID NO: 396 - DNA, 68 bp, Homo Sapiens
gggcagtgggactcttccaggggttgtttaatcccacttctgacacaatatgggatgggactagggga

SEQ ID NO: 397 - DNA, 66 bp, Homo Sapiens
agcacgagcagcaggtagcaggcatctgtgttagaatcctcccacccatttgctaagtggctgtct

SEQ ID NO: 398 - DNA, 70 bp, Homo Sapiens
ggcacatccaggggtgcagaggttgttgtcttcatgcaacatagtgaccccatctgctggtgctcccaat

SEQ ID NO: 399 - DNA, 66 bp, Homo Sapiens
ccaggcgggttggtatctttcagatgaaggcattgaagcttgcacaagctctccagacaaagtcaa

SEQ ID NO: 400 - DNA, 66 bp, Homo Sapiens
ggaagcacagagtaggtgctgttattgctttgaatggaactcagcttgcttgtgtgggtttggagt

SEQ ID NO: 401 - DNA, 64 bp, Homo Sapiens
accaccaaagatcaacagaagaacccagtggaagctgagcctgctagactgagtgactgcagtt

SEQ ID NO: 402 - DNA, 70 bp, Homo Sapiens
ggggatgtggactggagagttctggagaagtggtatttatgttgagagaaacaagagcaaagtgattaaa

SEQ ID NO: 403 - DNA, 68 bp, Homo Sapiens
ttctcgtagcgtgggttcctaccaaagagcatggcttggttcatgtactcggagacaggggcgttgga

SEQ ID NO: 404 - DNA, 67 bp, Homo Sapiens
acatttacttctttgggtacccattaaaatagttgcatttgcagaggatgtttaggggcagcgaaac

SEQ ID NO: 405 - DNA, 65 bp, Homo Sapiens
tcatttacaggtcctcaggagatgaccagtccctttggcatgcaggtccagtgcctgggtcttct

SEQ ID NO: 406 - DNA, 69 bp, Homo Sapiens
tagccttaggaaagctgaggggatgggattattaggaaaagaaagtgtgtgccagtaaccctgaggcct

SEQ ID NO: 407 - DNA, 67 bp, Homo Sapiens
actttggtgagtttcaggaactgtgggtcaactgaatgaatcagcttggtaataggacttcaggcat

SEQ ID NO: 408 - DNA, 70 bp, Homo Sapiens
caacaacattatccatgggtcagtggctgatactattattcctatttttcaggaggtggctggtctctcc

SEQ ID NO: 409 - DNA, 66 bp, Homo Sapiens
aggaggggctcttgtccacatctgtcaaagtatgaataacaattacaccctaactcgaaagtggct

SEQ ID NO: 410 - DNA, 70 bp, Homo Sapiens
accatggccaaaccgcgggcttgaggaggggagccccgtttctcacatttgtcccctttccttttacatt

SEQ ID NO: 411 - DNA, 68 bp, Homo Sapiens
tcaattcaccatgacatttaatagccttcaaaacaggatttttgtaatggctgcgtgttagtccatca

SEQ ID NO: 412 - DNA, 69 bp, Homo Sapiens
ttatggcaacgaccagagagtgtgccctttacacctacggacactgcactcgatgtgctcatccacaca

SEQ ID NO: 413 - DNA, 66 bp, Homo Sapiens
ctcatcttggcccagtagtttgccgctgttgctaagtttgggaacattcgtggtgacagctcccca

SEQ ID NO: 414 - DNA, 70 bp, Homo Sapiens
ttgattctgtgcccataagttccacagtcagactggtcactccattttctgagcccaaaaccgatcgcca

SEQ ID NO: 415 - DNA, 70 bp, Homo Sapiens
aaagatgggcctgccatgtgccaggctctgctctagatgctggggacacagcagggattcatactgacaa

SEQ ID NO: 416 - DNA, 67 bp, Homo Sapiens
atttcaggcctccactggcagttgctttcactgtacaaatgtaaagtgtggaaaagggtaaggacct

SEQ ID NO: 417 - DNA, 64 bp, Homo Sapiens
cctcaaagagagttttgagaactaccgcaggaagcgcgccctcaggaagatgcagaaaggatgg

SEQ ID NO: 418 - DNA, 67 bp, Homo Sapiens
tgggaaaaccaccggctgcttccgctggaataaacagtgttgaaagtaaccgcagacctgcccttgt

SEQ ID NO: 419 - DNA, 68 bp, Homo Sapiens
accagggattaccatcccttttgaacatccttccaaccatgcaggcctcttctgttgttgctgtgcat

SEQ ID NO: 420 - DNA, 67 bp, Homo Sapiens
ttgtcttcttctgtgctggagatgttttagagttaacaatgatatatggataatgccggtgagaata

SEQ ID NO: 421 - DNA, 65 bp, Homo Sapiens
aaaaaaagaaggtaagcaatgatgttttggtacttaaaaagttacgctttgcagagctgggaaat

SEQ ID NO: 422 - DNA, 65 bp, Homo Sapiens
acccctccgggaaccagctcatcgtgggagccaggaactacctcttcagactcagccttgccaat

SEQ ID NO: 423 - DNA, 67 bp, Homo Sapiens
ctcagccacaatctactgctccatacggaaacctcaggtggtgccaccaccacaacagaatgatctt

SEQ ID NO: 424 - DNA, 65 bp, Homo Sapiens
ctgccaaggctgctcccacactccctacactcaaatcccgggtggcagccataatccccaaagat

SEQ ID NO: 425 - DNA, 70 bp, Homo Sapiens
cacctggggtgtgcaacgtgtcagcctgctcattcccttctccagctcaggacagagaaacacaattcat

SEQ ID NO: 426 - DNA, 65 bp, Homo Sapiens
aaatgtctaacaatgtgagtgatccgaaggtccaccagccaagattgcccgcctggagcaaaacg

SEQ ID NO: 427 - DNA, 67 bp, Homo Sapiens
tacctgaggaggaattagcagaggatgttgaagagattgatcacgctgaaagggagttgcggcgtgg

SEQ ID NO: 428 - DNA, 70 bp, Homo Sapiens
ccaccccatgtccatttacatgtccttcaatgcccacctcaaaaggtacctcttctgtaaagctttccct

SEQ ID NO: 429 - DNA, 62 bp, Homo Sapiens
ttactgccactaaactgcctgtatttctgtatgtccttctatccaaacagacgttcactgcc

SEQ ID NO: 430 - DNA, 70 bp, Homo Sapiens
aaaaagatgaatgtcctgaaaagggtgttgggagggtggttcaacaaagaaacaaagatgttatggtgtt

SEQ ID NO: 431 - DNA, 55 bp, Homo Sapiens
ggacttctgccaattttgaatgatatagctgcaccaacaatatcccgcctcctct

SEQ ID NO: 432 - DNA, 69 bp, Homo Sapiens
gatgatttgcactttggttccctgtgttttgatttctcattaaagttcctttccttccccgttgtgaat

SEQ ID NO: 433 - DNA, 65 bp, Homo Sapiens
tgggctggagatacagagttggggacgacccctggaaaagtgaaccaaggtcgttttgcacggct

SEQ ID NO: 434 - DNA, 70 bp, Homo Sapiens
ggtggctggagaggaggcaagaaatgagttgggtttggggttaaaaagtgacttaagctctgagtagagg

SEQ ID NO: 435 - DNA, 66 bp, Homo Sapiens
ttatcagcaactccaggccatgacccagtgtcccattctccttcacggtttgctggtgtgtagcct

SEQ ID NO: 436 - DNA, 68 bp, Homo Sapiens
ttctggggtgctgtccaagcatggcttgctggggactcttgcactctaagtggtgctctaaatgtgct

SEQ ID NO: 437 - DNA, 70 bp, Homo Sapiens
ggggaagggttccccttccttctagagcagaaaagggagagaagtgttgtattcctgtttggtaacctca

SEQ ID NO: 438 - DNA, 70 bp, Homo Sapiens
cagtatcttagcccaagttctggatcagtcggcccgggccaggtgtaagtaacttagcacttgtaaagcc

SEQ ID NO: 439 - DNA, 64 bp, Homo Sapiens
aaggggtgaaaccatctcgcacacagcaatgaaggtagtcatccatagacttgaaatgagacca

SEQ ID NO: 440 - DNA, 70 bp, Homo Sapiens
ccaagtctgaagagaccctgggatgagggtccaccaaaatatacttaaatctgttctgaaaaagggagat

SEQ ID NO: 441 - DNA, 70 bp, Homo Sapiens
gataggaagatagggatttcctcagtcacagatgatattttgaaggaaagctgcaataaagccacaatga

SEQ ID NO: 442 - DNA, 70 bp, Homo Sapiens
agctctacagggtggtcttcgacatcaccttcttcttcttcgtcatcgtcatcctgttggccatcatcca

SEQ ID NO: 443 - DNA, 70 bp, Homo Sapiens
cacaagtcccggctcctgcctttggcacttactccatgctgctgctgctgttttacttttacttattcaa

SEQ ID NO: 444 - DNA, 70 bp, Homo Sapiens
atatttgggaacagatcaaagtaaatggaaggcttatgatgctacccaccttgtgaaatcctatccagga

SEQ ID NO: 445 - DNA, 70 bp, Homo Sapiens
tggatacctttaatgaagagcttgcaggttcaaatcttccgcctgtggtaaatggatgggtgttggttcg

SEQ ID NO: 446 - DNA, 55 bp, Homo Sapiens
caggtagaggaagatatggtgatggtgttgatggatggctctgtgaagctggtga

SEQ ID NO: 447 - DNA, 70 bp, Homo Sapiens
acataaataatccaagcttgcaccacattcagcattaatcactaggtcactttgcttgctgaccccttcc

SEQ ID NO: 448 - DNA, 64 bp, Homo Sapiens
cgcagctctgacctggtgctgtcgccctggcatcttaataaaacctgcttatacttccctggca

SEQ ID NO: 449 - DNA, 65 bp, Homo Sapiens
gggccttgccagggcgcgacaacatgacgttcaaggtcttcctgtggcttctgtaaagaaatgtt

SEQ ID NO: 450 - DNA, 65 bp, Homo Sapiens
tttataaattgtctaaggccaaacatttaacaggcatacaaggggaccttacaaagagggaggtg

SEQ ID NO: 451 - DNA, 55 bp, Homo Sapiens
ccacaaggaatttcctgtgaccatgagacctacagaagccagtgagaggtgccac

SEQ ID NO: 452 - DNA, 58 bp, Homo Sapiens
agccccactgcccttctcatttggaaactaaaacttcatcttccccaagtgcggggag

SEQ ID NO: 453 - DNA, 70 bp, Homo Sapiens
tcctctttgcctgatcaaatacgttctgtgacccaccccatgctaactccctattagagccagtgtcctt

SEQ ID NO: 454 - DNA, 70 bp, Homo Sapiens
tttcacatgacataaaattcgccattgtaaagaacacaattcatcacagggttgtacatccaccacctct

SEQ ID NO: 455 - DNA, 70 bp, Homo Sapiens
tcatctaagttgtaccgagccaggtcattccgaagcacacgcattagaatgacagccacaaaacccacca

SEQ ID NO: 456 - DNA, 55 bp, Homo Sapiens
tcttcctcctcatcaccatcgccctagggaccaagactgaatcctcctcacaaac

SEQ ID NO: 457 - DNA, 55 bp, Homo Sapiens
gatcaggacctccaagccactgagcaatgtataaccccaaagggaattcaaaaaa

SEQ ID NO: 458 - DNA, 67 bp, Homo Sapiens
ctcccattccagtcccgatgcacatggacaccactccgtatgccctcttcccacggcgcttcagctc

SEQ ID NO: 459 - DNA, 55 bp, Homo Sapiens
tgctccacccccagattgcaatgctaccaataaagccgcctggtgtttacaacta

SEQ ID NO: 460 - DNA, 70 bp, Homo Sapiens
tcaatgtgtcattgtgtcagatccctctccccttcaggttttcacggtgctcctcatgcctccgccctct

SEQ ID NO: 461 - DNA, 69 bp, Homo Sapiens
cacccaatagacaaggaacccagagatgacatacagtgtgctcacttccacccactgccactgagaaca

SEQ ID NO: 462 - DNA, 67 bp, Homo Sapiens
tttcaccttcttcctgaagtgtcccacctagcttacccagaaggccactgtccacactaccctcact

SEQ ID NO: 463 - DNA, 67 bp, Homo Sapiens
tctctacaaaaaccaactggaatacaggatagctctgtcctatagcaacctaaaccagcacctccct

SEQ ID NO: 464 - DNA, 55 bp, Homo Sapiens
gagacttctcatcctggccctccttggcatctgctctctcactgcatacattgtg

SEQ ID NO: 465 - DNA, 69 bp, Homo Sapiens
tcgacattctcctggccttcctcttcggcattctccaagtcatctagcccttcatcctcctccacatca

SEQ ID NO: 466 - DNA, 65 bp, Homo Sapiens
atggggttattgaacccttaaaacggggctagggggaattgggatgggctggaaatgtaaaaata

SEQ ID NO: 467 - DNA, 55 bp, Homo Sapiens
tggtgctggtctttatcaaaaaggtggccaagaagccaaccaataaggcccccca

SEQ ID NO: 468 - DNA, 70 bp, Homo Sapiens
ctctctctacccactggccaccccaaagatccctgtgcagctccgattttattcgcatccttgtgatctt

SEQ ID NO: 469 - DNA, 55 bp, Homo Sapiens
caggagagtgtcacagagcaggacagcaaggacagcacctacagcctcagcagca

SEQ ID NO: 470 - DNA, 70 bp, Homo Sapiens
ccctgggacccaagtggatatcaaacgaactgtggctgcaccatctgtcttcatcttcccgccatctgat

SEQ ID NO: 471 - DNA, 65 bp, Homo Sapiens
aggccctttcataaaaaccaaactgtagcaagatgcaaatgcatggcaaatcctgtcggtctcca

SEQ ID NO: 472 - DNA, 64 bp, Homo Sapiens
atgcaacctgaatcatggccacctaaaattccgctatcatgccaggtgcttgccccacctggga

SEQ ID NO: 473 - DNA, 65 bp, Homo Sapiens
ctgcactcttaagcttccgccgtctcaacccctcacaggagcttactggcaaacatgaaaaatcg

SEQ ID NO: 474 - DNA, 55 bp, Homo Sapiens
aaacgctactaactacaggaagcaaactaagcccccgctgtaatgaaacaccttc

SEQ ID NO: 475 - DNA, 70 bp, Homo Sapiens
caccttctctggagccaagtccagatttacactgggagaggtgccagcaactgaataaatacctctccca

SEQ ID NO: 476 - DNA, 66 bp, Homo Sapiens
cggtaactgcacttgggcagggaatatagccttcctgggcacaactagctgacaatgacaggttga

SEQ ID NO: 477 - DNA, 55 bp, Homo Sapiens
gttgagccttggaaaggtagacaagcgttgagccactaagcagaggaccttgggt

SEQ ID NO: 478 - DNA, 70 bp, Homo Sapiens
cctcgagttcagccaaaacctccccatggggcagcagaaaactcattgtccccttcctctaattaaaaaa

SEQ ID NO: 479 - DNA, 65 bp, Homo Sapiens
cagaagcccattattcagagcgagtatggagcagaaacgattgcagggtttcaccaggatccacc

SEQ ID NO: 480 - DNA, 69 bp, Homo Sapiens
agatgtgttcctccattttcttatccacaaagtactcctcacttttcaatttgtcatgttactaaaccc

SEQ ID NO: 481 - DNA, 70 bp, Homo Sapiens
gagataggaagccagactacactgcttacgttgccatgatccctcagtgcataaaggaggaagacacccc

SEQ ID NO: 482 - DNA, 64 bp, Homo Sapiens
gcccaaggccaaggaccccttcgctcacctgcccaagagtacctttgtgttggatgaatttaag

SEQ ID NO: 483 - DNA, 70 bp, Homo Sapiens
gagagttaggaaggaaaaaccaatagctttaaaacctgtgtgccattttaagagttacttaatgtttggt

SEQ ID NO: 484 - DNA, 68 bp, Homo Sapiens
gagaagagagtgaatactggagaagaaaggaggaaaatatctgaggaagcagcaagaaagagaaggct

SEQ ID NO: 485 - DNA, 66 bp, Homo Sapiens
ccacacatcttctaaagaaatggacaaggttctcactctccccagccaaggacccagatcaggtct

SEQ ID NO: 486 - DNA, 66 bp, Homo Sapiens
ccatttgaggcaaaggcagcttcccgagctgatgctaaagaagacagactttcccttcctcccagc

SEQ ID NO: 487 - DNA, 67 bp, Homo Sapiens
ccccagctcacactcagcgtgggaccccgaatgttaagcaatgataataaagtataacacggaaaaa

SEQ ID NO: 488 - DNA, 70 bp, Homo Sapiens
tatgcttttgaaactgccacgtccccaatccaccaccatgggtggcctccttcaaagccctcagctctcc

SEQ ID NO: 489 - DNA, 70 bp, Homo Sapiens
ccccacagccagcctgacagtccctccagttgctctcagagacttgcttcacatggatacacagagcaat

SEQ ID NO: 490 - DNA, 56 bp, Homo Sapiens
taaaacacacttcctgctgcgaaagacccacatgctacaagacgggcaaaataaag

SEQ ID NO: 491 - DNA, 57 bp, Homo Sapiens
agactcaaatgctcagaccagctcttccgaaaaccaggccttatctccaagaccaga

SEQ ID NO: 492 - DNA, 55 bp, Homo Sapiens
catgcctggctgggaatgtcagtgcatgtgacgcccctattctctcctcttccag

SEQ ID NO: 493 - DNA, 65 bp, Homo Sapiens
ccccatcccaaagtattccatctacttttctatcgccgtccccttttgcagccctctctggggat

SEQ ID NO: 494 - DNA, 69 bp, Homo Sapiens
cagctcattcccagccagggcaactgcctgacgttgcacgatttcagcttcattcctctgatagaacaa

SEQ ID NO: 495 - DNA, 70 bp, Homo Sapiens
tcttcacaataaggtcccaaccttaactaatcctgttgccaaaagaaactgagctcattcccagcttccg

SEQ ID NO: 496 - DNA, 69 bp, Homo Sapiens
tacccctaaacaacagcataactcaaataataatgacacacacgtcccgcccatatacacaataccact

SEQ ID NO: 497 - DNA, 70 bp, Homo Sapiens
tacaaactggactctcaggacaaaacgacaccagccaaaccagcagcccctcagcatccagcaacataag

SEQ ID NO: 498 - DNA, 65 bp, Homo Sapiens
actctgttgcccaatgaccagctctaccagcccctcaaggatcgagaagatgaccagtacagcca

SEQ ID NO: 499 - DNA, 68 bp, Homo Sapiens
gtatcctagaatctttgtgctctcgctgcagttccctttgggttccatgttttccttgttccctccca

SEQ ID NO: 500 - DNA, 70 bp, Homo Sapiens
caaagagcccttcgagcaccacgcaagaagatccatcgcagagtcctaaagaagaacccactgaaaaact

SEQ ID NO: 501 - DNA, 70 bp, Homo Sapiens
atagactgggaagatgcacaaccaaggggttacaggcatcgcccatgctcctcacctgtattttgtaatc

SEQ ID NO: 502 - DNA, 67 bp, Homo Sapiens
ccactgaatgggtaggagcaaccactgactggtcttaagctgttcttgcataggctcttaagcagca

SEQ ID NO: 503 - DNA, 68 bp, Homo Sapiens
tcatctcagccctgcctttctctggagcattctgaaaacagatattctggcccagggaatccagccat

SEQ ID NO: 504 - DNA, 68 bp, Homo Sapiens
cgaaactacagtgcccgcacagacttctactgcctggtgtccaaggatgatatgtagctcccacccca

SEQ ID NO: 505 - DNA, 68 bp, Homo Sapiens
tctgcccgcgtgtcgttcgtcgctttcaactccagtctgtcaatgcccctgtgtaggtggggtcccca

SEQ ID NO: 506 - DNA, 65 bp, Homo Sapiens
tgccgcaccattgaactgtaccatgtgctacaggggccagaagatgaacagactgaccttgatga

SEQ ID NO: 507 - DNA, 67 bp, Homo Sapiens
ggggagtctccatgcctttgaagggcccgactgccttaaatcttcctcaaccaaatacgtattttat

SEQ ID NO: 508 - DNA, 66 bp, Homo Sapiens
cgactgaagtttgccaggaagcccctgtgcatgttggacatctttgtgctgattgcctctgtgcca

SEQ ID NO: 509 - DNA, 70 bp, Homo Sapiens
tcggctcctgttttctatttgtcatgaaacagtgtatttggggagatgctgtgggaggatgtaaatatct

SEQ ID NO: 510 - DNA, 65 bp, Homo Sapiens
ggacgcccaggaccccatggcagtgggaaaaatgtaggagactgtttggaaattgattttgaacc

SEQ ID NO: 511 - DNA, 59 bp, Homo Sapiens
agccttggtactgcagagtctgggttgtagagaactctttgtaagcaataaagtttggg

SEQ ID NO: 512 - DNA, 68 bp, Homo Sapiens
ctttcacaaacactcagccccgaagatgttggagatctatgagcctgtgtcggacagccagatggtca

SEQ ID NO: 513 - DNA, 70 bp, Homo Sapiens
cccctcccctactgttctcatggtgttggcctcgtgtttgctatgccaactagtagaaccttctttccta

SEQ ID NO: 514 - DNA, 70 bp, Homo Sapiens
taccagaggacaaagcctttggctcttctaatcagagtgcaagctgggagcacaggcactgcaggagaga

SEQ ID NO: 515 - DNA, 64 bp, Homo Sapiens
caacccaagtggccttatccgtgcagtggtggcagttcagaaataaagggcccatttgtgggat

SEQ ID NO: 516 - DNA, 66 bp, Homo Sapiens
actgccaatgtgtgtatgggacttgctggaaaacggggatgcttgcccctctccaggactattgag

SEQ ID NO: 517 - DNA, 59 bp, Homo Sapiens
ctccccctcagatgatctctccacggtagcacttgaccttttcgacgcttaacctttcc

SEQ ID NO: 518 - DNA, 66 bp, Homo Sapiens
cctccacaaatgggaaaaccagactacttgggagcaccgcctgtgaaataccaacctgaagacacg

SEQ ID NO: 519 - DNA, 55 bp, Homo Sapiens
ctatacagaagtgtttccatgccacctaagggagactcaggacagccattatttc

SEQ ID NO: 520 - DNA, 69 bp, Homo Sapiens
acctacaatacatgcctggggtaagccacccggctacctaattcctcagtaacctccatctaaaatctc

SEQ ID NO: 521 - DNA, 69 bp, Homo Sapiens
gaaaacacaccttggaattcatgatgagacagtccaactctaccgctgccaccaatgaggttcctgagg

SEQ ID NO: 522 - DNA, 70 bp, Homo Sapiens
tggtccccagctcttgccaaagggcctggagtagaaggacaacagggcagcaacttggagggagttctct

SEQ ID NO: 523 - DNA, 58 bp, Homo Sapiens
tcctgtattcacccgtccagaccttgttcacactctcacatgtttagagttgcaatcg

SEQ ID NO: 524 - DNA, 55 bp, Homo Sapiens
gcactttaagccataaatcactctgttcattcctccaggtattcgttatctaata

SEQ ID NO: 525 - DNA, 69 bp, Homo Sapiens
agggtagaaccaaatgatagggtaggagcatgtgttctttagggccttgtaaggctgtttccttttgca

SEQ ID NO: 526 - DNA, 70 bp, Homo Sapiens
cttggatgagcctcttctcaagcactgggagtttgatgctccaagccctctcccagagactacagagaac

SEQ ID NO: 527 - DNA, 69 bp, Homo Sapiens
gccttttatcctctgttctcctttgaagaacccctcactgtcaccttcccgagaataccctaagaccaa

SEQ ID NO: 528 - DNA, 69 bp, Homo Sapiens
tgctcccctttgcctaaaccgtatggcctcccgtgcatctgtactcaccctgtacgacaaacacattac

SEQ ID NO: 529 - DNA, 69 bp, Homo Sapiens
cccctttgcctaaaccctatggcctcctgtgcatctgtactcaccctgtaccacaaacacattacatta

SEQ ID NO: 530 - DNA, 70 bp, Homo Sapiens
agagcagcagaggcggtcttcaacatcctgccagccccacacagctacagctttcttgctcccttcagcc

SEQ ID NO: 531 - DNA, 67 bp, Homo Sapiens
tgcctcatcttcaacttttgtgcttccctttacctaaactgtcctgcctcccatgcatctgtacccc

SEQ ID NO: 532 - DNA, 69 bp, Homo Sapiens
cagtgcctcatcttcaactttagtgctcccctttacctaaccctacggcctcccatgcatctgtactcc